(19) United States
(12) Patent Application Publication
(10) Pub. No.: US 2022/0323519 A1
(43) Pub. Date: Oct. 13, 2022

(54) COMPOSITIONS OF EXOSOMES AND AAV

(71) Applicant: Codiak BioSciences, Inc., Cambridge, MA (US)

(72) Inventors: Aaron NOYES, Melrose, MA (US); Russell E. MCCONNELL, Cambridge, MA (US); Ke XU, Belmont, MA (US); Jonathan FINN, Cambridge, MA (US)

(73) Assignee: Codiak BioSciences, Inc., Cambridge, MA (US)

(21) Appl. No.: 17/594,440

(22) PCT Filed: Apr. 17, 2020
(86) PCT No.: PCT/US2020/028849

§ 371 Date: Oct. 15, 2021

Related U.S. Application Data
(60) Provisional application No. 62/984,173, filed on Mar. 2, 2020.
(60) Provisional application No. 62/984,161, filed on Mar. 2, 2020.
(60) Provisional application No. 62/835,425, filed on Apr. 17, 2019.
(60) Provisional application No. 62/835,432, filed on Apr. 17, 2019.

(51) Int. Cl. A61K 35/761; A61K 47/42; A61K 47/64; A61K 9/00; A61P 27/02; A61P 3/10; C07K 14/005; C12N 15/86

(52) U.S. Cl. CPC A61K 35/761; A61K 47/42; A61K 47/64; A61K 9/0051; A61P 27/02; A61P 3/10; C07K 14/005; C12N 15/86

(57) ABSTRACT

The present disclosure relates to extracellular vesicles, e.g., exosomes, comprising an AAV and a scaffold protein. In some aspects, the AAV is in the lumen of the extracellular vesicle. In some aspects, the AAV is associated with the luminal surface of the extracellular vesicle. In some aspects, the AAV is associated with the exterior surface of the extracellular vesicle. Also provided herein are methods for producing the exosomes and methods for using the exosomes to treat and/or prevent diseases or disorders.

### CROSS-REFERENCE TO RELATED APPLICATIONS

This PCT application claims the priority benefit of U.S. Provisional Application Nos. 62/835,425 filed on Apr. 17, 2019; 62/835,432 filed on Apr. 17, 2019; 62/984,161 filed on Mar. 2, 2020; and 62/984,173 filed on Mar. 2, 2020, each of which is incorporated herein by reference in its entirety.

### REFERENCE TO SEQUENCE LISTING SUBMITTED ELECTRONICALLY VIA EFS-WEB

The content of the electronically submitted sequence listing (Name: 4000_033_PC02Seqlisting_ST25.txt, Size: 231,249 bytes; and Date of Creation: Apr. 17, 2020) submitted in this application is incorporated herein by reference in its entirety.

### FIELD OF DISCLOSURE

The present disclosure relates to extracellular vesicles (EVs), e.g., exosomes, comprising an adeno-associated virus (AAV). In certain aspects of the disclosure, the extracellular vesicle further comprises a scaffold protein.

### BACKGROUND

AAV has emerged as a useful vector for gene therapy applications. However, despite its many advantages, AAV stimulates a humoral, antibody-mediated immune response. As many as half of all potential patients that could benefit from AAV-mediated gene therapy cannot receive treatment because they possess pre-existing neutralizing antibodies developed after exposure to AAV serotypes in the wild. Additionally, after the initial dose of an AAV therapy, patients develop antibodies against the treatment and cannot be re-dosed, precluding dose-escalation regimes to find a dose that can achieve the desired therapeutic effect. The inability to re-dose is a major problem if the administered transgene loses expression over the life of the patient, which frequently occurs through cell division, transgene inactivation, or loss of transduced cells.

Exosomes are small extracellular vesicles that are naturally produced by eukaryotic cells. Exosomes comprise a membrane that encloses an internal space (i.e., lumen). As drug delivery vehicles, EVs, e.g., exosomes, offer many advantages over traditional drug delivery methods as a new treatment modality in many therapeutic areas. In particular, exosomes have intrinsically low immunogenicity. AAV associated with exosomes has improved delivery characteristics than free AAV, including transduction efficiency. Maguire et al., Molecular Therapy 20(5):960-71 (2012); Gyorgy et al., Biomaterials 35(26):7598-7609 (2014). However, AAV packaging in exosomes using current methods is highly inefficient, limiting the potential usefulness of exosomes in the delivery of AAV. Thus, there remains a need in the art to develop techniques for more efficiently associating AAV with exosomes.

### SUMMARY OF DISCLOSURE

Certain aspects of the present disclosure are directed to an extracellular vesicle (EV), e.g., an exosome, comprising an AAV and a scaffold protein. In some aspects, the AAV is in the lumen of the EV. In some aspects, the AAV is associated with the membrane of the EV, e.g., exosome. In some aspects, the AAV is associated with the luminal surface of the EV, e.g., exosome. In some aspects, the AAV is associated with the exterior surface of the EV, e.g., exosome. In some aspects, the AAV associated with the exosome has altered properties as compared to the free AAV alone.

Certain aspects of the present disclosure are directed to an extracellular vesicle (EV), e.g., an exosome, comprising an AAV and a scaffold protein, wherein the AAV is in the lumen of the EV, and wherein the AAV in the exosome has altered properties as compared to the free AAV alone.

In some embodiments, the altered property comprises a better therapeutic effect than AAV alone. In some embodiments, the better therapeutic effect comprises one or more of higher activity, increased transduction, increased transduction efficiency, greater potency, faster transduction kinetics, and evasion of immune responses.

In some embodiments, the altered properties of the AAV allow the AAV to be administered to a subject through two or more doses, wherein the activity of the AAV is retained in subsequent doses.

Certain aspects of the present disclosure are directed to an EV, e.g., an exosome, comprising an AAV, wherein the EV comprises a scaffold protein and at least 1 AAV, wherein the at least 1 AAV are in the lumen of the EV, e.g., an exosome.

In some aspects, the EV, e.g., an exosome, comprises at least 1 AAV, at least 2 AAVs, at least 3 AAVs, at least 4 AAVs, or at least 5 AAVs. In some embodiments, the EV, e.g., an exosome, comprises at least 6 AAVs, at least 7 AAVs, at least 8 AAVs, at least 9 AAVs, at least 10 AAVs, at least 11 AAVs, at least 12 AAVs, at least 13 AAVs, at least 14 AAVs, at least 15 AAVs, at least 16 AAVs, at least 17 AAVs, at least 18 AAVs, at least 19 AAVs, at least 20 AAVs, at least 201 AAVs, at least 22 AAVs, at least 23 AAVs, at least 24 AAVs, at least 25 AAVs, at least 26 AAVs, at least 27 AAVs, at least 28 AAVs, at least 29 AAVs, at least 30 AAVs, at least 35 AAVs, at least 40 AAVs, at least 45 AAVs, at least 50 AAVs, at least 60 AAVs, at least 70 AAVs, at least 80 AAVs, at least 90 AAVs, or at least 100 AAVs in the lumen of the EV.

In some aspects, the EV, e.g., an exosome, comprises at least 1 AAV to at least about 100 AAVs. In some embodiments, the EV, e.g., an exosome, comprises at least about 5 AAVs to at least about 100 AAVs, at least about 5 AAVs to at least about 75 AAVs, at least about 5 AAVs to at least about 50 AAVs, at least about 5 AAVs to at least about 45 AAVs, at least about 5 AAVs to at least about 40 AAVs, at least about 5 AAVs to at least about 35 AAVs, at least about 5 AAVs to at least about 30 AAVs, at least about 5 AAVs to at least about 25 AAVs, at least about 5 AAVs to at least about 20 AAVs, at least about 5 AAVs to at least about 15 AAVs, at least about 5 AAVs to at least about 10 AAVs, at least about 10 AAVs to at least about 100 AAVs, at least about 10 AAVs to at least about 75 AAVs, at least about 10 AAVs to at least about 50 AAVs, at least about 5 AAVs to at least about 45 AAVs, at least about 10 AAVs to at least about 40 AAVs, at least about 10 AAVs to at least about 35 AAVs, at least about 10 AAVs to at least about 30 AAVs, at least about 10 AAVs to at least about 25 AAVs, at least about 10 AAVs to at least about 20 AAVs, or at least about 10 AAVs to at least about 15 AAVs in the lumen of the EV.

In some aspects, the EV, e.g., an exosome, comprises at least about 1 AAV to at least about 20 AAVs.

In some embodiments, the EV, e.g., an exosome, comprises at least about 5 AAVs to at least about 20 AAVs.

In some embodiments, the EV, e.g., an exosome, comprises a bi-lipid membrane comprising a luminal surface and an external surface, wherein at least one of the AAVs is not linked to the luminal surface of the EV.

In some embodiments, the EV, e.g., an exosome, comprises a bi-lipid membrane comprising a luminal surface and an external surface, wherein at least one of the AAVs is linked to the luminal surface of the EV.

In some embodiments, the at least one AAV is linked to the luminal surface of the EV by a covalent bond or a non-covalent bond.

In some embodiments, the at least one AAV is linked to the luminal surface of the EV by both a covalent bond and a non-covalent bond.

Certain aspects of the present disclosure are directed to an extracellular vesicle (EV), e.g., an exosome, comprising (i) an AAV and (ii) a scaffold protein, wherein the AAV is associated with the scaffold protein on the external surface of the EV. In some embodiments, the scaffold protein comprises an extracellular domain, and wherein the AAV is associated with the extracellular domain of the scaffold protein. In some embodiments, the scaffold protein further comprises a transmembrane region, wherein the transmembrane region is anchored to the membrane of the EV, e.g., an exosome. In some embodiments, the scaffold protein further comprises an intracellular domain.

In some embodiments, the scaffold protein comprises a heterologous polypeptide, wherein the heterologous polypeptide is fused to an extracellular domain of the scaffold protein, and wherein the heterologous polypeptide associates with the AAV. In some embodiments, the scaffold protein is a type I transmembrane protein or a type II transmembrane protein. In some embodiments, the heterologous polypeptide is fused to the N-terminus or the C terminus of the extracellular domain of the scaffold protein.

In some embodiments, the heterologous polypeptide comprises a receptor, a ligand, an antigen-binding moiety, a substrate, a fragment thereof, or a combination thereof; and wherein the heterologous polypeptide specifically interacts with one or more proteins on the surface of the AAV. In some embodiments, the heterologous polypeptide comprises an antigen-binding moiety selected from the group consisting of an antigen-binding fragment of an antibody, a camelid antibody or an antigen-binding fragment thereof, a single-chain FAB, a nanobody, a shark IgNAR, and a combination thereof. In some embodiments, the antigen-binding moiety comprises a nanobody. In some embodiments, the antigen binding moiety specifically binds the one or more proteins on the surface of the AAV.

In some embodiments, the one or more proteins on the surface of the AAV comprise a capsid protein selected from the group consisting of VP1, VP2, VP3, and any combination thereof. In some embodiments, the one or more proteins on the surface of the AAV is a non-AAV sequence fused to a capsid protein of the AAV. In some embodiments, the capsid protein is selected from VP1, VP2, VP3, and any combination thereof. In some embodiments, the non-AAV sequence is fused to VP2. In some embodiments, the non-AAV sequence is fused to the N-terminus of VP2. In some embodiments, the non-AAV sequence is fused to an internal surface-exposed loop of VP2. In some embodiments, the non-AAV sequence is fused to VP3. In some embodiments, the non-AAV sequence is fused to the N-terminus of VP3. In some embodiments, the non-AAV sequence is fused to an internal surface-exposed loop of VP3. In some embodiments, the non-AAV sequence is fused to VP1. In some embodiments, the non-AAV sequence is fused to an internal surface-exposed loop of VP1.

In some embodiments, the interaction between the affinity ligand (receptor, ligand, antigen-binding moiety) is reversable under certain conditions including changes in pH (e.g. decreased pH in endo-lysosomal compartment), changes in redox conditions (increase or decrease in oxidation), change in ionic conditions, or change in concentration of divalent or trivalent cationic or anionic molecules.

In some embodiments, (i) the scaffold protein is fused to a heterologous polypeptide comprising an Fc receptor; and (ii) the AAV comprises at least one capsid protein fused to an Fc region of an immunoglobulin constant region (Fc). In some embodiments, the Fc receptor is an Fc gamma receptor selected from Fc gamma receptor I (FcγR1), FcγRIIA, FcγIIB, FcγIIIA, and FcγIIIB; and wherein the Fc is an Fc of an IgG. In some embodiments, the Fc receptor is an FcγR1 and the Fc is an Fc of an IgG. In some embodiments, the Fc receptor is an Fc alpha receptor I (FcαR1), and wherein the Fc is an Fc of an IgA. In some embodiments, the Fc receptor is an Fc epsilon receptor selected from Fc epsilon receptor I (FcεRI) and FcεRII, and wherein the Fc is an Fc of an IgE.

In some embodiments, (i) the scaffold protein is fused to a heterologous polypeptide comprising a nanobody; and (ii) the AAV comprises at least one capsid protein fused to an Fc region of an immunoglobulin constant region (Fc). In some embodiments, the nanobody specifically binds to the Fc fused to the capsid protein.

In some embodiments, the at least one capsid protein is selected from the group consisting of VP1, VP2, and VP3. In some embodiments, the AAV comprises at least one VP2 fused to an Fc. In some embodiments, the AAV comprises at least one VP2 that is not fused to an Fc. In some embodiments, the Fc is fused to the N-terminus of the at least one VP2. In some embodiments, the Fc is fused to an internal surface-exposed loop of the at least one VP2. In some embodiments, the AAV comprises at least one VP3 fused to an Fc. In some embodiments, the AAV comprises at least one VP3 that is not fused to the Fc. In some embodiments, the Fc is fused to the N-terminus of the at least one VP3. In some embodiments, the Fc is fused to an internal surface-exposed loop of the at least one VP3. In some embodiments, the AAV comprises at least one VP1 fused to an Fc. In some embodiments, the AAV comprises at least one VP1 that is not fused to an Fc.

In some embodiments, the Fc is fused to a surface-exposed loop of VP1. In some embodiments, the surface-exposed loop comprises the sequence GTTTQSR (SEQ ID NO: 43). In some embodiments, the surface-exposed loop comprises amino acid residues 453 to 459 of VP1 (SEQ ID NO: 44. In some embodiments, the at least one amino acid of the surface-exposed loop is replaced by the Fc. In some embodiments, the surface-exposed loop is replaced by the Fc.

In some embodiments, the scaffold protein is fused to an antigen-binding moiety, wherein the antigen-binding moiety specifically binds an antigen on the surface of an AAV. In some embodiments, the scaffold protein is fused to a heterologous polypeptide comprising an AAV receptor. In some embodiments, the AAV is designed to incorporate a specific heterologous sequence (epitope) on the AAV surface, and this epitope is then recognized by a specific antigen-binding moiety.

In some embodiments, the AAV further comprises a nucleotide sequence comprising a gene of interest. In some embodiments, the gene of interest encodes a protein selected from the group consisting of a secreted protein, a receptor, a structural protein, a signaling protein, a sensory protein, a regulatory protein, a transport protein, a storage protein, a defense protein, a motor protein, a clotting factor, a growth factor, an antioxidant, a cytokine, a chemokine, an enzyme, a tumor suppressor gene, a DNA repair protein, a structural protein, a low-density lipoprotein receptor, an alpha glucosidase, a cystic fibrosis transmembrane conductance regulator, or any combination thereof.

In some embodiments, the gene of interest encodes a factor VIII protein or a Factor IX protein. In some embodiments, the factor VIII protein is a wild-type factor VIII, a B-domain deleted factor VIII, a factor VIII fusion protein, or any combination thereof.

In some embodiments, the gene of interest encodes a Rab proteins geranylgeranyltransferase component A 1 (REP1) In some embodiments, the REP1 comprises an amino acid sequence at least about 70%, at least about 75%, at least about at least about 80%, at least about 85%, at least about 90%, at least about 95%, at least about 96%, at least about 97%, at least about 98%, at least about 99%, or about 100% identical to SEQ ID NO: 45.

In some embodiments, the AAV is selected from the group consisting of AAV type 1, AAV type 2, AAV type 3A, AAV type 3B, AAV type 4, AAV type 5, AAV type 6, AAV type 7, AAV type 8, AAV type 9, AAV type 10, AAV type 11, AAV type 12, AAV type 13, AAV type rh74, AAV type rh32.33, AAV type rh10, AAV type Anc80, AAV type PHP, snake AAV, avian AAV, bovine AAV, canine AAV, equine AAV, ovine AAV, goat AAV, shrimp AAV, primate AAV, human AAV, porcine AAV, a synthetic AAV, an any combination thereof.

In some embodiments, the scaffold protein is selected from the group consisting of prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), CD13, aminopeptidase N (ANPEP), neprilysin (membrane metalloendopeptidase; MME), ectonucleotide pyrophosphatase/phosphodiesterase family member 1 (ENPP1), neuropilin-1 (NRP1), CD9, CD63, CD81, PDGFR, GPI anchor proteins, lactadherin, LAMP2, LAMP2B, a fragment thereof, and any combination thereof. In some embodiments, the scaffold protein is PTGFRN.

In some embodiments, the AAV is linked to the scaffold protein.

In some embodiments, the scaffold protein comprises an N terminus domain (ND) and an effector domain (ED), wherein the ND and/or the ED are associated with the luminal surface of the EV. In some embodiments, the scaffold protein comprises an N terminus domain, an effector domain, and a transmembrane domain, wherein the ND is myristoylated, and wherein the N-terminus domain (ND) and/or the effector domain (ED) are associated with the luminal surface of the EV.

In some embodiments, the ND is associated with the luminal surface of the exosome via myristoylation.

In some embodiments, the ED is associated with the luminal surface of the exosome by an ionic interaction.

In some embodiments, the ED comprises (i) a basic amino acid or (ii) two or more basic amino acids in sequence, wherein the basic amino acid is selected from the group consisting of Lys, Arg, His, and any combination thereof.

In some embodiments, the basic amino acid is (Lys)n, wherein n is an integer between 1 and 10.

In some embodiments, the ED comprises Lys (K), KK, KKK, KKKK (SEQ ID NO: 11), KKKKK (SEQ ID NO: 12), Arg (R), RR, RRR, RRRR (SEQ ID NO: 13); RRRRR (SEQ ID NO: 14), KR, RK, KKR, KRK, RKK, KRR, RRK, (K/R)(K/R)(K/R)(K/R) (SEQ ID NO: 15), (K/R)(K/R)(K/R)(K/R)(K/R) (SEQ ID NO: 16), or any combination thereof.

In some embodiments, the ND comprises the amino acid sequence as set forth in G:X2:X3:X4:X5:X6, wherein G represents Gly; wherein “:” represents a peptide bond, wherein each of the X2 to the X6 is independently an amino acid, and wherein the X6 comprises a basic amino acid.

In some embodiments, the X2 is selected from the group consisting of Pro, Gly, Ala, and Ser; the X4 is selected from the group consisting of Pro, Gly, Ala, Ser, Val, Ile, Leu, Phe, Trp, Tyr, Gln and Met; the X5 is selected from the group consisting of Pro, Gly, Ala, and Ser; the X6 is selected from the group consisting of Lys, Arg, and His; or any combination thereof.

In some embodiments, the ND comprises the amino acid sequence of G:X2:X3:X4:X5:X6, wherein G represents Gly; “:” represents a peptide bond; the X2 is an amino acid selected from the group consisting of Pro, Gly, Ala, and Ser; the X3 is an amino acid; the X4 is an amino acid selected from the group consisting of Pro, Gly, Ala, Ser, Val, Ile, Leu, Phe, Trp, Tyr, Gln and Met; the X5 is an amino acid selected from the group consisting of Pro, Gly, Ala, and Ser; and the X6 is an amino acid selected from the group consisting of Lys, Arg, and His.

In some embodiments, the X3 is selected from the group consisting of Asn, Gln, Ser, Thr, Asp, Glu, Lys, His, and Arg.

In some embodiments, the ND and the ED are joined by a linker. In some embodiments, the linker comprises a peptide bond or one or more amino acids.

In some embodiments, the ND comprises an amino acid sequence selected from the group consisting of (i) GGKLSKK (SEQ ID NO: 17), (ii) GAKLSKK (SEQ ID NO: 18), (iii) GGKQSKK (SEQ ID NO: 19), (iv) GGKLAKK (SEQ ID NO: 20), (v) GGKLSKK (SEQ ID NO: 21), or (vi) any combination thereof. In some embodiments, the ND comprises an amino acid sequence selected from the group consisting of (i) GGKLSKKK (SEQ ID NO: 22), (ii) GGKLSKKS (SEQ ID NO: 23), (iii) GAKLSKKK (SEQ ID NO: 24), (iv) GAKLSKKS (SEQ ID NO: 25), (v) GGKQSKKK (SEQ ID NO: 26), (vi) GGKQSKKS (SEQ ID NO: 27), (vii) GGKLAKKK (SEQ ID NO: 28), (viii) GGKLAKKS (SEQ ID NO: 29), (ix) GGKLSKKK (SEQ ID NO: 30), (x) GGKLSKKS (SEQ ID NO: 31), and (xi) any combination thereof. In some embodiments, the ND comprises the amino acid sequence GGKLSKK (SEQ ID NO: 17).

In some embodiments, the scaffold protein is at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 105, at least about 110, at least about 120, at least about 130, at least about 140, at least about 150, at least about 160, at least about 170, at least about 180, at least about 190, or at least about 200 amino acids in length.

In some embodiments, the scaffold protein comprises (i) GGKLSKKKKGYNVN (SEQ ID NO: 32), (ii) GAKLSKKKKGYNVN (SEQ ID NO: 33), (iii) GGKQSKKKKGYNVN (SEQ ID NO: 34), (iv) GGKLAKKKKGYNVN (SEQ ID NO: 35), (v) GGKLSKKKKGYSGG (SEQ ID NO: 36), (vi) GGKLSKKKKGSGGS (SEQ ID NO: 37), (vii) GGKLSKKKKSGGSG (SEQ ID NO: 38), (viii) GGKLSKKKSGGSGG (SEQ ID NO: 39), (ix) GGKLSKKSGGSGGS (SEQ ID NO: 40), (x) GGKLSKSGGSGGSV (SEQ ID NO: 41), or (xi) GAKKSKKRFSFKKS (SEQ ID NO: 42).

In some embodiments, the scaffold protein does not comprise Met at the N terminus. In some embodiments, the scaffold protein comprises a myristoylated amino acid residue at the N terminus of the scaffold protein. In some embodiments, the amino acid residue at the N terminus of the scaffold protein is Gly. In some embodiments, the amino acid residue at the N terminus of the scaffold protein is synthetic. In some embodiments, the amino acid residue at the N terminus of the scaffold protein is a glycine analog.

In some embodiments, the scaffold protein comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 8, SEQ ID NO: 9, or SEQ ID NO: 10.

In some embodiments, the C-terminus of the scaffold protein is linked to a capsid protein of the AAV. In some embodiments, the EV is an exosome.

Certain aspects of the present disclosure are directed to an adeno-associated virus (AAV) comprising a capsid, wherein the capsid comprises at least one capsid protein selected from the group consisting of VP1, VP2, and VP3; wherein the at least one capsid protein is linked to a scaffold protein.

In some embodiments, the EV further comprises a second scaffold protein, which comprises prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), CD13, aminopeptidase N (ANPEP), neprilysin (membrane metalloendopeptidase; MME), ectonucleotide pyrophosphatase/phosphodiesterase family member 1 (ENPP1), neuropilin-1 (NRP1), CD9, CD63, CD81, PDGFR, GPI anchor proteins, lactadherin, LAMP2, LAMP2B, a fragment thereof, and any combination thereof.

In some embodiments, the AAV comprises at least one capsid protein fused to the scaffold protein. In some embodiments, the at least one capsid protein is selected from the group consisting of VP1, VP2, and VP3. In some embodiments, the AAV capsid protein comprises VP2. In some embodiments, the AAV comprises at least one VP2 that is not fused to the scaffold protein. In some embodiments, the scaffold protein is fused to the N-terminus of the VP2. In some embodiments, the scaffold protein is fused to the C-terminus of the VP2. In some embodiments, the number of the VP2 fused to the scaffold protein is less than the number of VP2 not fused to the scaffold protein. In some embodiments, the number of the VP2 fused to the scaffold protein is about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 30 fold, about 35 fold, about 40 fold, about 46 fold, about 50 fold, or about 100 fold less than the number of the at least one VP2 not fused to the scaffold protein.

In some embodiments, the scaffold protein is a type I transmembrane protein or a type II transmembrane protein. In some embodiments, the a type I transmembrane protein comprises prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), CD13, aminopeptidase N (ANPEP), neprilysin (membrane metalloendopeptidase; MME), ectonucleotide pyrophosphatase/phosphodiesterase family member 1 (ENPP1), neuropilin-1 (NRP1), CD9, CD63, CD81, PDGFR, GPI anchor proteins, lactadherin, LAMP2, LAMP2B, a fragment thereof, and any combination thereof.

In some embodiments, the C terminus of the type I transmembrane protein or the N terminus of the type II transmembrane protein is linked to a dimerizing agent, e.g., a binding partner of a chemically induced dimer. In some embodiments, the scaffold protein is linked to a binding partner of a chemically induced dimer. In some embodiments, the binding partner of the chemically induced dimer comprises one of binding partners selected from the group; consisting of (i) FKBP and FKBP (FK1012); (ii) FKBP and CalcineurinA (CNA) (FK506); (iii) FKBP and CyP-Fas (FKCsA); (iv) FKBP and FRB (Rapamycin); (v) GyrB and GyrB (Coumermycin); (vi) GAI and GID1 (Gibberellin); (vii) Snap-tag and HaloTag (HaXS); (viii) eDHFR and HaloTag (TMP-HTag); and (ix) BCL-xL and Fab (AZ1) (ABT-737). In some embodiments, the chemically induced dimer comprises an FRB-FKBP fusion complex. In some embodiments, the FRB is the FRB of mTOR. In some embodiments, the AAV comprises at least one capsid protein fused to one of the binding partners of the chemically induced dimer, thereby forming a dimer complex when the binding partners come in contact with the chemical compound.

In some embodiments, the at least one capsid protein is selected from the group consisting of VP1, VP2, and VP3. In some embodiments, the AAV capsid protein comprises VP2. In some embodiments, the AAV comprises at least one VP2 that is not fused to a binding partner of the chemically induced dimer. In some embodiments, the number of the VP2 fused to a binding partner of the chemically induced dimer is less than the at least one VPs that is not fused to a binding partner of the chemically induced dimer. In some embodiments, the number of the VP2 linked to the binding partner of the chemically induced dimer is about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 30 fold, about 35 fold, about 40 fold, about 46 fold, about 50 fold, or about 100 fold less than the number of the at least one VP2 not fused to the binding partner.

In some embodiments, the binding partner of the chemically induced dimer is inserted within an internal loop of the AAV capsid protein. In some embodiments, the internal loop comprises the sequence GTTTQSR (SEQ ID NO: 43). In some embodiments, the internal loop comprises amino acid residues 453 to 459 of SEQ ID NO: 44 (capsid protein VP1 of AAV2; Uniprot P03135). In some aspects, the binding partner of the chemically induced dimer is inserted into a site selected from R585, R587, R588, or any combination thereof of capsid protein VP2 of AAV2 or a homologous site in a similar capsid protein (see, e.g., Buning and Srivastava, Methods and Clinical Development 12:248-266 (March 2019), which is incorporated by reference herein in its entirety). In some embodiments, at least one amino acid of the internal loop is replaced by a binding partner of the chemically induced dimer. In some embodiments, the scaffold protein is linked to the binding partner of the chemically induced dimer by a linker.

In some embodiments, the AAV capsid protein is linked to the binding partner of the chemically induced dimer by a linker. In some embodiments, the linker comprises a covalent bond or one or more amino acids. In some embodiments, the linker is a cleavable linker.

In some embodiments, the scaffold protein is linked to an affinity agent that specifically binds to the AAV. In some embodiments, the affinity agent is an AAV receptor, a single-domain antibody, a nanobody, a camelid, a VHH fragment, an immunoglobulin new antigen receptor (IgNAR) an antibody or an antigen-binding portion thereof, or any combination thereof. In some embodiments, the antigen-binding portion thereof comprises a single chain Fab. In some embodiments, the affinity agent binds to one or more AAV capsid proteins. In some embodiments, the one or more AAV capsid proteins are AAV assembly activating proteins. In some embodiments, the affinity agent does not bind to an AAV capsid protein monomer. In some embodiments, the affinity agent comprises of an AAV receptor. In some embodiments, the AAV receptor is AAVR or GPR108.

In some embodiments, the AAV further comprises a genetic cassette. In some embodiments, the genetic cassette encodes a protein selected from the group consisting of a secreted protein, a receptor, a structural protein, a signaling protein, a sensory protein, a regulatory protein, a transport protein, a storage protein, a defense protein, a motor protein, a clotting factor, a growth factor, an antioxidant, a cytokine, a chemokine, an enzyme, a tumor suppressor gene, a DNA repair protein, a structural protein, a low-density lipoprotein receptor, an alpha glucosidase, a cystic fibrosis transmembrane conductance regulator, or any combination thereof. In some embodiments, the genetic cassette encodes a factor VIII protein or a factor IX protein. In some embodiments, the factor VIII protein is a wild-type factor VIII, a B-domain deleted factor VIII, a factor VIII fusion protein, or any combination thereof.

In some embodiments, the gene of interest encodes a Rab proteins geranylgeranyltransferase component A 1 (REP1). In some embodiments, the REP1 comprises an amino acid sequence at least about 70%, at least about 75%, at least about at least about 80%, at least about 85%, at least about 90%, at least about 95%, at least about 96%, at least about 97%, at least about 98%, at least about 99%, or about 100% identical to SEQ ID NO: 45.

In some embodiments, the AAV is selected from the group consisting of AAV type 1, AAV type 2, AAV type 3A, AAV type 3B, AAV type 4, AAV type 5, AAV type 6, AAV type 7, AAV type 8, AAV type 9, AAV type 10, AAV type 11, AAV type 12, AAV type 13, AAV type rh74, AAV type rh32.33, AAV type rh10, AAV type Anc80, AAV type PHP, snake AAV, avian AAV, bovine AAV, canine AAV, equine AAV, ovine AAV, goat AAV, shrimp AAV, primate AAV, human AAV, porcine AAV, a synthetic AAV, an any combination thereof.

Certain aspects of the present disclosure are directed to an AAV in the an EV disclosed herein.

Certain aspects of the present disclosure are directed to an AAV comprising VP2 linked to a scaffold protein comprising the amino acid sequence as set forth in G:X2:X3:X4:X5:X6, wherein G represents Gly; wherein “:” represents a peptide bond, wherein each of the X2 to the X6 is independently an amino acid, and wherein the X6 comprises a basic amino acid. In some embodiments, the scaffold protein is the scaffold protein disclosed herein.

Certain aspects of the present disclosure are directed to an AAV comprising VP2 linked to a binding partner of a chemically induced dimer. In some embodiments, the binding partner of the chemically induced dimer comprises any one of the binding partners disclosed.

Certain aspects of the present disclosure are directed to an AAV comprising one or more capsid proteins specifically bound to an affinity agent disclosed herein.

Certain aspects of the present disclosure are directed to a pharmaceutical composition comprising an EV, e.g., an exosome, or an AAV disclosed herein and a pharmaceutically acceptable carrier.

Certain aspects of the present disclosure are directed to a cell that produces an EV, e.g., an exosome, or an AAV disclosed herein.

Certain aspects of the present disclosure are directed to a cell comprising a first nucleotide sequence encoding an AAV protein linked to the scaffold protein as disclosed herein. In some embodiments, the cell further comprises a second nucleotide sequence comprising a gene of interest disclosed herein.

Certain aspects of the present disclosure are directed to a cell comprising a first nucleotide encoding an AAV protein linked to a binding partner of the chemically induced dimer as disclosed herein. In some embodiments, the cell further comprises a second nucleotide sequence encoding the corresponding binding partner of the chemically induced dimer, which is linked to a scaffold protein disclosed herein. In some embodiments, the cell further comprises a third nucleotide sequence comprising a gene of interest disclosed herein.

Certain aspects of the present disclosure are directed to a cell comprising a first nucleotide encoding an affinity agent disclosed herein linked to a scaffold protein disclosed herein. In some embodiments, the cell further comprises a second nucleotide sequence comprising the gene of interest disclosed herein.

Certain aspects of the present disclosure are directed to a kit comprising an isolated EV disclosed herein, e.g., an exosome, and instructions for use.

Certain aspects of the present disclosure are directed to a method of making EVs, e.g., exosomes, comprising culturing a cell disclosed herein under a suitable condition and obtaining the EVs.

Certain aspects of the present disclosure are directed to a method of preventing or treating a disease in a subject in need thereof, comprising administering to the subject an EV, an AAV, or a pharmaceutical composition disclosed herein. In some embodiments, the disease is selected from a cancer, a hemophilia, diabetes, a growth factor deficiency, an eye disease, a Pompe disease, Gaucher disease, a lysosomal storage disorder, mucovicidosis, cystic fibrosis, Duchenne and Becker muscular dystrophy, transthyretin amyloidosis, hemophilia A, hemophilia B, adenosine-deaminase deficiency, Leber's congenital amaurosis, X-linked adrenoleukodystrophy, metachromatic leukodystrophy, OTC deficiency, glycogen storage disease 1A, Criggler-Najjar syndrome, primary hyperoxaluria type 1, acute intermittent porphyria, phenylketonuria, familial hypercholesterolemia, mucopolysaccharidosis type VI, α1 antitrypsin deficiency, Retts Syndrome, Dravet Syndrome, Angelman Syndrome, DM1 disease, Fragile X disease, Huntingtons Disease, Friedreichs ataxia, and a hypercholesterolemia.

Certain aspects of the present disclosure are directed to a method of delivering an AAV to a subject, comprising administering to the subject a EV disclosed herein. In some embodiments, the EV is administered parenterally, orally, intravenously, intramuscularly, intra-tumorally, intranasally, subcutaneously, or intraperitoneally. In some embodiments, the method further comprises administering an additional therapeutic agent.

In some aspects, the EV administration is intraocular administration. In some aspects, the intraocular administration is intravitreal administration, intracameral administration, subconjunctival administration, subretinal administration, subscleral administration, intrachoroidal administration, and any combination thereof. In some aspects, the intraocular administration comprises the injection of the EV. In some aspects, the intraocular administration is intravitreal injection. In some aspects, the intraocular administration comprises the implantation of a delivery device comprising the composition. In some aspects, the delivery device is an intraocular delivery device. In some aspects, the intraocular delivery device is an intravitreal implant or a scleral plug. In some aspects, the delivery device is a sustained release delivery device. In some aspects, the delivery device is biodegradable. In some aspects, the intraocular administration of the EV is to treat a disease selected from the group consisting of selected from the group consisting of macular degeneration, cataract, diabetic retinopathy, glaucoma, amblyopia, strabismus, retinopathy, Leber congentical amaurosis, or any combination thereof.

### BRIEF DESCRIPTION OF FIGURES

FIG. 1 is a drawing of an AAV capsid protein (e.g., VP1, VP2, or VP3) fused to the C-terminus of a scaffold protein comprising the minimal sequence GGKLSKK (SEQ ID NO: 17).

FIG. 2 is a drawing of an AAV capsid VP2 fused to a dimerizing agent, e.g., an FKBP-rapamycin-binding (FRB) agent. This binding partner is fused to the N-terminus of the VP2 capsid protein. The corresponding binding partner (e.g., FKBP) is linked to the C-terminus of either PTGFRN or a scaffold protein comprising the minimal sequence GGKLSKK (SEQ ID NO: 17).

FIGS. 3A and 3B are drawings of an AAV capsid protein (e.g., VP1, VP2, or VP3) fused to a dimerization agent, e.g., binding partner of the chemically induced dimer FRB. The binding partner is inserted within an internal loop (e.g., VP1) at position 455. The AAV is produced in cells co-producing exosomes in the presence of the necessary chemical to induce dimerization (e.g., in the presence of rapamycin to induce dimerization of the FRB).

FIGS. 4A-4C show a scaffold protein comprising the minimal sequence GGKLSKK (SEQ ID NO: 17) linked to an AAV receptor (AAVR; FIG. 4A), to an AAV affinity agent shown here linked via Scaffold Y (FIG. 4C) and a Scaffold X protein linked to an AAV affinity agent (FIG. 4B).

FIG. 5 is a drawing of a scaffold protein (e.g., PTGFRN) linked at an extracellular domain to an antigen-binding domain (a nanobody). The antigen-binding domain (nanobody) specifically binds an epitope on the AAV capsid, such as VP1, VP2, or VP3.

FIG. 6 is a drawing of a capsid protein (e.g., VP1, VP2, or VP3) linked to an Fc region of IgG. A scaffold protein (e.g., PTGFRN) is linked to either an FcγR1 or an Fc nanobody that specifically binds Fc. The FcγR1 or the Fc nanobody is linked to an extracellular domain of the scaffold protein (e.g., PTGFRN).

FIG. 7A shows a diagram of a scaffold protein (e.g., PTGFRN) linked to an AAV receptor (AAVR). The AAVR is linked to the extracellular domain of the scaffold protein (e.g., PTGFRN) and binds an epitope on the AAV capsid such as VP1, VP2, or VP3. The AAVR can be PKD1, PKD2 or single chain antibodies. FIG. 7B is a gel illustrating that exosomes were successfully constructed having an AAV receptor fused to a scaffold X protein (“AAVR exosomes”).

FIGS. 8A and 8B show bio-layer interferometry (Octet) data showing that exosomes comprising AAVR fused to a scaffold protein bind to immobilized AAV2 (as illustrated in FIG. 8A), while control exosomes do not.

FIG. 9A is an image of a protein gel of equal amounts of cell lysate from the cytosol (left) and nucleus (right) loaded on a denaturing polyacrylamide gel. FIG. 9B shows western blotting for Etp-GFP, Etp-VP2, and FRB-VP2 using antibodies specific for αFLAG tag (expressed on all constructs). FIG. 9C shows western blotting using antibodies specific for αHistone H4 (a nuclear marker) in both cytosol and nucleus lysates.

FIGS. 10A-10D show the results of various AAV capsid serotypes transfected into HEK293T cells and HEK293 cells adapted for suspension culture (HEK293SF). FIGS. 10A-10D show that AAV1, AAV2, AAV3, AAV5, and AAV6 capsids are detected via Western Blot.

FIG. 11A shows the separation of mixture components via ultracentrifugation. FIG. 11B show the results of the NTA (particle/mL) and qPCR (gene copies/mL (GC/mL)) results in the collected fractions (1-10) as indicated in the diagram of FIG. 11A.

FIGS. 12A-12C show a western blot of ten collected fractions assayed for the presence of VP1, VP2, and VP3 protein using various exposure times. VP1, VP2, and VP3 polypeptides can be seen most prominently in fractions 8, 9, and 10, where they are not associated with exosomes. Fractions 1, 2, 3, and 5 also have detectable VP1, VP2, and VP3 and are found to be associated with higher exosome concentration in these fractions.

FIG. 13 shows a chromatogram of an elution of a culture harvest of HEK293T cells transfected with AAV9 using the triple transfection method. The separation used a linear gradient elution (LGE) with increasing concentrations of NaCl from 150 mM NaCl to 1 M NaCl across twenty column volumes.

FIG. 14A shows the collection of fractions F1-F8 in the primary elution peak as seen in FIG. 9 and the data resulting from NTA analysis to determine exosome count and particle size. FIGS. 14B-14D show cryogenic electron microscopy (cryoEM) images of exosomes comprising AAV5 serotype (FIGS. 14B-14C) and AAV9 serotype (FIG. 14D).

FIG. 15 shows a separation of each individual F1-F8 fractions using size-exclusion chromatography (SEC).

FIG. 16 shows the results from an experiment where cells were transfected with a fixed GC/well (fixed MOI: 6000) with either free (“AAV”) or encapsulated AAV9-GFP (“exo”) in the presence of anti-AAV IgG. FIG. 12 shows GFP expression as measured by fluorescence intensity as determined every three hours for a period of four days, run in triplicate.

FIG. 17B is a graphical representation showing GFP expression from exosome-associated AAV or free AAV in cell culure in the presence of increasing concentrations of inhibitory anti-AAV9 antibody. FIG. 17B shows a comparison study of GFP expression using various samples (Chrom F3-Chrom F9) of either free or encapsulated AAV9-GFP in the presence of anti-AAV monoclonal antibodies at various dilutions.

FIGS. 18A-18D show results of a head-to-head study of a sample derived from the F7 fraction showing the comparison of GFP expression fluorescence intensity in HeLa cell culture using free (“AAV”) or encapsulated AAV9-GFP (“exosome-AAV”) measured at time points 24h (FIG. 18A), 48h (FIG. 18B), 72h (FIG. 18C), and 96h (FIG. 18D) following addition of the sample to HeLa cell culture.

FIG. 19 shows a comparison study of luciferase expression using free (“free AAV9”) or encapsulated AAV (“exosome-AAV”) in response to increasing concentrations of intravenous immunoglobulin (“IVIG”).

FIG. 20 is a bar graph illustrating the level of nanoLuc expression (RLU/mg of protein) in homogenized mouse eyes 2 weeks after administration of a PBS control, free AAV9 encoding secreted nanoLuc, or an exosome comprising an AAV9 encoding secreted nanoLuc.

FIG. 21A is a drawing, illustrating association of an AAV with the luminal surface of an exosome membrane through the interaction of the AAV with a scaffold Y protein fused to an AAV affinity ligand. FIGS. 21B and 21C are bar graphs showing that luminal loading of exosomes using scaffold Y fused to an AAV affinity ligand leads to increased localization of AAV to exosomes as a percent of the total AAV released (FIG. 21B) and relative to enrichment of AAV in exosomes lacking the scaffold Y fusion construct (FIG. 21C).

### DETAILED DESCRIPTION OF DISCLOSURE

Certain aspects of the present disclosure are directed to an extracellular vesicle (EV), e.g., an exosome, comprising an AAV and a scaffold protein. In some aspects, the AAV is in the lumen of the EV. In some aspects, the AAV is associated with the membrane of the EV, e.g., exosome. In some aspects, the AAV is associated with the luminal surface of the EV, e.g., exosome. In some aspects, the AAV is associated with the exterior surface of the EV, e.g., exosome. In some aspects, the AAV associated with the exosome has altered properties as compared to the free AAV alone.

Certain aspects of the present disclosure are directed to an extracellular vesicle (EV), e.g., an exosome, comprising an adeno-associated virus (AAV) and a scaffold protein, wherein the AAV is present in the lumen of the EV. In certain embodiments, the number of the AAV in the lumen of the exosome is higher than the number of the AAV in the lumen of a reference EV, wherein the AAVs in the lumen of the reference EV were introduced without a scaffold protein. In certain embodiments, the percentage of EVs that contain AAV in the lumen is higher than that of the reference EV wherein the AAV's were introduced without a scaffold protein. Certain aspects of the present disclosure are directed to an EV, e.g., an exosome, comprising at least five AAV in the lumen of the EV. Some aspects of the present disclosure are directed to an AAV comprising at least one capsid protein (e.g., VP1, VP2, and VP3), linked to a scaffold protein. In some embodiments, the scaffold protein comprises the amino acid sequence as set forth in G:X2:X3:X4:X5:X6, wherein G represents Gly; wherein “:” represents a peptide bond, wherein each of the X2 to the X6 is independently an amino acid, and wherein the X6 comprises a basic amino acid.

Certain aspects of the present disclosure are directed to an extracellular vesicle (EV), e.g., an exosome, comprising an adeno-associated virus (AAV) and a scaffold protein, wherein the AAV is associated with the scaffold protein on the external surface of the EV. In some embodiments, the scaffold protein comprises an extracellular domain, and the AAV is associated with the extracellular domain of the scaffold protein. In certain embodiments, the AAV is associated with the scaffold protein by a covalent bond. In some embodiments, the AAV is associated with the scaffold protein by a non-covalent interaction.

Some aspects of the present disclosure are directed to an EV engineered to contain an AAV affinity ligand.

Non-limiting examples of the various embodiments are shown in the present disclosure.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the terms “extracellular vesicle” and “EV” are used interchangeably and refer to a cell-derived vesicle comprising a membrane that encloses an internal space (i.e., a lumen). Extracellular vesicles comprise all membrane-bound vesicles (e.g., exosomes, nanovesicles, microvesicles) that have a smaller diameter than the cell from which they are derived. In some embodiments, extracellular vesicles range in diameter from 20 nm to 1000 nm, and can comprise various macromolecular payloads either within the internal space (i.e., lumen), displayed on the external surface of the extracellular vesicle, and/or spanning the membrane, or a combination thereof. In some embodiments, the payload can comprise nucleic acids, proteins, carbohydrates, lipids, small molecules, and/or combinations thereof. In certain embodiments, the payload comprises an AAV. In some embodiments, the payload comprises an AAV and nucleic acids, proteins, carbohydrates, lipids, small molecules, and/or combinations thereof. In some aspects, the term extracellular vesicle or EV refers to a population of extracellular vesicles (EVs).

By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some embodiments, the extracellular vesicles are produced by cells that express one or more transgene products.

As used herein, the term “exosome” refers to an extracellular vesicle (EV) with a diameter between 20-300 nm (e.g., between 40-200 nm). Exosomes comprise a membrane that encloses an internal space (i.e., lumen), and, in some embodiments, can be generated from a cell (e.g., producer cell) by direct plasma membrane budding or by fusion of the late endosome or multi-vesicular body (MVB) with the plasma membrane. In certain embodiments, an exosome comprises a scaffold protein. As described infra, an exosome can be derived from a producer cell, and isolated from the producer cell based on its size, density, biochemical parameters, or a combination thereof. In some embodiments, the EVs, e.g., exosomes, of the present disclosure are produced by cells that express one or more transgene products. In certain embodiments, the EVs, e.g., exosomes, of the present disclosure are generated by cells that co-produce AAV. In some aspects, the term exosome refers to a population of exosomes.

As used herein, the term “nanovesicle” refers to an extracellular vesicle with a diameter between 20-250 nm (e.g., between 30-150 nm) and is generated from a cell (e.g., producer cell) by direct or indirect manipulation such that the nanovesicle would not be produced by the cell without the manipulation. Appropriate manipulations of the cell to produce the nanovesicles include but are not limited to serial extrusion, treatment with alkaline solutions, sonication, or combinations thereof. In some embodiments, production of nanovesicles can result in the destruction of the producer cell. In some embodiments, population of nanovesicles described herein are substantially free of vesicles that are derived from cells by way of direct budding from the plasma membrane or fusion of the late endosome with the plasma membrane. In certain embodiments, a nanovesicle comprises a scaffold protein. Nanovesicles, once derived from a producer cell, can be isolated from the producer cell based on its size, density, biochemical parameters, or a combination thereof.

As used herein, “microvesicles” refers to extracellular vesicles generated by the outward budding and fission of membrane vesicles from the cell surface.

As used herein, the term “scaffold protein” refers to a polypeptide that can be used to anchor a payload or any other compound of interest (e.g., an AAV) to the EV. In some aspects, the scaffold protein is a polypeptide that does not naturally exist in an EV. In some embodiments, the scaffold protein comprises a synthetic polypeptide. In some embodiments, the scaffold protein comprises a modified protein, wherein the corresponding unmodified protein naturally exists in the EV (an “EV protein”), e.g., the exosome. In some embodiments, the scaffold protein comprises a protein that naturally exists in the EV, or a fragment thereof, e.g., a fragment of an EV protein, where the protein is expressed at a higher level than naturally occuring. In some embodiments, a scaffold protein further comprises a non-polypeptide moiety. In other embodiments, a scaffold protein further comprises a lipid and/or a carbohydrate. In some embodiments, the scaffold protein comprises a fusion protein, comprising (i) a naturally occurring EV protein or a fragment thereof and (ii) a heterologous peptide (e.g., an antigen binding domain, a capsid protein, an Fc receptor, a binding partner of a chemically induced dimer, or any combination thereof).

As used herein, the term “binding partner” or “dimerizing agent” refers to one member of at least two elements that interact with each other to form a multimer (e.g., a dimer). In some embodiments, the binding partner is a first binding partner that interacts with a second binding partner. In some embodiments, the binding partner is a first binding partner that interacts with a second binding partner and/or a third binding partner. Any binding partners or dimerizing agents can be used in the compositions and methods disclosed herein. In some embodiments, the binding partner can be a polypeptide, a polynucleotide, a fatty acid, a small molecule, or any combination thereof. In certain embodiments, the binding partner (e.g., the first binding partner and/or the second binding partner) is selected from a first and a second binding partners of a chemically induced dimer selected from the group consisting of (i) FKBP and FKBP (FK1012); (ii) FKBP and CalcineurinA (CNA) (FK506); (iii) FKBP and CyP-Fas (FKCsA); (iv) FKBP and FRB (Rapamycin); (v) GyrB and GyrB (Coumermycin); (vi) GAI and GID1 (Gibberellin); (vii) Snap-tag and HaloTag (HaXS); (viii) eDHFR and HaloTag (TMP-HTag); and (ix) BCL-xL and Fab (AZ1) (ABT-737).

In some embodiments, the scaffold protein comprises a fusion comprising (i) a protein that naturally exists in the EV (an EV protein) or a fragment thereof and (ii) a second polypeptide sequence. The term “Scaffold X” refers to exosome proteins that have recently been identified on the surface of exosomes. In some embodiments, the EV protein is selected from an EV protein described in U.S. Pat. No. 10,195,290, which is incorporated herein by reference in its entirety. In some embodiments, the EV protein is selected from the group consisting of prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), CD13, aminopeptidase N (ANPEP), neprilysin (membrane metalloendopeptidase; MME), ectonucleotide pyrophosphatase/phosphodiesterase family member 1 (ENPP1), neuropilin-1 (NRP1), CD9, CD63, CD81, PDGFR, GPI anchor proteins, lactadherin, LAMP2, LAMP2B, a fragment thereof, and any combination thereof. As used herein, the term “Scaffold Y” refers to exosome proteins that were newly identified within the lumen of exosomes or a fragment thereof. See, e.g., International Appl. No. PCT/US2018/061679, which is incorporated herein by reference in its entirety. Non-limiting examples of Scaffold Y proteins include those selected from the group consisting of myristoylated alanine rich Protein Kinase C substrate (“MARCKS” or “MARCKS protein”); myristoylated alanine rich Protein Kinase C substrate like 1 (“MARCKSL1” or “MARCKSL1 protein”); and brain acid soluble protein 1 (“BASP1” or “BASP1 protein”).

In certain embodiments, the Scaffold Y protein comprises a fragment of an EV protein. In some embodiments, the scaffold protein comprises a fragment of MARCKS, MARCKSL1, or BASP1. In some embodiments, the scaffold protein comprises the amino acid sequence GGKLSKK (SEQ ID NO: 17). In some embodiments, the scaffold protein comprises the amino acid sequence GGKLSKK (SEQ ID NO: 17), wherein the C-terminal Glycine residue is myristoylated. In some embodiments, the scaffold protein comprises (a) (i) a fragment of MARCKS, MARCKSL1, or BASP or (ii) the amino acid sequence GGKLSKK (SEQ ID NO: 17), and (b) a transmembrane domain, wherein the transmembrane domain is linked (e.g., by a linker), to the C-terminus of the sequence of (a)(i) or (a)(ii). In some embodiments, the scaffold protein comprises (a) (i) a fragment of MARCKS, MARCKSL1, or BASP or (ii) the amino acid sequence GGKLSKK (SEQ ID NO: 17), (b) a transmembrane domain, and (c) an extracellular domain, wherein the transmembrane domain is linked (e.g., by a linker), to the C-terminus of the sequence of (a)(i) or (a)(ii), and wherein the extracellular domain is linked to the C-terminus of the transmembrane domains.

In some embodiments, the scaffold protein is a transmembrane protein. As used herein, a “transmembrane protein” refers to any protein that comprises an extracellular domain (e.g., at least one amino acid that is located external to the membrane of the EV, e.g., exosome, e.g., extra-vesicular), a transmembrane domain (e.g., at least one amino acid that is located within the membrane of an EV, e.g., within the membrane of an exosome), and an intracellular domain (e.g., at least one amino acid that is located internal to the membrane of the EV, e.g., exosome, e.g., intra-vesicular). In some embodiments, a scaffold protein described herein is a type I transmembrane protein, wherein the N-terminus of the transmembrane protein is located in the extracellular space, e.g., outside (or external to) the membrane that encloses the EV, e.g., exosome, e.g., extra-vesicular. In some embodiments, a scaffold protein described herein is a type II transmembrane protein, wherein the N-terminus of the transmembrane protein is located in the lumen, e.g., in the intracellular space, e.g., inside the membrane, e.g., on the luminal side of the membrane, that encloses the EV, e.g., exosome, e.g., intra-vesicular.

As used herein, the term “extracellular” can be used interchangeably with the terms “external,” “exterior,” and “extra-vesicular,” wherein each term refers to an element that is outside the membrane that encloses the EV. As used herein, the term “intracellular” can be used interchangeably with the terms “internal,” “interior,” and “intra-vesicular,” wherein each term refers to an element that is inside the membrane that encloses the EV. The term “lumen” refers to the space inside the membrane enclosing the EV. Accordingly, an element that is inside the lumen of an EV can be referred to herein as being “located in the lumen” or “luminal.”

The term “anchored,” as used herein, refers to an element that is associated with the membrane. In some embodiments, the element that is anchored to the membrane is associated with a transmembrane protein, wherein the transmembrane protein anchors the element to the membrane. In some embodiments, the element that is anchored to the membrane is associated with a scaffold protein that comprises a motif (e.g., a scaffold protein comprising GGKLSKK (SEQ ID NO: 17)) that interacts with the membrane, thereby anchoring the element to the membrane. In some embodiments, the scaffold protein comprises a myristoylated amino acid residue at the N terminus of the scaffold protein, wherein the myristoylated amino acid anchors the scaffold protein to the membrane of the EV. An element can be anchored directly (e.g. a peptide bond) or by a linker to the membrane.

As used herein the term “lumen-engineered exosome” refers to an EV, e.g., exosome, wherein the membrane or the lumen of the EV, e.g., exosome, is modified in its composition so that the lumen of the engineered EV, e.g., exosome, is different from that of the EV, e.g., exosome, prior to the modification or of the naturally occurring EV, e.g., exosome. The engineering can be directly in the lumen or in the membrane of the EV, e.g., exosome, so that the lumen of the EV, e.g., exosome, is changed. For example, the membrane is modified in its composition of a protein, a lipid, a small molecule, a carbohydrate, etc. so that the lumen of the EV, e.g., exosome is modified. The composition can be changed by a chemical, a physical, or a biological method or by being produced from a cell previously modified by a chemical, a physical, or a biological method. Specifically, the composition can be changed by a genetic engineering or by being produced from a cell previously modified by genetic engineering. In some embodiments, a lumen-engineered exosome comprises an exogenous protein (i.e., a protein that the EV, e.g., exosome does not naturally express) or a fragment or variant thereof that can be exposed in the lumen of the EV, e.g., exosome or can be an anchoring point (attachment) for a moiety exposed on the inner layer of the EV, e.g., exosome. In other embodiments, a lumen-engineered EV, e.g., exosome, comprises a higher expression of a natural exosome protein (e.g., any EV protein described herein) or a fragment or variant thereof that can be exposed to the lumen of the exosome or can be an anchoring point (attachment) for a moiety exposed in the lumen of the exosome as compared to a non-engineered or modified exosome.

As used herein the term “external surface-engineered exosome” refers to an EV, e.g., exosome, wherein the membrane of the EV, e.g., exosome, is modified in its composition so that the external surface of the engineered EV, e.g., exosome, is different from that of the EV, e.g., exosome, prior to the modification or of the naturally occurring EV, e.g., exosome. For example, the membrane is modified in its composition of a protein, a lipid, a small molecule, a carbohydrate, etc. so that the external surface of the EV, e.g., exosome is modified. The composition can be changed by a chemical, a physical, or a biological method or by being produced from a cell previously modified by a chemical, a physical, or a biological method. Specifically, the composition can be changed by a genetic engineering or by being produced from a cell previously modified by genetic engineering. In some embodiments, an external surface-engineered exosome comprises an exogenous protein (i.e., a protein that the EV, e.g., exosome does not naturally express) or a fragment or variant thereof that can be exposed on the external surface of the EV, e.g., exosome or can be an anchoring point (attachment) for a moiety exposed on the outer layer of the EV, e.g., exosome. In other embodiments, an external surface-engineered EV, e.g., exosome, comprises a higher expression of a natural exosome protein (e.g., any EV protein described herein) or a fragment or variant thereof that can be exposed to the external surface of the exosome or can be an anchoring point (attachment) for a moiety presented on the external surface of the exosome.

The term “modified,” when used in the context of scaffold proteins, described herein, refers to an alteration or engineering of a protein, e.g., an EV protein, such that the modified protein, e.g., the scaffold protein, is different from the naturally-occurring protein, e.g., the EV protein. In some embodiments, a modified protein, e.g., a scaffold protein, described herein comprises an amino acid sequence that is different from the amino acid sequence of the naturally-occurring protein, e.g., EV protein. In some embodiments, the modified protein, e.g., the scaffold protein, comprises a deletion of one or more amino acids relative to the naturally-occurring protein, e.g., EV protein. In some embodiments, the modified protein, e.g., the scaffold protein, is a fusion protein comprising an EV protein (or a fragment thereof) and a second peptide sequence. In some embodiments, the modified protein, e.g., the scaffold protein, retains one or more functions of the unmodified protein, e.g., the EV protein. In some embodiments, the scaffold protein retains only the ability of unmodified protein, e.g., the EV protein, to associate with the luminal or external surface of the EV membrane, e.g., the luminal or external surface of the exosome.

As used herein, the term “altered properties,” when used in the context of an EV, e.g., an exosome, and/or an AAV, described herein, refers to a change in the physical and/or functional properties of the EV, e.g., exosome, and/or AAV relative to an unmodified EV, e.g., exosome, and/or AAV. In some embodiments, the altered property comprises a better therapeutic effect. For example, in some embodiments, the AAV of the present disclosure have higher infectivity, higher activity, greater potency, faster transduction kinetics, and/or reduced immunogenicity (e.g., increased tolerance against immune invasion) than an unmodified AAV, e.g., AAV that is not present in the lumen of an EV disclosed herein. In some embodiments, the AAV of the present disclosure are less susceptible to immune response that an unmodified AAV, e.g., AAV that is not present in the lumen of an EV disclosed herein. In some embodiments, the AAV of the present disclosure are less likely to induce an immune response in a subject as compared to an unmodified AAV, e.g., AAV that is not present in the lumen of an EV disclosed herein. In some embodiments, the AAV of the present disclosure allow for multiple dosing of a subject, wherein the infectivity and/or activity of the AAV is retained after the first dose. In some embodiments, the AAV of the present disclosure allow for dose escalation studies without loss of AAV infectivity and/or activity.

As used herein, the term “fragment” of a protein (e.g., scaffold protein or therapeutic protein) refers to an amino acid sequence of a protein that is shorter than the naturally-occurring sequence, N- and/or C-terminally deleted or any part of the protein deleted in comparison to the naturally occurring protein. As used herein, the term “functional fragment” refers to a protein fragment that retains protein function. Accordingly, in some embodiments, a scaffold protein that comprises a functional fragment of an EV protein retains the ability to anchor a moiety on the luminal or external surface of an EV, e.g., exosome. Whether a fragment is a functional fragment can be assessed by methods known in the art to determine the protein content of EVs, e.g., exosomes, including Western Blots, FACS analysis and fusions of the fragments with autofluorescent proteins like, e.g., GFP. In certain embodiments, a scaffold protein comprising a functional fragment of an EV protein retains at least about 50%, at least about 60%, at least about 70%, at least about 80%, at least about 90% or at least about 100% of the ability, e.g., an ability to anchor a moiety, of the naturally occurring EV protein. A functional fragment does not necessarily retain every function of the full-length protein. Rather, in some embodiments, a fragment is a functional fragment if it retains the ability to anchor a moiety, of the naturally occurring EV protein, even if the fragment no longer retains any other function of the full-length protein.

As used herein, the term “variant” of a molecule (e.g., scaffold protein or a therapeutic protein) refers to a molecule that shares certain structural and functional identities with another molecule upon comparison by a method known in the art. For example, a variant of a protein can include a substitution, insertion, deletion, frameshift, or rearrangement in another protein.

In some embodiments, a variant of a scaffold protein comprises a variant having at least about 70% identity to the full-length, mature PTGFRN, BSG, IGSF2, IGSF3, IGSF8, ITGB1, ITGA4, SLC3A2, ATP1A1, ATP1A2, ATP1A3, ATP1A4, ATP1B3, ATP2B1, ATP2B2, ATP2B3, ATP2B4, CD13, ANPEP, MME, ENPP1, NRP1, CD9, CD63, CD81, PDGFR, GPI anchor proteins, lactadherin, LAMP2, LAMP2B, MARCKS, MARCKSL1, BASP1, or a fragment (e.g., functional fragment) of the PTGFRN, BSG, IGSF2, IGSF3, IGSF8, ITGB1, ITGA4, SLC3A2, ATP1A1, ATP1A2, ATP1A3, ATP1A4, ATP1B3, ATP2B1, ATP2B2, ATP2B3, ATP2B4, CD13, ANPEP, MME, ENPP1, NRP1, CD9, CD63, CD81, PDGFR, GPI anchor proteins, lactadherin, LAMP2, LAMP2B, MARCKS, MARCKSL1, or BASP1 proteins.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another embodiment, a string of amino acids can be conservatively replaced with a structurally similar string that differs in order and/or composition of side chain family members.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of programs available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

The polynucleotide variants can contain alterations in the coding regions, non-coding regions, or both. In one embodiment, the polynucleotide variants contain alterations which produce silent substitutions, additions, or deletions, but do not alter the properties or activities of the encoded polypeptide. In another embodiment, nucleotide variants are produced by silent substitutions due to the degeneracy of the genetic code. In other embodiments, variants in which 5-10, 1-5, or 1-2 amino acids are substituted, deleted, or added in any combination. Polynucleotide variants can be produced for a variety of reasons, e.g., to optimize codon expression for a particular host (change codons in the human mRNA to others, e.g., a bacterial host such as E. coli).

Naturally occurring variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the present disclosure. Alternatively, non-naturally occurring variants can be produced by mutagenesis techniques or by direct synthesis.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268: 2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J. Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, polypeptide variants include, e.g., modified polypeptides. Modifications include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination. In some embodiments, the scaffold protein is modified at any convenient location. In certain embodiments, the N-terminus of the scaffold protein is myristoylated.

The terms “associated with,” “linked to,” or “conjugated to” are used interchangeably herein to refer to a direct or indirect interaction between two or more elements. Two elements can be associated with each other by a covalent bond or a non-covalent bond and/or interaction. In some embodiments, a first element, e.g., an AAV, is associated with a second element, e.g., a scaffold protein, by a peptide bond. In some embodiments, a first element, e.g., an AAV, is associated with a second element, e.g., a scaffold protein, by one or more disulfide bonds. In some embodiments, a first element, e.g., an AAV, is associated with a second element, e.g., a scaffold protein, by a non-covalent interaction, e.g., an electrostatic interaction, a hydrogen bond, a van der Waals interaction, a hydrophobic interaction, an ion induced dipole, a dipole induced dipole, an ionic bond, a coordination bond, a chelation, or any combination thereof. The first element and the second element can be associated directly, e.g., wherein a scaffold protein is linked to an AAV capsid protein by a peptide bond, without any intervening amino acids that are not present part of the scaffold protein sequence (or conservative modifications thereof) or the AAV capsid protein (or conservative modifications thereof); or the first element can be associated with the second element through an indirect association, e.g., wherein the AAV is associated with the luminal membrane of an EV through the interaction of a scaffold protein, wherein the N-terminus of the scaffold protein interacts with the luminal membrane of the EV and the C-terminus of the scaffold protein is covalently linked a AAV capsid protein. A first element is “indirectly linked” to a second element where a linker of at least one amino acid is positioned between the first element and the second element. In some aspects, the first element and the second element are associated directly, e.g., wherein a scaffold protein is associated with an AAV by a peptide bond between the scaffold protein and an AAV capsid protein; or the first element is associated with the second element through an indirect association, e.g., wherein the AAV is associated with the external surface of an EV by way of a scaffold protein, wherein the scaffold protein is anchored to the external surface of the EV and the C-terminus or N-terminus of the scaffold protein is covalently linked a AAV capsid protein.

The term “encapsulated,” or grammatically different forms of the term (e.g., encapsulation, or encapsulating) refers to a status or process of having a first moiety (e.g., AAV) inside a second moiety (e.g., an EV, e.g., exosome) without chemically or physically linking the two moieties. In some embodiments, the term “encapsulated” can be used interchangeably with “in the lumen of” Non-limiting examples of encapsulating a first moiety (e.g., AAV) into a second moiety (e.g., EVs, e.g., exosomes) are disclosed elsewhere herein. In some embodiments of the present disclosure, the EV comprises a first AAV associated with the external surface of the EV and a second AAV encapsulated by the EV. In some embodiments of the present disclosure, the EV comprises a first AAV associated with the external surface of the EV, a second AAV encapsulated by the EV, and a targeting moiety associated with the external surface of the EV. In some embodiments of the present disclosure, the EV comprises an AAV associated with the external surface of the EV and a targeting moiety associated with the external surface of the EV. In some embodiments of the present disclosure, the EV comprises an AAV associated with the luminal surface of the EV and a targeting moiety associated with the external surface of the EV.

As used herein, the term “producer cell” refers to a cell used for generating an EV, e.g., exosome, and/or an AAV. A producer cell can be a cell cultured in vitro, or a cell in vivo. A producer cell includes, but not limited to, a cell known to be effective in generating EVs, e.g., exosomes, and/or AAV e.g., HEK293 cells, Chinese hamster ovary (CHO) cells, mesenchymal stem cells (MSCs), BJ human foreskin fibroblast cells, fHDF fibroblast cells, AGE.HN® neuronal precursor cells, CAP® amniocyte cells, adipose mesenchymal stem cells, RPTEC/TERT1 cells, sf9 insect cells, baby Hamster Kidney cells (BHK), PER.C6 cells, Vero cells, NS0 cells, HeLa cells. In some embodiments, the producer cell used to generate the EV is the same cell that is used to generate the AAV. In some embodiments, the EV is generated using a first producer cell, and the AAV is generated using a second producer cell, wherein the first producer cell is a different type of cell than the second producer cell. In some embodiments, the EV is generated using a first producer cell, and the AAV is generated using a second producer cell, wherein the first producer cell is of the same of cell as the second producer cell.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired EV preparation. In some embodiments, isolating or purifying as used herein is the process of removing, partially removing (e.g., a fraction) of the EVs from a sample containing producer cells. In some embodiments, an isolated EV composition has no detectable undesired activity or, alternatively, the level or amount of the undesired activity is at or below an acceptable level or amount. In other embodiments, an isolated EV composition has an amount and/or concentration of desired EVs at or above an acceptable amount and/or concentration. In other embodiments, the isolated EV composition is enriched as compared to the starting material (e.g., producer cell preparations) from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, or at least about 99.9999% as compared to the starting material. In some embodiments, isolated EV preparations are substantially free of residual biological products. In some embodiments, the isolated EV preparations are about 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites. Substantially free of residual biological products can also mean that the EV composition contains no detectable producer cells and that only EVs are detectable.

As used herein, the term “payload” refers to an agent that acts on a target (e.g., a target cell) that is contacted with the EV. A non-limiting example of a payload that can be included on the EV, e.g., exosome, is an AAV. Payloads that can be introduced into an EV or on the external surface of an EV, e.g., exosome, and/or a producer cell include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, and siRNA), amino acids (e.g., amino acids comprising a detectable moiety or a toxin or that disrupt translation), polypeptides (e.g., enzymes), lipids, carbohydrates, and small molecules (e.g., small molecule drugs and toxins). In certain embodiments, a payload comprises an AAV.

As used herein an “affinity agent” refers to a moiety that is capable of binding a second moiety. In some embodiments, the affinity agent is an antibody or an antigen-binding fragment thereof. In some embodiments, the affinity agent is a receptor, e.g., an AAV receptor. In some aspects, an expression cassette encoding the AAV affinity agent is transiently transfected into a target cell together with the AAV producing plasmids. In some aspects, AAV is produced

As used herein, the term “antibody” encompasses an immunoglobulin whether natural or partly or wholly synthetically produced, and fragments thereof. The term also covers any protein having a binding domain that is homologous to an immunoglobulin binding domain, e.g., an antigen-binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, camelid antibodies, shark IgNAR, anti-idiotype antibodies, antibody fragments, such as, e.g., scFv, (scFv)₂, Fab, Fab′, F(ab′)2, F(ab1)₂, Fv, dAb, single chain Fab, and Fd fragments, diabodies, minibodies, and antibody-related polypeptides. Antibody includes bispecific antibodies and multispecific antibodies so long as they exhibit the desired biological activity or function. In some aspects, the antibody or the antigen-binding fragment thereof is a nanobody.

The terms “individual,” “subject,” “host,” and “patient,” are used interchangeably herein and refer to any mammalian subject for whom diagnosis, treatment, or therapy is desired, particularly humans. The compositions and methods described herein are applicable to both human therapy and veterinary applications. In some embodiments, the subject is a mammal, and in other embodiments the subject is a human. As used herein, a “mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

As used herein, the term “substantially free” means that the sample comprising EVs, e.g., exosomes, comprise less than about 10% of macromolecules by mass/volume (m/v) percentage concentration. Some fractions can contain less than about 0.001%, less than about 0.01%, less than about 0.05%, less than about 0.1%, less than about 0.2%, less than about 0.3%, less than about 0.4%, less than about 0.5%, less than about 0.6%, less than about 0.7%, less than about 0.8%, less than about 0.9%, less than about 1%, less than about 2%, less than about 3%, less than about 4%, less than about 5%, less than about 6%, less than about 7%, less than about 8%, less than about 9%, or less than about 10% (m/v) of macromolecules.

As used herein, the term “macromolecule” means nucleic acids, contaminant proteins, lipids, carbohydrates, metabolites, or a combination thereof.

As used herein, the term “adeno-associated virus” or “AAV” includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers). AAV refers to a Dependoparvovirus (genus) within the Parvoviridae family of viruses. For example, the AAV can be an AAV derived from a naturally occurring “wild-type” virus, an AAV derived from a recombinant AAV (rAAV) genome packaged into a capsid derived from capsid proteins encoded by a naturally occurring cap gene and/or a rAAV genome packaged into a capsid derived from capsid proteins encoded by a non-natural capsid cap gene. As used herein, “AAV” can be used to refer to the virus itself or derivatives thereof. The term covers all subtypes and both naturally occurring and recombinant forms, except where specifically indicated otherwise. AAV includes AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), avian AAV, bovine AAV, canine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals, etc. See, e.g., Fields et al., VIROLOGY, volume 2, chapter 69 (3 d ed., Lippincott-Raven Publishers). In some aspects, the AAV is a non-replicating AAV, e.g., a non-infectious AAV. In some embodiments, the AAV comprises a viral vector.

In some aspects, the disclosure provides “isolated AAVs.” As used herein with respect to AAVs, the term “isolated” refers to an AAV that has been isolated from its natural environment (e.g., from a host cell, tissue, or subject) or artificially produced. Isolated AAVs can be produced using recombinant methods. Such AAVs are sometimes referred to herein as “recombinant AAVs” or “rAAVs.” In some embodiments, a recombinant AAV has an AAV genome in which part or all of the rep and cap genes have been replaced with heterologous sequences. An “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two AAV inverted terminal repeat sequences (ITRs). The term rAAV vector encompasses both rAAV vector particles and rAAV vector plasmids. Recombinant AAVs preferably have tissue-specific targeting capabilities, such that a transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected.

A “capsid-free” or “capsid-less” (or variations thereof) vector or nucleic acid molecule refers to a vector construct free from a capsid. In some embodiments, the capsid-less vector or nucleic acid molecule does not contain sequences encoding, e.g., an AAV Rep protein.

An “AAV virus” or “AAV viral particle” or “rAAV vector particle” refers to a viral particle composed of at least one AAV capsid protein (typically by all of the capsid proteins of a wild-type AAV) and an encapsidated polynucleotide rAAV vector. If the virus or particle comprises a heterologous polynucleotide (i.e. a polynucleotide other than a wild-type AAV genome such as a transgene to be delivered to a mammalian cell), it can be referred to as an “rAAV vector particle.” Thus, production of an rAAV particle necessarily includes the production of an rAAV vector, as such a vector is contained within an rAAV particle.

A “helper virus” for AAV refers to a virus that allows AAV (e.g., wild-type AAV) to be replicated and packaged by a mammalian cell. A variety of such helper viruses for AAV are known in the art, including adenoviruses, herpesviruses, baculoviruses, and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

As used herein, an “inverted terminal repeat” (or “ITR”) refers to a nucleic acid subsequence located at either the 5′ or 3′ end of a single stranded nucleic acid sequence, which comprises a set of nucleotides (initial sequence) followed downstream by its reverse complement, i.e., palindromic sequence. The intervening sequence of nucleotides between the initial sequence and the reverse complement can be any length.

The term “tropism” as used herein refers to the ability a first component to target a second component. In some aspects, tropism refers to the ability of an AAV vector or virion to transduce one or more specified cell types, but can also encompass how the vector functions to transduce the cell in the one or more specified cell types; i.e., tropism refers to preferential entry of the AAV vector or virion into certain cell or tissue type(s) and/or preferential interaction with the cell surface that facilitates entry into certain cell or tissue types, optionally and preferably followed by expression (e.g., transcription and, optionally, translation) of sequences carried by the AAV vector or virion in the cell, e.g., for a recombinant virus, expression of the heterologous nucleotide sequence(s). As used herein, the term “transduction” refers to the ability of an AAV vector or virion to infect one or more particular cell types; i.e., transduction refers to entry of the AAV vector or virion into the cell and the transfer of genetic material contained within the AAV vector or virion into the cell to obtain expression from the vector genome. In some cases, but not all cases, transduction and tropism can correlate.

In some aspects, the ability of an EV to have enhanced uptake by a particular cell, tissue, or organ can be modified by engineering a targeting moiety to be expressed on the EV. As used herein, the term a “targeting moiety” refers to an agent (i.e., payload) that can modify the distribution of extracellular vesicles (e.g., exosomes, nanovesicles) in vivo or in vitro. In some aspects, the targeting moiety, when expressed on an EV (e.g., exosome) alters and/or enhances the natural movement of the EV. The targeting moiety can be a biological molecule, such as a protein, a peptide, a lipid, or a carbohydrate, or a synthetic molecule. For example, the targeting moiety can be an affinity ligand (e.g., antibody, VHH domain, phage display peptide, fibronectin domain, camelid, VNAR), a synthetic polymer (e.g., PEG), a natural ligand/molecule (e.g., CD40L, albumin, CD47, CD24, CD55, CD59), a recombinant protein (e.g., XTEN), but not limited thereto. Non-limiting examples of targeting moieties that can be used with the present disclosure include those that can bind to a marker expressed specifically on a dendritic cell (e.g., Clec9A or DEC205) or T cells (e.g., CD3).

In certain aspects, the targeting moiety is displayed on the surface of EVs (e.g., exosomes). The targeting moiety can be displayed on the EV surface by being fused to a scaffold protein (e.g., Scaffold X) (e.g., as a genetically encoded fusion molecule). In some aspects, the targeting moiety can be displayed on the EV surface by chemical reaction attaching the bio-targeting moiety to an EV surface molecule. Non-limiting examples of targeting moieties that can be used with the present disclosure include a C-type lectin domain family 9 member A (Clec9a) protein, a dendritic cell-specific intercellular adhesion molecule-3-grabbing non-integrin (DC-SIGN), CD207, CD40, Clec6, dendritic cell immunoreceptor (DCIR), DEC-205, lectin-like oxidized low-density lipoprotein receptor-1 (LOX-1), MARCO, Clec12a, DC-asialoglycoprotein receptor (DC-ASGPR), DC immunoreceptor 2 (DCIR2), Dectin-1, macrophage mannose receptor (MMR), BDCA-1 (CD303, Clec4c), Dectin-2, Bst-2 (CD317), CD3, or any combination thereof. In certain aspects, the targeting moiety is Clec9a protein. In some aspects, the targeting moiety is a CD3 molecule.

As used herein, the term “C-type lectin domain family 9 member A” (Clec9a) protein refers to a group V C-type lectin-like receptor (CTLR) that functions as an activation receptor and is expressed on myeloid lineage cells (e.g., DCs). Huysamen et al., J Biol Chem 283(24):16693-701 (2008); U.S. Pat. No. 9,988,431 B2, each of which is herein incorporated by reference in its entirety. Synonyms of Clec9a are known and include CD370, DNGR-1, 5B5, HEEE9341, and C-type lectin domain containing 9A. In some aspects, Clec9a protein is expressed on human cDC1 cells. In some aspects, Clec9a protein is expressed on mouse cDC1 and pDC cells. Unless indicated otherwise, Clec9a, as used herein, can refer to Clec9a from one or more species (e.g., humans, non-human primates, dogs, cats, guinea pigs, rabbits, rats, mice, horses, cattle, and bears).

“Administering,” as used herein, means to give a composition comprising an EV, e.g., exosome, disclosed herein to a subject via a pharmaceutically acceptable route. Routes of administration can be intravenous, e.g., intravenous injection and intravenous infusion. Additional routes of administration include, e.g., subcutaneous, intramuscular, oral, nasal, and pulmonary administration. EVs, e.g., exosomes can be administered as part of a pharmaceutical composition comprising at least one excipient.

An “immune response,” as used herein, refers to a biological response within a vertebrate against foreign or abnormal agents, e.g., AAV, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4+ cell, a CD8+ T cell, or a Treg cell, or activation or inhibition of any other cell of the immune system, e.g., NK cell. Accordingly an immune response can comprise a humoral immune response (e.g., mediated by B-cells), cellular immune response (e.g., mediated by T cells), or both humoral and cellular immune responses. In some embodiments, an immune response is an “inhibitory” immune response. An inhibitory immune response is an immune response that blocks or diminishes the effects of a stimulus (e.g., an AAV therapy). In certain embodiments, the inhibitory immune response comprises the production of inhibitory antibodies against the AAV.

As used herein, the term “therapeutic protein” refers to any polypeptide known in the art that can be administered to a subject. In some embodiments, the therapeutic protein comprises a protein selected from a clotting factor, a growth factor, an antioxidant, an enzyme, a tumor suppressor gene, a DNA repair protein, a structural protein, an antibody, a functional fragment thereof, or a combination thereof. As used herein, the term “clotting factor,” refers to molecules, or analogs thereof, naturally occurring or recombinantly produced which prevent or decrease the duration of a bleeding episode in a subject. In other words, it means molecules having pro-clotting activity, i.e., are responsible for the conversion of fibrinogen into a mesh of insoluble fibrin causing the blood to coagulate or clot. “Clotting factor” as used herein includes an activated clotting factor, its zymogen, or an activatable clotting factor. An “activatable clotting factor” is a clotting factor in an inactive form (e.g., in its zymogen form) that is capable of being converted to an active form. The term “clotting factor” includes but is not limited to factor I (FI), factor II (FII), factor V (FV), FVII, FVIII, FIX, factor X (FX), factor XI (FXI), factor XII (FXII), factor XIII (FXIII), Von Willebrand factor (VWF), prekallikrein, high-molecular weight kininogen, fibronectin, antithrombin III, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI-1), plasminogen activator inhibitor-2 (PAI2), zymogens thereof, activated forms thereof, or any combination thereof.

Clotting activity, as used herein, means the ability to participate in a cascade of biochemical reactions that culminates in the formation of a fibrin clot and/or reduces the severity, duration or frequency of hemorrhage or bleeding episode.

A “growth factor,” as used herein, includes any growth factor known in the art including cytokines and hormones. In some embodiments, the growth factor is selected from adrenomedullin (AM), angiopoietin (Ang), autocrine motility factor, a bone morphogenetic protein (BMP) (e.g. BMP2, BMP4, BMP5, BMP7), a ciliary neurotrophic factor family member (e.g., ciliary neurotrophic factor (CNTF), leukemia inhibitory factor (LIF), interleukin-6 (IL-6)), a colony-stimulating factor (e.g., macrophage colony-stimulating factor (m-CSF), granulocyte colony-stimulating factor (G-CSF), granulocyte macrophage colony-stimulating factor (GM-CSF)), an epidermal growth factor (EGF), an ephrin (e.g., ephrin A1, ephrin A2, ephrin A3, ephrin A4, ephrin A5, ephrin B1, ephrin B2, ephrin B3), erythropoietin (EPO), a fibroblast growth factor (FGF) (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23), foetal bovine somatotrophin (FBS), a GDNF family member (e.g., glial cell line-derived neurotrophic factor (GDNF), neurturin, persephin, artemin), growth differentiation factor-9 (GDF9), hepatocyte growth factor (HGF), hepatoma-derived growth factor (HDGF), insulin, an insulin-like growth factors (e.g., insulin-like growth factor-1 (IGF-1) or IGF-2, an interleukin (IL) (e.g., IL-1, IL-2, IL-3, IL-4, IL-5, IL-6, IL-7), keratinocyte growth factor (KGF), migration-stimulating factor (MSF), macrophage-stimulating protein (MSP or hepatocyte growth factor-like protein (HGFLP)), myostatin (GDF-8), a neuregulin (e.g., neuregulin 1 (NRG1), NRG2, NRG3, NRG4), a neurotrophin (e.g., brain-derived neurotrophic factor (BDNF), nerve growth factor (NGF), a neurotrophin-3 (NT-3), NT-4, placental growth factor (PGF), platelet-derived growth factor (PDGF), renalase (RNLS), T-cell growth factor (TCGF), thrombopoietin (TPO), a transforming growth factor (e.g., transforming growth factor alpha (TGF-α), TGF-β, tumor necrosis factor-alpha (TNF-α), and vascular endothelial growth factor (VEGF).

In some embodiments, the therapeutic protein is encoded by a gene selected from dystrophin X-linked, MTM1 (myotubularin), tyrosine hydroxylase, AADC, cyclohydrolase, SMN1, FXN (frataxin), GUCY2D, RS1, CFH, HTRA, ARMS, CFB/CC2, CNGA/CNGB, Prf65, ARSA, PSAP, IDUA (MPS I), IDS (MPS II), PAH, GAA (acid alpha-glucosidase), low density lipoprotein receptor, cystic fibrosis transmembrane conductance regulator, GBA, MECP2. SCN1A, UBE3A, DMPK. FMR1, GJB1, CaMK2, HTT, ATX3, PMP22, CAPN3, DYSF, SGCA, SGCB, SGCG, SGCD, TNN, and AN05, or any combination thereof. In some aspects, the therapeutic protein is encoded by the human adenosine deaminase gene. In some aspects, the therapeutic protein is encoded by the human A1AT gene. In some aspects, the therapeutic protein is encoded by the human Hemoglobin (β-chain) gene. In some aspects, the therapeutic protein is encoded by the human p53 gene. In some aspects, the therapeutic protein is encoded by the human ABCD1 gene. In some aspects, the therapeutic protein is encoded by the human CHM gene. In some aspects, the therapeutic protein is encoded by the human Adenyl cyclase 6 gene. In some aspects, the therapeutic protein is encoded by the human CTFR gene. In some aspects, the therapeutic protein is encoded by the human Dystrophin gene. In some aspects, the therapeutic protein is encoded by the human alpha-galactosidase A gene. In some aspects, the therapeutic protein is encoded by the human BDNF pathway gene. In some aspects, the therapeutic protein is encoded by the human cytosine deaminase gene. In some aspects, the therapeutic protein is encoded by the human Factor VIII gene. In some aspects, the therapeutic protein is encoded by the human Factor IX gene. In some aspects, the therapeutic protein is encoded by the human LDLR gene. In some aspects, the therapeutic protein is encoded by the human Huntingtin gene. In some aspects, the therapeutic protein is encoded by the human Lipoprotein lipase gene. In some aspects, the therapeutic protein is encoded by the human ND4 gene. In some aspects, the therapeutic protein is encoded by the human ARSA gene. In some aspects, the therapeutic protein is encoded by the human IDUA gene. In some aspects, the therapeutic protein is encoded by the human IDS gene. In some aspects, the therapeutic protein is encoded by the human SGSH gene. In some aspects, the therapeutic protein is encoded by the human AADC gene. In some aspects, the therapeutic protein is encoded by the human acid alpha-glucosidase gene. In some aspects, the therapeutic protein is encoded by the human Colagen C7 gene. In some aspects, the therapeutic protein is encoded by the human RPE65 gene. In some aspects, the therapeutic protein is encoded by the human SMN1 gene. In some aspects, the therapeutic protein is encoded by the human VEGF gene. In some aspects, the therapeutic protein is encoded by the human WAS gene. In some aspects, the therapeutic protein is encoded by the human MTM1 gene. In some aspects, the therapeutic protein is encoded by the human RPGR gene.

As used herein the terms “heterologous” or “exogenous” refer to such molecules that are not normally found in a given context, e.g., in a cell or in a polypeptide. For example, an exogenous or heterologous molecule can be introduced into a cell and are only present after manipulation of the cell, e.g., by transfection or other forms of genetic engineering or a heterologous amino acid sequence can be present in a protein in which it is not naturally found.

As used herein, the term “heterologous nucleotide sequence” refers to a nucleotide sequence that does not naturally occur with a given polynucleotide sequence. In one embodiment, the heterologous nucleotide sequence encodes a polypeptide capable of extending the half-life of the therapeutic protein, e.g., the clotting factor, e.g., FVIII. In another embodiment, the heterologous nucleotide sequence encodes a polypeptide that increases the hydrodynamic radius of the therapeutic protein, e.g., the clotting factor, e.g., FVIII. In other embodiments, the heterologous nucleotide sequence encodes a polypeptide that improves one or more pharmacokinetic properties of the therapeutic protein without significantly affecting its biological activity or function (e.g., a procoagulant activity). In some embodiments, the therapeutic protein is linked or connected to the polypeptide encoded by the heterologous nucleotide sequence by a linker. Non-limiting examples of polypeptide moieties encoded by heterologous nucleotide sequences include an immunoglobulin constant region or a portion thereof, albumin or a fragment thereof, an albumin-binding moiety, a transferrin, the PAS polypeptides of U.S. Pat Application No. 20100292130, a HAP sequence, transferrin or a fragment thereof, the C-terminal peptide (CTP) of the β subunit of human chorionic gonadotropin, albumin-binding small molecule, an XTEN sequence, FcRn binding moieties (e.g., complete Fc regions or portions thereof which bind to FcRn), single chain Fc regions (ScFc regions, e.g., as described in US 2008/0260738, WO 2008/012543, or WO 2008/1439545), polyglycine linkers, polyserine linkers, peptides and short polypeptides of 6-40 amino acids of two types of amino acids selected from glycine (G), alanine (A), serine (S), threonine (T), glutamate (E) and proline (P) with varying degrees of secondary structure from less than 50% to greater than 50%, amongst others, or two or more combinations thereof. In some embodiments, the polypeptide encoded by the heterologous nucleotide sequence is linked to a non-polypeptide moiety. Non-limiting examples of the non-polypeptide moieties include polyethylene glycol (PEG), albumin-binding small molecules, polysialic acid, hydroxyethyl starch (HES), a derivative thereof, or any combinations thereof.

As used herein, the term “Fc region” is defined as the portion of a polypeptide which corresponds to the Fc region of native Ig, i.e., as formed by the dimeric association of the respective Fc domains of its two heavy chains. A native Fc region forms a homodimer with another Fc region. In contrast, the term “genetically-fused Fc region” or “single-chain Fc region” (scFc region), as used herein, refers to a synthetic dimeric Fc region comprised of Fc domains genetically linked within a single polypeptide chain (i.e., encoded in a single contiguous genetic sequence).

In one embodiment, the “Fc region” refers to the portion of a single Ig heavy chain beginning in the hinge region just upstream of the papain cleavage site (i.e., residue 216 in IgG, taking the first residue of heavy chain constant region to be 114) and ending at the C-terminus of the antibody. Accordingly, a complete Fc domain comprises at least a hinge domain, a CH2 domain, and a CH3 domain.

The Fc region of an Ig constant region, depending on the Ig isotype can include the CH2, CH3, and CH4 domains, as well as the hinge region. Chimeric proteins comprising an Fc region of an Ig bestow several desirable properties on a chimeric protein including increased stability, increased serum half-life (see Capon et al., 1989, Nature 337:525) as well as binding to Fc receptors such as the neonatal Fc receptor (FcRn) (U.S. Pat. Nos. 6,086,875, 6,485,726, 6,030,613; WO 03/077834; US2003-0235536A1), which are incorporated herein by reference in their entireties.

“Treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof.

“Prevent” or “preventing,” as used herein, refers to decreasing or reducing the occurrence or severity of a particular outcome. In some embodiments, preventing an outcome is achieved through prophylactic treatment.

### II. Compositions of the Disclosure

Certain aspects of the present disclosure are directed to EVs, e.g., exosomes, comprising an AAV and a scaffold protein, wherein the AAV is within the lumen of the exosome. In some embodiments, the EV contains a number of AAV in the lumen that is higher than the number of AAV in the lumen of a reference EV that lacks the scaffold protein. In some aspects, the EV is more likely to take up an AAV than an EV that lacks the scaffold protein, e.g., in a mixed population of EV either comprising or not comprising the scaffold protein, a higher percentage of EV comprise the scaffold protein and an AAV in the lumen than EV that comprise only the AAV in the lumen.

II.A. Extracellular Vesicles (EVs)

EVs, e.g., exosomes, described herein are extracellular vesicles with a diameter between about 20-300 nm. In certain embodiments, an EV, e.g., exosome, of the present disclosure has a diameter between about 20 nm and about 290 nm, between about 20 nm and about 280 nm, between about 20 nm and about 270 nm, between about 20 nm and about 260 nm, 20 nm and about 250 nm, between about 20 nm and about 240 nm, between about 20 nm and about 230 nm, between about 20 nm and about 220 nm, between about 20 nm and about 210 nm, between about 20 nm and about 200 nm, between about 20 nm and about 190 nm, between about 20 nm and about 180 nm, between about 20 nm and about 170 nm, between about 20 nm and about 160 nm, between about 20 nm and about 150 nm, between about 20 nm and about 140 nm, between about 20 nm and about 130 nm, between about 20 nm and about 120 nm, between about 20 nm and about 110 nm, between about 20 nm and about 100 nm, between about 20 nm and about 90 nm, between about 20 nm and about 80 nm, between about 20 nm and about 70 nm, between about 20 nm and about 60 nm, between about 20 nm and about 50 nm, between about 20 nm and about 40 nm, between about 20 nm and about 30 nm, between about 30 nm and about 300 nm, between about 30 nm and about 290 nm, between about 30 nm and about 280 nm, between about 30 nm and about 270 nm, between about 30 nm and about 260 nm, between about 30 nm and about 250 nm, between about 30 nm and about 240 nm, between about 30 nm and about 230 nm, between about 30 nm and about 220 nm, between about 30 nm and about 210 nm, between about 30 nm and about 200 nm, between about 30 nm and about 190 nm, between about 30 nm and about 180 nm, between about 30 nm and about 170 nm, between about 30 nm and about 160 nm, between about 30 nm and about 150 nm, between about 30 nm and about 140 nm, between about 30 nm and about 130 nm, between about 30 nm and about 120 nm, between about 30 nm and about 110 nm, between about 30 nm and about 100 nm, between about 30 nm and about 90 nm, between about 30 nm and about 80 nm, between about 30 nm and about 70 nm, between about 30 nm and about 60 nm, between about 30 nm and about 50 nm, between about 30 nm and about 40 nm, between about 40 nm and about 300 nm, between about 40 nm and about 290 nm, between about 40 nm and about 280 nm, between about 40 nm and about 270 nm, between about 40 nm and about 260 nm, between about 40 nm and about 250 nm, between about 40 nm and about 240 nm, between about 40 nm and about 230 nm, between about 40 nm and about 220 nm, between about 40 nm and about 210 nm, between about 40 nm and about 200 nm, between about 40 nm and about 190 nm, between about 40 nm and about 180 nm, between about 40 nm and about 170 nm, between about 40 nm and about 160 nm, between about 40 nm and about 150 nm, between about 40 nm and about 140 nm, between about 40 nm and about 130 nm, between about 40 nm and about 120 nm, between about 40 nm and about 110 nm, between about 40 nm and about 100 nm, between about 40 nm and about 90 nm, between about 40 nm and about 80 nm, between about 40 nm and about 70 nm, between about 40 nm and about 60 nm, between about 40 nm and about 50 nm, between about 50 nm and about 300 nm, between about 50 nm and about 290 nm, between about 50 nm and about 280 nm, between about 50 nm and about 270 nm, between about 50 nm and about 260 nm, between about 50 nm and about 250 nm, between about 50 nm and about 240 nm, between about 50 nm and about 230 nm, between about 50 nm and about 220 nm, between about 50 nm and about 210 nm, between about 50 nm and about 200 nm, between about 50 nm and about 190 nm, between about 50 nm and about 180 nm, between about 50 nm and about 170 nm, between about 50 nm and about 160 nm, between about 50 nm and about 150 nm, between about 50 nm and about 140 nm, between about 50 nm and about 130 nm, between about 50 nm and about 120 nm, between about 50 nm and about 110 nm, between about 50 nm and about 100 nm, between about 50 nm and about 90 nm, between about 50 nm and about 80 nm, between about 50 nm and about 70 nm, between about 50 nm and about 60 nm, between about 60 nm and about 300 nm, between about 60 nm and about 290 nm, between about 60 nm and about 280 nm, between about 60 nm and about 270 nm, between about 60 nm and about 260 nm, between about 60 nm and about 250 nm, between about 60 nm and about 240 nm, between about 60 nm and about 230 nm, between about 60 nm and about 220 nm, between about 60 nm and about 210 nm, between about 60 nm and about 200 nm, between about 60 nm and about 190 nm, between about 60 nm and about 180 nm, between about 60 nm and about 170 nm, between about 60 nm and about 160 nm, between about 60 nm and about 150 nm, between about 60 nm and about 140 nm, between about 60 nm and about 130 nm, between about 60 nm and about 120 nm, between about 60 nm and about 110 nm, between about 60 nm and about 100 nm, between about 60 nm and about 90 nm, between about 60 nm and about 80 nm, between about 60 nm and about 70 nm, between about 70 nm and about 300 nm, between about 70 nm and about 290 nm, between about 70 nm and about 280 nm, between about 70 nm and about 270 nm, between about 70 nm and about 260 nm, between about 70 nm and about 250 nm, between about 70 nm and about 240 nm, between about 70 nm and about 230 nm, between about 70 nm and about 220 nm, between about 70 nm and about 210 nm, between about 70 nm and about 200 nm, between about 70 nm and about 190 nm, between about 70 nm and about 180 nm, between about 70 nm and about 170 nm, between about 70 nm and about 160 nm, between about 70 nm and about 150 nm, between about 70 nm and about 140 nm, between about 70 nm and about 130 nm, between about 70 nm and about 120 nm, between about 70 nm and about 110 nm, between about 70 nm and about 100 nm, between about 70 nm and about 90 nm, between about 70 nm and about 80 nm, between about 80 nm and about 300 nm, between about 80 nm and about 290 nm, between about 80 nm and about 280 nm, between about 80 nm and about 270 nm, between about 80 nm and about 260 nm, between about 80 nm and about 250 nm, between about 80 nm and about 240 nm, between about 80 nm and about 230 nm, between about 80 nm and about 220 nm, between about 80 nm and about 210 nm, between about 80 nm and about 200 nm, between about 80 nm and about 190 nm, between about 80 nm and about 180 nm, between about 80 nm and about 170 nm, between about 80 nm and about 160 nm, between about 80 nm and about 150 nm, between about 80 nm and about 140 nm, between about 80 nm and about 130 nm, between about 80 nm and about 120 nm, between about 80 nm and about 110 nm, between about 80 nm and about 100 nm, between about 80 nm and about 90 nm, between about 90 nm and about 300 nm, between about 90 nm and about 290 nm, between about 90 nm and about 280 nm, between about 90 nm and about 270 nm, between about 90 nm and about 260 nm, between about 90 nm and about 250 nm, between about 90 nm and about 240 nm, between about 90 nm and about 230 nm, between about 90 nm and about 220 nm, between about 90 nm and about 210 nm, between about 90 nm and about 200 nm, between about 90 nm and about 190 nm, between about 90 nm and about 180 nm, between about 90 nm and about 170 nm, between about 90 nm and about 160 nm, between about 90 nm and about 150 nm, between about 90 nm and about 140 nm, between about 90 nm and about 130 nm, between about 90 nm and about 120 nm, between about 90 nm and about 110 nm, between about 90 nm and about 100 nm, between about 100 nm and about 300 nm, between about 110 nm and about 290 nm, between about 120 nm and about 280 nm, between about 130 nm and about 270 nm, between about 140 nm and about 260 nm, between about 150 nm and about 250 nm, between about 160 nm and about 240 nm, between about between about 170 nm and about 230 nm, between about 180 nm and about 220 nm, or between about 190 nm and about 210 nm. The size of the EV, e.g., exosome, described herein can be measured according to methods described, infra.

EVs, e.g., exosomes, of the present disclosure comprise a membrane (“EV membrane”), comprising an external surface (e.g., an extracellular surface) and an internal surface (e.g., a luminal surface). In certain embodiments, the internal surface faces the inner core (i.e., lumen) of the EV, e.g., exosome. In certain embodiments, the external surface can be in contact with the endosome, the multivesicular bodies, or the membrane/cytoplasm of a producer cell.

In some embodiments, the EV, e.g., exosome, membrane comprises a bi-lipid membrane, e.g., a lipid bilayer. In some embodiments, the EV, e.g., exosome, membrane comprises lipids and fatty acids. In some embodiments, the EV, e.g., exosome, membrane comprises phospholipids, glycolipids, fatty acids, sphingolipids, phosphoglycerides, sterols, cholesterols, and phosphatidylserines.

In some embodiments, the EV, e.g., exosome, membrane comprises an inner leaflet and an outer leaflet. The composition of the inner and outer leaflet can be determined by transbilayer distribution assays known in the art, see, e.g., Kuypers et al., Biochem Biophys Acta 1985 819:170. In some embodiments, the composition of the outer leaflet is between approximately 70-90% choline phospholipids, between approximately 0-15% acidic phospholipids, and between approximately 5-30% phosphatidylethanolamine. In some embodiments, the composition of the inner leaflet is between approximately 15-40% choline phospholipids, between approximately 10-50% acidic phospholipids, and between approximately 30-60% phosphatidylethanolamine.

In some embodiments, the EV, e.g., exosome, membrane comprises one or more polysaccharides, such as glycan.

In some embodiments, the EV, e.g., exosome, comprises one or more multilamellar bodies within the lumen of the EV, e.g., exosome. In some embodiments, an AAV of the present disclosure is within a multilamellar body. In some embodiments, an AAV of the present disclosure is not within a multilamellar body.

In some aspects, the EV comprises a surface antigen that inhibits uptake of the EV by a macrophage. In some aspects, the surface antigen is associated with the exterior surface of the EV (e.g., exosome). In some aspects, the surface antigen is selected from CD47, CD24, a fragment thereof, and any combination thereof. In certain aspects, the surface antigen comprises CD47, e.g., human CD47 (UniProtKB—Q08722). In some aspects, the surface antigen comprises a fragment of CD47, e.g., human CD47. In certain aspects, the surface antigen comprises CD24, e.g., human CD24. In some aspects, the surface antigen comprises a fragment of CD24, e.g., human CD24.

II.A.1. Targeting Moieties

In some aspects, the EV, e.g., exosome, is further modified to display an additional protein (or fragment thereof) that can help direct EV uptake (e.g., targeting moiety). In certain aspects, the EV, e.g., exosome, disclosed herein further comprises a targeting moiety that can modify the distribution of the EVs in vivo or in vitro. In some aspects, the targeting moiety can be a biological molecule, such as a protein, a peptide, a lipid, or a synthetic molecule.

In some aspects, a targeting moiety of the present disclosure specifically binds to a marker for a dendritic cell. In certain aspects, the marker is expressed only on dendritic cells. In some aspects, dendritic cells comprise a progenitor (Pre) dendritic cells, inflammatory mono dendritic cells, plasmacytoid dendritic cell (pDC), a myeloid/conventional dendritic cell 1 (cDC1), a myeloid/conventional dendritic cell 2 (cDC2), inflammatory monocyte derived dendritic cells, Langerhans cells, dermal dendritic cells, lysozyme-expressing dendritic cells (LysoDCs), Kupffer cells, nonclassical monocytes, or any combination thereof. Markers that are expressed on these dendritic cells are known in the art. See, e.g., Collin et al., Immunology 154(1):3-20 (2018). In some aspects, the targeting moiety is a protein, wherein the protein is an antibody or a fragment thereof that can specifically bind to a marker selected from DEC205, CLEC9A, CLEC6, DCIR, DC-SIGN, LOX-1, MARCO, Clec12a, Clec10a, DC-asialoglycoprotein receptor (DC-ASGPR), DC immunoreceptor 2 (DCIR2), Dectin-1, macrophage mannose receptor (MMR), BDCA-2 (CD303, Clec4c), Dectin-2, Bst-2 (CD317), Langerin, CD206, CD11b, CD11c, CD123, CD304, XCR1, AXL, Siglec 6, CD209, SIRPA, CX3CR1, GPR182, CD14, CD16, CD32, CD34, CD38, CD10, or any combination thereof. In some aspects, a marker useful for the present disclosure comprises a C-type lectin like domain. In certain aspects, a marker is Clec9a and the dendritic cell is cDC1.

In some aspects, a targeting moiety disclosed herein can bind to both human and mouse Clec9a, including any variants thereof. In some aspects, a targeting moiety of the present disclosure can bind to Clec9a from other species, including but not limited to chimpanzee, rhesus monkey, dog, cow, horse, or rat. Sequences for such Clec9a protein are known in the art. See, e.g., U.S. Pat. No. 8,426,565 B2, which is herein incorporated by reference in its entirety.

In some aspects, a targeting moiety of the present disclosure specifically binds to a marker for a T cell. In certain aspects, the T cell is a CD4+ T cell. In some aspects, the T cell is a CD8+ T cell.

In some aspects, a targeting moiety of the present disclosure specifically binds to a marker on a muscle cell. In some aspects, the muscle cell is a smooth muscle cell. In some aspects, the muscle cell is a skeletal muscle cell. In some aspects, the muscle cell is a cardiac muscle cell. In some aspects, the marker on the muscle cell is selected from alpha-smooth muscle actin, VE-cadherin, caldesmon/CALD1, calponin 1, hexim 1, histamine H2 R; motilin R/GPR38, transgelin/TAGLN, and any combination thereof. In some aspects, the marker on the muscle cell is selected from alpha-sarcoglycan, beta-sarcoglycan, calpain inhibitors, creatine kinase MM/CKMM, eIF5A, enolase 2/neuron-specific enolase, epsilon-sarcoglycan, FABP3/H-FABP, GDF-8/Myostatin, GDF-11/GDF-8, integrin alpha 7, integrin alpha 7 beta 1, integrin beta 1/CD29, MCAM/CD146, MyoD, myogenin, myosin light chain kinase inhibitors, NCAM-1/CD56, troponin I, and any combination thereof. In some aspects, the marker on the muscle cell is myosin heavy chain, myosin light chain, or a combination thereof.

In some aspects, the targeting moiety of the present disclosure specifically binds to a marker specific to a target tissue, such as the liver, brain, bladder, kidney, lung, or eye. In some aspects, the targeting moiety of the present disclosure specifically binds to a marker expressed on a tumor cell. In some aspects, the EV, e.g., the exosome, targets a tumor cell, dendritic cell, T cell, B cell, macrophage, monocyte, Schwann cell, neuron, hepatocyte, Kupffer cell, myeloid-lineage cell (e.g., a neutrophil, myeloid-derived suppressor cell (MDSC, e.g., a monocytic MDSC or a granulocytic MDSC), myocyte, monocyte, macrophage, hematopoietic stem cell, basophil, neutrophil, or eosinophil), or any combination thereof. In some aspects, the EV, e.g., the exosome, targets a myeloid-lineage cell. In some aspects, the EV, e.g., the exosome, targets a macrophage. In certain aspects, the EV, e.g., the exosome, targets the liver, heart, lungs, brain, kidneys, central nervous system, peripheral nervous system, muscle, bone, joint, skin, intestine, bladder, pancreas, lymph nodes, spleen, blood, bone marrow, or any combination thereof.

In some aspects, a targeting moiety disclosed herein binds to human CD3 protein or a fragment thereof. Sequences for human CD3 protein are known in the art.

In some aspects, a targeting moiety disclosed herein can bind to both human and mouse CD3, including any variants thereof. In some aspects, a targeting moiety of the present disclosure can bind to CD3 from other species, including but not limited to chimpanzee, rhesus monkey, dog, cow, horse, or rat. Sequences for such CD3 protein are also known in the art.

In some aspects, a targeting moiety disclosed herein can allow for greater uptake of an EV (e.g., exosome) by a cell expressing a marker specific for the targeting moiety (e.g., CD3: CD4+ T cell and/or CD8+ T cell; Clec9a: dendritic cells; or a muscle cell marker). In some aspects, the uptake of an EV is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 2,000-fold, at least about 3,000-fold, at least about 4,000-fold, at least about 5,000-fold, at least about 6,000-fold, at least about 7,000-fold, at least about 8,000-fold, at least about 9,000-fold, at least about 10,000-fold or more, compared to a reference (e.g., corresponding EV without the targeting moiety or a non-EV delivery vehicle). In some aspects, a reference comprises an EV (e.g., exosome) that does not express a targeting moiety disclosed herein.

A targeting moiety disclosed herein can comprise a peptide, an antibody or an antigen binding fragment thereof, a chemical compound, or any combination thereof.

In some aspects, the targeting moiety is a peptide that can specifically bind to Clec9a. See, e.g., Yan et al., Oncotarget 7(26): 40437-40450 (2016). For example, in certain aspects, the peptide comprises a soluble fragment of Clec9a. A non-limiting example of such a peptide is described in U.S. Pat. No. 9,988,431 B2, which is herein incorporated by reference in its entirety. In certain aspects, the peptide comprises a ligand (natural or synthetic) of Clec9a, such as those described in Ahrens et al., Immunity 36(4): 635-45 (2012); and Zhang et al., Immunity 36(4): 646-57 (2012). A non-limiting example of a peptide comprising a Clec9a ligand is described in International Publ. No. WO 2013/053008 A2, which is herein incorporated by reference in its entirety.

In some aspects, the targeting moiety is a peptide that can specifically bind to CD3. For example, in certain aspects, the peptide comprises a soluble fragment of CD3. In certain aspects, the peptide comprises a ligand (natural or synthetic) of CD3.

In some aspects, the targeting moiety is an antibody or an antigen binding fragment thereof. In certain aspects, a targeting moiety is a single-chain Fv antibody fragment. In certain aspects, a targeting moiety is a single-chain F(ab) antibody fragment. In certain aspects, a targeting moiety is a nanobody. In certain aspects, a targeting moiety is a monobody.

In some aspects, an EV (e.g., exosome) disclosed herein comprises one or more (e.g., 2, 3, 4, 5, or more) targeting moieties. In certain aspects, the one or more targeting moieties are expressed in combination with other exogenous biologically active molecules disclosed herein (e.g., therapeutic molecule, adjuvant, or immune modulator). In some aspects, the one or more targeting moieties can be expressed on the exterior surface of the EV, e.g., exosome. Accordingly, in certain aspects, the one or more targeting moieties are linked to a scaffold moiety (e.g., Scaffold X) on the exterior surface of the EV, e.g., exosome. When the one or more targeting moieties are expressed in combination with other exogenous biologically active molecules (e.g., therapeutic molecule, adjuvant, or immune modulator), the other exogenous biologically active molecules can be expressed on the surface (e.g., exterior surface or luminal surface) or in the lumen of the EV, e.g., exosome.

II.B. Adeno-Associated Virus (AAV)

Certain aspects of the present disclosure are directed to an EV, e.g., exosome, comprising an AAV, wherein the AAV is present in the lumen of the EV.

AAV is a non-enveloped, single-stranded DNA virus of the Parvoviridae family. In contrast to most other members of the Parvoviridae family, AAV is replication defective and is only able to replicate efficiently in the presence of a helper virus such as adenovirus or herpes virus.

AAV was first reported in the mid 1960's as a contaminant of viral preparations of adenovirus. See Atchison et al. Science 149(3685), 754-756 (1965). Since then, progressively safer and more effective methods to use AAV as a recombinant DNA vector have been developed. See, e.g., Hermonat and Muzyczka Proc Natl Acad Sci USA. 81(20), 6466-6470 (1984); Laughlin et al. Gene, 23(1), 65-73 (1983). Matsushita T., et al. Gene Ther. 5(7), 938-945 (1998); Xiao et al. Journal of Virology. 72(3) 2224-2232 (1998). It has been reported that low numbers of AAV genomes can integrate into the host chromosome (Cheung et al. J. Virol. 1980; 33:739-748). AAV is immunologically distinct from any known adenovirus antigen. The AAV capsid contains a single-stranded DNA (ssDNA) genome (Rose et al. Proc Natl Acad Sci USA 1969; 64:863-869.

AAV has a single stranded, 4.7 kb DNA genome encoding replication (rep) genes and a capsid (cap) genes flanked by two ITRs. It is predominantly non-integrating and forms stable episomes in non-dividing tissue. In spite of its high sero-prevalence in the adult human population, it has not been associated with any human disease. See Gonçalves, M. Virol. J. 2, 43 (2005). AAV's stable expression in tissues, its lack of pathogenicity, and its ease of high titer production have made it a very attractive and popular gene transfer platform.

A recombinant AAV is a genetically manipulated AAV in which part or all of the rep and cap genes have been replaced with heterologous sequences. Just as wild-type AAV, rAAV can trigger long-term transgene expression in postmitotic tissues, most likely because the rAAV's recombinant genome persists as largely circular episomes within the nucleus. rAAVs only cis-element required for the production of rAAVs is the AAV ITRs, whereas rep, cap, and adenoviral helper genes can be provided in trans. Thus, in some embodiments disclosed herein, rAAVs contain only the transgene DNA flanked by the ITRs, and this genome is encapsidated within a serotype-specific capsid.

AAV possesses unique features that make it attractive as a vector for delivering foreign DNA to cells. AAV infection of cells in culture has generally been noncytopathic, and natural infection of humans and other animals is silent and asymptomatic. Moreover, AAV infects many different types of mammalian cells allowing the possibility of targeting many different tissues in vivo. AAV also possess additional advantages that make it a particularly attractive viral system for gene delivery, including promotion of a milder immune response compared to other forms of gene delivery and persistent expression in both dividing and quiescent cells as a non-integrating vector. Also, AAV withstands the conditions used to inactivate adenovirus (56° to 65° C. for several hours), making cold preservation of rAAV-based vaccines less critical.

Helper virus is not required for AAV transduction and entry of the AAV genome into the target cell. Furthermore, because the signals directing AAV replication, genome encapsidation and integration are contained within the ITRs of the AAV genome, the internal approximately 4.7 kb of the genome (encoding replication and structural capsid proteins, rep-cap) can thus be replaced with foreign DNA such as a gene cassette containing a promoter, a DNA of interest and a polyadenylation signal, without loss of any functionality critical for AAV use as gene-therapeutic agent.

AAV vectors can include additional elements that function in cis or in trans. In particular embodiments, an AAV vector that includes a vector genome also has one or more ITR sequences that flank the 5′ or 3′ terminus of the donor sequence; an expression control element that drives transcription (e.g., a promoter or enhancer) of the donor sequence, such as a constitutive or regulatable control element, or tissue-specific expression control element; an intron sequence, a stuffer or filler polynucleotide sequence; and/or a poly-Adenine sequence located 3′ of the donor sequence.

In some embodiments, AAV replicates using a helper virus. A variety of such helper viruses for AAV are known in the art, including adenoviruses, herpesviruses, baculoviruses, and poxviruses such as vaccinia. Individual adenovirus types encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

During EV production, molecules present in the cytosol of the producing cell in the vicinity of the forming EV are naturally captured by the forming EV. As a result, a cell that is producing both EV and AAV naturally yields some EVs with at least one AAV in the lumen of the EVs. Certain aspects of the present disclosure are directed to EVs that have more AAVs in the lumen of the EVs than are naturally, e.g., passively, captured by a forming EV. In some embodiments, the number of AAVs in the lumen of the EV is higher than the number of AAV in the lumen of a reference EV. In some embodiments, the reference EV comprises AAV that was associated with the AAV through this natural process. The precise number of AAV that is naturally captured in the lumen of a forming EV, e.g., a reference EV lacking a scaffold protein, will vary. In some embodiments, the number of AAV present in the reference EV by this mechanism is about 1 AAV per EV, about 2 AAV per EV, about 3 AAV per EV, or about 4 AAV per EV. In some embodiments, the number of AAV present in the reference EV is less than about 7, less than about 6, less than about 5, less than about 4, less than about 3, or less than about 2 AAV per EV.

In some embodiments, the number of AAVs in the lumen EV of the present disclosure is at least about 2 fold, at least about 3 fold, at least about 4 fold, at least about 5 fold, at least about 6 fold, at least about 7 fold, at least about 8 fold, at least about 9 fold, or at least about 10 fold higher than the number of AAVs in the lumen of the reference EV. In some embodiments, the number of the AAV in the EV is about 2 fold to about 10 fold, about 3 fold to about 10 fold, about 4 fold to about 10 fold, about 5 fold to about 10 fold, about 2 fold to about 9 fold, about 2 fold to about 8 fold, about 2 fold to about 7 fold, about 2 fold to about 6 fold, about 2 fold to about 5 fold, about 2 fold to about 4 fold, about 2 fold to about 3 fold, about 3 fold to about 9 fold, about 3 fold to about 8 fold, about 3 fold to about 7 fold, about 3 fold to about 6 fold, about 3 fold to about 5 fold, about 3 fold to about 4 fold, about 4 fold to about 9 fold, about 4 fold to about 8 fold, about 4 fold to about 7 fold, about 4 fold to about 6 fold, about 4 fold to about 5 fold, about 5 fold to about 9 fold, about 5 fold to about 8 fold, about 5 fold to about 7 fold, or about 5 fold to about 6 fold higher than the number of AAVs in the lumen of the reference EV. In some embodiments, the number of the AAV in the EV of the present disclosure is at least about 2 fold higher than the number of AAVs in the lumen of the reference EV. In some embodiments, the number of the AAV in the EV of the present disclosure is at least about 3 fold higher than the number of AAVs in the lumen of the reference EV. In some embodiments, the number of the AAV in the EV of the present disclosure is at least about 4 fold higher than the number of AAVs in the lumen of the reference EV. In some embodiments, the EV and the reference EV are about the same size.

In some aspects, at least about 0.01% to about 100% of EVs, e.g., exosomes, comprise an AAV in the lumen of the exosome. In some aspects, at least about 0.1% to about 100%, at least about 1% to about 100%, at least about 5% to about 100%, at least about 10% to about 100%, at least about 15% to about 100%, at least about 20% to about 100% at least about 25% to about 100%, at least about 30% to about 100%, at least about 40% to about 100%, at least about 50% to about 100%, at least about 60% to about 100%, at least about 70% to about 100%, at least about 80% to about 100%, at least about 90% to about 100% of EVs, e.g., exosomes, comprise an AAV in the lumen of the exosome. In some aspects, at least about 0.1%, at least about 1%, at least about 5%, at least about 10%, at least about 15%, at least about 20% at least about 25%, at least about 30%, at least about 40%, at least about 50%, at least about 60%, at least about 70%, at least about 80%, at least about 90% of EVs, e.g., exosomes, comprise an AAV in the lumen of the exosome.

In some aspects, the percent of EVs, e.g., exosomes, comprising a scaffold moiety and at least one AAV molecule in the lumen of the EV, e.g., exosome, in a sample comprising more than one EV is increased relative to the percent of EVs, e.g., exosomes, comprising an AAV but lacking a scaffold moiety. In some aspects, at least about 0.01% to about 100% of EVs, e.g., exosomes, comprise an AAV in the lumen of the exosome and a scaffold moiety. In some aspects, at least about 0.1% to about 100%, at least about 1% to about 100%, at least about 5% to about 100%, at least about 10% to about 100%, at least about 15% to about 100%, at least about 20% to about 100% at least about 25% to about 100%, at least about 30% to about 100%, at least about 40% to about 100%, at least about 50% to about 100%, at least about 60% to about 100%, at least about 70% to about 100%, at least about 80% to about 100%, at least about 90% to about 100% of EVs, e.g., exosomes, comprise an AAV in the lumen of the exosome and a scaffold moiety. In some aspects, at least about 0.1%, at least about 1%, at least about 5%, at least about 10%, at least about 15%, at least about 20% at least about 25%, at least about 30%, at least about 40%, at least about 50%, at least about 60%, at least about 70%, at least about 80%, at least about 90% of EVs, e.g., exosomes, comprise an AAV in the lumen of the exosome and a scaffold moiety.

In some embodiments, the EV comprises at least about 2 AAVs, at least about 3 AAVs, at least about 4 AAVs, at least about 5 AAVs, at least about 6 AAVs, at least about 7 AAVs, at least about 8 AAVs, at least about 9 AAVs, at least about 10 AAVs, at least about 11 AAVs, at least about 12 AAVs, at least about 13 AAVs, at least about 14 AAVs, at least about 15 AAVs, at least about 16 AAVs, at least about 17 AAVs, at least about 18 AAVs, at least about 19 AAVs, at least about 20 AAVs, at least about 21 AAVs, at least about 22 AAVs, at least about 23 AAVs, at least about 24 AAVs, at least about 25 AAVs, at least about 26 AAVs, at least about 27 AAVs, at least about 28 AAVs, at least about 29 AAVs, at least about 30 AAVs, at least about 35 AAVs, at least about 40 AAVs, at least about 45 AAVs, at least about 50 AAVs, at least about 60 AAVs, at least about 70 AAVs, at least about 80 AAVs, at least about 90 AAVs, at least about 100 AAVs, at least about 150 AAVs, at least about 200 AAVs, at least about 250AAVs, at least about 300 AAVs, at least about 350 AAVs, at least about 400 AAVs, at least about 450 AAVS, or at least about 500 AAVs in the lumen of the EV. In some embodiments, the EV comprises at least about 600 AAVs, at least about 700 AAVs, at least about 800 AAVs, at least about 900 AAVs, or at least about 1000 AAVs in the lumen of the EV. In some embodiments, the EV comprises at least about 5 AAVs to at least about 1000 AAVs, at least about 5 AAVs to at least about 900 AAVs, at least about 5 AAVs to at least about 800 AAVs, at least about 5 AAVs to at least about 700 AAVs, at least about 5 AAVs to at least about 600 AAVs, at least about 5 AAVs to at least about 500 AAVs, at least about 5 AAVs to at least about 400 AAVs, at least about 5 AAVs to at least about 300 AAVs, at least about 5 AAVs to at least about 200 AAVs, at least about 5 AAVs to at least about 100 AAVs in the lumen of the EV. In some embodiments, the EV comprises at least about 10 AAVs to at least about 1000 AAVs, at least about 10 AAVs to at least about 900 AAVs, at least about 10 AAVs to at least about 800 AAVs, at least about 10 AAVs to at least about 700 AAVs, at least about 10 AAVs to at least about 600 AAVs, at least about 10 AAVs to at least about 500 AAVs, at least about 10 AAVs to at least about 400 AAVs, at least about 10 AAVs to at least about 300 AAVs, at least about 10 AAVs to at least about 200 AAVs, at least about 10 AAVs to at least about 100 AAVs in the lumen of the EV. In some embodiments, the EV comprises at least about 100 AAVs to at least about 1000 AAVs, at least about 100 AAVs to at least about 900 AAVs, at least about 100 AAVs to at least about 800 AAVs, at least about 100 AAVs to at least about 700 AAVs, at least about 100 AAVs to at least about 600 AAVs, at least about 100 AAVs to at least about 500 AAVs, at least about 100 AAVs to at least about 400 AAVs, at least about 100 AAVs to at least about 300 AAVs, or at least about 100 AAVs to at least about 200 AAVs in the lumen of the EV. In some embodiments, the EV comprises at least about 10 AAVs to at least about 20 AAVs, at least about 10 AAVs to at least about 30 AAVs, at least about 10 AAVs to at least about 40 AAVs, at least about 10 AAVs to at least about 50 AAVs, at least about 10 AAVs to at least about 60 AAVs, at least about 10 AAVs to at least about 70 AAVs, at least about 10 AAVs to at least about 80 AAVs, or at least about 10 AAVs to at least about 90 AAVs in the lumen of the EV.

In some embodiments, the EV comprises at least about 5 AAVs to at least about 75 AAVs, at least about 5 AAVs to at least about 50 AAVs, at least about 5 AAVs to at least about 45 AAVs, at least about 5 AAVs to at least about 40 AAVs, at least about 5 AAVs to at least about 35 AAVs, at least about 5 AAVs to at least about 30 AAVs, at least about 5 AAVs to at least about 25 AAVs, at least about 5 AAVs to at least about 20 AAVs, at least about 5 AAVs to at least about 15 AAVs, at least about 5 AAVs to at least about 10 AAVs, at least about 10 AAVs to at least about 100 AAVs, at least about 10 AAVs to at least about 75 AAVs, at least about 10 AAVs to at least about 50 AAVs, at least about 5 AAVs to at least about 45 AAVs, at least about 10 AAVs to at least about 40 AAVs, at least about 10 AAVs to at least about 35 AAVs, at least about 10 AAVs to at least about 30 AAVs, at least about 10 AAVs to at least about 25 AAVs, at least about 10 AAVs to at least about 20 AAVs, or at least about 10 AAVs to at least about 15 AAVs in the lumen of the EV. In some embodiments, the EV comprises at least about 5 to at least about 20 AAVs in the lumen of the EV. In some embodiments, the EV comprises at least about 5 to at least about 10 AAVs in the lumen of the EV.

In some embodiments, the EV comprises at least about 4 AAV in the lumen of the EV. In some embodiments, the EV comprises at least about 5 AAV in the lumen of the EV. In some embodiments, the EV comprises at least about 6 AAV in the lumen of the EV. In some embodiments, the EV comprises at least about 7 AAV in the lumen of the EV. In some embodiments, the EV comprises at least about 8 AAV in the lumen of the EV. In some embodiments, the EV comprises at least about 9 AAV in the lumen of the EV. In some embodiments, the EV comprises at least about 10 AAV in the lumen of the EV. In some embodiments, the EV comprises at least about 11 AAV in the lumen of the EV. In some embodiments, the EV comprises at least about 12 AAV in the lumen of the EV. In some embodiments, the EV comprises at least about 13 AAV in the lumen of the EV. In some embodiments, the EV comprises at least about 14 AAV in the lumen of the EV. In some embodiments, the EV comprises at least about 15 AAV in the lumen of the EV.

In some embodiments, the EVs of the present disclosure contain AAVs in the EVs in a more uniform way (e.g., the number of AAVs in the EVs) compared to the reference EVs prepared without the scaffold protein. In some embodiments, the EVs of the present disclosure contain about 5 to about 10, about 6 to about 10, about 7 to about 10, about 8 to about 10 AAVs in the EVs while the reference EVs can vary in the number of AAVs from 0 to from 5. In other embodiments, the EVs of the present disclosure can control the number of AAVs in the EVs by using the scaffold protein disclosed herein. For example, the use of the scaffold protein allows AAVs to be attached in the luminal surface of the EVs when the EVs are produced from the cells, and to be detached from the EVs at the site of the injury or the target. In other embodiments, the use of the scaffold protein (e.g., chemically induced dimer partners) allows AAVs to be attached in the luminal surface of the EVs when the EVs are produced from the cells, and to be detached from the EVs after the chemical is removed from the EV. Therefore, the EVs of the present disclosure allow efficient and uniform loading of the AAVs in the EVs.

Certain aspects of the present disclosure are directed to an EV, e.g., exosome, comprising an AAV and a scaffold protein, wherein the AAV is associated with the external surface of the EV. In some embodiments, the EVs of the present disclosure comprise AAVs associated with the surface of the AAV in a more uniform way than other methods of associating an AAV with an EV, e.g., as a luminal payload. In some embodiments, more AAVs are able to be associated with the EV surface than are able to be loaded in the lumen of the AAV, increasing the number of AAV that can be delivered to a subject. In some embodiments, at least about 100 AAVs are associated with the external surface of the EV, e.g., exosome. In some embodiments, at least about 200 AAVs are associated with the external surface of the EV, e.g., exosome. In some embodiments, at least about 300 AAVs are associated with the external surface of the EV, e.g., exosome. In some embodiments, at least about 400 AAVs are associated with the external surface of the EV, e.g., exosome. In some embodiments, at least about 500 AAVs are associated with the external surface of the EV, e.g., exosome. In some embodiments, at least about 600 AAVs are associated with the external surface of the EV, e.g., exosome. In some embodiments, at least about 700 AAVs are associated with the external surface of the EV, e.g., exosome. In some embodiments, at least about 800 AAVs are associated with the external surface of the EV, e.g., exosome. In some embodiments, at least about 900 AAVs are associated with the external surface of the EV, e.g., exosome. In some embodiments, at least about 1000 AAVs are associated with the external surface of the EV, e.g., exosome. In some embodiments, at least about 1100 AAVs are associated with the external surface of the EV, e.g., exosome. In some embodiments, at least about 1200 AAVs are associated with the external surface of the EV, e.g., exosome. In some embodiments, at least about 1300 AAVs are associated with the external surface of the EV, e.g., exosome. In some embodiments, at least about 1400 AAVs are associated with the external surface of the EV, e.g., exosome. In some embodiments, at least about 1500 AAVs are associated with the external surface of the EV, e.g., exosome. In some embodiments, at least about 1600 AAVs are associated with the external surface of the EV, e.g., exosome. In some embodiments, at least about 1700 AAVs are associated with the external surface of the EV, e.g., exosome. In some embodiments, at least about 1800 AAVs are associated with the external surface of the EV, e.g., exosome. In some embodiments, at least about 1900 AAVs are associated with the external surface of the EV, e.g., exosome. In some embodiments, at least about 2000 AAVs are associated with the external surface of the EV, e.g., exosome. In some embodiments, at least about 1 to at least about 2000 AAVs are associated with the external surface of the EV, e.g., exosome. In some embodiments, at least about 1 to at least about 1000 AAVs are associated with the external surface of the EV, e.g., exosome. In some embodiments, at least about 1 to at least about 900, at least about 1 to at least about 800, at least about 1 to at least about 700, at least about 1 to at least about 600, at least about 1 to at least about 500, at least about 1 to at least about 450, at least about 1 to at least about 400, at least about 1 to at least about 350, at least about 1 to at least about 325, at least about 1 to at least about 300, at least about 1 to at least about 275, at least about 1 to at least about 250, at least about 1 to at least about 225, at least about 1 to at least about 200, at least about 1 to at least about 175, at least about 1 to at least about 150, at least about 1 to at least about 125, at least about 1 to at least about 100, at least about 1 to at least about 90, at least about 1 to at least about 80, at least about 1 to at least about 70, at least about 1 to at least about 60, at least about 1 to at least about 50, at least about 1 to at least about 45, at least about 1 to at least about 40, at least about 1 to at least about 35, at least about 1 to at least about 30, at least about 1 to at least about 25, at least about 1 to at least about 20, at least about 1 to at least about 15, at least about 1 to at least about 14, at least about 1 to at least about 13, at least about 1 to at least about 12, at least about 1 to at least about 11, at least about 1 to at least about 10, at least about 10 to at least about 500, at least about 10 to at least about 450, at least about 10 to at least about 400, at least about 10 to at least about 350, at least about 10 to at least about 325, at least about 10 to at least about 300, at least about 10 to at least about 275, at least about 10 to at least about 250, at least about 10 to at least about 225, at least about 10 to at least about 200, at least about 10 to at least about 175, at least about 10 to at least about 150, at least about 10 to at least about 125, at least about 10 to at least about 100, at least about 10 to at least about 90, at least about 10 to at least about 80, at least about 10 to at least about 70, at least about 10 to at least about 60, at least about 10 to at least about 50, at least about 10 to at least about 45, at least about 10 to at least about 40, at least about 10 to at least about 35, at least about 10 to at least about 30, at least about 10 to at least about 25, at least about 10 to at least about 20, at least about 100 to at least about 1000, at least about 100 to at least about 900, at least about 100 to at least about 800, at least about 100 to at least about 700, at least about 100 to at least about 600, at least about 100 to at least about 500, at least about 100 to at least about 400, at least about 100 to at least about 300, at least about 100 to at least about 200 AAVs are associated with the external surface of the EV.

Any AAV known in the art can be used in the compositions of the present disclosure. In some embodiments, the AAV is selected from the group consisting of AAV type 1, AAV type 2, AAV type 3A, AAV type 3B, AAV type 4, AAV type 5, AAV type 6, AAV type 7, AAV type 8, AAV type 9, AAV type 10, AAV type 11, AAV type 12, AAV type 13, Rh10, Rh74, AAV-2i8, snake AAV, avian AAV, bovine AAV, canine AAV, equine AAV, ovine AAV, goat AAV, shrimp AAV, a synthetic AAV, an any combination thereof. In certain embodiments, the AAV is an AAV type 2, e.g., AAV2. In certain embodiments, the AAV is an AAV type 3A, e.g., AAV3A. In certain embodiments, the AAV is an AAV type 3B, e.g., AAV3B. In certain embodiments, the AAV is an AAV type 4, e.g., AAV4. In certain embodiments, the AAV is an AAV type 5, e.g., AAV5. In certain embodiments, the AAV is an AAV type 6, e.g., AAV6. In certain embodiments, the AAV is an AAV type 7, e.g., AAV7. In certain embodiments, the AAV is an AAV type 8, e.g., AAV8. In certain embodiments, the AAV is an AAV type 9, e.g., AAV9. In certain embodiments, the AAV is an AAV type 10, e.g., AAV10. In certain embodiments, the AAV is a synthetic AAV.

In some aspects, the AAV has distinct tissue targeting capabilities (e.g., tissue tropisms). In some embodiments, the AAV further exhibits increased transduction or tropism in one or more human stem cell types as compared to non-variant parent capsid polypeptides. In some embodiments, the human stem cell types include but are not limited to embryonic stem cells, adult tissue stem cells (i.e., somatic stem cells), bone marrow, progenitor cells, induced pluripotent stem cells, and reprogrammed stem cells. In some embodiments, adult stem cells can include organoid stem cells (i.e., stem cells derived from any organ or organ system of interest within the body). In some embodiments, the target tissue of an AAV is gonad, diaphragm, heart, stomach, liver, spleen, pancreas, or kidney. In some embodiments, the AAV targets organs of the body include, but are not limited to, skin, hair, nails, sense receptors, sweat gland, oil glands, bones, muscles, brain, spinal cord, nerve, pituitary gland, pineal gland, hypothalamus, thyroid gland, parathyroid, thymus, adrenals, pancreas (islet tissue), heart, blood vessels, lymph nodes, lymph vessels, thymus, spleen, tonsils, nose, pharynx, larynx, trachea, bronchi, lungs, mouth, pharynx, esophagus, stomach, small intestine, large intestine, rectum, anal canal, teeth, salivary glands, tongue, liver, gallbladder, pancreas, appendix, kidneys, ureters, urinary bladder, urethra, testes, ductus (vas) deferens, urethra, prostate, penis, scrotum, ovaries, uterus, uterine (fallopian) tubes, vagina, vulva, and mammary glands (breasts). Organ systems of the body include but are not limited to the integumentary system, skeletal system, muscular system, nervous system, endocrine system, cardiovascular system, lymphatic system, respiratory system, digestive system, urinary system, and reproductive system. In some embodiments, transduction and/or tropism is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%%, at least about 75%, at least about 80%, at least about 85%, at least about 90%, at least about 95%, at least about 99%, or at least about 100%. In some embodiments, transduction and/or tropism is increased by at least about 5% to at least about 80%, at least about 10% to at least about 70%, at least about 20% to at least about 60%, or at least about 30% to at least about 60%.

In some aspects, the AAV of the present disclosure has one or more altered properties as compared to an AAV not associated with an EV, as disclosed herein. In some embodiments, the altered property comprises a better therapeutic effect than an AAV alone. In some embodiments, the better therapeutic effect comprises improved immune evasion, improved ability to redose, improved ability to titrate dose, or any combination thereof. In some embodiments, the AAV of the present disclosure are less likely to induce an immune response in a subject. In particular, this allows for the AAV of the present disclosure to be administered to a subject with pre-existing neutralizing antibodies. In some embodiments, the AAV of the present disclosure illicit faster uptake and/or improved transduction kinetics as compared to an AAV not associated with an EV, as disclosed herein.

II.B.1. AAV Fusion Constructs

In some embodiments, the AAV is linked to a scaffold protein described herein. In some embodiments, the scaffold protein is linked to a protein of the AAV. In some aspects, the EV, e.g., exosome, comprises an AAV and a scaffold protein, wherein the AAV is associated with the luminal surface of the exosome.

In some aspects, the EV, e.g., exosome, comprises an AAV and a scaffold protein, wherein the AAV is associated with the external surface of the exosome. In some embodiments, the scaffold protein comprises an external domain, e.g., a domain that is located external to the EV membrane, wherein the AAV is associated with the external domain of the scaffold protein. In some embodiments, the scaffold protein further comprises a transmembrane region, wherein the transmembrane region is anchored to the membrane of the EV.

The AAV can be directly or indirectly associated with the scaffold protein. In some embodiments, the AAV is associated with the scaffold protein by one or more covalent bonds. In other embodiments, the AAV is associated with the scaffold protein by one or more non-covalent interactions.

In certain embodiments, the association between the scaffold protein and the AAV is between the scaffold protein and a protein of the AAV.

The single-stranded genome of AAV comprises three genes, rep (Replication), cap (Capsid), and aap (Assembly). These three genes give rise to at least nine gene products through the use of three promoters, alternative translation start sites, and differential splicing, including three capsid proteins.

Cap gene expression gives rise to the viral capsid proteins (VP1, VP2, and VP3), which form the outer capsid shell that protects the viral genome, as well as being actively involved in cell binding and internalization. It is estimated that the viral coat is comprised of 60 proteins arranged into an icosahedral structure. In some embodiments, AAV capsids are composed of 60 copies of 3 proteins VP1, VP2, and VP3 in a ratio of 1:1:10, e.g., 5 VP1 proteins, 5 VP2 proteins, and 50 VP3 proteins.

The rep gene encodes four proteins (Rep78, Rep68, Rep52, and Rep40), which are required for viral genome replication and packaging. The aap gene encodes the assembly-activating protein (AAP) in an alternate reading frame overlapping the cap gene. This nuclear protein is thought to provide a scaffolding function for capsid assembly and plays a role in nucleolar localization of VP proteins in some AAV serotypes.

In some embodiments, one or more of the rep, cap, or aap genes are naturally occurring, e.g. the rep, cap, or app genes comprise all or a portion of Parvovirus rep, cap, or aap genes. In some embodiments, the one or more of the rep, cap, or aap genes comprise a synthetic sequence.

In one embodiment, the rep gene comprises a synthetic sequence. In one embodiment, the cap gene comprises a synthetic sequence. In one embodiment, the aap gene comprises a synthetic sequence. In one embodiment, the rep and cap genes comprise a synthetic sequence. In one embodiment, the rep and aap genes comprise a synthetic sequence. In one embodiment, the cap and aap genes comprise a synthetic sequence. In one embodiment, the rep, cap, and aap genes comprise a synthetic sequence.

In some embodiments, rep is from an AAV genome selected from AAV1, AAV2, AAV3, AAV4, AAV5, AAV6, AAV7, AAV8, AAV9, AAV10, AAV11, and any combination thereof. In a particular embodiment, rep is from the AAV1 genome. In a particular embodiment, rep is from the AAV2 genome. In a particular embodiment, rep is from the AAV3 genome. In a particular embodiment, rep is from the AAV4 genome. In a particular embodiment, rep is from the AAV5 genome. In a particular embodiment, rep is from the AAV6 genome. In a particular embodiment, rep is from the AAV7 genome. In a particular embodiment, rep is from the AAV8 genome. In a particular embodiment, rep is from the AAV9 genome. In a particular embodiment, rep is from the AAV10 genome. In a particular embodiment, rep is from the AAV11 genome.

In some embodiments, cap is from an AAV genome selected from AAV1, AAV2, AAV3, AAV4, AAV5, AAV6, AAV7, AAV8, AAV9, AAV10, AAV11, and any combination thereof. In a particular embodiment, cap is from the AAV1 genome. In a particular embodiment, cap is from the AAV2 genome. In a particular embodiment, cap is from the AAV3 genome. In a particular embodiment, cap is from the AAV4 genome. In a particular embodiment, cap is from the AAV5 genome. In a particular embodiment, cap is from the AAV6 genome. In a particular embodiment, cap is from the AAV7 genome. In a particular embodiment, cap is from the AAV8 genome. In a particular embodiment, cap is from the AAV9 genome. In a particular embodiment, cap is from the AAV10 genome. In a particular embodiment, cap is from the AAV11 genome.

In some embodiments, aap is from an AAV genome selected from AAV1, AAV2, AAV3, AAV4, AAV5, AAV6, AAV7, AAV8, AAV9, AAV10, AAV11, and any combination thereof. In a particular embodiment, aap is from the AAV1 genome. In a particular embodiment, aap is from the AAV2 genome. In a particular embodiment, aap is from the AAV3 genome. In a particular embodiment, aap is from the AAV4 genome. In a particular embodiment, aap is from the AAV5 genome. In a particular embodiment, aap is from the AAV6 genome. In a particular embodiment, aap is from the AAV7 genome. In a particular embodiment, aap is from the AAV8 genome. In a particular embodiment, aap is from the AAV9 genome. In a particular embodiment, aap is from the AAV10 genome. In a particular embodiment, aap is from the AAV11 genome.

It is to be understood that a particular AAV genome described herein could have genes from different AAV genomes (e.g., genomes from AAV1, AAV2, AAV3, AAV4, AAV5, AAV6, AAV7, AAV8, AAV9, AAV10, and AAV11). Thus, disclosed herein are AAVs that comprise any possible permutation of rep, cap, or aap.

In some embodiments disclosed herein, the AAV is recombinant AAV (rAAV). In some embodiments, the rAAV lacks one or more of the rep gene, the cap gene, and the aap gene. In some embodiments, the rAAV lacks a rep gene. In some embodiments, the rAAV lacks a cap gene. In some embodiments, the rAAV lacks an aap gene. In some embodiments, the rAAV lacks a rep gene and lacks a cap gene. In some embodiments, the rAAV lacks a rep gene and lacks an aap gene. In some embodiments, the rAAV lacks a cap gene and lacks an aap gene. In some embodiments, the rAAV lacks a rep gene, a cap gene, and an aap gene.

In some embodiments disclosed herein, the rAAV is modified so that one or more of the rep gene, the cap gene, and the aap gene is mutated so that expression of one or more of the AAV genes is modified. In some embodiments, the rep gene is mutated. In some embodiments, the cap gene is mutated. In some embodiments, the aap gene is mutated. In some embodiments, the rep gene and the cap gene are mutated. In some embodiments, the rep gene and the aap gene are mutated. In some embodiments, the cap gene and the aap gene are mutated. In some embodiments, the cap gene, the rep gene, and the aap gene are mutated.

In some embodiments, the scaffold protein is linked to or associated with a capsid protein of the AAV. In some embodiments, the scaffold protein is linked to or associated with at least one VP1 protein of the AAV. In some embodiments, a scaffold protein is linked to or associated with each of the 5 VP1 proteins of the AAV. In some embodiments, a scaffold protein is linked to or associated with each of 4 of the VP1 proteins of the AAV. In some embodiments, a scaffold protein is linked to or associated with each of 3 of the VP1 proteins of the AAV. In some embodiments, a scaffold protein is linked to or associated with each of 2 of the VP1 proteins of the AAV. In some embodiments, a scaffold protein is linked to or associated with 1 of the VP1 proteins of the AAV. In some embodiments, the AAV comprises one VP1 protein that is not linked to or associated with a scaffold protein. In some embodiments, the AAV comprises two VP1 proteins that are not linked to or associated with a scaffold protein. In some embodiments, the AAV comprises three VP1 proteins that are not linked to or associated with a scaffold protein. In some embodiments, the AAV comprises four VP1 proteins that are not linked to or associated with a scaffold protein.

In some embodiments, the scaffold protein is linked to or associated with at least one VP2 protein of the AAV. In some embodiments, a scaffold protein is linked to or associated with each of the 5 VP2 proteins of the AAV. In some embodiments, a scaffold protein is linked to or associated with each of 4 of the VP2 proteins of the AAV. In some embodiments, a scaffold protein is linked to or associated with each of 3 of the VP2 proteins of the AAV. In some embodiments, a scaffold protein is linked to or associated with each of 2 of the VP2 proteins of the AAV. In some embodiments, a scaffold protein is linked to or associated with 1 of the VP2 proteins of the AAV. In some embodiments, the AAV comprises one VP2 protein that is not linked to or associated with a scaffold protein. In some embodiments, the AAV comprises two VP2 proteins that are not linked to or associated with a scaffold protein. In some embodiments, the AAV comprises three VP2 proteins that are not linked to or associated with a scaffold protein. In some embodiments, the AAV comprises four VP2 proteins that are not linked to or associated with a scaffold protein.

In some embodiments, the scaffold protein is linked to or associated with at least one VP3 protein of the AAV. In some embodiments, a scaffold protein is linked to or associated with each of the VP3 proteins of the AAV. In some embodiments, a scaffold protein is linked to or associated with each of a subset of the VP3 proteins of the AAV. In some embodiments, a scaffold protein is linked to or associated with each of at least about 40 of the VP3 proteins of the AAV. In some embodiments, a scaffold protein is linked to or associated with each of at least about 35 of the VP3 proteins of the AAV. In some embodiments, a scaffold protein is linked to or associated with each of at least about 30 of the VP3 proteins of the AAV. In some embodiments, a scaffold protein is linked to or associated with each of at least about 25 of the VP3 proteins of the AAV. In some embodiments, a scaffold protein is linked to or associated with each of at least about 20 of the VP3 proteins of the AAV. In some embodiments, a scaffold protein is linked to or associated with each of at least about 15 of the VP3 proteins of the AAV. In some embodiments, a scaffold protein is linked to or associated with each of at least about 10 of the VP3 proteins of the AAV. In some embodiments, a scaffold protein is linked to or associated with each of at least about 9 of the VP3 proteins of the AAV. In some embodiments, a scaffold protein is linked to or associated with each of at least about 8 of the VP3 proteins of the AAV. In some embodiments, a scaffold protein is linked to or associated with each of at least about 7 of the VP3 proteins of the AAV. In some embodiments, a scaffold protein is linked to or associated with each of at least about 6 of the VP3 proteins of the AAV. In some embodiments, a scaffold protein is linked to or associated with each of at least about 5 of the VP3 proteins of the AAV. In some embodiments, a scaffold protein is linked to or associated with each of at least about 4 of the VP3 proteins of the AAV. In some embodiments, a scaffold protein is linked to or associated with each of at least about 3 of the VP3 proteins of the AAV. In some embodiments, a scaffold protein is linked to or associated with each of at least about 2 of the VP3 proteins of the AAV. In some embodiments, a scaffold protein is linked to or associated with 1 of the VP3 proteins of the AAV. In some embodiments, the AAV comprises at least 1 VP3 protein that is not linked to or associated with a scaffold protein. In some embodiments, the AAV comprises at least 2 VP3 proteins that are not linked to or associated with a scaffold protein. In some embodiments, the AAV comprises at least 3 VP3 proteins that are not linked to or associated with a scaffold protein. In some embodiments, the AAV comprises at least 4 VP3 proteins that are not linked to or associated with a scaffold protein. In some embodiments, the AAV comprises at least 5 VP3 proteins that are not linked to or associated with a scaffold protein. In some embodiments, the AAV comprises at least 10 VP3 proteins that are not linked to or associated with a scaffold protein. In some embodiments, the AAV comprises at least 15 VP3 proteins that are not linked to or associated with a scaffold protein. In some embodiments, the AAV comprises at least 20 VP3 proteins that are not linked to or associated with a scaffold protein. In some embodiments, the AAV comprises at least 25 VP3 proteins that are not linked to or associated with a scaffold protein. In some embodiments, the AAV comprises at least 30 VP3 proteins that are not linked to or associated with a scaffold protein. In some embodiments, the AAV comprises at least 35 VP3 proteins that are not linked to or associated with a scaffold protein. In some embodiments, the AAV comprises at least 40 VP3 proteins that are not linked to or associated with a scaffold protein. In some embodiments, the AAV comprises at least 45 VP3 proteins that are not linked to or associated with a scaffold protein.

In some embodiments, the number of the VP3 linked to or associated with the scaffold protein is at least about 2 fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14 fold, at least about 15 fold, at least about 20 fold, at least about 30 fold, at least about 35 fold, at least about 40 fold, at least about 45 fold, at least about 50 fold less than the number of the at least one VP3 protein not linked to or associated with the scaffold protein.

In certain embodiments, the AAV comprises 1 VP2 protein linked to or associated with a scaffold protein. In some embodiments, the AAV comprises 2 VP2 proteins linked to or associated with scaffold proteins. In some embodiments, the AAV comprises 3 VP2 proteins linked to or associated with scaffold proteins. In some embodiments, the AAV comprises 4 VP2 proteins linked to or associated with scaffold proteins. In some embodiments, the AAV comprises 5 VP2 proteins linked to or associated with scaffold proteins.

In certain embodiments, the AAV comprises 1 VP1 protein linked to or associated with a scaffold protein. In some embodiments, the AAV comprises 2 VP1 proteins linked to or associated with scaffold proteins. In some embodiments, the AAV comprises 3 VP1 proteins linked to or associated with scaffold proteins. In some embodiments, the AAV comprises 4 VP1 proteins linked to or associated with scaffold proteins. In some embodiments, the AAV comprises 5 VP1 proteins linked to or associated with scaffold proteins.

In certain embodiments, the AAV comprises 1 VP3 protein linked to or associated with a scaffold protein. In some embodiments, the AAV comprises 2 VP3 proteins linked to or associated with scaffold proteins. In some embodiments, the AAV comprises 3 VP3 proteins linked to or associated with scaffold proteins. In some embodiments, the AAV comprises 4 VP3 proteins linked to or associated with scaffold proteins. In some embodiments, the AAV comprises 5 VP3 proteins linked to or associated with scaffold proteins.

In some embodiments, the scaffold protein is linked to the AAV, e.g., a capsid protein of the AAV, by one or more peptide bonds. The scaffold protein can be linked to or associated with the AAV capsid protein at the N-terminus or the C-terminus of the capsid protein or between the N-terminus and the C-terminus of the capsid protein. In some embodiments, the scaffold protein is linked to or associated with the N-terminus of the capsid protein. In other embodiments, the scaffold protein is linked to or associated with the C-terminus of the capsid protein. In some embodiments, the N-terminus of the scaffold protein is linked to the C-terminus of a capsid protein of the AAV. In other embodiments, the C-terminus of the scaffold protein is linked to the N-terminus of a capsid protein of the AAV.

The scaffold protein can be linked to or associated with the capsid protein of the AAV either directly or indirectly, e.g., by a linker. In some embodiments, the scaffold protein is linked to or associated with the capsid protein by a linker. In some embodiments, the linker comprises one or more amino acids. In some embodiments, the linker is a cleavable linker. In some embodiments, the linker is a flexible linker. In some embodiments, the linker is a rigid linker. In certain embodiments, the linker is at least about 2 amino acids, at least about 3 amino acids, at least about 4 amino acids, at least about 5 amino acids, at least about 6 amino acids, at least about 7 amino acids, at least about 8 amino acids, at least about 9 amino acids, at least about 10 amino acids, at least about 11 amino acids, at least about 12 amino acids, at least about 13 amino acids, at least about 14 amino acids, at least about 15 amino acids, at least about 16 amino acids, at least about 17 amino acids, at least about 18 amino acids, at least about 19 amino acids, at least about 20 amino acids, at least about 25 amino acids, at least about 30 amino acids, at least about 35 amino acids, at least about 40, amino acids, at least about 45 amino acids, or at least about 50.

Certain aspects of the present disclosure are directed to an EV comprising an AAV and a scaffold protein, wherein the AAV is linked to or associated with a binding partner or dimerizing agent of a chemically induced dimer. In some embodiments, the binding partner is linked to or associated with a capsid protein of the AAV. In some embodiments, the binding partner is linked to or associated with at least one VP1 protein of the AAV. In some embodiments, a binding partner is linked to or associated with each of the 5 VP1 proteins of the AAV. In some embodiments, a binding partner is linked to or associated with each of 4 of the VP1 proteins of the AAV. In some embodiments, a binding partner is linked to or associated with each of 3 of the VP1 proteins of the AAV. In some embodiments, a binding partner is linked to or associated with each of 2 of the VP1 proteins of the AAV. In some embodiments, a binding partner is linked to or associated with 1 of the VP1 proteins of the AAV. In some embodiments, the AAV comprises one VP1 protein that is not linked to or associated with a binding partner. In some embodiments, the AAV comprises two VP1 proteins that are not linked to or associated with a binding partner. In some embodiments, the AAV comprises three VP1 proteins that are not linked to or associated with a binding partner. In some embodiments, the AAV comprises four VP1 proteins that are not linked to or associated with a binding partner.

In some embodiments, the binding partner is linked to or associated with at least one VP2 protein of the AAV. In some embodiments, a binding partner is linked to or associated with each of the 5 VP2 proteins of the AAV. In some embodiments, a binding partner is linked to or associated with each of 4 of the VP2 proteins of the AAV. In some embodiments, a binding partner is linked to or associated with each of 3 of the VP2 proteins of the AAV. In some embodiments, a binding partner is linked to or associated with each of 2 of the VP2 proteins of the AAV. In some embodiments, a binding partner is linked to or associated with 1 of the VP2 proteins of the AAV. In some embodiments, the AAV comprises one VP2 protein that is not linked to or associated with a binding partner. In some embodiments, the AAV comprises two VP2 proteins that are not linked to or associated with a binding partner. In some embodiments, the AAV comprises three VP2 proteins that are not linked to or associated with a binding partner. In some embodiments, the AAV comprises four VP2 proteins that are not linked to or associated with a binding partner.

In some embodiments, the binding partner is linked to or associated with at least one VP3 protein of the AAV. In some embodiments, a binding partner is linked to or associated with each of the VP3 proteins of the AAV. In some embodiments, a binding partner is linked to or associated with each of a subset of the VP3 proteins of the AAV. In some embodiments, a binding partner is linked to or associated with each of at least about 40 of the VP3 proteins of the AAV. In some embodiments, a binding partner is linked to or associated with each of at least about 35 of the VP3 proteins of the AAV. In some embodiments, a binding partner is linked to or associated with each of at least about 30 of the VP3 proteins of the AAV. In some embodiments, a binding partner is linked to or associated with each of at least about 25 of the VP3 proteins of the AAV. In some embodiments, a binding partner is linked to or associated with each of at least about 20 of the VP3 proteins of the AAV. In some embodiments, a binding partner is linked to or associated with each of at least about 15 of the VP3 proteins of the AAV. In some embodiments, a binding partner is linked to or associated with each of at least about 10 of the VP3 proteins of the AAV. In some embodiments, a binding partner is linked to or associated with each of at least about 9 of the VP3 proteins of the AAV. In some embodiments, a binding partner is linked to or associated with each of at least about 8 of the VP3 proteins of the AAV. In some embodiments, a binding partner is linked to or associated with each of at least about 7 of the VP3 proteins of the AAV. In some embodiments, a binding partner is linked to or associated with each of at least about 6 of the VP3 proteins of the AAV. In some embodiments, a binding partner is linked to or associated with each of at least about 5 of the VP3 proteins of the AAV. In some embodiments, a binding partner is linked to or associated with each of at least about 4 of the VP3 proteins of the AAV. In some embodiments, a binding partner is linked to or associated with each of at least about 3 of the VP3 proteins of the AAV. In some embodiments, a binding partner is linked to or associated with each of at least about 2 of the VP3 proteins of the AAV. In some embodiments, a binding partner is linked to or associated with 1 of the VP3 proteins of the AAV. In some embodiments, the AAV comprises at least about 1 VP3 protein that is not linked to or associated with a binding partner. In some embodiments, the AAV comprises at least about 2 VP3 proteins that are not linked to or associated with a binding partner. In some embodiments, the AAV comprises at least about 3 VP3 proteins that are not linked to or associated with a binding partner. In some embodiments, the AAV comprises at least about 4 VP3 proteins that are not linked to or associated with a binding partner. In some embodiments, the AAV comprises at least about 5 VP3 proteins that are not linked to or associated with a binding partner. In some embodiments, the AAV comprises at least about 10 VP3 proteins that are not linked to or associated with a binding partner. In some embodiments, the AAV comprises at least about 15 VP3 proteins that are not linked to or associated with a binding partner. In some embodiments, the AAV comprises at least about 20 VP3 proteins that are not linked to or associated with a binding partner. In some embodiments, the AAV comprises at least about 25 VP3 proteins that are not linked to or associated with a binding partner. In some embodiments, the AAV comprises at least about 30 VP3 proteins that are not linked to or associated with a binding partner. In some embodiments, the AAV comprises at least about 35 VP3 proteins that are not linked to or associated with a binding partner. In some embodiments, the AAV comprises at least about 40 VP3 proteins that are not linked to or associated with a binding partner. In some embodiments, the AAV comprises at least about 45 VP3 proteins that are not linked to or associated with a binding partner.

In some embodiments, the number of the VP3 linked to or associated with the binding partner is about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 20 fold, about 30 fold, about 35 fold, about 40 fold, about 45 fold, about 50 fold less than the number of the at least one VP3 protein not linked to or associated with the binding partner.

In certain embodiments, the AAV comprises 1 VP2 protein linked to or associated with a binding partner. In some embodiments, the AAV comprises 2 VP2 proteins linked to or associated with binding partners. In some embodiments, the AAV comprises 3 VP2 proteins linked to or associated with binding partners. In some embodiments, the AAV comprises 4 VP2 proteins linked to or associated with binding partners. In some embodiments, the AAV comprises 5 VP2 proteins linked to or associated with binding partners.

In certain embodiments, the AAV comprises 1 VP1 protein linked to or associated with a binding partner. In some embodiments, the AAV comprises 2 VP1 proteins linked to or associated with binding partners. In some embodiments, the AAV comprises 3 VP1 proteins linked to or associated with binding partners. In some embodiments, the AAV comprises 4 VP1 proteins linked to or associated with binding partners. In some embodiments, the AAV comprises 5 VP1 proteins linked to or associated with binding partners.

In certain embodiments, the AAV comprises 1 VP3 protein linked to or associated with a binding partner. In some embodiments, the AAV comprises 2 VP3 proteins linked to or associated with binding partners. In some embodiments, the AAV comprises 3 VP3 proteins linked to or associated with binding partners. In some embodiments, the AAV comprises 4 VP3 proteins linked to or associated with binding partners. In some embodiments, the AAV comprises 5 VP3 proteins linked to or associated with binding partners.

In some embodiments, the binding partner is linked to or associated with the N-terminus of the capsid protein. In other embodiments, the binding partner is linked to or associated with the C-terminus of the capsid protein. In other embodiments, the binding partner is inserted within the capsid protein, e.g., between the N-terminus and the C-terminus of the capsid protein. In some embodiments, the binding partner is inserted within the capsid protein. In certain embodiments, the binding partner is inserted within the capsid protein, e.g., VP1, VP2, and/or VP3, within an internal loop, e.g., an series of amino acids which form a loop structure that is on the surface of the capsid protein. In certain embodiments, the binding partner is inserted within the capsid protein, e.g., VP1, VP2, and/or VP3, immediately downstream of amino acid 455 (relative to the numbering of SEQ ID NO:44). I In some embodiments, the first binding partner is inserted within the capsid protein, e.g., VP1, VP2, and/or VP3, by replacing Gly₄₅₃ (relative to the numbering of SEQ ID NO:44). In some embodiments, the first binding partner is inserted within the capsid protein, e.g., VP1, VP2, and/or VP3, by replacing Thr₄₅₄ (relative to the numbering of SEQ ID NO:44). In some embodiments, the binding partner is inserted within the capsid protein, e.g., VP1, VP2, and/or VP3, by replacing Thr₄₅₅ (relative to the numbering of SEQ ID NO:44). In some aspects, the first binding partner is inserted within the capsid protein, e.g., VP1, VP2, and/or VP3, by replacing Thr₄₅₆ (relative to the numbering of SEQ ID NO:44). In some embodiments, the first binding partner is inserted within the capsid protein, e.g., VP1, VP2, and/or VP3, by replacing Gln₄₅₇ (relative to the numbering of SEQ ID NO:44). In some embodiments, the first binding partner is inserted within the capsid protein, e.g., VP1, VP2, and/or VP3, by replacing Ser₄₅₈ (relative to the numbering of SEQ ID NO:44). In some embodiments, the first binding partner is inserted within the capsid protein, e.g., VP1, VP2, and/or VP3, by replacing Arg₄₅₉ (relative to the numbering of SEQ ID NO:44). In some embodiments, the binding partner is inserted within the capsid protein, e.g., VP1, VP2, and/or VP3, by replacing ₄₅₃GTTTQSR₄₅₉ (relative to the numbering of SEQ ID NO:44), or into a homologous region of a VP proteins of other AAV serotypes. In particular embodiments, a binding partner is inserted within at least one VP3 protein by replacing Thr₄₅₅ (relative to the numbering of SEQ ID NO:44). In particular embodiments, a binding partner is inserted within at least one VP3 protein by replacing ₄₅₃GTTTQSR₄₅₉ (relative to the numbering of SEQ ID NO:44), or into a homologous region of a VP proteins of other AAV serotypes. In some aspects, the first binding partner is inserted within the capsid protein, e.g., VP1, VP2, and/or VP3, at a cite selected from Arg₅₈₅, Arg₅₈₇, and Arg₅₈₈, or any combination thereof relative to the amino acid sequence of VP2 of AAV2. In some aspects, the capsid protein, e.g., VP1, VP2, and/or VP3, is modified to comprise an internal myristylation site. In some aspects, the capsid protein, e.g., VP1, VP2, and/or VP3, is modified to comprise an internal myristylation site within an internal surface loop.

The binding partner can be linked to or associated with the capsid protein of the AAV either directly or indirectly, e.g., by a linker. In some embodiments, the binding partner is linked to or associated with the capsid by a linker. In some embodiments, the linker comprises one or more amino acids. In some embodiments, the linker is a cleavable linker. In some embodiments, the linker is a flexible linker. In some embodiments, the linker is a rigid linker. In certain embodiments, the linker is at least about 2 amino acids, at least about 3 amino acids, at least about 4 amino acids, at least about 5 amino acids, at least about 6 amino acids, at least about 7 amino acids, at least about 8 amino acids, at least about 9 amino acids, at least about 10 amino acids, at least about 12 amino acids, at least about amino acids, at least about 13 amino acids, at least about 14 amino acids, at least about 15 amino acids, at least about 16 amino acids, at least about 17 amino acids, at least about 18 amino acids, at least about 19 amino acids, at least about 20 amino acids, at least about 25 amino acids, at least about 30 amino acids, at least about 35 amino acids, at least about 40, amino acids, at least about 45 amino acids, or at least about 50.

In some embodiments, the binding partner linked to or associated with the AAV capsid protein is selected from one binding partner of a chemically induced dimer selected from the group consisting of (i) FKBP and FKBP (FK1012); (ii) FKBP and CalcineurinA (CNA) (FK506); (iii) FKBP and CyP-Fas (FKCsA); (iv) FKBP and FRB (Rapamycin); (v) GyrB and GyrB (Coumermycin); (vi) GAI and GID1 (Gibberellin); (vii) Snap-tag and HaloTag (HaXS); (viii) eDHFR and HaloTag (TMP-HTag); and (ix) BCL-xL and Fab (AZ1) (ABT-737). In certain embodiments, the AAV capsid protein is linked to or associated with an FKBP. In certain embodiments, the AAV capsid protein is linked to or associated with an FRB. IN some embodiments, the FRB is the FRB of mTOR. In some embodiments, the AAV capsid protein is linked to or associated with CalcineurinA. In some embodiments, the AAV capsid protein is linked to or associated with CyP-Fas. In some embodiments, the AAV capsid protein is linked to or associated with GyrB. In some embodiments, the AAV capsid protein is linked to or associated with CyP-Fas. In some embodiments, the AAV capsid protein is linked to or associated with GAI. In some embodiments, the AAV capsid protein is linked to or associated with GID1. In some embodiments, the AAV capsid protein is linked to or associated with GAI. In some embodiments, the AAV capsid protein is linked to or associated with Snap-tag. In some embodiments, the AAV capsid protein is linked to or associated with HaloTag. In some embodiments, the AAV capsid protein is linked to or associated with GAI. In some embodiments, the AAV capsid protein is linked to or associated with eDHFR. In some embodiments, the AAV capsid protein is linked to or associated with BCL-xL. In some embodiments, the AAV capsid protein is linked to or associated with eDHFR. In some embodiments, the AAV capsid protein is linked to or associated with Fab.

In particular embodiments, the AAV comprises at least one capsid protein (e.g., VP1, VP2, and/or VP3) linked to or associated with an FRB, wherein the FRB is linked to or associated with the N-terminus of the capsid protein. In some embodiments, the AAV comprises at least one capsid protein (e.g., VP1, VP2, and/or VP3) linked to or associated with an FRB, wherein the FRB is linked to or associated with the C-terminus of the capsid protein. In particular embodiments, the AAV comprises at least one capsid protein (e.g., VP1, VP2, and/or VP3) linked to or associated with an FRB, wherein the FRB is inserted within the capsid protein. In some embodiments, the FRB is inserted within the capsid protein at any location disclosed herein.

II.B.2. AAV Nucleic Acid Molecule

Certain aspects of the present disclosure are directed to an EV comprising an AAV, wherein the AAV comprises a genetic cassette, e.g., a heterologous sequence encoding a gene of interest. In some embodiments, the genetic cassette encodes a therapeutic protein. In some embodiments, the genetic cassette encodes a protein selected from the group consisting of clotting factor, a growth factor, a cytokine, a chemokine, or any combination thereof. In some embodiments, the gene of interest encodes an antioxidant. In some embodiments, the gene of interest encodes an enzyme. In some embodiments, the gene of interest encodes a tumor suppressor. In some embodiments, the gene of interest encodes a DNA repair protein. In some embodiments, the gene of interest encodes a structural protein. In some embodiments, the gene of interest encodes a low-density lipoprotein receptor (LDLR). In some embodiments, the gene of interest encodes alpha glucosidase. In some embodiments, the gene of interest encodes a cystic fibrosis transmembrane conductance regulator.

II.B.2.a. Therapeutic Proteins

In some embodiments, the genetic cassette encodes one therapeutic protein. In some embodiments, the genetic cassette encodes more than one therapeutic protein. In some embodiments, the genetic cassette encodes two or more copies of the same therapeutic protein. In some embodiments, the genetic cassette encodes two or more variants of the same therapeutic protein. In some embodiments, the genetic cassette encodes two or more different therapeutic proteins.

In some embodiments, the EV is associated with at least two AAVs, wherein each of the at least two AAV comprises a different genetic cassette, wherein each of the different genetic cassettes encodes a different therapeutic protein. In some embodiments, the EV is associated with at least three AAVs, at least four AAVs, or at least five AAVs.

In some embodiments, the therapeutic protein comprises a clotting factor. In some embodiments, the clotting factor is selected from the group consisting of FI, FII, FIII, FIV, FV, FVI, FVII, FVIII, FIX, FX, FXI, FXII, FXIII), VWF, prekallikrein, high-molecular weight kininogen, fibronectin, antithrombin III, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI-1), plasminogen activator inhibitor-2 (PAI2), any zymogen thereof, any active form thereof, and any combination thereof. In one embodiments, the clotting factor comprises FVIII or a variant or fragment thereof. In another embodiment, the clotting factor comprises FIX or a variant or fragment thereof. In another embodiment, the clotting factor comprises FVII or a variant or fragment thereof. In another embodiment, the clotting factor comprises VWF or a variant or fragment thereof.

II.B.2.a.i. Factor VIII

“Factor VIII,” abbreviated throughout the instant application as “FVIII,” as used herein, means functional FVIII polypeptide in its normal role in coagulation, unless otherwise specified. Thus, the term FVIII includes variant polypeptides that are functional. “A FVIII protein” is used interchangeably with FVIII polypeptide (or protein) or FVIII. Examples of the FVIII functions include, but are not limited to, an ability to activate coagulation, an ability to act as a cofactor for factor IX, or an ability to form a tenase complex with factor IX in the presence of Ca²⁺ and phospholipids, which then converts Factor X to the activated form Xa. The FVIII protein can be the human, porcine, canine, rat, or murine FVIII protein. In addition, comparisons between FVIII from humans and other species have identified conserved residues that are likely to be required for function (Cameron et al., Thromb. Haemost. 79:317-22 (1998); U.S. Pat. No. 6,251,632). The full length polypeptide and polynucleotide sequences are known, as are many functional fragments, mutants and modified versions. Various FVIII amino acid and nucleotide sequences are disclosed in, e.g., US Publication Nos. 2015/0158929 A1, 2014/0308280 A1, and 2014/0370035 A1 and International Publication No. WO 2015/106052 A1. FVIII polypeptides include, e.g., full-length FVIII, full-length FVIII minus Met at the N-terminus, mature FVIII (minus the signal sequence), mature FVIII with an additional Met at the N-terminus, and/or FVIII with a full or partial deletion of the B domain. FVIII variants include B domain deletions, whether partial or full deletions.

In some embodiments, the genetic cassette comprises a nucleotide sequence encoding a FVIII polypeptide, wherein the nucleotide sequence is codon optimized. In certain embodiments, the genetic cassette comprises a nucleotide sequence which is disclosed in International Application Publication No. WO 2019/032898; WO/2017/136358; or WO2017/136358; or U.S. Published Application No. 2015-0361158; which are incorporated by reference in their entirety.

In some embodiments, the genetic cassette comprises a nucleotide sequence encoding a FVIII polypeptide, wherein the nucleotide sequence is codon optimized. In some embodiments, the codon optimized nucleotide sequence encodes a full-length FVIII polypeptide. In other embodiments, the codon optimized nucleotide sequence encodes a B domain-deleted (BDD) FVIII polypeptide, wherein all or a portion of the B domain of FVIII is deleted.

II.B.2.a.ii Factor IX

In some embodiments, the therapeutic protein comprises a FIX polypeptide. In some embodiments, the FIX polypeptide comprises FIX or a variant or fragment thereof, wherein the FIX or the variant or fragment thereof has a FIX activity.

Human FIX is a serine protease that is an important component of the intrinsic pathway of the blood coagulation cascade. “Factor IX” or “FIX,” as used herein, refers to a coagulation factor protein and species and sequence variants thereof, and includes, but is not limited to, the 461 single-chain amino acid sequence of human FIX precursor polypeptide (“prepro”), the 415 single-chain amino acid sequence of mature human FIX, and the R338L FIX (Padua) variant. FIX includes any form of FIX molecule with the typical characteristics of blood coagulation FIX (see, for example, Choo et al., Nature 299:178-180 (1982); Fair et al., Blood 64:194-204 (1984); and Kurachi et al., Proc. Natl. Acad. Sci., U.S.A. 79:6461-6464 (1982); U.S. Pat. No. 7,939,632, each of which is incorporated herein by reference in its entirety.

Many functional FIX variants are known in the art. International publication number WO 02/040544 A3 discloses mutants that exhibit increased resistance to inhibition by heparin at page 4, lines 9-30 and page 15, lines 6-31. International publication number WO 03/020764 A2 discloses FIX mutants with reduced T cell immunogenicity in Tables 2 and 3 (on pages 14-24), and at page 12, lines 1-27. International publication number WO 2007/149406 A2 discloses functional mutant FIX molecules that exhibit increased protein stability, increased in vivo and in vitro half-life, and increased resistance to proteases at page 4, line 1 to page 19, line 11. WO 2007/149406 A2 also discloses chimeric and other variant FIX molecules at page 19, line 12 to page 20, line 9. International publication number WO 08/118507 A2 discloses FIX mutants that exhibit increased clotting activity at page 5, line 14 to page 6, line 5. International publication number WO 09/051717 A2 discloses FIX mutants having an increased number of N-linked and/or O-linked glycosylation sites, which results in an increased half-life and/or recovery at page 9, line 11 to page 20, line 2. International publication number WO 09/137254 A2 also discloses Factor IX mutants with increased numbers of glycosylation sites at page 2, paragraph [006] to page 5, paragraph [011] and page 16, paragraph [044] to page 24, paragraph [057]. International publication number WO 09/130198 A2 discloses functional mutant FIX molecules that have an increased number of glycosylation sites, which result in an increased half-life, at page 4, line 26 to page 12, line 6. International publication number WO 09/140015 A2 discloses functional FIX mutants that an increased number of Cys residues, which can be used for polymer (e.g., PEG) conjugation, at page 11, paragraph [0043] to page 13, paragraph [0053]. The FIX polypeptides described in International Application No. PCT/US2011/043569 filed Jul. 11, 2011 and published as WO 2012/006624 on Jan. 12, 2012 are also incorporated herein by reference in its entirety. In some embodiments, the FIX polypeptide comprises a FIX polypeptide fused to an albumin, e.g., FIX-albumin. In certain embodiments, the FIX polypeptide is IDELVION® or rIX-FP.

In some embodiments, the FIX is selected from a FIX disclosed in U.S. Pat. Nos. 7,404,956; 9,062,299; or 9,670,475; U.S. Published Application No. 2015-0252345; 2016-0000888; or 2017-0260516; or International Publication No. WO/2017/024060.

II.B.2.a.iv. Growth Factors

In some embodiments, therapeutic protein comprises a growth factor. The growth factor can be selected from any growth factor known in the art. In some embodiments, the growth factor is a hormone. In other embodiments, the growth factor is a cytokine. In some embodiments, the growth factor is a chemokine.

In some embodiments, the growth factor is adrenomedullin (AM). In some embodiments, the growth factor is angiopoietin (Ang). In some embodiments, the growth factor is autocrine motility factor. In some embodiments, the growth factor is a Bone morphogenetic protein (BMP). In some embodiments, the BMP is selects from BMP2, BMP4, BMP5, and BMP7. In some embodiments, the growth factor is a ciliary neurotrophic factor family member. In some embodiments, the ciliary neurotrophic factor family member is selected from ciliary neurotrophic factor (CNTF), leukemia inhibitory factor (LIF), interleukin-6 (IL-6). In some embodiments, the growth factor is a colony-stimulating factor. In some embodiments, the colony-stimulating factor is selected from macrophage colony-stimulating factor (m-CSF), granulocyte colony-stimulating factor (G-CSF), and granulocyte macrophage colony-stimulating factor (GM-CSF). In some embodiments, the growth factor is an epidermal growth factor (EGF). In some embodiments, the growth factor is an ephrin. In some embodiments, the ephrin is selected from ephrin A1, ephrin A2, ephrin A3, ephrin A4, ephrin A5, ephrin B 1, ephrin B2, and ephrin B3. In some embodiments, the growth factor is erythropoietin (EPO). In some embodiments, the growth factor is a fibroblast growth factor (FGF). In some embodiments, the FGF is selected from FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, and FGF23. In some embodiments, the growth factor is foetal bovine somatotrophin (FBS). In some embodiments, the growth factor is a GDNF family member. In some embodiments, the GDNF family member is selected from glial cell line-derived neurotrophic factor (GDNF), neurturin, persephin, and artemin. In some embodiments, the growth factor is growth differentiation factor-9 (GDF9). In some embodiments, the growth factor is hepatocyte growth factor (HGF). In some embodiments, the growth factor is hepatoma-derived growth factor (HDGF). In some embodiments, the growth factor is insulin. In some embodiments, the growth factor is an insulin-like growth factor. In some embodiments, the insulin-like growth factor is insulin-like growth factor-1 (IGF-1) or IGF-2. In some embodiments, the growth factor is an interleukin (IL). In some embodiments, the IL is selected from IL-1, IL-2, IL-3, IL-4, IL-5, IL-6, and IL-7. In some embodiments, the growth factor is keratinocyte growth factor (KGF). In some embodiments, the growth factor is migration-stimulating factor (MSF). In some embodiments, the growth factor is macrophage-stimulating protein (MSP or hepatocyte growth factor-like protein (HGFLP)). In some embodiments, the growth factor is myostatin (GDF-8). In some embodiments, the growth factor is a neuregulin. In some embodiments, the neuregulin is selected from neuregulin 1 (NRG1), NRG2, NRG3, and NRG4. In some embodiments, the growth factor is a neurotrophin. In some embodiments, the growth factor is brain-derived neurotrophic factor (BDNF). In some embodiments, the growth factor is nerve growth factor (NGF). In some embodiments, the NGF is neurotrophin-3 (NT-3) or NT-4. In some embodiments, the growth factor is placental growth factor (PGF). In some embodiments, the growth factor is platelet-derived growth factor (PDGF). In some embodiments, the growth factor is renalase (RNLS). In some embodiments, the growth factor is T-cell growth factor (TCGF). In some embodiments, the growth factor is thrombopoietin (TPO). In some embodiments, the growth factor is a transforming growth factor. In some embodiments, the transforming growth factor is transforming growth factor alpha (TGF-α) or TGF-β. In some embodiments, the growth factor is tumor necrosis factor-alpha (TNF-α). In some embodiments, the growth factor is vascular endothelial growth factor (VEGF).

In certain embodiments, the therapeutic protein comprises a subunit of the Rab geranylgeranyltransferase (GGTase) complex. In some embodiments, the therapeutic protein comprises Rab proteins GGTase component A 1 (REP1). In some embodiments, the REP1 comprises an amino acid sequence at least about 70%, at least about 75%, at least about at least about 80%, at least about 85%, at least about 90%, at least about 95%, at least about 96%, at least about 97%, at least about 98%, at least about 99%, or about 100% identical to SEQ ID NO: 45. REP1 deficiency is associated with Choroideremia (CHM), a rare X-linked progressive degeneration of the choroid, retinal pigment epithelium and photoreceptors of the eye. The typical natural history in afflicted males is onset of nightblindness during teenage years, and then progressive loss of peripheral vision during the 20's and 30's leading to complete blindness in the 40's. Female carriers have mild symptoms most notably nightblindness but can occasionally have a more severe phenotype.

TABLE 1
REP1 Amino Acid Sequence (SEQ ID NO: 45)

MADTLPSEFDVIVIGTGLPESIIAAACSRSGRRVLHVDSRSYYGGNWAS
FSFSGLLSWLKEYQENSDIVSDSPVWQDQILENEEAIALSRKDKTIQHV
EVFCYASQDLHEDVEEAGALQKNHALVTSANSTEAADSAFLPTEDESLS
TMSCEMLTEQTPSSDPENALEVNGAEVTGEKENHCDDKTCVPSTSAEDM
SENVPIAEDTTEQPKKNRITYSQIIKEGRRFNIDLVSKLLYSRGLLIDL
LIKSNVSRYAEFKNITRILAFREGRVEQVPCSRADVFNSKQLTMVEKRM
LMKFLTFCMEYEKYPDEYKGYEEITFYEYLKTQKLTPNLQYIVMHSIAM
TSETASSTIDGLKATKNFLHCLGRYGNTPFLFPLYGQGELPQCFCRMCA
VFGGIYCLRHSVQCLVVDKESRKCKAIIDQFGQRIISEHFLVEDSYFPE
NMCSRVQYRQISRAVLITDRSVLKTDSDQQISILTVPAEEPGTFAVRVI
ELCSSTMTCMKGTYLVHLTCTSSKTAREDLESVVQKLFVPYTEMEIENE
QVEKPRILWALYFNMRDSSDISRSCYNDLPSNVYVCSGPDCGLGNDNAV
KQAETLFQEICPNEDFCPPPPNPEDIILDGDSLQPEASESSAIPEANSE
TFKESTNLGNLEESSE

The disease is caused by mutations in the REP1 gene, (Rab escort protein 1), which is located on the X chromosome 21q region. In most cells in the body, the REP2 protein, which is 75% homologous to REP1, compensates for the REP1 deficiency. In the eye, however, for reasons that are not yet clear, REP2 is unable to compensate for the REP1 deficiency. Hence in the eye, REP polypeptide activity is insufficient to maintain normal prenylation of the target proteins (Rab GTPases) leading to cellular dysfunction and ultimate death, primarily affecting the outer retina and choroid.

II.B.2.b. AAV Sequence

In certain embodiments, the AAV further comprises a first ITR, e.g., a 5′ ITR, and second ITR, e.g., a 3′ ITR. Typically, ITRs are involved in parvovirus (e.g., AAV) DNA replication and rescue, or excision, from prokaryotic plasmids (Samulski et al., 1983, 1987; Senapathy et al., 1984; Gottlieb and Muzyczka, 1988). In addition, ITRs are reported to be the minimum sequences required for AAV proviral integration and for packaging of AAV DNA into virions (McLaughlin et al., 1988; Samulski et al., 1989). These elements are essential for efficient multiplication of a Parvovirus genome.

In some embodiments, the ITR comprises a naturally occurring ITR, e.g., the ITR comprises all or a portion of a Parvovirus ITR. In some embodiments, the ITR comprises a synthetic sequence. In one embodiment, the first ITR or the second ITR comprises a synthetic sequence. In another embodiment, each of the first ITR and the second ITR comprises a synthetic sequence. In some embodiments, the first ITR or the second ITR comprises a naturally occurring sequence. In another embodiment, each of the first ITR and the second ITR comprises a naturally occurring sequence.

In some embodiments, the ITR comprises an ITR from an AAV genome. In some embodiments, the ITR is an ITR of an AAV genome selected from AAV1, AAV2, AAV3, AAV4, AAV5, AAV6, AAV7, AAV8, AAV9, AAV10 AAV11, and any combination thereof. In a particular embodiment, the ITR is an ITR of the AAV2 genome. In certain embodiments, the ITR is an ITR of AAV2, and the capsid protein is a capsid protein of AAV5. In another embodiment, the ITR is a synthetic sequence genetically engineered to include at its 5′ and 3′ ends ITRs or fragments thereof derived from one or more of AAV genomes. In some embodiments, the ITRs are derived from the same genome, e.g., from the genome of the same virus, or from different genomes, e.g., from the genomes of two or more different AAV genomes. In certain embodiments, the ITRs are derived from the same AAV genome. In a specific embodiment, the two ITRs present in the nucleic acid molecule of the disclosure are the same, and can in particular be AAV2 ITRs, AAV5 ITRs or AAV9 ITRs. In one particular embodiment, the first ITR and the second ITR are identical.

In some embodiments, the ITRs form hairpin loop structures. In one embodiment, the first ITR forms a hairpin structure. In another embodiment, the second ITR forms a hairpin structure. Still in another embodiment, both the first ITR and the second ITR form hairpin structures.

In some embodiments, an ITR in a nucleic acid molecule described herein is a transcriptionally activated ITR. A transcriptionally-activated ITR can comprise all or a portion of a wild-type ITR that has been transcriptionally activated by inclusion of at least one transcriptionally active element. Various types of transcriptionally active elements are suitable for use in this context. In some embodiments, the transcriptionally active element is a constitutive transcriptionally active element. Constitutive transcriptionally active elements provide an ongoing level of gene transcription, and are preferred when it is desired that the transgene be expressed on an ongoing basis. In other embodiments, the transcriptionally active element is an inducible transcriptionally active element. Inducible transcriptionally active elements generally exhibit low activity in the absence of an inducer (or inducing condition), and are up-regulated in the presence of the inducer (or switch to an inducing condition). Inducible transcriptionally active elements can be preferred when expression is desired only at certain times or at certain locations, or when it is desirable to titrate the level of expression using an inducing agent. Transcriptionally active elements can also be tissue-specific; that is, they exhibit activity only in certain tissues or cell types.

Transcriptionally active elements can be incorporated into an ITR in a variety of ways. In some embodiments, a transcriptionally active element is incorporated 5′ to any portion of an ITR or 3′ to any portion of an ITR. In other embodiments, a transcriptionally active element of a transcriptionally-activated ITR lies between two ITR sequences. If the transcriptionally active element comprises two or more elements which must be spaced apart, those elements can alternate with portions of the ITR. In some embodiments, a hairpin structure of an ITR is deleted and replaced with inverted repeats of a transcriptional element. This latter arrangement would create a hairpin mimicking the deleted portion in structure. Multiple tandem transcriptionally active elements can also be present in a transcriptionally-activated ITR, and these can be adjacent or spaced apart. In addition, protein binding sites (e.g., Rep binding sites) can be introduced into transcriptionally active elements of the transcriptionally-activated ITRs. A transcriptionally active element can comprise any sequence enabling the controlled transcription of DNA by RNA polymerase to form RNA, and can comprise, for example, a transcriptionally active element, as defined below.

Transcriptionally-activated ITRs provide both transcriptional activation and ITR functions to the nucleic acid molecule in a relatively limited nucleotide sequence length which effectively maximizes the length of a transgene which can be carried and expressed from the nucleic acid molecule. Incorporation of a transcriptionally active element into an ITR can be accomplished in a variety of ways. A comparison of the ITR sequence and the sequence requirements of the transcriptionally active element can provide insight into ways to encode the element within an ITR. For example, transcriptional activity can be added to an ITR through the introduction of specific changes in the ITR sequence that replicates the functional elements of the transcriptionally active element. A number of techniques exist in the art to efficiently add, delete, and/or change particular nucleotide sequences at specific sites (see, for example, Deng and Nickoloff (1992) Anal. Biochem. 200:81-88). Another way to create transcriptionally-activated ITRs involves the introduction of a restriction site at a desired location in the ITR. In addition, multiple transcriptionally activate elements can be incorporated into a transcriptionally-activated ITR, using methods known in the art.

By way of illustration, transcriptionally-activated ITRs can be generated by inclusion of one or more transcriptionally active elements such as: TATA box, GC box, CCAAT box, Sp1 site, Inr region, CRE (cAMP regulatory element) site, ATF-1/CRE site, APBβ box, APBα box, CArG box, CCAC box, or any other element involved in transcription as known in the art.

In some embodiments, the AAV comprises a genetic cassette encoding more than one therapeutic protein. In AAVs encoding more than one therapeutic protein, some embodiments include elements such as IRES or 2A, to co-express them from one promoter. In some embodiments, the AAV comprises protein coding regions separated by an IRES element. In some embodiments, the AAV comprises two protein coding regions separated by a 2A element. In some embodiments, the AAV comprises three protein coding regions separated by an IRES element between the protein coding regions. In some embodiments, the AAV comprises three protein coding regions separated by 2A elements between the protein coding regions.

In some embodiments, the AAV comprises a regulatory sequence. In some embodiments, the AAV comprises non-coding regulatory DNA. In some embodiments, the AAV genome comprises regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the AAV, including the selection of regulatory sequences, can depend on such factors as the choice of the host cell to be transformed, the level of expression of protein desired, etc. In some embodiments, the AAV genome comprises mRNA splice donor/splice acceptor sites. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences can be used, such as the ubiquitin promoter or β-globin promoter. Still further, regulatory elements composed of sequences from different sources, such as the SRa promoter system, which contains sequences from the SV40 early promoter and the long terminal repeat of human T cell leukemia virus type 1 (Takebe et al. (1988) Mol. Cell. Biol. 8:466-472). In certain embodiments, the regulatory sequence comprises a tissue specific promoter. In some embodiments, the tissue specific promoter drives expression of the gene of interest in a tissue selected from the group consisting of heart, liver, lungs, eyes, nervous system, lymphatic system, muscle and stem cells.

II.B.2.c. Methods and Uses of AAVs

Methods for obtaining recombinant AAVs having a desired capsid protein are well known in the art. (See, for example, US 2003/0138772, the contents of which are incorporated herein by reference in their entirety). Typically the methods involve culturing a host cell which contains a nucleic acid sequence encoding an AAV capsid protein or fragment thereof; a functional rep gene; a recombinant AAV vector composed of, AAV ITRs and a transgene; and sufficient helper functions to permit packaging of the recombinant AAV vector into the AAV capsid proteins. Helper functions to increase AAV production can be supplied through transfection with plasmid DNA, or by co-infection with adenovirus.

The components to be cultured in the host cell to package a rAAV vector in an AAV capsid can be provided to the host cell in trans. Alternatively, any one or more of the required components (e.g., recombinant AAV vector, rep sequences, cap sequences, and/or helper functions) can be provided by a stable host cell which has been engineered to contain one or more of the required components using methods known to those of skill in the art. Most suitably, such a stable host cell will contain the required component(s) under the control of an inducible promoter. However, the required component(s) can be under the control of a constitutive promoter. Examples of suitable inducible and constitutive promoters are provided herein, in the discussion of regulatory elements suitable for use with the transgene. In still another alternative, a selected stable host cell can contain selected component(s) under the control of a constitutive promoter and other selected component(s) under the control of one or more inducible promoters. For example, a stable host cell can be generated which is derived from 293 cells (which contain E1 helper functions under the control of a constitutive promoter), but which contain the rep and/or cap proteins under the control of inducible promoters. Still other stable host cells can be generated by one of skill in the art.

The recombinant AAV vector, rep sequences, cap sequences, and helper functions required for producing the rAAV of the disclosure can be delivered to the packaging host cell using any appropriate genetic element (vector). The selected genetic element can be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, recombinant AAVs are be produced using the triple transfection method (described in detail in U.S. Pat. No. 6,001,650). Typically, the recombinant AAVs are produced by transfecting a host cell with an recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), baculovirus, and vaccinia virus. In some embodiments, recombinant AAVs are produced using transient transfection of HEK293 cells with a triple plasmid system described herein.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell can be used as a recipient of an AAV helper construct, an accessory function vector, or other transfer DNA associated with the production of recombinant AAVs. The term includes the progeny of the original cell which has been transfected. Thus, a “host cell” as used herein can refer to a cell which has been transfected with an exogenous DNA sequence. In some embodiments, AAV is produced using transient or stable expression. In some embodiments, the host cell is HEK293, HeLa cells, BHK cells, or sf9 cells. In a particular embodiment, the host cell is a HEK293 cell. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA (e.g., shRNA, miRNA, miRNA inhibitor) from a transcribed gene.

In some embodiments, AAV or rAAV is purified using an Iodixanol density gradient. In some embodiments, empty capsids migrate with a density of about 1.3±0.05 g/mL (e.g., 1.3-1.32 g/mL). In some embodiments, genome-containing capsids migrate with a density of about 1.4±0.05 g/mL (e.g., 1.35-1.42 g/mL).

II.C. Scaffold Proteins

In certain aspects of the disclosure, the EV comprises an AAV and one or more scaffold proteins. In some embodiments, EVs of the present disclosure comprise a membrane modified in its composition. For example, their membrane compositions can be modified by changing the protein, lipid, or glycan content of the membrane.

In some embodiments, the surface-engineered EVs, e.g., exosomes, are generated by chemical and/or physical methods, such as PEG-induced fusion and/or ultrasonic fusion. In other embodiments, the surface-engineered EVs, e.g., exosomes, are generated by genetic engineering. EVs, e.g., exosomes, produced from a genetically-modified producer cell or a progeny of the genetically-modified cell can contain modified membrane compositions. In some embodiments, surface-engineered EVs, e.g., exosomes, have scaffold protein at a higher or lower density (e.g., higher number) or include a variant or a fragment of the scaffold protein.

In some embodiments, surface-engineered EVs are produced from a cell (e.g., HEK293 cells) transformed with an exogenous sequence encoding a scaffold protein (e.g., exosome proteins or a scaffold protein disclosed herein) or a variant or a fragment thereof. EVs including a scaffold protein expressed from the exogenous sequence can include modified membrane compositions.

Various modifications or fragments of the scaffold protein can be used for the embodiments of the present disclosure. For example, scaffold protein modified to have enhanced affinity to a binding agent can be used for generating surface-engineered EV that can be purified using the binding agent. Scaffold proteins modified to be more effectively targeted to EVs and/or membranes can be used. Scaffold proteins modified to comprise a minimal fragment required for specific and effective targeting to exosome membranes can be also used.

A scaffold protein can be engineered to be expressed as a fusion molecule, e.g., fusion molecule of an exosome membrane protein to an AAV. For example, the fusion molecule can comprise a scaffold protein disclosed herein (e.g., PTGFRN, BSG, IGSF2, IGSF3, IGSF8, ITGB1, ITGA4, SLC3A2, ATP transporter, or a fragment or a variant thereof) linked to a capsid protein of an AAV, either directly or through an intermediate (e.g., a chemically inducible dimer, an antigen binding domain, or a receptor). In case of the fusion molecule, the chemically inducible dimer, the antigen binding domain, and/or the receptor can be a natural peptide, a recombinant peptide, a synthetic peptide, or any combination thereof.

In some embodiments, the surface-engineered EVs described herein demonstrate superior characteristics compared to EVs known in the art. For example, surface-engineered EVs contain modified proteins more highly enriched on their surface than naturally occurring EVs or the EVs produced using conventional exosome proteins. Moreover, the surface-engineered EVs of the present disclosure can have greater, more specific, or more controlled biological activity compared to naturally occurring EVs or the EVs produced using conventional exosome proteins.

Scaffold proteins of the present disclosure can be used for external or luminal (interior) anchoring. In some embodiments, the scaffold protein is capable of anchoring a heterologous polypeptide to the external surface of the EV, e.g., the scaffold protein has an extracellular domain. In some embodiments, the scaffold protein is capable of anchoring a heterologous polypeptide to the interior surface of the EV, e.g., the scaffold protein has an intracellular (luminal) domain. In some embodiments, the scaffold protein is capable of anchoring a heterologous polypeptide to either the external surface of the EV or the luminal surface of the EV, or both, e.g., the scaffold protein has an extracellular domain and an intracellular domain, e.g., the EV is a transmembrane protein.

II.C.1. Transmembrane Scaffold Proteins

In some embodiments the scaffold protein (e.g., Scaffold X) comprises Prostaglandin F2 receptor negative regulator (the PTGFRN polypeptide). The PTGFRN protein can be also referred to as CD9 partner 1 (CD9P-1), Glu-Trp-Ile EWI motif-containing protein F (EWI-F), Prostaglandin F2-alpha receptor regulatory protein, Prostaglandin F2-alpha receptor-associated protein, or CD315. The full length amino acid sequence of the human PTGFRN protein (Uniprot Accession No. Q9P2B2) is shown at TABLE 3 as SEQ ID NO: 1. The PTGFRN polypeptide contains a signal peptide (amino acids 1 to 25 of SEQ ID NO: 1), the extracellular domain (amino acids 26 to 832 of SEQ ID NO: 1), a transmembrane domain (amino acids 833 to 853 of SEQ ID NO: 1), and a cytoplasmic domain (amino acids 854 to 879 of SEQ ID NO: 1). The mature PTGFRN polypeptide consists of SEQ ID NO: 1 without the signal peptide, i.e., amino acids 26 to 879 of SEQ ID NO: 1. In some embodiments, a PTGFRN polypeptide fragment useful for the present disclosure comprises a transmembrane domain of the PTGFRN polypeptide. In other embodiments, a PTGFRN polypeptide fragment useful for the present disclosure comprises the transmembrane domain of the PTGFRN polypeptide and (i) at least about five, at least about 10, at least about 15, at least about 20, at least about 25, at least about 30, at least about 40, at least about 50, at least about 70, at least about 80, at least about 90, at least about 100, at least about 110, at least about 120, at least about 130, at least about 140, at least about 150 amino acids at the N terminus of the transmembrane domain, (ii) at least about five, at least about 10, at least about 15, at least about 20, or at least about 25 amino acids at the C terminus of the transmembrane domain, or both (i) and (ii).

In some embodiments, the fragments of PTGFRN polypeptide lack one or more functional or structural domains, such as IgV.

In other embodiments, the scaffold protein comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 26 to 879 of SEQ ID NO: 1. In other embodiments, the scaffold protein comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 2 (corresponding to positions 687 to 878 of SEQ ID NO: 1).

In other embodiments, the scaffold protein comprises the amino acid sequence of SEQ ID NO: 2, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some embodiments, the scaffold protein comprises the amino acid sequence of SEQ ID NO: 2 and one amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 2.

In other embodiments, the scaffold protein comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 186, 187, 188, 189, 190, or 191. In other embodiments, the scaffold protein comprises the amino acid sequence of SEQ ID NO: 186, 187, 188, 189, 190, or 191, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some embodiments, the scaffold protein comprises the amino acid sequence of SEQ ID NO: 186, 187, 188, 189, 190, or 191 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 186, 187, 188, 189, 190, or 191.

TABLE 2
Exemplary Scaffold Protein Sequences

| Protein | Sequence |
|---|---|
| PTGFRN | MGRLASRPLLLALLSLALCRGRVVRVPTATLVRVVGTELVIPCNVSDYDGPSEQN |
| Protein | FDWSFSSLGSSFVELASTWEVGFPAQLYQERLQRGEILLRRTANDAVELHIKNVQ |
| (SEQ ID | PSDQGHYKCSTPSTDATVQGNYEDTVQVKVLADSLHVGPSARPPPSLSLREGEPF |
| NO: 1) | ELRCTAASASPLHTHLALLWEVHRGPARRSVLALTHEGRFHPGLGYEQRYHSGDV |
| | RLDTVGSDAYRLSVSRALSADQGSYRCIVSEWIAEQGNWQEIQEKAVEVATVVIQ |
| | PSVLRAAVPKNVSVAEGKELDLTCNITTDRADDVRPEVTWSFSRMPDSTLPGSRV |
| | LARLDRDSLVHSSPHVALSHVDARSYHLLVRDVSKENSGYYYCHVSLWAPGHNRS |
| | WHKVAEAVSSPAGVGVTWLEPDYQVYLNASKVPGFADDPTELACRVVDTKSGEAN |
| | VRFTVSWYYRMNRRSDNVVTSELLAVMDGDWTLKYGERSKQRAQDGDFIFSKEHT |
| | DTFNFRIQRTTEEDRGNYYCVVSAWTKQRNNSWVKSKDVFSKPVNIFWALEDSVL |
| | VVKARQPKPFFAAGNTFEMTCKVSSKNIKSPRYSVLIMAEKPVGDLSSPNETKYI |
| | ISLDQDSVVKLENWTDASRVDGVVLEKVQEDEFRYRMYQTQVSDAGLYRCMVTAW |
| | SPVRGSLWREAATSLSNPIEIDFQTSGPIFNASVHSDTPSVIRGDLIKLFCIITV |
| | EGAALDPDDMAFDVSWFAVHSFGLDKAPVLLSSLDRKGIVTTSRRDWKSDLSLER |
| | VSVLEFLLQVHGSEDQDFGNYYCSVTPWVKSPTGSWQKEAEIHSKPVFITVKMDV |
| | LNAFKYPLLIGVGLSTVIGLLSCLIGYCSSHWCCKKEVQETRRERRRLMSMEMD |
| PTGFRN | GPIFNASVHSDTPSVIRGDLIKLFCIITVEGAALDPDDMAFDVSWFAVHSFGLDK |
| protein | APVLLSSLDRKGIVTTSRRDWKSDLSLERVSVLEFLLQVHGSEDQDFGNYYCSVT |
| Fragment | PWVKSPTGSWQKEAEIHSKPVFITVKMDVLNAFKYPLLIGVGLSTVIGLLSCLIG |
| (SEQ ID | YCSSHWCCKKEVQETRRERRRLMSMEM |
| NO: 2) | 687-878 of SEQ ID NO: 1 |
| BSG | MAAALFVLLGFALLGTHGASGAAGFVQAPLSQQRWVGGSVELHCEAVGSPVPEIQ |
| protein | WWFEGQGPNDTCSQLWDGARLDRVHIHATYHQHAASTISIDTLVEEDTGTYECRA |
| (SEQ ID | SNDPDRNHLTRAPRVKWVRAQAVVLVLEPGTVFTTVEDLGSKILLTCSLNDSATE |
| NO: 3) | VTGHRWLKGGVVLKEDALPGQKTEFKVDSDDQWGEYSCVFLPEPMGTANIQLHGP |
| | PRVKAVKSSEHINEGETAMLVCKSESVPPVTDWAWYKITDSEDKALMNGSESRFF |
| | VSSSQGRSELHIENLNMEADPGQYRCNGTSSKGSDQAIITLRVRSHLAALWPFLG |
| | IVAEVLVLVTIIFIYEKRRKPEDVLDDDDAGSAPLKSSGQHQNDKGKNVRQRNSS |
| IGSF8 | MGALRPTLLPPSLPLLLLLMLGMGCWAREVLVPEGPLYRVAGTAVSISCNVTGYE |
| protein | GPAQQNFEWFLYRPEAPDTALGIVSTKDTQFSYAVFKSRVVAGEVQVQRLQGDAV |
| (SEQ ID | VLKIARLQAQDAGIYECHTPSTDTRYLGSYSGKVELRVLPDVLQVSAAPPGPRGR |
| NO: 4) | QAPTSPPRMTVHEGQELALGCLARTSTQKHTHLAVSFGRSVPEAPVGRSTLQEVV |
| | GIRSDLAVEAGAPYAERLAAGELRLGKEGTDRYRMVVGGAQAGDAGTYHCTAAEW |
| | IQDPDGSWAQIAEKRAVLAHVDVQTLSSQLAVTVGPGERRIGPGEPLELLCNVSG |
| | ALPPAGRHAAYSVGWEMAPAGAPGPGRLVAQLDTEGVGSLGPGYEGRHIAMEKVA |
| | SRTYRLRLEAARPGDAGTYRCLAKAYVRGSGTRLREAASARSRPLPVHVREEGVV |
| | LEAVAWLAGGTVYRGETASLLCNISVRGGPPGLRLAASWWVERPEDGELSSVPAQ |
| | LVGGVGQDGVAELGVRPGGGPVSVELVGPRSHRLRLHSLGPEDEGVYHCAPSAWV |
| | QHADYSWYQAGSARSGPVTVYPYMHALDTLFVPLLVGTGVALVTGATVLGTITCC |
| | FMKRLRKR |
| ITGB1 | MNLQPIFWIGLISSVCCVFAQTDENRCLKANAKSCGECIQAGPNCGWCTNSTFLQ |
| protein | EGMPTSARCDDLEALKKKGCPPDDIENPRGSKDIKKNKNVTNRSKGTAEKLKPED |
| (SEQ ID | ITQIQPQQLVLRLRSGEPQTFTLKFKRAEDYPIDLYYLMDLSYSMKDDLENVKSL |
| NO: 5) | GTDLMNEMRRITSDFRIGFGSFVEKTVMPYISTTPAKLRNPCTSEQNCTSPFSYK |
| | NVLSLTNKGEVFNELVGKQRISGNLDSPEGGFDAIMQVAVCGSLIGWRNVTRLLV |
| | FSTDAGFHFAGDGKLGGIVLPNDGQCHLENNMYTMSHYYDYPSIAHLVQKLSENN |
| | IQTIFAVTEEFQPVYKELKNLIPKSAVGTLSANSSNVIQLIIDAYNSLSSEVILE |
| | NGKLSEGVTISYKSYCKNGVNGTGENGRKCSNISIGDEVQFEISITSNKCPKKDS |
| | DSFKIRPLGFTEEVEVILQYICECECQSEGIPESPKCHEGNGTFECGACRCNEGR |
| | VGRHCECSTDEVNSEDMDAYCRKENSSEICSNNGECVCGQCVCRKRDNTNEIYSG |
| | ASNGQICNGRGICECGVCKCTDPKFQGQTCEMCQTCLGVCAEHKECVQCRAFNKG |
| | EKKDTCTQECSYFNITKVESRDKLPQPVQPDPVSHCKEKDVDDCWFYFTYSVNGN |
| | NEVMVHVVENPECPTGPDIIPIVAGVVAGIVLIGLALLLIWKLLMIIHDRREFAK |
| | FEKEKMNAKWDTGENPIYKSAVTTVVNPKYEGK |
| ITGA4 | MAWEARREPGPRRAAVRETVMLLLCLGVPTGRPYNVDTESALLYQGPHNTLFGYS |
| protein | VVLHSHGANRWLLVGAPTANWLANASVINPGAIYRCRIGKNPGQTCEQLQLGSPN |
| (SEQ ID | GEPCGKTCLEERDNQWLGVTLSRQPGENGSIVTCGHRWKNIFYIKNENKLPTGGC |
| NO: 6) | YGVPPDLRTELSKRIAPCYQDYVKKFGENFASCQAGISSFYTKDLIVMGAPGSSY |
| | WTGSLFVYNITTNKYKAFLDKQNQVKFGSYLGYSVGAGHFRSQHTTEVVGGAPQH |
| | EQIGKAYIFSIDEKELNILHEMKGKKLGSYFGASVCAVDLNADGFSDLLVGAPMQ |
| | STIREEGRVFVYINSGSGAVMNAMETNLVGSDKYAARFGESIVNLGDIDNDGFED |
| | VAIGAPQEDDLQGAIYIYNGRADGISSTFSQRIEGLQISKSLSMFGQSISGQIDA |
| | DNNGYVDVAVGAFRSDSAVLLRTRPVVIVDASLSHPESVNRTKFDCVENGWPSVC |
| | IDLTLCFSYKGKEVPGYIVLFYNMSLDVNRKAESPPRFYFSSNGTSDVITGSIQV |
| | SSREANCRTHQAFMRKDVRDILTPIQIEAAYHLGPHVISKRSTEEFPPLQPILQQ |
| | KKEKDIMKKTINFARFCAHENCSADLQVSAKIGFLKPHENKTYLAVGSMKTLMLN |
| | VSLFNAGDDAYETTLHVKLPVGLYFIKILELEEKQINCEVTDNSGVVQLDCSIGY |
| | IYVDHLSRIDISFLLDVSSLSRAEEDLSITVHATCENEEEMDNLKHSRVTVAIPL |
| | KYEVKLTVHGFVNPTSFVYGSNDENEPETCMVEKMNLTFHVINTGNSMAPNVSVE |
| | IMVPNSFSPQTDKLFNILDVQTTTGECHFENYQRVCALEQQKSAMQTLKGIVRFL |
| | SKTDKRLLYCIKADPHCLNFLCNFGKMESGKEASVHIQLEGRPSILEMDETSALK |
| | FEIRATGFPEPNPRVIELNKDENVAHVLLEGLHHQRPKRYFTIVIISSSLLLGLI |
| | VLLLISYVMWKAGFFKRQYKSILQEENRRDSWSYINSKSNDD |
| SLC3A2 | MELQPPEASIAVVSIPRQLPGSHSEAGVQGLSAGDDSELGSHCVAQTGLELLASG |
| Protein, | DPLPSASQNAEMIETGSDCVTQAGLQLLASSDPPALASKNAEVTGTMSQDTEVDM |
| where | KEVELNELEPEKQPMNAASGAAMSLAGAEKNGLVKIKVAEDEAEAAAAAKFTGLS |
| the first | KEELLKVAGSPGWVRTRWALLLLFWLGWLGMLAGAVVIIVRAPRCRELPAQKWWH |
| Met is | TGALYRIGDLQAFQGHGAGNLAGLKGRLDYLSSLKVKGLVLGPIHKNQKDDVAQT |
| processed. | DLLQIDPNFGSKEDFDSLLQSAKKKSIRVILDLTPNYRGENSWFSTQVDTVATKV |
| (SEQ ID | KDALEFWLQAGVDGFQVRDIENLKDASSFLAEWQNITKGFSEDRLLIAGTNSSDL |
| NO: 7) | QQILSLLESNKDLLLTSSYLSDSGSTGEHTKSLVTQYLNATGNRWCSWSLSQARL |
| | LTSFLPAQLLRLYQLMLFTLPGTPVFSYGDEIGLDAAALPGQPMEAPVMLWDESS |
| | FPDIPGAVSANMTVKGQSEDPGSLLSLFRRLSDQRSKERSLLHGDFHAFSAGPGL |
| | FSYIRHWDQNERFLVVLNFGDVGLSAGLQASDLPASASLPAKADLLLSTQPGREE |
| | GSPLELERLKLEPHEGLLLRFPYAA |
| PTGFRN | PSARPPPSLSLREGEPFELRCTAASASPLHTHLALLWEVHRGPARRSVLALTHEG |
| fragment 1 | RFHPGLGYEQRYHSGDVRLDTVGSDAYRLSVSRALSADQGSYRCIVSEWIAEQGN |
| (SEQ ID | WQEIQEKAVEVATVVIQPSVLRAAVPKNVSVAEGKELDLTCNITTDRADDVRPEV |
| NO: 186) | TWSFSRMPDSTLPGSRVLARLDRDSLVHSSPHVALSHVDARSYHLLVRDVSKENS |
| | GYYYCHVSLWAPGHNRSWHKVAEAVSSPAGVGVTWLEPDYQVYLNASKVPGFADD |
| | PTELACRVVDTKSGEANVRFTVSWYYRMNRRSDNVVTSELLAVMDGDWTLKYGER |
| | SKQRAQDGDFIFSKEHTDTFNFRIQRTTEEDRGNYYCVVSAWTKQRNNSWVKSKD |
| | VFSKPVNIFWALEDSVLVVKARQPKPFFAAGNTFEMTCKVSSKNIKSPRYSVLIM |
| | AEKPVGDLSSPNETKYIISLDQDSVVKLENWTDASRVDGVVLEKVQEDEFRYRMY |
| | QTQVSDAGLYRCMVTAWSPVRGSLWREAATSLSNPIEIDFQTSGPIFNASVHSDT |
| | PSVIRGDLIKLFCIITVEGAALDPDDMAFDVSWFAVHSFGLDKAPVLLSSLDRKG |
| | IVTTSRRDWKSDLSLERVSVLEFLLQVHGSEDQDFGNYYCSVTPWVKSPTGSWQK |
| | EAEIHSKPVFITVKMDVLNAFKYPLLIGVGLSTVIGLLSCLIGYCSSHWCCKKEV |
| | QETRRERRRLMSMEMD |
| PTGFRN | VATVVIQPSVLRAAVPKNVSVAEGKELDLTCNITTDRADDVRPEVTWSFSRMPDS |
| fragment 2 | TLPGSRVLARLDRDSLVHSSPHVALSHVDARSYHLLVRDVSKENSGYYYCHVSLW |
| (SEQ ID | APGHNRSWHKVAEAVSSPAGVGVTWLEPDYQVYLNASKVPGFADDPTELACRVVD |
| NO: 187) | TKSGEANVRFTVSWYYRMNRRSDNVVTSELLAVMDGDWTLKYGERSKQRAQDGDF |
| | IFSKEHTDTFNFRIQRTTEEDRGNYYCVVSAWTKQRNNSWVKSKDVFSKPVNIFW |
| | ALEDSVLVVKARQPKPFFAAGNTFEMTCKVSSKNIKSPRYSVLIMAEKPVGDLSS |
| | PNETKYIISLDQDSVVKLENWTDASRVDGVVLEKVQEDEFRYRMYQTQVSDAGLY |
| | RCMVTAWSPVRGSLWREAATSLSNPIEIDFQTSGPIFNASVHSDTPSVIRGDLIK |
| | LFCIITVEGAALDPDDMAFDVSWFAVHSFGLDKAPVLLSSLDRKGIVTTSRRDWK |
| | SDLSLERVSVLEFLLQVHGSEDQDFGNYYCSVTPWVKSPTGSWQKEAEIHSKPVF |
| | ITVKMDVLNAFKYPLLIGVGLSTVIGLLSCLIGYCSSHWCCKKEVQETRRERRRL |
| | MSMEMD |
| PTGFRN | SPAGVGVTWLEPDYQVYLNASKVPGFADDPTELACRVVDTKSGEANVRFTVSWYY |
| fragment 3 | RMNRRSDNVVTSELLAVMDGDWTLKYGERSKQRAQDGDFIFSKEHTDTFNFRIQR |
| (SEQ ID | TTEEDRGNYYCVVSAWTKQRNNSWVKSKDVFSKPVNIFWALEDSVLVVKARQPKP |
| NO: 188) | FFAAGNTFEMTCKVSSKNIKSPRYSVLIMAEKPVGDLSSPNETKYIISLDQDSVV |
| | KLENWTDASRVDGVVLEKVQEDEFRYRMYQTQVSDAGLYRCMVTAWSPVRGSLWR |
| | EAATSLSNPIEIDFQTSGPIFNASVHSDTPSVIRGDLIKLFCIITVEGAALDPDD |
| | MAFDVSWFAVHSFGLDKAPVLLSSLDRKGIVTTSRRDWKSDLSLERVSVLEFLLQ |
| | VHGSEDQDFGNYYCSVTPWVKSPTGSWQKEAEIHSKPVFITVKMDVLNAFKYPLL |
| | IGVGLSTVIGLLSCLIGYCSSHWCCKKEVQETRRERRRLMSMEMD |
| PTGFRN | KPVNIFWALEDSVLVVKARQPKPFFAAGNTFEMTCKVSSKNIKSPRYSVLIMAEK |
| fragment 4 | PVGDLSSPNETKYIISLDQDSVVKLENWTDASRVDGVVLEKVQEDEFRYRMYQTQ |
| (SEQ ID | VSDAGLYRCMVTAWSPVRGSLWREAATSLSNPIEIDFQTSGPIFNASVHSDTPSV |
| NO: 189) | IRGDLIKLFCIITVEGAALDPDDMAFDVSWFAVHSFGLDKAPVLLSSLDRKGIVT |
| | TSRRDWKSDLSLERVSVLEFLLQVHGSEDQDFGNYYCSVTPWVKSPTGSWQKEAE |
| | IHSKPVFITVKMDVLNAFKYPLLIGVGLSTVIGLLSCLIGYCSSHWCCKKEVQET |
| | RRERRRLMSMEMD |
| PTGFRN | VRGSLWREAATSLSNPIEIDFQTSGPIFNASVHSDTPSVIRGDLIKLFCIITVEG |
| fragment 5 | AALDPDDMAFDVSWFAVHSFGLDKAPVLLSSLDRKGIVTTSRRDWKSDLSLERVS |
| (SEQ ID | VLEFLLQVHGSEDQDFGNYYCSVTPWVKSPTGSWQKEAEIHSKPVFITVKMDVLN |
| NO: 190) | AFKYPLLIGVGLSTVIGLLSCLIGYCSSHWCCKKEVQETRRERRRLMSMEMD |
| PTGFRN | SKPVFITVKMDVLNAFKYPLLIGVGLSTVIGLLSCLIGYCSSHWCCKKEVQETRR |
| fragment 6 | ERRRLMSMEMD |
| (SEQ ID | |
| NO: 191) | |
| PTGFRN | MGRLASRPLLLALLSLALCRG |
| Signal | |
| peptide | |
| (SEQ ID | |
| NO: 192) | |
| BSG Protein | PGTVFTTVEDLGSKILLTCSLNDSATEVTGHRWLKGGVVLKEDALPGQKTEFKVDSDDQW |
| Fragment 1 | GEYSCVFLPEPMGTANIQLHGPPRVKAVKSSEHINEGETAMLVCKSESVPPVTDWAWYKI |
| (SEQ ID NO: | TDSEDKALMNGSESRFFVSSSQGRSELHIENLNMEADPGQYRCNGTSSKGSDQAIITLRV |
| 193) | RSHLAALWPFLGIVAEVLVLVTIIFIYEKRRKPEDVLDDDDAGSAPLKSSGQHQNDKGKN |
| | VRQRNSS |
| BSG Protein | HGPPRVKAVKSSEHINEGETAMLVCKSESVPPVTDWAWYKITDSEDKALMNGSESRFFVS |
| Fragment 2 | SSQGRSELHIENLNMEADPGQYRCNGTSSKGSDQAIITLRVRSHLAALWPFLGIVAEVLV |
| (SEQ ID NO: | LVTIIFIYEKRRKPEDVLDDDDAGSAPLKSSGQHQNDKGKNVRQRNSS |
| 194) | |
| BSG Protein | SHLAALWPFLGIVAEVLVLVTIIFIYEKRRKPEDVLDDDDAGSAPLKSSGQHQNDKGKNV |
| Fragment 3 | RQRNSS |
| (SEQ ID NO: | |
| 195) | |
| BSG Protein | MAAALFVLLGFALLGTHG |
| Signal | |
| peptide | |
| (SEQ ID NO: | |
| 196) | |
| IGSF8 | APPGPRGRQAPTSPPRMTVHEGQELALGCLARTSTQKHTHLAVSFGRSVPEAPVGRSTLQ |
| Protein | EVVGIRSDLAVEAGAPYAERLAAGELRLGKEGTDRYRMVVGGAQAGDAGTYHCTAAEWIQ |
| Fragment #1 | DPDGSWAQIAEKRAVLAHVDVQTLSSQLAVTVGPGERRIGPGEPLELLCNVSGALPPAGR |
| (SEQ ID NO: | HAAYSVGWEMAPAGAPGPGRLVAQLDTEGVGSLGPGYEGRHIAMEKVASRTYRLRLEAAR |
| 197) | PGDAGTYRCLAKAYVRGSGTRLREAASARSRPLPVHVREEGVVLEAVAWLAGGTVYRGET |
| | ASLLCNISVRGGPPGLRLAASWWVERPEDGELSSVPAQLVGGVGQDGVAELGVRPGGGPV |
| | SVELVGPRSHRLRLHSLGPEDEGVYHCAPSAWVQHADYSWYQAGSARSGPVTVYPYMHAL |
| | DTLFVPLLVGTGVALVTGATVLGTITCCFMKRLRKR |
| IGSF8 | AHVDVQTLSSQLAVTVGPGERRIGPGEPLELLCNVSGALPPAGRHAAYSVGWEMAPAGAP |
| Protein | GPGRLVAQLDTEGVGSLGPGYEGRHIAMEKVASRTYRLRLEAARPGDAGTYRCLAKAYVR |
| Fragment #2 | GSGTRLREAASARSRPLPVHVREEGVVLEAVAWLAGGTVYRGETASLLCNISVRGGPPGL |
| (SEQ ID NO: | RLAASWWVERPEDGELSSVPAQLVGGVGQDGVAELGVRPGGGPVSVELVGPRSHRLRLHS |
| 198) | LGPEDEGVYHCAPSAWVQHADYSWYQAGSARSGPVTVYPYMHALDTLFVPLLVGTGVALV |
| | TGATVLGTITCCFMKRLRKR |
| IGSF8 | REEGVVLEAVAWLAGGTVYRGETASLLCNISVRGGPPGLRLAASWWVERPEDGELSSVPA |
| Protein | QLVGGVGQDGVAELGVRPGGGPVSVELVGPRSHRLRLHSLGPEDEGVYHCAPSAWVQHAD |
| Fragment #3 | YSWYQAGSARSGPVTVYPYMHALDTLFVPLLVGTGVALVTGATVLGTITCCFMKRLRKR |
| (SEQ ID NO: | |
| 199) | |
| IGSF8 | VALVTGATVLGTITCCFMKRLRKR |
| Protein | |
| Fragment #4 | |
| (SEQ ID NO: | |
| 200) | |
| IGSF8 | MGALRPTLLPPSLPLLLLLMLGMGCWA |
| Protein- | |
| Signal | |
| Peptide | |
| (SEQ ID NO: | |
| 201) | |
| IGSF2 | MAGISYVASFFLLLTKLSIGQREVTVQKGPLFRAEGYPVSIGCNVTGHQGPSEQHFQWSV |
| protein | YLPTNPTQEVQIISTKDAAFSYAVYTQRVRSGDVYVERVQGNSVLLHISKLQMKDAGEYE |
| (SEQ ID NO: | CHTPNTDEKYYGSYSAKTNLIVIPDTLSATMSSQTLGKEEGEPLALTCEASKATAQHTHL |
| 202) | SVTWYLTQDGGGSQATEIISLSKDFILVPGPLYTERFAASDVQLNKLGPTTFRLSIERLQ |
| | SSDQGQLFCEATEWIQDPDETWMFITKKQTDQTTLRIQPAVKDFQVNITADSLFAEGKPL |
| | ELVCLVVSSGRDPQLQGIWFFNGTEIAHIDAGGVLGLKNDYKERASQGELQVSKLGPKAF |
| | SLKIFSLGPEDEGAYRCVVAEVMKTRTGSWQVLQRKQSPDSHVHLRKPAARSVVMSTKNK |
| | QQVVWEGETLAFLCKAGGAESPLSVSWWHIPRDQTQPEFVAGMGQDGIVQLGASYGVPSY |
| | HGNTRLEKMDWATFQLEITFTAITDSGTYECRVSEKSRNQARDLSWTQKISVTVKSLESS |
| | LQVSLMSRQPQVMLTNTFDLSCVVRAGYSDLKVPLTVTWQFQPASSHIFHQLIRITHNGT |
| | IEWGNFLSRFQKKTKVSQSLFRSQLLVHDATEEETGVYQCEVEVYDRNSLYNNRPPRASA |
| | ISHPLRIAVTLPESKLKVNSRSQVQELSINSNTDIECSILSRSNGNLQLAIIWYFSPVST |
| | NASWLKILEMDQTNVIKTGDEFHTPQRKQKFHTEKVSQDLFQLHILNVEDSDRGKYHCAV |
| | EEWLLSTNGTWHKLGEKKSGLTELKLKPTGSKVRVSKVYWTENVTEHREVAIRCSLESVG |
| | SSATLYSVMWYWNRENSGSKLLVHLQHDGLLEYGEEGLRRHLHCYRSSSTDFVLKLHQVE |
| | MEDAGMYWCRVAEWQLHGHPSKWINQASDESQRMVLTVLPSEPTLPSRICSSAPLLYFLF |
| | ICPFVLLLLLLISLLCLYWKARKLSTLRSNTRKEKALWVDLKEAGGVTTNRREDEEEDEG |
| | N |
| IGSF3 | MKCFFPVLSCLAVLGVVSAQRQVTVQEGPLYRTEGSHITIWCNVSGYQGPSEQNFQWSIY |
| protein | LPSSPEREVQIVSTMDSSFPYAIYTQRVRGGKIFIERVQGNSTLLHITDLQARDAGEYEC |
| (SEQ ID NO: | HTPSTDKQYFGSYSAKMNLVVIPDSLQTTAMPQTLHRVEQDPLELTCEVASETIQHSHLS |
| 203) | VAWLRQKVGEKPVEVISLSRDFMLHSSSEYAQRQSLGEVRLDKLGRTTFRLTIFHLQPSD |
| | QGEFYCEAAEWIQDPDGSWYAMTRKRSEGAVVNVQPTDKEFTVRLETEKRLHTVGEPVEF |
| | RCILEAQNVPDRYFAVSWAFNSSLIATMGPNAVPVLNSEFAHREARGQLKVAKESDSVFV |
| | LKIYHLRQEDSGKYNCRVTEREKTVTGEFIDKESKRPKNIPIIVLPLKSSISVEVASNAS |
| | VILEGEDLRFSCSVRTAGRPQGRFSVIWQLVDRQNRRSNIMWLDRDGTVQPGSSYWERSS |
| | FGGVQMEQVQPNSFSLGIFNSRKEDEGQYECHVTEWVRAVDGEWQIVGERRASTPISITA |
| | LEMGFAVTAISRTPGVTYSDSFDLQCIIKPHYPAWVPVSVTWRFQPVGTVEFHDLVTFTR |
| | DGGVQWGDRSSSFRTRTAIEKAESSNNVRLSISRASDTEAGKYQCVAELWRKNYNNTWTR |
| | LAERTSNLLEIRVLQPVTKLQVSKSKRTLTLVENKPIQLNCSVKSQTSQNSHFAVLWYVH |
| | KPSDADGKLILKTTHNSAFEYGTYAEEEGLRARLQFERHVSGGLFSLTVQRAEVSDSGSY |
| | YCHVEEWLLSPNYAWYKLAEEVSGRTEVTVKQPDSRLRLSQAQGNLSVLETRQVQLECVV |
| | LNRTSITSQLMVEWFVWKPNHPERETVARLSRDATFHYGEQAAKNNLKGRLHLESPSPGV |
| | YRLFIQNVAVQDSGTYSCHVEEWLPSPSGMWYKRAEDTAGQTALTVMRPDASLQVDTVVP |
| | NATVSEKAAFQLDCSIVSRSSQDSRFAVAWYSLRTKAGGKRSSPGLEEQEEEREEEEEEE |
| | EDDDDDDPTERTALLSVGPDAVFGPEGSPWEGRLRFQRLSPVLYRLTVLQASPQDTGNYS |
| | CHVEEWLPSPQKEWYRLTEEESAPIGIRVLDTSPTLQSIICSNDALFYFVFFYPFPIFGI |
| | LIITILLVRFKSRNSSKNSDGKNGVPLLWIKEPHLNYSPTCLEPPVLSIHPGAID |
| ATP1A1 | MGKGVGRDKYEPAAVSEQGDKKGKKGKKDRDMDELKKEVSMDDHKLSLDELHRKYGTDLS |
| protein | RGLTSARAAEILARDGPNALTPPPTTPEWIKFCRQLFGGFSMLLWIGAILCFLAYSIQAA |
| (SEQ ID NO: | TEEEPQNDNLYLGVVLSAVVIITGCFSYYQEAKSSKIMESFKNMVPQQALVIRNGEKMSI |
| 204) | NAEEVVVGDLVEVKGGDRIPADLRIISANGCKVDNSSLTGESEPQTRSPDFTNENPLETR |
| | NIAFFSTNCVEGTARGIVVYTGDRTVMGRIATLASGLEGGQTPIAAEIEHFIHIITGVAV |
| | FLGVSFFILSLILEYTWLEAVIFLIGIIVANVPEGLLATVTVCLTLTAKRMARKNCLVKN |
| | LEAVETLGSTSTICSDKTGTLTQNRMTVAHMWFDNQIHEADTTENQSGVSFDKTSATWLA |
| | LSRIAGLCNRAVFQANQENLPILKRAVAGDASESALLKCIELCCGSVKEMRERYAKIVEI |
| | PFNSTNKYQLSIHKNPNTSEPQHLLVMKGAPERILDRCSSILLHGKEQPLDEELKDAFQN |
| | AYLELGGLGERVLGFCHLFLPDEQFPEGFQFDTDDVNFPIDNLCFVGLISMIDPPRAAVP |
| | DAVGKCRSAGIKVIMVTGDHPITAKAIAKGVGIISEGNETVEDIAARLNIPVSQVNPRDA |
| | KACVVHGSDLKDMTSEQLDDILKYHTEIVFARTSPQQKLIIVEGCQRQGAIVAVTGDGVN |
| | DSPALKKADIGVAMGIAGSDVSKQAADMILLDDNFASIVTGVEEGRLIFDNLKKSIAYTL |
| | TSNIPEITPFLIFIIANIPLPLGTVTILCIDLGTDMVPAISLAYEQAESDIMKRQPRNPK |
| | TDKLVNERLISMAYGQIGMIQALGGFFTYFVILAENGFLPIHLLGLRVDWDDRWINDVED |
| | SYGQQWTYEQRKIVEFTCHTAFFVSIVVVQWADLVICKTRRNSVFQQGMKNKILIFGLFE |
| | ETALAAFLSYCPGMGVALRMYPLKPTWWFCAFPYSLLIFVYDEVRKLIIRRRPGGWVEKE |
| | TYY |
| ATP1A2 | MGRGAGREYSPAATTAENGGGKKKQKEKELDELKKEVAMDDHKLSLDELGRKYQVDLSKG |
| protein | LTNQRAQDVLARDGPNALTPPPTTPEWVKFCRQLFGGFSILLWIGAILCFLAYGIQAAME |
| (SEQ ID NO: | DEPSNDNLYLGVVLAAVVIVTGCFSYYQEAKSSKIMDSFKNMVPQQALVIREGEKMQINA |
| 205) | EEVVVGDLVEVKGGDRVPADLRIISSHGCKVDNSSLTGESEPQTRSPEFTHENPLETRNI |
| ATP1A3 | CFFSTNCVEGTARGIVIATGDRTVMGRIATLASGLEVGRTPIAMEIEHFIQLITGVAVFL |
| protein | GVSFFVLSLILGYSWLEAVIFLIGIIVANVPEGLLATVTVCLTLTAKRMARKNCLVKNLE |
| (SEQ ID NO: | AVETLGSTSTICSDKTGTLTQNRMTVAHMWFDNQIHEADTTEDQSGATFDKRSPTWTALS |
| 206) | RIAGLCNRAVFKAGQENISVSKRDTAGDASESALLKCIELSCGSVRKMRDRNPKVAEIPF |
| | NSTNKYQLSIHEREDSPQSHVLVMKGAPERILDRCSTILVQGKEIPLDKEMQDAFQNAYM |
| | ELGGLGERVLGFCQLNLPSGKFPRGFKFDTDELNFPTEKLCFVGLMSMIDPPRAAVPDAV |
| | GKCRSAGIKVIMVTGDHPITAKAIAKGVGIISEGNETVEDIAARLNIPMSQVNPREAKAC |
| | VVHGSDLKDMTSEQLDEILKNHTEIVFARTSPQQKLIIVEGCQRQGAIVAVTGDGVNDSP |
| | ALKKADIGIAMGISGSDVSKQAADMILLDDNFASIVTGVEEGRLIFDNLKKSIAYTLTSN |
| | IPEITPFLLFIlANIPLPLGTVTILCIDLGTDMVPAISLAYEAAESDIMKRQPRNSQTDK |
| | LVNERLISMAYGQIGMIQALGGFFTYFVILAENGFLPSRLLGIRLDWDDRTMNDLEDSYG |
| | QEWTYEQRKVVEFTCHTAFFASIVVVQWADLIICKTRRNSVFQQGMKNKILIFGLLEETA |
| | LAAFLSYCPGMGVALRMYPLKVTWWFCAFPYSLLIFIYDEVRKLILRRYPGGWVEKETYY |
| ATP1A4 | MGSGGSDSYRIATSQDKKDDKDSPKKNKGKERRDLDDLKKEVAMTEHKMSVEEVCRKYNT |
| protein | DCVQGLTHSKAQEILARDGPNALTPPPTTPEWVKFCRQLFGGFSILLWIGAILCFLAYGI |
| (SEQ ID NO: | QAGTEDDPSGDNLYLGIVLAAVVIITGCFSYYQEAKSSKIMESFKNMVPQQALVIREGEK |
| 207) | MQVNAEEVVVGDLVEIKGGDRVPADLRIISAHGCKVDNSSLTGESEPQTRSPDCTHDNPL |
| | ETRNITFFSTNCVEGTARGVVVATGDRTVMGRIATLASGLEVGKTPIAIEIEHFIQLITG |
| | VAVFLGVSFFILSLILGYTWLEAVIFLIGIIVANVPEGLLATVTVCLTLTAKRMARKNCL |
| | VKNLEAVETLGSTSTICSDKTGTLTQNRMTVAHMWFDNQIHEADTTEDQSGTSFDKSSHT |
| | WVALSHIAGLCNRAVFKGGQDNIPVLKRDVAGDASESALLKCIELSSGSVKLMRERNKKV |
| | AEIPFNSTNKYQLSIHETEDPNDNRYLLVMKGAPERILDRCSTILLQGKEQPLDEEMKEA |
| | FQNAYLELGGLGERVLGFCHYYLPEEQFPKGFAFDCDDVNFTTDNLCFVGLMSMIDPPRA |
| | AVPDAVGKCRSAGIKVIMVTGDHPITAKAIAKGVGIISEGNETVEDIAARLNIPVSQVNP |
| | RDAKACVIHGTDLKDFTSEQIDEILQNHTEIVFARTSPQQKLIIVEGCQRQGAIVAVTGD |
| | GVNDSPALKKADIGVAMGIAGSDVSKQAADMILLDDNFASIVTGVEEGRLIFDNLKKSIA |
| | YTLTSNIPEITPFLLFIMANIPLPLGTITILCIDLGTDMVPAISLAYEAAESDIMKRQPR |
| | NPRTDKLVNERLISMAYGQIGMIQALGGFFSYFVILAENGFLPGNLVGIRLNWDDRTVND |
| | LEDSYGQQWTYEQRKVVEFTCHTAFFVSIVVVQWADLIICKTRRNSVFQQGMKNKILIFG |
| | LFEETALAAFLSYCPGMDVALRMYPLKPSWWFCAFPYSFLIFVYDEIRKLILRRNPGGWV |
| | EKETYY |
| ATP1B3 | MGLWGKKGTVAPHDQSPRRRPKKGLIKKKMVKREKQKRNMEELKKEVVMDDHKLTLEELS |
| protein | TKYSVDLTKGHSHQRAKEILTRGGPNTVTPPPTTPEWVKFCKQLFGGFSLLLWTGAILCF |
| (SEQ ID NO: | VAYSIQIYFNEEPTKDNLYLSIVLSVVVIVTGCFSYYQEAKSSKIMESFKNMVPQQALVI |
| 208) | RGGEKMQINVQEVVLGDLVEIKGGDRVPADLRLISAQGCKVDNSSLTGESEPQSRSPDFT |
| | HENPLETRNICFFSTNCVEGTARGIVIATGDSTVMGRIASLTSGLAVGQTPIAAEIEHFI |
| | HLITVVAVFLGVTFFALSLLLGYGWLEAIIFLIGIIVANVPEGLLATVTVCLTLTAKRMA |
| | RKNCLVKNLEAVETLGSTSTICSDKTGTLTQNRMTVAHMWFDMTVYEADTTEEQTGKTFT |
| | KSSDTWFMLARIAGLCNRADFKANQEILPIAKRATTGDASESALLKFIEQSYSSVAEMRE |
| | KNPKVAEIPFNSTNKYQMSIHLREDSSQTHVLMMKGAPERILEFCSTFLLNGQEYSMNDE |
| | MKEAFQNAYLELGGLGERVLGFCFLNLPSSFSKGFPFNTDEINFPMDNLCFVGLISMIDP |
| | PRAAVPDAVSKCRSAGIKVIMVTGDHPITAKAIAKGVGIISEGTETAEEVAARLKIPISK |
| | VDASAAKAIVVHGAELKDIQSKQLDQILQNHPEIVFARTSPQQKLIIVEGCQRLGAVVAV |
| | TGDGVNDSPALKKADIGIAMGISGSDVSKQAADMILLDDNFASIVTGVEEGRLIFDNLKK |
| | SIMYTLTSNIPEITPFLMFIILGIPLPLGTITILCIDLGTDMVPAISLAYESAESDIMKR |
| | LPRNPKTDNLVNHRLIGMAYGQIGMIQALAGFFTYFVILAENGFRPVDLLGIRLHWEDKY |
| | LNDLEDSYGQQWTYEQRKVVEFTCQTAFFVTIVVVQWADLIISKTRRNSLFQQGMRNKVL |
| | IFGILEETLLAAFLSYTPGMDVALRMYPLKITWWLCAIPYSILIFVYDEIRKLLIRQHPD |
| | GWVERETYY |
| ATP2B1 | MTKNEKKSLNQSLAEWKLFIYNPTTGEFLGRTAKSWGLILLFYLVFYGFLAALFSFTMWV |
| protein | MLQTLNDEVPKYRDQIPSPGLMVFPKPVTALEYTFSRSDPTSYAGYIEDLKKFLKPYTLE |
| (SEQ ID NO: | EQKNLTVCPDGALFEQKGPVYVACQFPISLLQACSGMNDPDFGYSQGNPCILVKMNRIIG |
| 209) | LKPEGVPRIDCVSKNEDIPNVAVYPHNGMIDLKYFPYYGKKLHVGYLQPLVAVQVSFAPN |
| | NTGKEVTVECKIDGSANLKSQDDRDKFLGRVMFKITARA |
| ATP2B2 | MGDMANNSVAYSGVKNSLKEANHDGDFGITLAELRALMELRSTDALRKIQESYGDVYGIC |
| protein | TKLKTSPNEGLSGNPADLERREAVFGKNFIPPKKPKTFLQLVWEALQDVTLIILEIAAIV |
| (SEQ ID NO: | SLGLSFYQPPEGDNALCGEVSVGEEEGEGETGWIEGAAILLSVVCVVLVTAFNDWSKEKQ |
| 210) | FRGLQSRIEQEQKFTVIRGGQVIQIPVADITVGDIAQVKYGDLLPADGILIQGNDLKIDE |
| | SSLTGESDHVKKSLDKDPLLLSGTHVMEGSGRMVVTAVGVNSQTGIIFTLLGAGGEEEEK |
| | KDEKKKEKKNKKQDGAIENRNKAKAQDGAAMEMQPLKSEEGGDGDEKDKKKANLPKKEKS |
| | VLQGKLTKLAVQIGKAGLLMSAITVIILVLYFVIDTFWVQKRPWLAECTPIYIQYFVKFF |
| | IIGVTVLVVAVPEGLPLAVTISLAYSVKKMMKDNNLVRHLDACETMGNATAICSDKTGTL |
| | TMNRMTVVQAYINEKHYKKVPEPEAIPPNILSYLVTGISVNCAYTSKILPPEKEGGLPRH |
| | VGNKTECALLGLLLDLKRDYQDVRNEIPEEALYKVYTFNSVRKSMSTVLKNSDGSYRIFS |
| | KGASEIILKKCFKILSANGEAKVFRPRDRDDIVKTVIEPMASEGLRTICLAFRDFPAGEP |
| | EPEWDNENDIVTGLTCIAVVGIEDPVRPEVPDAIKKCQRAGITVRMVTGDNINTARAIAT |
| | KCGILHPGEDFLCLEGKDFNRRIRNEKGEIEQERIDKIWPKLRVLARSSPTDKHTLVKGI |
| | IDSTVSDQRQVVAVTGDGTNDGPALKKADVGFAMGIAGTDVAKEASDIILTDDNFTSIVK |
| | AVMWGRNVYDSISKFLQFQLTVNVVAVIVAFTGACITQDSPLKAVQMLWVNLIMDTLASL |
| | ALATEPPTESLLLRKPYGRNKPLISRTMMKNILGHAFYQLVVVFTLLFAGEKFFDIDSGR |
| | NAPLHAPPSEHYTIVFNTFVLMQLFNEINARKIHGERNVFEGIFNNAIFCTIVLGTFVVQ |
| | IIIVQFGGKPFSCSELSIEQWLWSIFLGMGTLLWGQLISTIPTSRLKFLKEAGHGTQKEE |
| | IPEEELAEDVEEIDHAERELRRGQILWFRGLNRIQTQMDVVNAFQSGSSIQGALRRQPSI |
| | ASQHHDVTNISTPTHIRVVNAFRSSLYEGLEKPESRSSIHNFMTHPEFRIEDSEPHIPLI |
| | DDTDAEDDAPTKRNSSPPPSPNKNNNAVDSGIHLTIEMNKSATSSSPGSPLHSLETSL |
| ATP2B3 | MGDMTNSDFYSKNQRNESSHGGEFGCTMEELRSLMELRGTEAVVKIKETYGDTEAICRRL |
| protein | KTSPVEGLPGTAPDLEKRKQIFGQNFIPPKKPKTFLQLVWEALQDVTLIILEIAAIISLG |
| (SEQ ID NO: | LSFYHPPGEGNEGCATAQGGAEDEGEAEAGVVIEGAAILLSVICWLVTAFNDWSKEKQFR |
| 211) | GLQSRIEQEQKFTVVRAGQVVQIPVAEIVVGDIAQVKYGDLLPADGLFIQGNDLKIDESS |
| | LTGESDQVRKSVDKDPMLLSGTHVMEGSGRMLVTAVGVNSQTGIIFTLLGAGGEEEEKKD |
| | KKGVKKGDGLQLPAADGAAASNAADSANASLVNGKMQDGNVDASQSKAKQQDGAAAMEMQ |
| | PLKSAEGGDADDRKKASMHKKEKSVLQGKLTKLAVQIGKAGLVMSAITVIILVLYFTVDT |
| | FVVNKKPWLPECTPVYVQYFVKFFIIGVTVLVVAVPEGLPLAVTISLAYSVKKMMKDNNL |
| | VRHLDACETMGNATAICSDKTGTLTTNRMTVVQAYVGDVHYKEIPDPSSINTKTMELLIN |
| | AIAINSAYTTKILPPEKEGALPRQVGNKTECGLLGFVLDLKQDYEPVRSQMPEEKLYKVY |
| | TFNSVRKSMSTVIKLPDESFRMYSKGASEIVLKKCCKILNGAGEPRVFRPRDRDEMVKKV |
| | IEPMACDGLRTICVAYRDFPSSPEPDWDNENDILNELTCICVVGIEDPVRPEVPEAIRKC |
| | QRAGITVRMVTGDNINTARAIAIKCGIIHPGEDFLCLEGKEFNRRIRNEKGEIEQERIDK |
| | IWPKLRVLARSSPTDKHTLVKGIIDSTHTEQRQVVAVTGDGTNDGPALKKADVGFAMGIA |
| | GTDVAKEASDIILTDDNFSSIVKAVMWGRNVYDSISKFLQFQLTVNVVAVIVAFTGACIT |
| | QDSPLKAVQMLWVNLIMDTFASLALATEPPTETLLLRKPYGRNKPLISRTMMKNILGHAV |
| | YQLALIFTLLFVGEKMFQIDSGRNAPLHSPPSEHYTIIFNTFVMMQLFNEINARKIHGER |
| | NVFDGIFRNPIFCTIVLGTFAIQIVIVQFGGKPFSCSPLQLDQWMWCIFIGLGELVWGQV |
| | IATIPTSRLKFLKEAGRLTQKEEIPEEELNEDVEEIDHAERELRRGQILWFRGLNRIQTQ |
| | IEVVNTFKSGASFQGALRRQSSVTSQSQDIRVVKAFRSSLYEGLEKPESRTSIHNFMAHP |
| | EFRIEDSQPHIPLIDDTDLEEDAALKQNSSPPSSLNKNNSAIDSGINLTTDTSKSATSSS |
| | PGSPIHSLETSL |
| ATP2B4 | MGDMANSSIEFHPKPQQQRDVPQAGGFGCTLAELRTLMELRGAEALQKIEEAYGDVSGLC |
| protein | RRLKTSPTEGLADNTNDLEKRRQIYGQNFIPPKQPKTFLQLVWEALQDVTLIILEVAAIV |
| (SEQ ID NO: | SLGLSFYAPPGEESEACGNVSGGAEDEGEAEAGVVIEGAAILLSVICWLVTAFNDWSKEK |
| 212) | QFRGLQSRIEQEQKFTVIRNGQLLQVPVAALVVGDIAQVKYGDLLPADGVLIQANDLKID |
| | ESSLTGESDHVRKSADKDPMLLSGTHVMEGSGRMVVTAVGVNSQTGIIFTLLGAGGEEEE |
| | KKDKKGKQQDGAMESSQTKAKKQDGAVAMEMQPLKSAEGGEMEEREKKKANAPKKEKSVL |
| | QGKLTKLAVQIGKAGLVMSAITVIILVLYFVIETFVVEGRTWLAECTPVYVQYFVKFFII |
| | GVTVLVVAVPEGLPLAVTISLAYSVKKMMKDNNLVRHLDACETMGNATAICSDKTGTLTT |
| | NRMTVVQSYLGDTHYKEIPAPSALTPKILDLLVHAISINSAYTTKILPPEKEGALPRQVG |
| | NKTECALLGFVLDLKRDFQPVREQIPEDKLYKVYTFNSVRKSMSTVIRMPDGGFRLFSKG |
| | ASEILLKKCTNILNSNGELRGFRPRDRDDMVRKIIEPMACDGLRTICIAYRDFSAGQEPD |
| | WDNENEVVGDLTCIAVVGIEDPVRPEVPEAIRKCQRAGITVRMVTGDNINTARAIAAKCG |
| | IIQPGEDFLCLEGKEFNRRIRNEKGEIEQERLDKVWPKLRVLARSSPTDKHTLVKGIIDS |
| | TTGEQRQVVAVTGDGTNDGPALKKADVGFAMGIAGTDVAKEASDIILTDDNFTSIVKAVM |
| | WGRNVYDSISKFLQFQLTVNVVAVIVAFTGACIT |

In some embodiments, the scaffold protein comprises Basigin (the BSG protein), represented by SEQ ID NO: 3. The BSG protein is also known as 5F7, Collagenase stimulatory factor, Extracellular matrix metalloproteinase inducer (EMMPRIN), Leukocyte activation antigen M6, OK blood group antigen, Tumor cell-derived collagenase stimulatory factor (TCSF), or CD147. The Uniprot number for the human BSG protein is P35613. The signal peptide of the BSG protein is amino acid 1 to 21 of SEQ ID NO: 3. Amino acids 138-323 of SEQ ID NO: 3 is the extracellular domain, amino acids 324 to 344 is the transmembrane domain, and amino acids 345 to 385 of SEQ ID NO: 3 is the cytoplasmic domain.

In other embodiments, the scaffold protein comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 22 to 385 of SEQ ID NO: 3. In some embodiments, the fragments of BSG polypeptide lack one or more functional or structural domains, such as IgV, e.g., amino acids 221 to 315 of SEQ ID NO: 3. In other embodiments, the scaffold protein comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 193, 194, or 195. In other embodiments, the scaffold protein comprises the amino acid sequence of SEQ ID NO: 193, 194, or 195, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some embodiments, the scaffold protein comprises the amino acid sequence of SEQ ID NO: 193, 194, or 195 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 193, 194, or 195.

In some embodiments, the scaffold protein comprises Immunoglobulin superfamily member 8 (IgSF8 or the IGSF8 protein), which is also known as CD81 partner 3, Glu-Trp-Ile EWI motif-containing protein 2 (EWI-2), Keratinocytes-associated transmembrane protein 4 (KCT-4), LIR-D1, Prostaglandin regulatory-like protein (PGRL) or CD316. The full length human IGSF8 protein is accession no. Q969P0 in Uniprot and is shown as SEQ ID NO: 4 herein. The human IGSF8 protein has a signal peptide (amino acids 1 to 27 of SEQ ID NO: 4), an extracellular domain (amino acids 28 to 579 of SEQ ID NO: 4), a transmembrane domain (amino acids 580 to 600 of SEQ ID NO: 4), and a cytoplasmic domain (amino acids 601 to 613 of SEQ ID NO: 4).

In other embodiments, the scaffold protein comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 28 to 613 of SEQ ID NO: 4. In some embodiments, the IGSF8 protein lack one or more functional or structural domains, such as IgV. In other embodiments, the scaffold protein comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 197, 198, 199, or 200. In other embodiments, the scaffold protein comprises the amino acid sequence of SEQ ID NO: 197, 198, 199, or 200, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some embodiments, the scaffold protein comprises the amino acid sequence of SEQ ID NO: 197, 198, 199, or 200 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 197, 198, 199, or 200.

In some embodiments, the scaffold protein comprises Immunoglobulin superfamily member 3 (IgSF3 or the IGSF3 protein), which is also known as Glu-Trp-Ile EWI motif-containing protein 3 (EWI-3), and is shown as the amino acid sequence of SEQ ID NO: 203. The human IGSF3 protein has a signal peptide (amino acids 1 to 19 of SEQ ID NO: 203), an extracellular domain (amino acids 20 to 1124 of SEQ ID NO: 203), a transmembrane domain (amino acids 1125 to 1145 of SEQ ID NO: 203), and a cytoplasmic domain (amino acids 1146 to 1194 of SEQ ID NO: 203).

In other embodiments, the scaffold protein comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 28 to 613 of SEQ ID NO: 203. In some embodiments, the IGSF3 protein lack one or more functional or structural domains, such as IgV.

In other aspects, the scaffold protein comprises the IGSF2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 202 without the signal peptide. In some aspects, the IGSF2 protein lacks one or more functional or structural domains, such as IgV.

In some embodiments, a scaffold protein comprises Integrin beta-1 (the ITGB1 protein), which is also known as Fibronectin receptor subunit beta, Glycoprotein IIa (GPIIA), VLA-4 subunit beta, or CD29, and is shown as the amino acid sequence of SEQ ID NO: 5. The human ITGB1 protein has a signal peptide (amino acids 1 to 20 of SEQ ID NO: 5), an extracellular domain (amino acids 21 to 728 of SEQ ID NO: 5), a transmembrane domain (amino acids 729 to 751 of SEQ ID NO: 5), and a cytoplasmic domain (amino acids 752 to 798 of SEQ ID NO: 5).

In other embodiments, the scaffold protein comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 21 to 798 of SEQ ID NO: 5. In some embodiments, the ITGB1 protein lack one or more functional or structural domains, such as IgV.

In other embodiments, the scaffold protein comprises the ITGA4 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 6 without the signal peptide (amino acids 1 to 33 of SEQ ID NO: 6). In some embodiments, the ITGA4 protein lacks one or more functional or structural domains, such as IgV.

In other embodiments, the scaffold protein comprises the SLC3A2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 7 without the signal peptide. In some embodiments, the SLC3A2 protein lacks one or more functional or structural domains, such as IgV.

In other embodiments, the scaffold protein comprises the ATP1A1 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 204 without the signal peptide. In some embodiments, the ATP1A1 protein lacks one or more functional or structural domains, such as IgV.

In other embodiments, the scaffold protein comprises the ATP1A2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 205 without the signal peptide. In some embodiments, the ATP1A2 protein lacks one or more functional or structural domains, such as IgV.

In other embodiments, the scaffold protein comprises the ATP1A3 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 206 without the signal peptide. In some embodiments, the ATP1A3 protein lacks one or more functional or structural domains, such as IgV.

In other embodiments, the scaffold protein comprises the ATP1A4 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 207 without the signal peptide. In some embodiments, the ATP1A4 protein lacks one or more functional or structural domains, such as IgV.

In other embodiments, the scaffold protein comprises the ATP1B3 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 208 without the signal peptide. In some embodiments, the ATP1B3 protein lacks one or more functional or structural domains, such as IgV.

In other embodiments, the scaffold protein comprises the ATP2B1 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 209 without the signal peptide. In some embodiments, the ATP2B1 protein lacks one or more functional or structural domains, such as IgV.

In other embodiments, the scaffold protein comprises the ATP2B2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 210 without the signal peptide. In some embodiments, the ATP2B2 protein lacks one or more functional or structural domains, such as IgV.

In other embodiments, the scaffold protein comprises the ATP2B3 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 211 without the signal peptide. In some embodiments, the ATP2B3 protein lacks one or more functional or structural domains, such as IgV.

In other embodiments, the scaffold protein comprises the ATP2B4 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 212 without the signal peptide. In some embodiments, the ATP2B4 protein lacks one or more functional or structural domains, such as IgV.

Non-limiting examples of other scaffold protein proteins can be found at U.S. Pat. No. 10,195,290B1, issued Feb. 5, 2019, which is incorporated by reference in its entireties.

In some embodiments, the sequence encodes a fragment of the scaffold protein lacking at least about 5, at least about 10, at least about 50, at least about 100, at least about 200, at least about 300, at least about 400, at least about 500, at least about 600, at least about 700, or at least about 800 amino acids from the N-terminus of the native protein. In some embodiments, the sequence encodes a fragment of the scaffold protein lacking at least about 5, at least about 10, at least about 50, at least about 100, at least about 200, at least about 300, at least about 400, at least about 500, at least about 600, at least about 700, or at least about 800 amino acids from the C-terminus of the native protein. In some embodiments, the sequence encodes a fragment of the scaffold protein lacking at least about 5, at least about 10, at least about 50, at least about 100, at least about 200, at least about 300, at least about 400, at least about 500, at least about 600, at least about 700, or at least about 800 amino acids from both the N-terminus and C-terminus of the native protein. In some embodiments, the sequence encodes a fragment of the scaffold protein lacking one or more functional or structural domains of the native protein.

II.C.2. Luminal Anchoring Scaffold Proteins

In some embodiments, the scaffold protein (e.g., Scaffold Y) interacts with the luminal surface of the EV membrane. In some embodiments, the scaffold protein is anchored to the luminal surface of the EV membrane. In some aspects, the scaffold protein of the present disclosure comprises an “N-terminus domain” (ND) and an “effector domain,” wherein the ND and/or the ED are associated with the luminal surface of the EV, e.g., an exosome.

In some embodiments, the scaffold protein comprises an intracellular (luminal) domain, a transmembrane domain, and an extracellular domain, wherein the AAV is associated with the extracellular domain of the scaffold protein, and wherein the intracellular (luminal) domain of the scaffold protein interacts with the luminal surface of the EV membrane. In some embodiments, the scaffold protein is anchored to the luminal surface of the EV membrane. In some aspects, the scaffold protein of the present disclosure comprises an intracellular domain, a transmembrane domain, and an extracellular domain; wherein the intracellular domain comprises an “N-terminus domain” (ND) and an “effector domain;” wherein the ND and/or the ED are associated with the luminal surface of the EV, e.g., an exosome.

In some aspects, the scaffolds of the present disclosure can be associated with the luminal surface of the EV, e.g., via a lipid anchor (e.g., myristic acid), and/or a polybasic domain that interacts electrostatically with the negatively charged head of membrane phospholipids. In other aspects, the scaffold protein comprises an N-terminus domain (ND) and an effector domain (ED), wherein the ND is associated with the luminal surface of the EV and the ED are associated with the luminal surface of the EV by an ionic interaction, wherein the ED comprises at least two, at least three, at least four, at least five, at least six, or at least seven contiguous lysines (Lys) in sequence.

In other embodiments, the scaffold protein (e.g., the intracellular (luminal) domain of the scaffold protein) comprises an N-terminus domain (ND) and an effector domain (ED), wherein the ND is associated with the luminal surface of the EV, and the ED is associated with the luminal surface of the EV by an ionic interaction, wherein the ED comprises at least two, at least three, at least four, at least five, at least six, or at least seven contiguous lysines (Lys) in sequence.

In other embodiments, the ED further comprises one or more low complexity regions, e.g., a PEST motif. A PEST sequence is a peptide sequence that is rich in proline (P), glutamic acid (E), serine (S), and threonine (T). In some embodiments, the ED further comprises negatively charged residues (for example, Glu) and many Ser and Thr that undergo transient phosphorylation (thus, both adding negative charges to the areas out of ED).

In some aspects, the ND is associated with the luminal surface of the EV, e.g., an exosome, via lipidation, e.g., via myristoylation. In some aspects, the ND has Gly at the N terminus. In some aspects, the N-terminal Gly is myristoylated.

In some aspects, the ED is associated with the luminal surface of the EV, e.g., an exosome, by an ionic interaction. In some aspects, the ED is associated with the luminal surface of the EV, e.g., an exosome, by an electrostatic interaction, in particular, an attractive electrostatic interaction.

In some aspects, the ED comprises (i) a basic amino acid (e.g., lysine), or (ii) two or more basic amino acids (e.g., lysine) next to each other in a polypeptide sequence. In some aspects, the basic amino acid is lysine (Lys; K), arginine (Arg, R), or Histidine (His, H). In some aspects, the basic amino acid is (Lys)n, wherein n is an integer between 1 and 10.

In some embodiments, the ED comprises (i) a lysine repeat in the ED or (ii) a lysine repeat with the ND, e.g., K at the C terminus in the ND and K at the N terminus in the ED, wherein the ND and ED are linked directly, i.e., by a peptide bond. In some embodiments, the minimum number of the amino acids that are capable of anchoring a heterologous moiety, e.g., a biologically active molecule, in the lumen of the EV, e.g., exosome, e.g., about seven to about 15, about seven to about 14, about seven to about 13, about seven to about 12, about seven to about 11, about seven to about 10, about seven to about 9, or about seven to about 8 amino acid fragments.

In other aspects, the ED comprises at least a lysine and the ND comprises a lysine at the C terminus if the N terminus of the ED is directly linked to lysine at the C terminus of the ND, i.e., the lysine is in the N terminus of the ED and is fused to the lysine in the C terminus of the ND. In other embodiments, the ED comprises at least two lysines, at least three lysines, at least four lysines, at least five lysines, at least six lysines, or at least seven lysines when the N terminus of the ED is linked to the C terminus of the ND by a linker, e.g., one or more amino acids. In some embodiments, the ED comprises at least two contiguous lysines (Lys) in sequence.

In some aspects, the ED comprises K, KK, KKK, KKKK (SEQ ID NO: 11), KKKKK (SEQ ID NO: 12), R, RR, RRR, RRRR (SEQ ID NO: 13); RRRRR (SEQ ID NO: 14), KR, RK, KKR, KRK, RKK, KRR, RRK, (K/R)(K/R)(K/R)(K/R) (SEQ ID NO:15), (K/R)(K/R)(K/R)(K/R)(K/R) (SEQ ID NO:16), or any combination thereof. In some aspects, the ED comprises KK, KKK, KKKK (SEQ ID NO: 11), KKKKK (SEQ ID NO: 12), or any combination thereof. In some aspects, the ED comprises Arg (R), RR, RRR, RRRR (SEQ ID NO: 13); RRRRR (SEQ ID NO: 14), KR, RK, KKR, KRK, RKK, KRR, RRK, (K/R)(K/R)(K/R)(K/R) (SEQ ID NO: 15), (K/R)(K/R)(K/R)(K/R)(K/R) (SEQ ID NO: 16), or any combination thereof. In some aspects, the ND comprises the amino acid sequence as set forth in G:X2:X3:X4:X5:X6, wherein G represents Gly; wherein “:” represents a peptide bond, wherein each of the X2 to the X6 independently represents an amino acid; and wherein the X6 represents a basic amino acid. In some aspects, the X6 amino acid is selected is selected from the group consisting of Lys, Arg, and His. In some aspects, the X5 amino acid is selected from the group consisting of Pro, Gly, Ala, and Ser. In some aspects, the X2 amino acid is selected from the group consisting of Pro, Gly, Ala, and Ser. In some aspects, the X4 is selected from the group consisting of Pro, Gly, Ala, Ser, Val, Ile, Leu, Phe, Trp, Tyr, Gln, and Met.

In some aspects, the scaffold protein comprises an N-terminus domain (ND) and an effector domain (ED), wherein the ND comprises the amino acid sequence as set forth in G:X2:X3:X4:X5:X6, wherein G is a glycine, wherein G represents Gly; wherein “:” represents a peptide bond, wherein each of the X2 to the X6 is independently an amino acid; wherein the X6 comprises a basic amino acid, and wherein the ED is linked to X6 by a peptide bond and comprises at least one lysine at the N terminus of the ED.

In some aspects, the ND of the scaffold protein comprises the amino acid sequence of G:X2:X3:X4:X5:X6, wherein G represents Gly; “:” represents a peptide bond; the X2 represents an amino acid selected from the group consisting of Pro, Gly, Ala, and Ser; the X3 represents any amino acid; the X4 represents an amino acid selected from the group consisting of Pro, Gly, Ala, Ser, Val, Ile, Leu, Phe, Trp, Tyr, Gln, and Met; the X5 represents an amino acid selected from the group consisting of Pro, Gly, Ala, and Ser; and the X6 represents an amino acid selected from the group consisting of Lys, Arg, and His.

In some aspects, the X3 amino acid is selected from the group consisting of Asn, Gln, Ser, Thr, Asp, Glu, Lys, His, and Arg.

In some aspects, the ND and ED are joined by a linker. In some aspects, the linker comprises one or more amino acids. In some aspects, the term “linker” refers to a peptide or polypeptide sequence (e.g., a synthetic peptide or polypeptide sequence) or to a non-polypeptide, e.g., an alkyl chain. In some aspects, two or more linkers can be linked in tandem. Generally, linkers provide flexibility or prevent/ameliorate steric hindrances. Linkers are not typically cleaved; however in certain aspects, such cleavage can be desirable. Accordingly, in some aspects a linker can comprise one or more protease-cleavable sites, which can be located within the sequence of the linker or flanking the linker at either end of the linker sequence. When the ND and ED are joined by a linker, the ED comprise at least two lysines, at least three lysines, at least four lysines, at least five lysines, at least six lysines, or at least seven lysines.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the linker is a glycine/serine linker. In some aspects, the peptide linker is glycine/serine linker according to the formula [(Gly)n-Ser]m (SEQ ID NO: 46) where n is any integer from 1 to 100 and m is any integer from 1 to 100. In other aspects, the glycine/serine linker is according to the formula [(Gly)x-Sery]z (SEQ ID NO: 47) wherein x in an integer from 1 to 4, y is 0 or 1, and z is an integers from 1 to 50. In some aspects, the peptide linker comprises the sequence Gn (SEQ ID NO: 48), where n can be an integer from 1 to 100. In some aspects, the peptide linker can comprise the sequence (GlyAla)n (SEQ ID NO: 49), wherein n is an integer between 1 and 100. In other aspects, the peptide linker can comprise the sequence (GlyGlySer)n (SEQ ID NO: 50), wherein n is an integer between 1 and 100.

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion).

In other aspects, the peptide linker can comprise non-naturally occurring amino acids. In yet other aspects, the peptide linker can comprise naturally occurring amino acids occurring in a linear sequence that does not occur in nature. In still other aspects, the peptide linker can comprise a naturally occurring polypeptide sequence.

The present disclosure also provides an isolated EV, e.g., an exosome, comprising a biologically active molecule linked to a scaffold protein, wherein the scaffold protein comprises ND-ED, wherein: a. ND comprises G:X2:X3:X4:X5:X6; wherein: i. G represents Gly; ii. “:” represents a peptide bond; iii. X2 represents an amino acid selected from the group consisting of Pro, Gly, Ala, and Ser; iv. X3 represents any amino acid; v. X4 represents an amino acid selected from the group consisting of Pro, Gly, Ala, Ser, Val, Ile, Leu, Phe, Trp, Tyr, Glu, and Met; vi. X5 represents an amino acid selected from the group consisting of Pro, Gly, Ala, and Ser; vii. X6 represents an amino acid selected from the group consisting of Lys, Arg, and His; b. “-” represents an optional linker; and c. ED is an effector domain comprising (i) at least two contiguous lysines (Lys), which is linked to the X6 by a peptide bond or one or more amino acids or (ii) at least one lysine, which is directly linked to the X6 by a peptide bond.

In some aspects, the X2 amino acid is selected from the group consisting of Gly and Ala. In some aspects, the X3 amino acid is Lys. In some aspects, the X4 amino acid is Leu or Glu. In some aspects, the X5 amino acid is selected from the group consisting of Ser and Ala. In some aspects, the X6 amino acid is Lys. In some aspects, the X2 amino acid is Gly, Ala, or Ser; the X3 amino acid is Lys or Glu; the X4 amino acid is Leu, Phe, Ser, or Glu; the X5 amino acid is Ser or Ala; and X6 amino acid is Lys. In some aspects, the “-” linker comprises a peptide bond or one or more amino acids.

In some aspects, the ED in the scaffold protein comprises Lys (K), KK, KKK, KKKK (SEQ ID NO: 11), KKKKK (SEQ ID NO: 12), Arg (R), RR, RRR, RRRR (SEQ ID NO: 13); RRRRR (SEQ ID NO: 14), KR, RK, KKR, KRK, RKK, KRR, RRK, (K/R)(K/R)(K/R)(K/R) (SEQ ID NO: 15), (K/R)(K/R)(K/R)(K/R)(K/R) (SEQ ID NO: 16), or any combination thereof.

In some aspects, the scaffold protein comprises an amino acid sequence selected from the group consisting of (i) GGKLSKK (SEQ ID NO: 17), (ii) GAKLSKK (SEQ ID NO: 18), (iii) GGKQSKK (SEQ ID NO:19), (iv) GGKLAKK (SEQ ID NO: 20), or (v) any combination thereof.

In some aspects, the ND in the scaffold protein comprises an amino acid sequence selected from the group consisting of (i) GGKLSK (SEQ ID NO: 51), (ii) GAKLSK (SEQ ID NO: 52), (iii) GGKQSK (SEQ ID NO: 53), (iv) GGKLAK (SEQ ID NO: 54), or (v) any combination thereof and the ED in the scaffold protein comprises (i) K, KK, KKK, KKKG (SEQ ID NO: 55), KKKGY (SEQ ID NO: 56), KKKGYN (SEQ ID NO: 57), KKKGYNV (SEQ ID NO: 58), KKKGYNVN (SEQ ID NO: 59), KKKGYS (SEQ ID NO: 60), KKKGYG (SEQ ID NO: 61), KKKGYGG (SEQ ID NO: 62), KKKGS (SEQ ID NO: 63), KKKGSG (SEQ ID NO: 64), KKKGSG (SEQ ID NO: 65), KKKGSGS (SEQ ID NO: 66), KKKS (SEQ ID NO: 67), KKKSG (SEQ ID NO: 68), KKKSGG (SEQ ID NO: 69), KKKSGGS (SEQ ID NO: 70), KKKSGGSG (SEQ ID NO: 71), KKSGGSGG (SEQ ID NO: 72), KKKSGGSGGS (SEQ ID NO: 73), and KRFSFKKS (SEQ ID NO: 74).

In some aspects, the polypeptide sequence of a scaffold protein of the present disclosure consists of an amino acid sequence selected from the group consisting of (i) GGKLSKK (SEQ ID NO: 21), (ii) GAKLSKK (SEQ ID NO: 18), (iii) GGKQSKK (SEQ ID NO: 19), (iv) GGKLAKK (SEQ ID NO: 20), or (v) any combination thereof.

In some aspects, the scaffold protein comprises an amino acid sequence selected from the group consisting of (i) GGKLSKKK (SEQ ID NO: 22), (ii) GGKLSKKS (SEQ ID NO: 23), (iii) GAKLSKKK (SEQ ID NO: 24), (iv) GAKLSKKS (SEQ ID NO: 25), (v) GGKQSKKK (SEQ ID NO: 26), (vi) GGKQSKKS (SEQ ID NO: 27), (vii) GGKLAKKK (SEQ ID NO: 28), (viii) GGKLAKKS (SEQ ID NO:29), and (ix) any combination thereof.

In some aspects, the polypeptide sequence of a scaffold protein of the present disclosure consists of an amino acid sequence selected from the group consisting of (i) GGKLSKKK (SEQ ID NO: 22), (ii) GGKLSKKS (SEQ ID NO: 23), (iii) GAKLSKKK (SEQ ID NO: 24), (iv) GAKLSKKS (SEQ ID NO: 25), (v) GGKQSKKK (SEQ ID NO: 26), (vi) GGKQSKKS (SEQ ID NO: 27), (vii) GGKLAKKK (SEQ ID NO: 28), (viii) GGKLAKKS (SEQ ID NO: 29), and (ix) any combination thereof. In some embodiments, the scaffold protein of the present disclosure comprises at least two contiguous lysines (Lys) in sequence.

In some aspects, the scaffold protein is at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 55, at least about 60, at least about 65, at least about 70, at least about 75, at least about 80, at least about 85, at least about 90, at least about 95, at least about 100, at least about 105, at least about 110, at least about 115, at least about 120, at least about 125, at least about 130, at least about 135, at least about 140, at least about 145, at least about 150, at least about 155, at least about 160, at least about 165, at least about 170, at least about 175, at least about 180, at least about 185, at least about 190, at least about 195, at least about 200, at least about 205, at least about 210, at least about 215, at least about 220, at least about 225, at least about 230, at least about 235, at least about 240, at least about 245, at least about 250, at least about 255, at least about 260, at least about 265, at least about 270, at least about 275, at least about 280, at least about 285, at least about 290, at least about 295, at least about 300, at least about 305, at least about 310, at least about 315, at least about 320, at least about 325, at least about 330, at least about 335, at least about 340, at least about 345, or at least about 350 amino acids in length.

In some aspects, the scaffold protein is between about 5 and about 10, between about 10 and about 20, between about 20 and about 30, between about 30 and about 40, between about 40 and about 50, between about 50 and about 60, between about 60 and about 70, between about 70 and about 80, between about 80 and about 90, between about 90 and about 100, between about 100 and about 110, between about 110 and about 120, between about 120 and about 130, between about 130 and about 140, between about 140 and about 150, between about 150 and about 160, between about 160 and about 170, between about 170 and about 180, between about 180 and about 190, between about 190 and about 200, between about 200 and about 210, between about 210 and about 220, between about 220 and about 230, between about 230 and about 240, between about 240 and about 250, between about 250 and about 260, between about 260 and about 270, between about 270 and about 280, between about 280 and about 290, between about 290 and about 300, between about 300 and about 310, between about 310 and about 320, between about 320 and about 330, between about 330 and about 340, or between about 340 and about 250 amino acids in length.

In some aspects, the scaffold protein comprises (i) GGKLSKKKKGYNVN (SEQ ID NO: 32), (ii) GAKLSKKKKGYNVN (SEQ ID NO: 33), (iii) GGKQSKKKKGYNVN (SEQ ID NO: 34), (iv) GGKLAKKKKGYNVN (SEQ ID NO: 35), (v) GGKLSKKKKGYSGG (SEQ ID NO: 36), (vi) GGKLSKKKKGSGGS (SEQ ID NO: 37), (vii) GGKLSKKKKSGGSG (SEQ ID NO: 38), (viii) GGKLSKKKSGGSGG (SEQ ID NO: 39), (ix) GGKLSKKSGGSGGS (SEQ ID NO: 40, (x) GGKLSKSGGSGGSV (SEQ ID NO: 41), or (xi) GAKKSKKRFSFKKS (SEQ ID NO: 42).

In some aspects, the polypeptide sequence of a scaffold protein of the present disclosure consists of (i) GGKLSKKKKGYNVN (SEQ ID NO: 32), (ii) GAKLSKKKKGYNVN (SEQ ID NO: 33), (iii) GGKQSKKKKGYNVN (SEQ ID NO: 34), (iv) GGKLAKKKKGYNVN (SEQ ID NO: 35), (v) GGKLSKKKKGYSGG (SEQ ID NO: 36), (vi) GGKLSKKKKGSGGS (SEQ ID NO: 37), (vii) GGKLSKKKKSGGSG (SEQ ID NO: 38), (viii) GGKLSKKKSGGSGG (SEQ ID NO: 39), (ix) GGKLSKKSGGSGGS (SEQ ID NO: 40), (x) GGKLSKSGGSGGSV (SEQ ID NO: 41), or (xi) GAKKSKKRFSFKKS (SEQ ID NO: 42).

Non-limiting examples of the scaffold protein useful for the present disclosure is listed below. In some embodiments, the scaffold protein comprises an amino acid sequence set forth in TABLE 4. In some embodiments, the scaffold protein consists of an amino acid sequence set forth in TABLE 4.

TABLE 4
Exemplary Scaffold Proteins

| SEQ ID NO: | Scaffold Protein: GX2X3X4X5X6-ED |
|---|---|
| 75 | GGKLSKKKKGYNVNDEKAKEKDKKAEGAA |
| 76 | GGKLSKKKKGYNVNDEKAKEKDKKAEGA |
| 77 | GGKLSKKKKGYNVNDEKAKEKDKKAEG |
| 78 | GGKLSKKKKGYNVNDEKAKEKDKKAE |
| 79 | GGKLSKKKKGYNVNDEKAKEKDKKA |
| 80 | GGKLSKKKKGYNVNDEKAKEKDKK |
| 81 | GGKLSKKKKGYNVNDEKAKEKDK |
| 82 | GGKLSKKKKGYNVNDEKAKEKD |
| 83 | GGKLSKKKKGYNVNDEKAKEK |
| 84 | GGKLSKKKKGYNVNDEKAKE |
| 85 | GGKLSKKKKGYNVNDEKAK |
| 86 | GGKLSKKKKGYNVNDEKA |
| 87 | GGKLSKKKKGYNVNDEK |
| 88 | GGKLSKKKKGYNVNDE |
| 89 | GGKLSKKKKGYNVND |
| 32 | GGKLSKKKKGYNVN |
| 90 | GGKLSKKKKGYNV |
| 91 | GGKLSKKKKGYN |
| 92 | GGKLSKKKKGY |
| 93 | GGKLSKKKKG |
| 94 | GGKLSKKKK |
| 22 | GGKLSKKK |
| 17 | GGKLSKK |
| 95 | GAKKSKKRFSFKKSFKLSGFSFKKNKKEA |
| 96 | GAKKSKKRFSFKKSFKLSGFSFKKNKKE |
| 97 | GAKKSKKRFSFKKSFKLSGFSFKKNKK |
| 98 | GAKKSKKRFSFKKSFKLSGFSFKKNK |
| 99 | GAKKSKKRFSFKKSFKLSGFSFKKN |
| 100 | GAKKSKKRFSFKKSFKLSGFSFKK |
| 101 | GAKKSKKRFSFKKSFKLSGFSFK |
| 102 | GAKKSKKRFSFKKSFKLSGFSF |
| 103 | GAKKSKKRFSFKKSFKLSGFS |
| 104 | GAKKSKKRFSFKKSFKLSGF |
| 105 | GAKKSKKRFSFKKSFKLSG |
| 106 | GAKKSKKRFSFKKSFKLS |
| 107 | GAKKSKKRFSFKKSFKL |
| 108 | GAKKSKKRFSFKKSFK |
| 109 | GAKKSKKRFSFKKSF |
| 42 | GAKKSKKRFSFKKS |
| 110 | GAKKSKKRFSFKK |
| 111 | GAKKSKKRFSFK |
| 112 | GAKKSKKRFSF |
| 113 | GAKKSKKRFS |
| 114 | GAKKSKKRF |
| 115 | GAKKSKKR |
| 116 | GAKKSKK |
| 117 | GAKKAKKRFSFKKSFKLSGFSFKKNKKEA |
| 118 | GAKKAKKRFSFKKSFKLSGFSFKKNKKE |
| 119 | GAKKAKKRFSFKKSFKLSGFSFKKNKK |
| 120 | GAKKAKKRFSFKKSFKLSGFSFKKNK |
| 121 | GAKKAKKRFSFKKSFKLSGFSFKKN |
| 122 | GAKKAKKRFSFKKSFKLSGFSFKK |
| 123 | GAKKAKKRFSFKKSFKLSGFSFK |
| 124 | GAKKAKKRFSFKKSFKLSGFSF |
| 125 | GAKKAKKRFSFKKSFKLSGFS |
| 126 | GAKKAKKRFSFKKSFKLSGF |
| 127 | GAKKAKKRFSFKKSFKLSG |
| 128 | GAKKAKKRFSFKKSFKLS |
| 129 | GAKKAKKRFSFKKSFKL |
| 130 | GAKKAKKRFSFKKSFK |
| 131 | GAKKAKKRFSFKKSF |
| 132 | GAKKAKKRFSFKKS |
| 133 | GAKKAKKRFSFKK |
| 134 | GAKKAKKRFSFK |
| 135 | GAKKAKKRFSF |
| 136 | GAKKAKKRFS |
| 137 | GAKKAKKRF |
| 138 | GAKKAKKR |
| 139 | GAKKAKK |
| 140 | GAQESKKKKKKRFSFKKSFKLSGFSFKK |
| 141 | GAQESKKKKKKRFSFKKSFKLSGFSFK |
| 142 | GAQESKKKKKKRFSFKKSFKLSGFSF |
| 143 | GAQESKKKKKKRFSFKKSFKLSGFS |
| 144 | GAQESKKKKKKRFSFKKSFKLSGF |
| 145 | GAQESKKKKKKRFSFKKSFKLSG |
| 146 | GAQESKKKKKKRFSFKKSFKLS |
| 147 | GAQESKKKKKKRFSFKKSFKL |
| 148 | GAQESKKKKKKRFSFKKSFK |
| 149 | GAQESKKKKKKRFSFKKSF |
| 150 | GAQESKKKKKKRFSFKKS |
| 151 | GAQESKKKKKKRFSFKK |
| 152 | GAQESKKKKKKRFSFK |
| 153 | GAQESKKKKKKRFSF |
| 154 | GAQESKKKKKKRFS |
| 155 | GAQESKKKKKKRF |
| 156 | GAQESKKKKKKR |
| 157 | GAQESKKKKKK |
| 158 | GAQESKKKKK |
| 159 | GAQESKKKK |
| 160 | GAQESKKK |
| 161 | GAQESKK |
| 162 | GSQSSKKKKKKFSFKKPFKLSGLSFKRNRK |
| 163 | GSQSSKKKKKKFSFKKPFKLSGLSFKRNR |
| 164 | GSQSSKKKKKKFSFKKPFKLSGLSFKRN |
| 165 | GSQSSKKKKKKFSFKKPFKLSGLSFKR |
| 166 | GSQSSKKKKKKFSFKKPFKLSGLSFK |
| 167 | GSQSSKKKKKKFSFKKPFKLSGLSF |
| 168 | GSQSSKKKKKKFSFKKPFKLSGLS |
| 169 | GSQSSKKKKKKFSFKKPFKLSGL |
| 170 | GSQSSKKKKKKFSFKKPFKLSG |
| 171 | GSQSSKKKKKKFSFKKPFKLS |
| 172 | GSQSSKKKKKKFSFKKPFKL |
| 173 | GSQSSKKKKKKFSFKKPFK |
| 174 | GSQSSKKKKKKFSFKKPF |
| 175 | GSQSSKKKKKKFSFKKP |
| 176 | GSQSSKKKKKKFSFKK |
| 177 | GSQSSKKKKKKFSFK |
| 178 | GSQSSKKKKKKFSF |
| 179 | GSQSSKKKKKKFS |
| 180 | GSQSSKKKKKKF |
| 181 | GSQSSKKKKKK |
| 182 | GSQSSKKKKK |
| 183 | GSQSSKKKK |
| 184 | GSQSSKKK |
| 185 | GSQSSKK |

In some aspects, the scaffold protein of the present disclosure does not contain an N-terminal Met. In some aspects, the scaffold protein comprises a lipidated amino acid, e.g., a myristoylated amino acid, at the N-terminus of the scaffold protein, which functions as a lipid anchor. In some aspects, the amino acid residue at the N-terminus of the scaffold protein is Gly. The presence of an N-terminal Gly is an absolute requirement for N-myristoylation. In some aspects, the amino acid residue at the N-terminus of the scaffold protein is synthetic. In some aspects, the amino acid residue at the N-terminus of the scaffold protein is a glycine analog, e.g., allylglycine, butylglycine, or propargylglycine.

In other aspects, the lipid anchor can be any lipid anchor known in the art, e.g., palmitic acid or glycosylphosphatidylinositols. Under unusual circumstances, e.g., by using a culture medium where myristic acid is limiting, some other fatty acids including shorter-chain and unsaturated, can be attached to the N-terminal glycine. For example, in BK channels, myristate has been reported to be attached posttranslationally to internal serine/threonine or tyrosine residues via a hydroxyester linkage. Membrane anchors known in the art are presented in the following table.

TABLE 5
Modification groups

| Modification | Modifying Group |
|---|---|
| S-Palmitoylation | 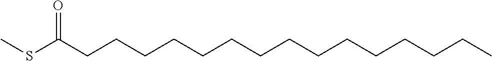 [SMILES: CCCCCCCCCCCCCCCC(=O)SC] |
| N-Palmitoylation | 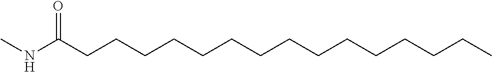 [SMILES: CCCCCCCCCCCCCCCC(=O)NC] |
| N-Myristoylation | 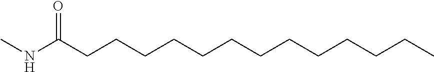 [SMILES: CCCCCCCCCCCCCC(=O)NC] |
| O-Acylation | 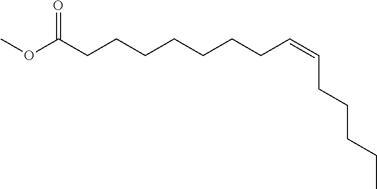 [SMILES: CCCCC/C=C\CCCCCCCC(=O)OC] |
| Farnesylation | 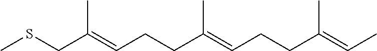 [SMILES: C/C=C(\C)CC/C=C(\C)CC/C=C(\C)CSC] |
| Geranylgeranylation | 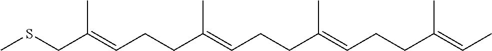 [SMILES: C/C=C(\C)CC/C=C(\C)CC/C=C(\C)CC/C=C(\C)CSC] |
| Cholesterol | 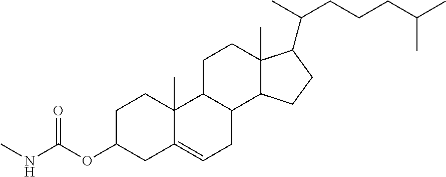 [SMILES: CNC(=O)OC1CCC2(C)C(=CCC3C2CCC2(C)C(C(C)CCCC(C)C)CCC32)C1] |

In some embodiments, the scaffold protein is selected from the group consisting of MARCKS, MARKSL1, BASP1, any functional fragment, variant, or derivative thereof, or any combination thereof. In some embodiments, the scaffold protein comprises an Src protein or a fragment thereof. In some embodiments, the scaffold protein comprises a sequence disclosed, e.g., in U.S. Pat. No. 9,611,481, which is incorporated by reference herein in its entirety.

TABLE 6
Exemplary Scaffold Protein Sequences

| Protein | Sequence |
|---|---|
| MARCKS | MGAQFSKTAAKGEAAAERPGEAAVASSPSKANGQENGHVKVNGDASPAAAES |
| protein | GAKEELQANGSAPAADKEEPAAAGSGAASPSAAEKGEPAAAAAPEAGASPVE |
| (SEQ ID | KEAPAEGEAAEPGSPTAAEGEAASAASSTSSPKAEDGATPSPSNETPKKKKK |
| NO: 8) | RFSFKKSFKLSGFSFKKNKKEAGEGGEAEAPAAEGGKDEAAGGAAAAAAEAG |
| | AASGEQAAAPGEEAAAGEEGAAGGDPQEAKPQEAAVAPEKPPASDETKAAEE |
| | PSKVEEKKAEEAGASAAACEAPSAAGPGAPPEQEAAPAEEPAAAAASSACAA |
| | PSQEAQPECSPEAPPAEAAE |
| MARCKSL1 | MGSQSSKAPRGDVTAEEAAGASPAKANGQENGHVKSNGDLSPKGEGESPPV |
| protein | NGTDEAAGATGDAIEPAPPSQGAEAKGEVPPKETPKKKKKFSFKKPFKLSG |
| (SEQ ID | LSFKRNRKEGGGDSSASSPTEEEQEQGEIGACSDEGTAQEGKAAATPESQE |
| NO: 9) | PQAKGAEASAASEEEAGPQATEPSTPSGPESGPTPASAEQNE |
| BASP1 | MGGKLSKKKKGYNVNDEKAKEKDKKAEGAATEEEGTPKESEPQAAAEPAEA |
| protein | KEGKEKPDQDAEGKAEEKEGEKDAAAAKEEAPKAEPEKTEGAAEAKAEPPK |
| (SEQ ID | APEQEQAAPGPAAGGEAPKAAEAAAAPAESAAPAAGEEPSKEEGEPKKTEA |
| NO: 10) | PAAPAAQETKSDGAPASDSKPGSSEAAPSSKETPAATEAPSSTPKAQGPAA |
| | SAEEPKPVEAPAANSDQTVTVKE |

In some embodiments, the scaffold protein of the present disclosure comprises the MARCKS protein, or a fragment, variant, or derivative thereof. The MARCKS protein (Uniprot accession no. P29966) is also known as protein kinase C substrate, 80 kDa protein, light chain. The full-length human MARCKS protein is 332 amino acids in length and comprises a calmodulin-binding domain at amino acid residues 152-176. In some aspects, the scaffold protein of the present disclosure comprises a mature MARCKS protein (i.e., without N-terminal methionine). In some aspects, the scaffold protein of the present disclosure is derived from a mature MARCKS protein, i.e., it is a fragment, variant, or derivate of a mature MARCKS protein and therefore it lacks the N-terminal protein present in the nonmature protein.

In some aspects, the scaffold protein of the present disclosure comprises the MARCKSL1 protein (Uniprot accession no. P49006), also known as MARCKS-like protein 1, and macrophage myristoylated alanine-rich C kinase substrate. The full-length human MARCKSL1 protein is 195 amino acids in length. The MARCKSL1 protein has an effector domain involved in lipid-binding and calmodulin-binding at amino acid residues 87-110. In some aspects, the scaffold protein of the present disclosure comprises a mature MARCKSL1 protein (i.e., without N-terminal methionine). In some aspects, the scaffold protein of the present disclosure is derived from a mature MARCKSL1 protein, i.e., it is a fragment, variant, or derivate of a mature MARCKSL1 protein and therefore it lacks the N-terminal protein present in the non-mature protein.

In some aspects, the scaffold of the present disclosure comprises the BASP1 protein (Uniprot accession number P80723), also known as 22 kDa neuronal tissue-enriched acidic protein or neuronal axonal membrane protein NAP-22. The full-length human BASP1 protein sequence (isomer 1) is 227 amino acids in length. An isomer produced by an alternative splicing is missing amino acids 88 to 141 from isomer 1. In some aspects, the scaffold protein of the present disclosure comprises a mature BASP1 protein (i.e., without N-terminal methionine). In some aspects, the scaffold protein of the present disclosure is derived from a mature BASP1 protein, i.e., it is a fragment, variant, or derivate of a mature BASP1 protein and therefore it lacks the N-terminal protein present in the non-mature protein.

In some aspects, the scaffold protein comprises an amino acid sequence having at least about 65%, at least about 70%, at least about 75%, at least about 80%, at least about 85%, at least about 90%, at least about 95%, at least about 96%, at least about 97%, at least about 98%, at least about 99%, or about 100% sequence identity to the mature form of SEQ ID NO: 8 (MARCKS), i.e., without the N-terminal methionine amino acid present in SEQ ID NO: 8. In some aspects, the scaffold protein comprises an amino acid sequence having at least about 65%, at least about 70%, at least about 75%, at least about 80%, at least about 85%, at least about 90%, at least about 95%, at least about 96%, at least about 97%, at least about 98%, at least about 99%, or about 100% sequence identity to a functional fragment of the mature form of SEQ ID NO: 8 (MARCKS), i.e., without the N-terminal methionine amino acid present in SEQ ID NO: 8.

In some aspects, the scaffold protein comprises an amino acid sequence having at least about 65%, at least about 70%, at least about 75%, at least about 80%, at least about 85%, at least about 90%, at least about 95%, at least about 96%, at least about 97%, at least about 98%, at least about 99%, or about 100% sequence identity to the mature form of SEQ ID NO: 9 (MARCKSL1), i.e., without the N-terminal methionine amino acid present in SEQ ID NO: 9. In some aspects, the scaffold protein comprises an amino acid sequence having at least about 65%, at least about 70%, at least about 75%, at least about 80%, at least about 85%, at least about 90%, at least about 95%, at least about 96%, at least about 97%, at least about 98%, at least about 99%, or about 100% sequence identity to a functional fragment of the mature form of SEQ ID NO: 9 (MARCKSL1), i.e., without the N-terminal methionine amino acid present in SEQ ID NO: 9.

In some aspects, the scaffold protein comprises an amino acid sequence having at least about 65%, at least about 70%, at least about 75%, at least about 80%, at least about 85%, at least about 90%, at least about 95%, at least about 96%, at least about 97%, at least about 98%, at least about 99%, or about 100% sequence identity to the mature form of SEQ ID NO: 10 (BASP1), i.e., without the N-terminal methionine amino acid present in SEQ ID NO: 10. In some aspects, the scaffold protein comprises an amino acid sequence having at least about 65%, at least about 70%, at least about 75%, at least about 80%, at least about 85%, at least about 90%, at least about 95%, at least about 96%, at least about 97%, at least about 98%, at least about 99%, or about 100% sequence identity to a functional fragment of the mature form of SEQ ID NO: 10 (BASP1), i.e., without the N-terminal methionine amino acid present in SEQ ID NO: 10.

II.C.3. Scaffold Protein Fusion Constructs

In some embodiments, the scaffold protein is linked to one or more heterologous proteins. The one or more heterologous proteins can be linked to the N-terminus of the scaffold moieties. The one or more heterologous proteins can be linked to the C-terminus of the scaffold moieties. In some embodiments, the one or more heterologous proteins are linked to both the N-terminus and the C-terminus of the scaffold moieties. In some embodiments, the heterologous protein is a mammalian protein. In some embodiments, the heterologous protein is a human protein.

In some embodiments, the scaffold protein can be used to link any moiety to the luminal surface and/or the external surface of the exosome. For example, the PTGFRN polypeptide can be used to link an AAV, e.g., a capsid protein of an AAV, inside the lumen (e.g., on the luminal surface) in addition to the external surface of the EV, e.g., exosome. Therefore, in certain embodiments, the scaffold protein can be used for dual purposes, e.g., an AAV on the luminal surface and a second payload on the external surface of the EV, e.g., exosome, or an AAV on the external surface of the exosome and a second payload on the luminal surface of the EV, e.g., exosome.

In some embodiments, the scaffold protein is linked to an AAV. In some embodiments, the scaffold protein is linked to the AAV, e.g., a capsid protein of the AAV, by a linker. In some embodiments, the linker comprises one or more amino acids. In some embodiments, the linker is a cleavable linker. In some embodiments, the linker is flexible linker. In some embodiments, the linker is a rigid linker. In certain embodiments, the linker is at least about 2 amino acids, at least about 3 amino acids, at least about 4 amino acids, at least about 5 amino acids, at least about 6 amino acids, at least about 7 amino acids, at least about 8 amino acids, at least about 9 amino acids, at least about 10 amino acids, at least about 11 amino acids, at least about 12 amino acids, at least about 13 amino acids, at least about 14 amino acids, at least about 15 amino acids, at least about 16 amino acids, at least about 17 amino acids, at least about 18 amino acids, at least about 19 amino acids, at least about 20 amino acids, at least about 25 amino acids, at least about 30 amino acids, at least about 35 amino acids, at least about 40, amino acids, at least about 45 amino acids, or at least about 50.

In some embodiments, the scaffold protein is linked to a binding partner of a chemically induced dimer. In some embodiments, the scaffold protein is linked to a binding partner of a chemically induced dimer, and the AAV, e.g., a capsid protein of an AAV, is linked to a corresponding binding partner. In these embodiments, the scaffold protein and the AAV, e.g., the capsid protein of an AAV, associate with each other in the presence of the chemical that induces dimerization of the binding partners. In some embodiments, the binding partner is linked to the N-terminus of the scaffold protein. In some embodiments, the binding partner is linked to the C-terminus of the scaffold protein. In some embodiments, the binding partner is linked to a luminal domain of the scaffold protein.

In some embodiments, the binding partner linked to the scaffold protein is selected from one binding partner of a chemically induced dimer selected from the group consisting of (i) FKBP and FKBP (FK1012); (ii) FKBP and CalcineurinA (CNA) (FK506); (iii) FKBP and CyP-Fas (FKCsA); (iv) FKBP and FRB (Rapamycin); (v) GyrB and GyrB (Coumermycin); (vi) GAI and GID1 (Gibberellin); (vii) Snap-tag and HaloTag (HaXS); (viii) eDHFR and HaloTag (TMP-HTag); and (ix) BCL-xL and Fab (AZ1) (ABT-737); wherein the AAV, e.g., a capsid protein of the AAV, is linked to the corresponding binding partner, as described herein. In certain embodiments, the scaffold protein is linked to an FKBP. In certain embodiments, the scaffold protein is linked to an FRB. In some embodiments, the FRB is the FRB of mTOR. In some embodiments, the scaffold protein is linked to CalcineurinA. In some embodiments, the scaffold protein is linked to CyP-Fas. In some embodiments, the scaffold protein is linked to GyrB. In some embodiments, the scaffold protein is linked to CyP-Fas. In some embodiments, the scaffold protein is linked to GAI. In some embodiments, the scaffold protein is linked to GID1. In some embodiments, the scaffold protein is linked to Snap-tag. In some embodiments, the scaffold protein is linked to HaloTag. In some embodiments, the scaffold protein is linked to eDHFR. In some embodiments, the scaffold protein is linked to BCL-xL. In some embodiments, the AAV capsid protein is linked to Fab.

In certain embodiments, the scaffold protein is linked to an FKBP, and a capsid protein of the AAV is linked to an FKBP. In certain embodiments, the scaffold protein is linked to an FRB, and a capsid protein of the AAV is linked to an FKBP. In certain embodiments, the scaffold protein is linked to an FKBP, and a capsid protein of the AAV is linked to an FRB. In some embodiments, the scaffold protein is linked to CalcineurinA, and a capsid protein of the AAV is linked to an FKBP. In some embodiments, the scaffold protein is linked to an FKBP, and a capsid protein of the AAV is linked to CalcineurinA. In some embodiments, the scaffold protein is linked to a CyP-Fas, and a capsid protein of the AAV is linked to an FKBP. In some embodiments, the scaffold protein is linked to an FKBP, and a capsid protein of the AAV is linked to a CyP-Fas. In some embodiments, the scaffold protein is linked to a GyrB, and a capsid protein of the AAV is linked to a GyrB. In some embodiments, the scaffold protein is linked to a GAI, and a capsid protein of the AAV is linked to a GID1. In some embodiments, the scaffold protein is linked to a GID1, and a capsid protein of the AAV is linked to a GAI. In some embodiments, the scaffold protein is linked to a Snap-tag, and a capsid protein of the AAV is linked to a HaloTag. In some embodiments, the scaffold protein is linked to a HaloTag, and a capsid protein of the AAV is linked to a Snap-tag. In some embodiments, the scaffold protein is linked to an eDHFR, and a capsid protein of the AAV is linked to a HaloTag. In some embodiments, the scaffold protein is linked to a HaloTag, and a capsid protein of the AAV is linked to an eDHFR. In some embodiments, the scaffold protein is linked to a BCL-xL, and a capsid protein of the AAV is linked to an Fab (AZ1). In some embodiments, the AAV capsid protein is linked to a Fab (AZ1), and a capsid protein of the AAV is linked to a BCL-xL.

In some embodiments, the scaffold protein is linked to an affinity agent. In some embodiments, the affinity agent is linked to the N-terminus of the scaffold protein. In some embodiments, the affinity agent is linked to the C-terminus of the scaffold protein. In some embodiments, the affinity agent is linked to a luminal domain of the scaffold protein. In some embodiments, the affinity agent comprises an AAV binding polypeptide. In some embodiments, the affinity agent comprises an AAV receptor. In some embodiments, the affinity agent comprises an antibody or an antigen binding domain, as disclosed herein. In some embodiments, the affinity agent binds to one or more AAV capsid proteins. In some embodiments, the one or more AAV capsid proteins is AAV assembly activating proteins. In some embodiments, the affinity agent does not bind to an AAV capsid protein monomer.

In some embodiments, the interaction between the affinity agent and the AAV is transient. In some embodiments, the AAV is dissociated form the affinity agent under certain conditions. In certain embodiments, the affinity of the affinity agent to the AAV is dependent on pH. In some embodiments, the AAV dissociates from the affinity agent at a pH of at least about 3, at least about 4, at least about 5, at least about 6, at least about 7, at least about 8, at least about 9, at least about 10, at least about 11, or at least about 12. In some embodiments, the affinity of the affinity agent for the AAV is dependent on the concentration of calcium, magnesium, sulfate, phosphate, or any combination thereof in the solution comprising the AAV and the affinity agent. In some embodiments, the affinity of the affinity agent for the AAV is dependent on the salt concentration and/or ionic strength of the solution comprising the AAV and the affinity agent. In some embodiments, the AAV and the affinity agent are dissociable under reducing conditions.

In some embodiments, the scaffold protein is linked to an AAV binding polypeptide. In some embodiments, the AAV binding polypeptide is linked to the N-terminus of the scaffold protein. In some embodiments, the AAV binding polypeptide is linked to the C-terminus of the scaffold protein. In some embodiments, the AAV binding polypeptide is linked to a luminal domain of the scaffold protein.

In some embodiments, the AAV binding polypeptide comprises an “antigen-binding domain.” In some embodiments, the antigen-binding domain comprises an antigen-binding fragment of an antibody. In some embodiments, the antigen-binding domain comprises a single-chain antibody or an antigen-binding fragment thereof. In some embodiments, the antigen-binding domain comprises a humanized antibody or an antigen-binding fragment thereof. In some embodiments, the antigen-binding domain comprises a murine antibody or an antigen-binding fragment thereof. In some embodiments, the antigen-binding domain comprises a chimeric antibody (e.g., a mouse-human, a mouse-primate, or a primate-human monoclonal antibody) or an antigen binding fragment thereof. In some embodiments, the antigen-binding domain comprises an antigen-binding fragment of a camelid antibody, a shark IgNAR, or an anti-idiotype antibody. In some embodiments, the antigen-binding domain comprises a camelid antibody, or an antigen-binding fragment thereof. In some embodiments, the antigen-binding domain comprises a single-domain antibody or an antigen-binding fragment thereof. In some embodiments, the antigen-binding domain comprises a shark IgNAR or an antigen-binding fragment thereof. In some embodiments, the antigen-binding domain comprises an anti-idiotype antibody or an antigen-binding fragment thereof.

In some embodiments, the antigen-binding domain comprises a single chain antibody. In some embodiments, the antigen-binding domain comprises an scFv. In some embodiments, the antigen-binding domain comprises an (scFv)₂. In some embodiments, the antigen-binding domain comprises an Fab. In some embodiments, the antigen-binding domain comprises an Fab′. In some embodiments, the antigen-binding domain comprises an F(ab′)₂. In some embodiments, the antigen-binding domain comprises an F(ab1)₂. In some embodiments, the antigen-binding domain comprises an Fv. In some embodiments, the antigen-binding domain comprises a dAb. In some embodiments, the antigen-binding domain comprises a single chain Fab. In some embodiments, the antigen-binding domain comprises an Fd fragment.

In some embodiments, the antigen-binding domain comprises a diabody. In some embodiments, the antigen-binding domain comprises a minibody. In some embodiments, the antigen-binding domain comprises an antibody-related polypeptide. In particular embodiments, the antigen-binding domain comprises a nanobody.

In some embodiments, the scaffold protein is linked to a receptor. In some embodiments, the receptor binds AAV, e.g., the AAV binding peptide is an AAV receptor. In some embodiments, the receptor is linked to the N-terminus of the scaffold protein. In some embodiments, the receptor is linked to the C-terminus of the scaffold protein. In some embodiments, the receptor is linked to a luminal domain of the scaffold protein.

In some embodiments, the receptor is an AAV receptor or a fragment thereof (see, e.g., Pillay et al., Nature 530(7588):108-12 (2016), which is incorporated by reference herein in its entirety). Any AAV receptor known in the art, or an AAV-binding fragment thereof, can be linked to the scaffold proteins described herein. In certain embodiments, the AAV receptor is the AAV receptor encoded by the gene KIAA0319L, e.g., the AAV receptor is AAVR (Pillay et al., 2016). AAVR is an N-linked glycosylated protein of about 150 kDa. Full-length AAVR is a type 1 transmembrane protein comprising five Ig-like domains referred to as polycystic kidney disease (PKD) domains. In some embodiments, the scaffold protein is linked to an AAVR fragment, comprising at least the PKD1 domain of the AAVR. In some embodiments, the scaffold protein is linked to an AAVR fragment, comprising at least the PKD2 domain of the AAVR. In some embodiments, the scaffold protein is linked to an AAVR fragment, comprising at least the PKD3 domain of the AAVR. In some embodiments, the scaffold protein is linked to an AAVR fragment, comprising at least the PKD4 domain of the AAVR. In some embodiments, the scaffold protein is linked to an AAVR fragment, comprising at least the PKD5 domain of the AAVR. In some embodiments, the scaffold protein is linked to an AAVR fragment, comprising at least the PKD1 and PKD2 domains of the AAVR. In some embodiments, the scaffold protein is linked to an AAVR fragment, comprising at least the PKD1, PKD2, and PKD3 domains of the AAVR. In some embodiments, the scaffold protein is linked to an AAVR fragment, comprising at least the PKD1, PKD2, PKD3, and PKD4 domains of the AAVR. In some embodiments, the scaffold protein is linked to an AAVR fragment, comprising the PKD1, PKD2, PKD3, PKD4, PKD5 domains of the AAVR. In some embodiments, the scaffold protein is linked an AAVR fragment, wherein the AAVR fragment does not comprise a PKD5 domain. In some embodiments, the scaffold protein is linked an AAVR fragment, wherein the AAVR fragment does not comprise a PKD4 domain. In some embodiments, the scaffold protein is linked an AAVR fragment, wherein the AAVR fragment does not comprise a PKD3 domain. In some embodiments, the scaffold protein is linked an AAVR fragment, wherein the AAVR fragment does not comprise a PKD2 domain. In some embodiments, the scaffold protein is linked an AAVR fragment, wherein the AAVR fragment does not comprise a PKD1 domain. In some embodiments, the scaffold protein is linked an AAVR fragment, wherein the AAVR fragment does not comprise a PKD5 domain or a PKD4 domain. In some embodiments, the scaffold protein is linked an AAVR fragment, wherein the AAVR fragment does not comprise a PKD5 domain, a PKD4 domain, or a PKD3 domain. In some embodiments, the scaffold protein is linked an AAVR fragment, wherein the AAVR fragment does not comprise a PKD5 domain, a PKD4 domain, a PKD3 domain, or a PKD2 domain.

In some embodiments, the scaffold protein is linked to an Fc receptor, and the AAV, e.g., a capsid protein of the AAV, is linked to an Fc. In certain embodiments, the Fc receptor is an Fc gamma receptor selected from Fc gamma receptor I (FcγR1), FcγRIIA, FcγIIB, FcγIIIA, and FcγIIIB; and the Fc is an Fc of an IgG. In certain embodiments, the Fc receptor is an FcγR1 and the Fc is an Fc of an IgG. In some embodiments, the Fc receptor is an Fc alpha receptor I (FcαR1), and wherein the Fc is an Fc of an IgA. In some embodiments, the Fc receptor is an Fc epsilon receptor selected from Fc epsilon receptor I (FcεRI) and FcεRII, and wherein the Fc is an Fc of an IgE.

In some embodiments, the scaffold protein is linked to a nanobody; and the AAV, e.g., a capsid protein of the AAV, is linked an immunoglobulin constant region (Fc). In certain embodiments, the nanobody specifically binds to the Fc.

II.C.4. Additional Modes of Association

In some embodiments, the scaffold protein and the AAV, e.g., a capsid protein of the AAV, are associated through an intermediary. In some embodiments, the AAV, e.g., a capsid protein of the AAV, is linked to an Fc, and the scaffold protein is linked to an Fc receptor, wherein the Fc receptor of the scaffold protein associates with the Fc of the AAV. In other embodiments, the AAV comprises an antigen, and the scaffold protein is linked to an antigen-binding domain that specifically binds the AAV antigen. In other embodiments, the AAV, e.g., a capsid protein of the AAV, is linked to an Fc, and the scaffold protein is linked to an antigen-binding domain that specifically binds the Fc. In other embodiments, the scaffold protein is linked to a receptor, wherein the AAV comprises a ligand of the receptor. In certain embodiments, the scaffold protein is linked to an AAV receptor, wherein the AAV receptor specifically interacts with a ligand on AAV. In some embodiments, the AAV is incubated with an antibody or a fragment thereof, e.g., an IgG, and the scaffold domain is linked to an Fc receptor, wherein the antibody or a fragment thereof binds the AAV, and wherein the Fc receptor binds the Fc portion of the antibody.

II.C.4.i. Ligand-Receptor

In certain aspects of the present disclosure, the scaffold protein is linked to a receptor and the AAV is linked to a ligand. Any ligand-receptor pairing known in the art can be used. In some embodiments, the ligand it an Fc and the receptor is an Fc receptor. In some embodiments, the ligand, e.g., Fc, is linked to a capsid protein of the AAV. In some embodiments, the ligand, e.g., Fc, is linked to at least one VP1 protein of the AAV. In some embodiments, a ligand, e.g., Fc, is linked to each of the 5 VP1 proteins of the AAV. In some embodiments, a ligand, e.g., Fc, is linked to each of 4 of the VP1 proteins of the AAV. In some embodiments, a ligand, e.g., Fc, is linked to each of 3 of the VP1 proteins of the AAV. In some embodiments, a ligand, e.g., Fc, is linked to each of 2 of the VP1 proteins of the AAV. In some embodiments, a ligand, e.g., Fc, is linked to 1 of the VP1 proteins of the AAV. In some embodiments, the AAV comprises one VP1 protein that is not linked to a ligand, e.g., Fc. In some embodiments, the AAV comprises two VP1 proteins that are not linked to a ligand, e.g., Fc. In some embodiments, the AAV comprises three VP1 proteins that are not linked to a ligand, e.g., Fc. In some embodiments, the AAV comprises four VP1 proteins that are not linked to a ligand, e.g., Fc.

In some embodiments, the ligand, e.g., Fc, is linked to at least one VP2 protein of the AAV. In some embodiments, a ligand, e.g., Fc, is linked to each of the 5 VP2 proteins of the AAV. In some embodiments, a ligand, e.g., Fc, is linked to each of 4 of the VP2 proteins of the AAV. In some embodiments, a ligand, e.g., Fc, is linked to each of 3 of the VP2 proteins of the AAV. In some embodiments, a ligand, e.g., Fc, is linked to each of 2 of the VP2 proteins of the AAV. In some embodiments, a ligand, e.g., Fc, is linked to 1 of the VP2 proteins of the AAV. In some embodiments, the AAV comprises one VP2 protein that is not linked to a ligand, e.g., Fc. In some embodiments, the AAV comprises two VP2 proteins that are not linked to a ligand, e.g., Fc. In some embodiments, the AAV comprises three VP2 proteins that are not linked to a ligand, e.g., Fc. In some embodiments, the AAV comprises four VP2 proteins that are not linked to a ligand, e.g., Fc.

In some embodiments, the ligand, e.g., Fc, is linked to at least one VP3 protein of the AAV. In some embodiments, a ligand, e.g., Fc, is linked to each of the VP3 proteins of the AAV. In some embodiments, a ligand, e.g., Fc, is linked to each of a subset of the VP3 proteins of the AAV. In some embodiments, a ligand, e.g., Fc, is linked to each of at least about 40 of the VP3 proteins of the AAV. In some embodiments, a ligand, e.g., Fc, is linked to each of at least about 35 of the VP3 proteins of the AAV. In some embodiments, a ligand, e.g., Fc, is linked to each of at least about 30 of the VP3 proteins of the AAV. In some embodiments, a ligand, e.g., Fc, is linked to each of at least about 25 of the VP3 proteins of the AAV. In some embodiments, a ligand, e.g., Fc, is linked to each of at least about 20 of the VP3 proteins of the AAV. In some embodiments, a ligand, e.g., Fc, is linked to each of at least about 15 of the VP3 proteins of the AAV. In some embodiments, a ligand, e.g., Fc, is linked to each of at least about 10 of the VP3 proteins of the AAV. In some embodiments, a ligand, e.g., Fc, is linked to each of at least about 9 of the VP3 proteins of the AAV. In some embodiments, a ligand, e.g., Fc, is linked to each of at least about 8 of the VP3 proteins of the AAV. In some embodiments, a ligand, e.g., Fc, is linked to each of at least about 7 of the VP3 proteins of the AAV. In some embodiments, a ligand, e.g., Fc, is linked to each of at least about 6 of the VP3 proteins of the AAV. In some embodiments, a ligand, e.g., Fc, is linked to each of at least about 5 of the VP3 proteins of the AAV. In some embodiments, a ligand, e.g., Fc, is linked to each of at least about 4 of the VP3 proteins of the AAV. In some embodiments, a ligand, e.g., Fc, is linked to each of at least about 3 of the VP3 proteins of the AAV. In some embodiments, a ligand, e.g., Fc, is linked to each of at least about 2 of the VP3 proteins of the AAV. In some embodiments, a ligand, e.g., Fc, is linked to 1 of the VP3 proteins of the AAV. In some embodiments, the AAV comprises at least 1 VP3 protein that is not linked to a ligand, e.g., Fc. In some embodiments, the AAV comprises at least about 2 VP3 proteins that are not linked to a ligand, e.g., Fc. In some embodiments, the AAV comprises at least about 3 VP3 proteins that are not linked to a ligand, e.g., Fc. In some embodiments, the AAV comprises at least about 4 VP3 proteins that are not linked to a ligand, e.g., Fc. In some embodiments, the AAV comprises at least about 5 VP3 proteins that are not linked to a ligand, e.g., Fc. In some embodiments, the AAV comprises at least about 10 VP3 proteins that are not linked to a ligand, e.g., Fc. In some embodiments, the AAV comprises at least about 15 VP3 proteins that are not linked to a ligand, e.g., Fc. In some embodiments, the AAV comprises at least about 20 VP3 proteins that are not linked to a ligand, e.g., Fc. In some embodiments, the AAV comprises at least about 25 VP3 proteins that are not linked to a ligand, e.g., Fc. In some embodiments, the AAV comprises at least about 30 VP3 proteins that are not linked to a ligand, e.g., Fc. In some embodiments, the AAV comprises at least about 35 VP3 proteins that are not linked to a ligand, e.g., Fc. In some embodiments, the AAV comprises at least about 40 VP3 proteins that are not linked to a ligand, e.g., Fc. In some embodiments, the AAV comprises at least about 45 VP3 proteins that are not linked to a ligand, e.g., Fc.

In some embodiments, the number of the VP3 linked to the ligand, e.g., Fc, is a at least bout 2 fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14 fold, at least about 15 fold, at least about 20 fold, at least about 30 fold, at least about 35 fold, at least about 40 fold, at least about 45 fold, at least about 50 fold less than the number of the at least one VP3 protein not linked to the ligand, e.g., Fc.

In certain embodiments, the AAV comprises 1 VP2 protein linked to a ligand, e.g., Fc. In some embodiments, the AAV comprises 2 VP2 proteins linked to ligand, e.g., Fc. In some embodiments, the AAV comprises 3 VP2 proteins linked to ligand, e.g., Fc. In some embodiments, the AAV comprises 4 VP2 proteins linked to ligand, e.g., Fc. In some embodiments, the AAV comprises 5 VP2 proteins linked to ligand, e.g., Fc.

In certain embodiments, the AAV comprises 1 VP1 protein linked to a ligand, e.g., Fc. In some embodiments, the AAV comprises 2 VP1 proteins linked to ligand, e.g., Fc. In some embodiments, the AAV comprises 3 VP1 proteins linked to ligand, e.g., Fc. In some embodiments, the AAV comprises 4 VP1 proteins linked to ligand, e.g., Fc. In some embodiments, the AAV comprises 5 VP1 proteins linked to ligand, e.g., Fc.

In certain embodiments, the AAV comprises 1 VP3 protein linked to a ligand, e.g., Fc. In some embodiments, the AAV comprises 2 VP3 proteins linked to ligand, e.g., Fc. In some embodiments, the AAV comprises 3 VP3 proteins linked to ligand, e.g., Fc. In some embodiments, the AAV comprises 4 VP3 proteins linked to ligand, e.g., Fc. In some embodiments, the AAV comprises 5 VP3 proteins linked to ligand, e.g., Fc.

In some embodiments, the AAV, e.g., a capsid protein of an AAV, is linked to an Fc, and the scaffold protein is linked to an Fc receptor. As use herein, the term “Fc receptor” includes without limitation a fragment of the naturally occurring Fc receptor, wherein the fragment retains the ability to associate with an Fc. In certain embodiments, the Fc receptor linked to the scaffold moiety is an Fc gamma receptor, and the Fc linked to the AAV is an Fc of an IgG. In some embodiments, the Fc gamma receptor is selected from Fc gamma receptor I (FcγR1), FcγRIIA, FcγIIB, FcγIIIA, and FcγIIIB. In some embodiments, the scaffold protein is linked to an FcγR1, and a capsid protein of the AAV is linked to an Fc of an IgG. In some embodiments, the scaffold protein is linked to an FcγRIIA, and a capsid protein of the AAV is linked to an Fc of an IgG. In some embodiments, the scaffold protein is linked to an FcγIIB, and a capsid protein of the AAV is linked to an Fc of an IgG. In some embodiments, the scaffold protein is linked to an FcγIIIA, and a capsid protein of the AAV is linked to an Fc of an IgG. In some embodiments, the scaffold protein is linked to an FcγIIIB, and a capsid protein of the AAV is linked to an Fc of an IgG.

In some embodiments, the scaffold protein is linked to an Fc alpha receptor I (FcαR1), and a capsid protein of the AAV is linked to an Fc of an IgA.

In some embodiments, the Fc receptor is an Fc epsilon receptor selected from Fc epsilon receptor I (FcεRI) and FcεRII, and the Fc is an Fc of an IgE. In certain embodiments, the scaffold protein is linked to an FcεRI, and a capsid protein of the AAV is linked to an Fc of an IgE. In some embodiments, the scaffold protein is linked to an FcεRII, and a capsid protein of the AAV is linked to an Fc of an IgE.

II.C.4.ii. Antigen-Antigen-Binding Domain

In certain aspects of the present disclosure, the scaffold protein is linked to an antigen-binding domain and the AAV comprises an antigen. In some embodiments, the antigen-binding domain specifically binds an antigen on the AAV. In some embodiments, the antigen on the AAV is a capsid protein. In some embodiments, the antigen-binding domain specifically binds VP1 on the surface of the AAV. In some embodiments, the antigen-binding domain specifically binds VP2 on the surface of the AAV. In some embodiments, the antigen-binding domain specifically binds VP3 on the surface of the AAV. In some embodiments, the antigen-binding domain specifically binds both VP1 and VP2. In some embodiments, the antigen-binding domain specifically binds both VP2 and VP3. In some embodiments, the antigen-binding domain specifically binds both VP1 and VP3. In some embodiments, the antigen-binding domain specifically binds VP1, VP2, and VP3.

In some embodiments, the antigen-binding domain specifically binds an antigen on the AAV, wherein the antigen on the AAV is not a naturally occurring AAV protein. In some embodiments, the antigen is heterologously expressed by the AAV. In some embodiments, the antigen is present in the capsid of the AAV. In some embodiments, the antigen is linked to an AAV protein, e.g., a capsid protein.

In some embodiments, the antigen is linked to at least one VP1 protein of the AAV. In some embodiments, an antigen is linked to each of the 5 VP1 proteins of the AAV. In some embodiments, an antigen is linked to each of 4 of the VP1 proteins of the AAV. In some embodiments, an antigen is linked to each of 3 of the VP1 proteins of the AAV. In some embodiments, an antigen is linked to each of 2 of the VP1 proteins of the AAV. In some embodiments, an antigen is linked to 1 of the VP1 proteins of the AAV. In some embodiments, the AAV comprises one VP1 protein that is not linked to an antigen. In some embodiments, the AAV comprises two VP1 proteins that are not linked to an antigen. In some embodiments, the AAV comprises three VP1 proteins that are not linked to an antigen. In some embodiments, the AAV comprises four VP1 proteins that are not linked to an antigen.

In some embodiments, the antigen is linked to at least one VP2 protein of the AAV. In some embodiments, an antigen is linked to each of the 5 VP2 proteins of the AAV. In some embodiments, an antigen is linked to each of 4 of the VP2 proteins of the AAV. In some embodiments, an antigen is linked to each of 3 of the VP2 proteins of the AAV. In some embodiments, an antigen is linked to each of 2 of the VP2 proteins of the AAV. In some embodiments, an antigen is linked to 1 of the VP2 proteins of the AAV. In some embodiments, the AAV comprises one VP2 protein that is not linked to an antigen. In some embodiments, the AAV comprises two VP2 proteins that are not linked to an antigen. In some embodiments, the AAV comprises three VP2 proteins that are not linked to an antigen. In some embodiments, the AAV comprises four VP2 proteins that are not linked to an antigen.

In some embodiments, the antigen is linked to at least one VP3 protein of the AAV. In some embodiments, an antigen is linked to each of the VP3 proteins of the AAV. In some embodiments, an antigen is linked to each of a subset of the VP3 proteins of the AAV. In some embodiments, an antigen is linked to each of at least about 40 of the VP3 proteins of the AAV. In some embodiments, an antigen is linked to each of at least about 35 of the VP3 proteins of the AAV. In some embodiments, an antigen is linked to each of at least about 30 of the VP3 proteins of the AAV. In some embodiments, an antigen is linked to each of at least about 25 of the VP3 proteins of the AAV. In some embodiments, an antigen is linked to each of at least about 20 of the VP3 proteins of the AAV. In some embodiments, an antigen is linked to each of at least about 15 of the VP3 proteins of the AAV. In some embodiments, an antigen is linked to each of at least about 10 of the VP3 proteins of the AAV. In some embodiments, an antigen is linked to each of at least about 9 of the VP3 proteins of the AAV. In some embodiments, an antigen is linked to each of at least about 8 of the VP3 proteins of the AAV. In some embodiments, an antigen is linked to each of at least about 7 of the VP3 proteins of the AAV. In some embodiments, an antigen is linked to each of at least about 6 of the VP3 proteins of the AAV. In some embodiments, an antigen is linked to each of at least about 5 of the VP3 proteins of the AAV. In some embodiments, an antigen is linked to each of at least about 4 of the VP3 proteins of the AAV. In some embodiments, an antigen is linked to each of at least about 3 of the VP3 proteins of the AAV. In some embodiments, an antigen is linked to each of at least about 2 of the VP3 proteins of the AAV. In some embodiments, an antigen is linked to 1 of the VP3 proteins of the AAV. In some embodiments, the AAV comprises at least 1 VP3 protein that is not linked to an antigen. In some embodiments, the AAV comprises at least about 2 VP3 proteins that are not linked to an antigen. In some embodiments, the AAV comprises at least about 3 VP3 proteins that are not linked to an antigen. In some embodiments, the AAV comprises at least about 4 VP3 proteins that are not linked to an antigen. In some embodiments, the AAV comprises at least about 5 VP3 proteins that are not linked to an antigen. In some embodiments, the AAV comprises at least about 10 VP3 proteins that are not linked to an antigen. In some embodiments, the AAV comprises at least about 15 VP3 proteins that are not linked to an antigen. In some embodiments, the AAV comprises at least about 20 VP3 proteins that are not linked to an antigen. In some embodiments, the AAV comprises at least about 25 VP3 proteins that are not linked to an antigen. In some embodiments, the AAV comprises at least about 30 VP3 proteins that are not linked to an antigen. In some embodiments, the AAV comprises at least about 35 VP3 proteins that are not linked to an antigen. In some embodiments, the AAV comprises at least about 40 VP3 proteins that are not linked to an antigen. In some embodiments, the AAV comprises at least about 45 VP3 proteins that are not linked to an antigen.

In some embodiments, the number of the VP3 linked to the antigen is at least about 2 fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14 fold, at least about 15 fold, at least about 20 fold, at least about 30 fold, at least about 35 fold, at least about 40 fold, at least about 45 fold, at least about 50 fold less than the number of the at least one VP3 protein not linked to the antigen.

In certain embodiments, the AAV comprises 1 VP2 protein linked to an antigen. In some embodiments, the AAV comprises 2 VP2 proteins linked to antigen. In some embodiments, the AAV comprises 3 VP2 proteins linked to antigen. In some embodiments, the AAV comprises 4 VP2 proteins linked to antigen. In some embodiments, the AAV comprises 5 VP2 proteins linked to antigen.

In certain embodiments, the AAV comprises 1 VP1 protein linked to an antigen. In some embodiments, the AAV comprises 2 VP1 proteins linked to antigen. In some embodiments, the AAV comprises 3 VP1 proteins linked to antigen. In some embodiments, the AAV comprises 4 VP1 proteins linked to antigen. In some embodiments, the AAV comprises 5 VP1 proteins linked to antigen.

In certain embodiments, the AAV comprises 1 VP3 protein linked to an antigen. In some embodiments, the AAV comprises 2 VP3 proteins linked to antigen. In some embodiments, the AVV comprises 3 VP3 proteins linked to antigen. In some embodiments, the AVV comprises 4 VP3 proteins linked to antigen. In some embodiments, the AVV comprises 5 VP3 proteins linked to antigen.

Any antigen-binding domain/antigen pairing known in the art can be used in the present disclosure. In some embodiments, the antigen is an Fc and the antigen-binding domain specifically binds Fc. In certain embodiments, the AAV, e.g., a capsid protein of the AAV, is linked to an Fc, and the scaffold protein is linked to an antigen-binding domain, wherein the antigen-binding domain specifically binds the Fc. In some embodiments, the Fc is an Fc of IgG. In some embodiments, the Fc is an Fc of IgA. In some embodiments, the Fc is an Fc of IgE.

In some embodiments, the antigen-binding domain comprises an antigen-binding fragment of an antibody. In some embodiments, the antigen-binding domain comprises a single-chain antibody or an antigen-binding fragment thereof. In some embodiments, the antigen-binding domain comprises a humanized antibody or an antigen-binding fragment thereof. In some embodiments, the antigen-binding domain comprises a murine antibody or an antigen-binding fragment thereof. In some embodiments, the antigen-binding domain comprises a chimeric antibody (e.g., a mouse-human, a mouse-primate, or a primate-human monoclonal antibody) or an antigen binding fragment thereof. In some embodiments, the antigen-binding domain comprises an antigen-binding fragment of a camelid antibody, a shark IgNAR, or an anti-idiotype antibody. In some embodiments, the antigen-binding domain comprises a camelid antibody or an antigen-binding fragment thereof. In some embodiments, the antigen-binding domain comprises a shark IgNAR or an antigen-binding fragment thereof. In some embodiments, the antigen-binding domain comprises an anti-idiotype antibody or an antigen-binding fragment thereof.

In some embodiments, the antigen-binding domain comprises a single chain antibody. In some embodiments, the antigen-binding domain comprises an scFv. In some embodiments, the antigen-binding domain comprises an (scFv)₂. In some embodiments, the antigen-binding domain comprises an Fab. In some embodiments, the antigen-binding domain comprises an Fab′. In some embodiments, the antigen-binding domain comprises an F(ab′)₂. In some embodiments, the antigen-binding domain comprises an F(ab1)₂. In some embodiments, the antigen-binding domain comprises an Fv. In some embodiments, the antigen-binding domain comprises a dAb. In some embodiments, the antigen-binding domain comprises a single chain Fab. In some embodiments, the antigen-binding domain comprises an Fd fragment.

In some embodiments, the antigen-binding domain comprises a diabody. In some embodiments, the antigen-binding domain comprises a minibody. In some embodiments, the antigen-binding domain comprises an antibody-related polypeptide. In particular embodiments, the antigen-binding domain comprises a nanobody.

In some embodiments, the antigen-binding domain specifically binds a conformational epitope on the surface of the AAV. In some embodiments, the antigen-binding domain specifically binds an antigen on the surface of the AAV that is only present when the AAV is intact and/or infectious.

II.C.4.iii. AAV Receptor

In certain aspects, the scaffold protein is associated with an AAV binding polypeptide. In some embodiments, the AAV binding polypeptide comprises an AAV receptor. In some embodiments, the scaffold protein is associated with the AAV through an AAV receptor. In some embodiments, the AAV receptor is linked to the scaffold protein. In some embodiments, the AAV receptor is linked to the scaffold protein by a linker. In some embodiments, the receptor is linked to the N-terminus of the scaffold protein. In some embodiments, the receptor is linked to the C-terminus of the scaffold protein. In some embodiments, the receptor is linked to an extracellular domain of the scaffold protein.

Any AAV receptor known in the art or an AAV-binding fragment thereof can be linked to a scaffold protein described herein. In certain embodiments, the AAV receptor is the AAV receptor encoded by the gene KIAA0319L, e.g., the AAV receptor is AAVR (see, e.g., Pillay et al., Nature 530(7588):108-12 (2016), which is incorporated by reference herein in its entirety). AAVR is an N-linked glycosylated protein of about 150 kDa. Full-length AAVR is a type 1 transmembrane protein comprising five Ig-like domains referred to as polycystic kidney disease (PKD) domains. In some embodiments, the scaffold protein is linked to an AAVR fragment, comprising at least the PKD1 domain of the AAVR. In some embodiments, the scaffold protein is linked to an AAVR fragment, comprising at least the PKD2 domain of the AAVR. In some embodiments, the scaffold protein is linked to an AAVR fragment, comprising at least the PKD3 domain of the AAVR. In some embodiments, the scaffold protein is linked to an AAVR fragment, comprising at least the PKD4 domain of the AAVR. In some embodiments, the scaffold protein is linked to an AAVR fragment, comprising at least the PKD5 domain of the AAVR. In some embodiments, the scaffold protein is linked to an AAVR fragment, comprising at least the PKD1 and PKD2 domains of the AAVR. In some embodiments, the scaffold protein is linked to an AAVR fragment, comprising at least the PKD1, PKD2, and PKD3 domains of the AAVR. In some embodiments, the scaffold protein is linked to an AAVR fragment, comprising at least the PKD1, PKD2, PKD3, and PKD4 domains of the AAVR. In some embodiments, the scaffold protein is linked to an AAVR fragment, comprising the PKD1, PKD2, PKD3, PKD4, PKD5 domains of the AAVR. In some embodiments, the scaffold protein is linked an AAVR fragment, wherein the AAVR fragment does not comprise a PKD5 domain. In some embodiments, the scaffold protein is linked an AAVR fragment, wherein the AAVR fragment does not comprise a PKD4 domain. In some embodiments, the scaffold protein is linked an AAVR fragment, wherein the AAVR fragment does not comprise a PKD3 domain. In some embodiments, the scaffold protein is linked an AAVR fragment, wherein the AAVR fragment does not comprise a PKD2 domain. In some embodiments, the scaffold protein is linked an AAVR fragment, wherein the AAVR fragment does not comprise a PKD1 domain. In some embodiments, the scaffold protein is linked an AAVR fragment, wherein the AAVR fragment does not comprise a PKD5 domain or a PKD4 domain. In some embodiments, the scaffold protein is linked an AAVR fragment, wherein the AAVR fragment does not comprise a PKD5 domain, a PKD4 domain, or a PKD3 domain. In some embodiments, the scaffold protein is linked an AAVR fragment, wherein the AAVR fragment does not comprise a PKD5 domain, a PKD4 domain, a PKD3 domain, or a PKD2 domain.

In some embodiments, the receptor is a docking receptor of AAV. In some embodiments, the receptor is selected from heparin sulfate proteoglycan, N-linked sialic acid, O-linked sialic acid, N-linked galactose, CD9, ganglioside GM1, LamR, EGFR, PDGFR, FGFR1, HGFR, and any combination thereof.

II.C.4.iv. Chemically Induced Dimers

Certain aspects of the present disclosure are directed to an EV comprising an AAV and a scaffold protein, wherein the AAV is linked to a first binding partner or dimerizing agent of a chemically induced dimer, and the scaffold protein is linked to a second binding partner or dimerizing agent of the chemically induced dimer. In some embodiments, the first binding partner is linked to a capsid protein of the AAV. In some embodiments, the first binding partner is linked to at least one VP1 protein of the AAV. In some embodiments, a first binding partner is linked to each of the 5 VP1 proteins of the AAV. In some embodiments, a first binding partner is linked to each of 4 of the VP1 proteins of the AAV. In some embodiments, a first binding partner is linked to each of 3 of the VP1 proteins of the AAV. In some embodiments, a first binding partner is linked to each of 2 of the VP1 proteins of the AAV. In some embodiments, a first binding partner is linked to 1 of the VP1 proteins of the AAV. In some embodiments, the AAV comprises one VP1 protein that is not linked to a binding partner. In some embodiments, the AAV comprises two VP1 proteins that are not linked to a binding partner. In some embodiments, the AAV comprises three VP1 proteins that are not linked to a binding partner. In some embodiments, the AAV comprises four VP1 proteins that are not linked to a binding partner.

In some embodiments, the first binding partner is linked to at least one VP2 protein of the AAV. In some embodiments, a first binding partner is linked to each of the 5 VP2 proteins of the AAV. In some embodiments, a first binding partner is linked to each of 4 of the VP2 proteins of the AAV. In some embodiments, a first binding partner is linked to each of 3 of the VP2 proteins of the AAV. In some embodiments, a first binding partner is linked to each of 2 of the VP2 proteins of the AAV. In some embodiments, a first binding partner is linked to 1 of the VP2 proteins of the AAV. In some embodiments, the AAV comprises one VP2 protein that is not linked to a binding partner. In some embodiments, the AAV comprises two VP2 proteins that are not linked to a binding partner. In some embodiments, the AAV comprises three VP2 proteins that are not linked to a binding partner. In some embodiments, the AAV comprises four VP2 proteins that are not linked to a binding partner.

In some embodiments, the first binding partner is linked to at least one VP3 protein of the AAV. In some embodiments, a first binding partner is linked to each of the VP3 proteins of the AAV. In some embodiments, a first binding partner is linked to each of a subset of the VP3 proteins of the AAV. In some embodiments, a first binding partner is linked to each of at least about 40 of the VP3 proteins of the AAV. In some embodiments, a first binding partner is linked to each of at least about 35 of the VP3 proteins of the AAV. In some embodiments, a first binding partner is linked to each of at least about 30 of the VP3 proteins of the AAV. In some embodiments, a first binding partner is linked to each of at least about 25 of the VP3 proteins of the AAV. In some embodiments, a first binding partner is linked to each of at least about 20 of the VP3 proteins of the AAV. In some embodiments, a first binding partner is linked to each of at least about 15 of the VP3 proteins of the AAV. In some embodiments, a first binding partner is linked to each of at least about 10 of the VP3 proteins of the AAV. In some embodiments, a first binding partner is linked to each of at least about 9 of the VP3 proteins of the AAV. In some embodiments, a first binding partner is linked to each of at least about 8 of the VP3 proteins of the AAV. In some embodiments, a first binding partner is linked to each of at least about 7 of the VP3 proteins of the AAV. In some embodiments, a first binding partner is linked to each of at least about 6 of the VP3 proteins of the AAV. In some embodiments, a first binding partner is linked to each of at least about 5 of the VP3 proteins of the AAV. In some embodiments, a first binding partner is linked to each of at least about 4 of the VP3 proteins of the AAV. In some embodiments, a first binding partner is linked to each of at least about 3 of the VP3 proteins of the AAV. In some embodiments, a first binding partner is linked to each of at least about 2 of the VP3 proteins of the AAV. In some embodiments, a first binding partner is linked to 1 of the VP3 proteins of the AAV. In some embodiments, the AAV comprises at least 1 VP3 protein that is not linked to a binding partner. In some embodiments, the AAV comprises at least 2 VP3 proteins that are not linked to a binding partner. In some embodiments, the AAV comprises at least 3 VP3 proteins that are not linked to a binding partner. In some embodiments, the AAV comprises at least 4 VP3 proteins that are not linked to a binding partner. In some embodiments, the AAV comprises at least 5 VP3 proteins that are not linked to a binding partner. In some embodiments, the AAV comprises at least 10 VP3 proteins that are not linked to a binding partner. In some embodiments, the AAV comprises at least 15 VP3 proteins that are not linked to a binding partner. In some embodiments, the AAV comprises at least 20 VP3 proteins that are not linked to a binding partner. In some embodiments, the AAV comprises at least 25 VP3 proteins that are not linked to a binding partner. In some embodiments, the AAV comprises at least 30 VP3 proteins that are not linked to a binding partner. In some embodiments, the AAV comprises at least 35 VP3 proteins that are not linked to a binding partner. In some embodiments, the AAV comprises at least 40 VP3 proteins that are not linked to a binding partner. In some embodiments, the AAV comprises at least 45 VP3 proteins that are not linked to a binding partner.

In some embodiments, the number of the VP3 linked to the first binding partner is at least about 2 fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14 fold, at least about 15 fold, at least about 20 fold, at least about 30 fold, at least about 35 fold, at least about 40 fold, at least about 45 fold, at least about 50 fold less than the number of the at least one VP3 protein not linked to a binding partner.

In certain embodiments, the AVV comprises 1 VP2 protein linked to a first binding partner. In some embodiments, the AVV comprises 2 VP2 proteins linked to first binding partners. In some embodiments, the AVV comprises 3 VP2 proteins linked to first binding partners. In some embodiments, the AVV comprises 4 VP2 proteins linked to first binding partners. In some embodiments, the AVV comprises 5 VP2 proteins linked to first binding partners.

In certain embodiments, the AVV comprises 1 VP1 protein linked to a first binding partner. In some embodiments, the AVV comprises 2 VP1 proteins linked to first binding partners. In some embodiments, the AVV comprises 3 VP1 proteins linked to first binding partners. In some embodiments, the AVV comprises 4 VP1 proteins linked to first binding partners. In some embodiments, the AVV comprises 5 VP1 proteins linked to first binding partners.

In certain embodiments, the AVV comprises 1 VP3 protein linked to a first binding partner. In some embodiments, the AVV comprises 2 VP3 proteins linked to first binding partners. In some embodiments, the AVV comprises 3 VP3 proteins linked to first binding partners. In some embodiments, the AVV comprises 4 VP3 proteins linked to first binding partners. In some embodiments, the AVV comprises 5 VP3 proteins linked to first binding partners.

In some embodiments, the binding partner is linked to the N-terminus of the capsid protein. In other embodiments, the first binding partner is linked to the C-terminus of the capsid protein. In other embodiments, the first binding partner is inserted within the capsid protein, e.g., between the N-terminus and the C-terminus of the capsid protein. In some embodiments, the first binding partner is inserted within the capsid protein. In certain embodiments, the first binding partner is inserted within the capsid protein, e.g., VP1, VP2, and/or VP3, within an internal loop, e.g., an series of amino acids which form a loop structure that is on the surface of the capsid protein. In certain embodiments, the first binding partner is inserted within the capsid protein, e.g., VP1, VP2, and/or VP3, immediately downstream of amino acid 455 (relative to the numbering of SEQ ID NO:44). In some embodiments, the first binding partner is inserted within the capsid protein, e.g., VP1, VP2, and/or VP3, by replacing Gly₄₅₃ (relative to the numbering of SEQ ID NO:44). In some embodiments, the first binding partner is inserted within the capsid protein, e.g., VP1, VP2, and/or VP3, by replacing Thr₄₅₄ (relative to the numbering of SEQ ID NO:44). In some embodiments, the first binding partner is inserted within the capsid protein, e.g., VP1, VP2, and/or VP3, by replacing Thr₄₅₅ (relative to the numbering of SEQ ID NO:44). In some aspects, the first binding partner is inserted within the capsid protein, e.g., VP1, VP2, and/or VP3, by replacing Thr₄₅₆ (relative to the numbering of SEQ ID NO:44). In some embodiments, the first binding partner is inserted within the capsid protein, e.g., VP1, VP2, and/or VP3, by replacing Gln₄₅₇ (relative to the numbering of SEQ ID NO:44). In some embodiments, the first binding partner is inserted within the capsid protein, e.g., VP1, VP2, and/or VP3, by replacing Ser₄₅₈ (relative to the numbering of SEQ ID NO:44). In some embodiments, the first binding partner is inserted within the capsid protein, e.g., VP1, VP2, and/or VP3, by replacing Arg₄₅₉ (relative to the numbering of SEQ ID NO:44). In some embodiments, the first binding partner is inserted within the capsid protein, e.g., VP1, VP2, and/or VP3, by replacing ₄₅₃GTTTQSR₄₅₉ (relative to the numbering of SEQ ID NO:44). In particular embodiments, a first binding partner is inserted within at least one VP3 protein by replacing Thr₄₅₅ (relative to the numbering of SEQ ID NO:44), or into a homologous region of a VP proteins of other AAV serotypes. In particular embodiments, a first binding partner is inserted within at least one VP3 protein by replacing ₄₅₃GTTTQSR₄₅₉ (relative to the numbering of SEQ ID NO:44), or into a homologous region of a VP proteins of other AAV serotypes.

The first binding partner can be linked to the capsid protein of the AAV. In some embodiments, the first binding partner is linked to the capsid by a linker.

In some embodiments, the second binding partner is linked to the scaffold protein. In some embodiments, the AAV receptor is linked to the scaffold protein. In some embodiments, the AAV receptor is linked to the scaffold protein by a linker. In some embodiments, the receptor is linked to the N-terminus of the scaffold protein. In some embodiments, the receptor is linked to the C-terminus of the scaffold protein. In some embodiments, the receptor is linked to an extracellular domain of the scaffold protein.

In some embodiments, the first binding partner linked to the AAV capsid protein and the second binding partner linked to the scaffold protein are selected from a first and a second binding partners of a chemically induced dimer selected from the group consisting of (i) FKBP and FKBP (FK1012); (ii) FKBP and CalcineurinA (CNA) (FK506); (iii) FKBP and CyP-Fas (FKCsA); (iv) FKBP and FRB (Rapamycin); (v) GyrB and GyrB (Coumermycin); (vi) GAI and GID1 (Gibberellin); (vii) Snap-tag and HaloTag (HaXS); (viii) eDHFR and HaloTag (TMP-HTag); and (ix) BCL-xL and Fab (AZ1) (ABT-737). In some embodiments, the AAV capsid protein is linked to FKBP, and the scaffold protein is linked to FKBP. In some embodiments, the AAV capsid protein is linked to FKBP, and the scaffold protein is linked to CalcineurinA (CNA). In some embodiments, the AAV capsid protein is linked to CalcineurinA (CNA), and the scaffold protein is linked to FKBP. In some embodiments, the AAV capsid protein is linked to FKBP, and the scaffold protein is linked to CyP-Fas. In some embodiments, the AAV capsid protein is linked to CyP-Fas, and the scaffold protein is linked to FKBP. In some embodiments, the AAV capsid protein is linked to FKBP, and the scaffold protein is linked to FRB. In some embodiments, the AAV capsid protein is linked to FRB, and the scaffold protein is linked to FKBP. In some embodiments, the AAV capsid protein is linked to GyrB, and the scaffold protein is linked to GyrB. In some embodiments, the AAV capsid protein is linked to GAI, and the scaffold protein is linked to GID1. In some embodiments, the AAV capsid protein is linked GID1, and the scaffold protein is linked to GAI. In some embodiments, the AAV capsid protein is linked to Snap-tag, and the scaffold protein is linked to HaloTag. In some embodiments, the AAV capsid protein is linked to HaloTag, and the scaffold protein is linked to Snap-tag. In some embodiments, the AAV capsid protein is linked to HaloTag, and the scaffold protein is linked to eDHFR. In some embodiments, the AAV capsid protein is linked to eDHFR, and the scaffold protein is linked to HaloTag. In some embodiments, the AAV capsid protein is linked to BCL-xL, and the scaffold protein is linked to Fab (AZ1). In some embodiments, the AAV capsid protein is linked to Fab (AZ1), and the scaffold protein is linked to BCL-xL.

In particular embodiments, the AAV comprises at least one capsid protein (e.g., VP1, VP2, and/or VP3) linked to an FRB, wherein the FRB is linked to the N-terminus of the capsid protein. In some embodiments, the AAV comprises at least one capsid protein (e.g., VP1, VP2, and/or VP3) linked to an FRB, wherein the FRB is linked to the C-terminus of the capsid protein. In particular embodiments, the AAV comprises at least one capsid protein (e.g., VP1, VP2, and/or VP3) linked to an FRB, wherein the FRB is inserted within the capsid protein. In some embodiments, the FRB is inserted within the capsid protein at any location disclosed herein.

II.C.4.v. Affinity Agents

In some embodiments, the scaffold protein is linked to an affinity agent. In some embodiments, the affinity agent is linked to the N-terminus of the scaffold protein. In some embodiments, the affinity agent is linked to the C-terminus of the scaffold protein. In some embodiments, the affinity agent is linked to an extracellular domain of the scaffold protein. In some embodiments, the affinity agent comprises an AAV binding polypeptide. In some embodiments, the affinity agent comprises an AAV receptor. In some embodiments, the affinity agent comprises an antibody or an antigen binding domain, as disclosed herein. In some embodiments, the affinity agent binds to one or more AAV capsid proteins. In some embodiments, the one or more AAV capsid proteins is AAV assembly activating proteins. In some embodiments, the affinity agent does not bind to an AAV capsid protein monomer.

In some aspects, the affinity agent is capable of binding more than one AAV serotype. In some aspects, the affinity agent is capable of binding more than AAV serotype selected from AAV type 1, AAV type 2, AAV type 3A, AAV type 3B, AAV type 4, AAV type 5, AAV type 6, AAV type 7, AAV type 8, AAV type 9, AAV type 10, AAV type 11, AAV type 12, AAV type 13, Rh10, Rh74, AAV-2i8, snake AAV, avian AAV, bovine AAV, canine AAV, equine AAV, ovine AAV, goat AAV, shrimp AAV, a synthetic AAV, an any combination thereof. In some aspects, the affinity agent specifically binds an AAV9 serotype. In some aspects, the affinity agent can bind any AAV serotype. In some aspects, the affinity agent specifically binds an AAV2 serotype. In some aspects, the affinity agent specifically binds an AAV4 serotype. In some aspects, the affinity agent specifically binds an AAV5 serotype.

In some embodiments, the interaction between the affinity agent and the AAV is transient. In some embodiments, the AAV is dissociated form the affinity agent under certain conditions. In certain embodiments, the affinity of the affinity agent to the AAV is dependent on pH. In some embodiments, the AAV dissociates from the affinity agent at a pH of at least about 3, at least about 4, at least about 5, at least about 6, at least about 7, at least about 8, at least about 9, at least about 10, at least about 11, or at least about 12. In some embodiments, the affinity of the affinity agent for the AAV is dependent on the concentration of calcium, magnesium, sulfate, phosphate, or any combination thereof in the solution comprising the AAV and the affinity agent. In some embodiments, the affinity of the affinity agent for the AAV is dependent on the salt concentration and/or ionic strength of the solution comprising the AAV and the affinity agent. In some embodiments, the AAV and the affinity agent are dissociable under reducing conditions.

In some embodiments, the scaffold protein is linked to an AAV binding polypeptide. In some embodiments, the AAV binding polypeptide is linked to the N-terminus of the scaffold protein. In some embodiments, the AAV binding polypeptide is linked to the C-terminus of the scaffold protein. In some embodiments, the AAV binding polypeptide is linked to a extracellular domain of the scaffold protein.

In some embodiments, the AAV binding polypeptide comprises an antigen-binding domain. In some embodiments, the antigen-binding domain comprises an antigen-binding fragment of an antibody. In some embodiments, the antigen-binding domain comprises a single-chain antibody or an antigen-binding fragment thereof. In some embodiments, the antigen-binding domain comprises a humanized antibody or an antigen-binding fragment thereof. In some embodiments, the antigen-binding domain comprises a murine antibody or an antigen-binding fragment thereof. In some embodiments, the antigen-binding domain comprises a chimeric antibody (e.g., a mouse-human, a mouse-primate, or a primate-human monoclonal antibody) or an antigen binding fragment thereof. In some embodiments, the antigen-binding domain comprises an antigen-binding fragment of a camelid antibody, a shark IgNAR, or an anti-idiotype antibody. In some embodiments, the antigen-binding domain comprises a camelid antibody or an antigen-binding fragment thereof. In some embodiments, the antigen-binding domain comprises a shark IgNAR or an antigen-binding fragment thereof. In some embodiments, the antigen-binding domain comprises an anti-idiotype antibody or an antigen-binding fragment thereof.

In some embodiments, the antigen-binding domain comprises a single chain antibody. In some embodiments, the antigen-binding domain comprises an scFv. In some embodiments, the antigen-binding domain comprises an (scFv)₂. In some embodiments, the antigen-binding domain comprises an Fab. In some embodiments, the antigen-binding domain comprises an Fab′. In some embodiments, the antigen-binding domain comprises an F(ab′)₂. In some embodiments, the antigen-binding domain comprises an F(ab1)₂. In some embodiments, the antigen-binding domain comprises an Fv. In some embodiments, the antigen-binding domain comprises a dAb. In some embodiments, the antigen-binding domain comprises a single chain Fab. In some embodiments, the antigen-binding domain comprises an Fd fragment.

In some embodiments, the antigen-binding domain comprises a diabody. In some embodiments, the antigen-binding domain comprises a minibody. In some embodiments, the antigen-binding domain comprises an antibody-related polypeptide. In particular embodiments, the antigen-binding domain comprises a nanobody.

II.D. Linkers

As described supra, EVs of the present disclosure (e.g., exosomes and nanovesicles) can comprises one or more linkers that link a first element to a second element (e.g., a scaffold protein to a capsid protein, a scaffold protein to a binding partner, a capsid protein to a binding partner, a scaffold protein to a nanobody, a scaffold protein to a receptor (e.g., an Fc receptor), an Fc to a scaffold protein, a scaffold protein to an antigen-binding domain, an AAVR to a scaffold protein, an antigen to a capsid protein, an Fc to a capsid protein, or any combination thereof).

The linker can be any chemical moiety known in the art to join two elements. As used herein, the term “linker” refers to a peptide or polypeptide sequence (e.g., a synthetic peptide or polypeptide sequence) or to a non-polypeptide, e.g., an alkyl chain. In some aspects, two or more linkers can be linked in tandem. When multiple linkers are present, each of the linkers can be the same or different. Generally, linkers provide flexibility or prevent/ameliorate steric hindrances. Linkers are not typically cleaved; however in certain aspects, such cleavage can be desirable. Accordingly, in some aspects, a linker can comprise one or more protease-cleavable sites, which can be located within the sequence of the linker or flanking the linker at either end of the linker sequence. In some aspects, the cleavable linker allows for the release of the AAV.

In some embodiments, the linker is a peptide linker. In some embodiments, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some embodiments, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides, which are modified forms of naturally occurring polypeptides (e.g., comprising a mutation such as an addition, substitution or deletion).

Linkers can be susceptible to cleavage (“cleavable linker”) thereby facilitating release of the AAV or the scaffold protein. In some embodiments, the scaffold protein is linked to a capsid protein by a cleavable linker, wherein cleavage of the cleavable linker releases the AAV. In some embodiments, the scaffold protein is linked to a binding partner of a chemically induced dimer, as described herein, by a cleavable linker, wherein cleavage of the cleavable linker releases the scaffold protein from the binding partner. In some embodiments, a capsid protein of an AAV is linked to a binding partner of a chemically induced dimer, as described herein, by a cleavable linker, wherein cleavage of the cleavable linker releases the capsid protein from the binding partner. In some embodiments, the scaffold protein is linked to a nanobody by a cleavable linker, wherein cleavage of the cleavable linker releases the scaffold protein from the nanobody. In some embodiments, the scaffold protein is linked to an antigen-binding domain, as described herein, by a cleavable linker, wherein cleavage of the cleavable linker releases the scaffold protein from the antigen-binding domain. In some embodiments, the scaffold protein is linked to a receptor (e.g., an Fc receptor), as described herein, by a cleavable linker, wherein cleavage of the cleavable linker releases the scaffold protein from the receptor (e.g., the Fc receptor). In some embodiments, the scaffold protein is linked to an AAVR, as described herein, by a cleavable linker, wherein cleavage of the cleavable linker releases the scaffold protein from the AAVR. In some embodiments, a capsid protein of the AAV is linked to an antigen, as described herein, by a cleavable linker, wherein cleavage of the cleavable linker releases the capsid protein from the antigen. In some embodiments, a capsid protein of an AAV is linked to an Fc by a cleavable linker, wherein cleavage of the cleavable linker releases the capsid protein from the Fc.

In some aspects, the linker is a “reduction-sensitive linker.” In some aspects, the reduction-sensitive linker contains a disulfide bond. In some aspects, the linker is an “acid labile linker.” In some aspects, the acid labile linker contains hydrazone. Suitable acid labile linkers also include, for example, a cis-aconitic linker, a hydrazide linker, a thiocarbamoyl linker, or any combination thereof.

In some aspects, the cleavable linker comprises a dinucleotide or trinucleotide linker, a disulfide, an imine, a thioketal, a val-cit dipeptide, or any combination thereof.

In some aspects, the cleavable linker comprises valine-alanine-p-aminobenzylcarbamate, valine-citrulline-p-aminobenzylcarbamate, or both.

In some aspects, the cleavable linker comprises redox cleavable linkers, reactive oxygen species (ROS) cleavable linkers, pH dependent cleavable linkers, enzymatic cleavable linkers, protease cleavable linkers, esterase cleavable linkers, phosphatase cleavable linkers, photoactivated cleavable linkers, self-immolative linkers, or combinations thereof. Additional disclosure relating to one or more of these cleavable linkers are provided further below and also known in the art, see, e.g., US 2018/0037639 A1; Trout et al., 79 Proc. Natl. Acad. Sci. USA, 626-629 (1982); Umemoto et al. 43 Int. J. Cancer, 677-684 (1989); Cancer Res. 77(24):7027-7037 (2017); Doronina et al. Nat. Biotechnol. 21:778-784 (2003); U.S. Pat. No. 7,754,681 B2; US 2006/0269480; US 2010/0092496; US 2010/0145036; US 2003/0130189; US 2005/0256030, each of which is herein incorporated by reference in its entirety.

In some aspects, the linker combination comprises a redox cleavable linker. In certain aspects, such a linker can comprise a redox cleavable linking group that is cleaved upon reduction or upon oxidation.

In some aspects, the redox cleavable linker contains a disulfide bond, i.e., it is a disulfide cleavable linker. In some aspects, the redox cleavable linker can be reduced, e.g., by intracellular mercaptans, oxidases, reductases, or combinations thereof.

In some aspects, the linker combination can comprise a cleavable linker which can be cleaved by a reactive oxygen species (ROS), such as superoxide (Of) or hydrogen peroxide (H2O2), generated, e.g., by inflammation processes such as activated neutrophils. In some aspects, the ROS cleavable linker is a thioketal cleavable linker. See, e.g., U.S. Pat. No. 8,354,455B2, which is herein incorporated by reference in its entirety.

In some aspects, the linker is an acid labile linker comprising an acid cleavable linking group, which is a linking group that is selectively cleaved under acidic conditions (pH<7).

In some aspects, the acid cleavable linking group is cleaved in an acidic environment, e.g., about 6.0, about 5.5, about 5.0 or less. In some aspects, the pH is about 6.5 or less. In some aspects, the linker is cleaved by an agent such as an enzyme that can act as a general acid, e.g., a peptidase (which can be substrate specific) or a phosphatase. Within cells, certain low pH organelles, such as endosomes and lysosomes, can provide a cleaving environment to the acid cleavable linking group. Although the pH of human serum is 7.4, the average pH in cells is slightly lower, ranging from about 7.1 to 7.3. Endosomes also have an acidic pH, ranging from 5.5 to 6.0, and lysosomes are about 5.0 at an even more acidic pH. Accordingly, pH dependent cleavable linkers are sometimes called endosomically labile linkers in the art.

In some aspects, the acid cleavable group can have the general formula —C═NN—, C (O) O, or —OC(O). In another non-limiting example, when the carbon attached to the ester oxygen (alkoxy group) is attached to an aryl group, a substituted alkyl group, or a tertiary alkyl group such as dimethyl pentyl or t-butyl, for example. Examples of acid cleavable linking groups include, but are not limited to, amine, imine, amino ester, benzoic imine, diortho ester, polyphosphoester, polyphosphazene, acetal, vinyl ether, hydrazone, cis-aconitate, hydrazide, thiocarbamoyl, imizine, azidomethyl-methylmaleic anhydride, thiopropionate, a masked endosomolytic agent, a citraconyl group, or any combination thereof. Disulfide linkages are also susceptible to pH.

In some aspects, the linker comprises a low pH-labile hydrazone bond. Such acid-labile bonds have been extensively used in the field of conjugates, e.g., antibody-drug conjugates. See, for example, Zhou et al, Biomacromolecules 2011, 12, 1460-7; Yuan et al, Acta Biomater. 2008, 4, 1024-37; Zhang et al, Acta Biomater. 2007, 6, 838-50; Yang et al, J. Pharmacol. Exp. Ther. 2007, 321, 462-8; Reddy et al, Cancer Chemother. Pharmacol. 2006, 58, 229-36; Doronina et al, Nature Biotechnol. 2003, 21, 778-84, each of which are hereby incorporated by reference in its entirety.

In some aspects, the linker comprises a low pH-labile bond selected from the following: ketals that are labile in acidic environments (e.g., pH less than 7, greater than about 4) to form a diol and a ketone; acetals that are labile in acidic environments (e.g., pH less than 7, greater than about 4) to form a diol and an aldehyde; imines or iminiums that are labile in acidic environments (e.g., pH less than 7, greater than about 4) to form an amine and an aldehyde or a ketone; silicon-oxygen-carbon linkages that are labile under acidic condition; silicon-nitrogen (silazane) linkages; silicon-carbon linkages (e.g., arylsilanes, vinylsilanes, and allylsilanes); maleamates (amide bonds synthesized from maleic anhydride derivatives and amines); ortho esters; hydrazones; activated carboxylic acid derivatives (e.g., esters, amides) designed to undergo acid catalyzed hydrolysis); or vinyl ethers.

Further examples can be found in U.S. Pat. Nos. 9,790,494 B2 and 8,137,695 B2, the contents of which are incorporated herein by reference in their entireties.

In some aspects, the linker combination can comprise a linker cleavable by intracellular or extracellular enzymes, e.g., proteases, esterases, nucleases, amidades. The range of enzymes that can cleave a specific linker in a linker combination depends on the specific bonds and chemical structure of the linker. Accordingly, peptidic linkers can be cleaved, e.g., by peptidades, linkers containing ester linkages can be cleaved, e.g., by esterases; linkers containing amide linkages can be cleaved, e.g., by amidades; etc.

Some linkers are cleaved by esterases (“esterase cleavable linkers”). Only certain esters can be cleaved by esterases and amidases present inside or outside of cells. Esters are formed by the condensation of a carboxylic acid and an alcohol. Simple esters are esters produced with simple alcohols, such as aliphatic alcohols, and small cyclic and small aromatic alcohols. Examples of ester-based cleavable linking groups include, but are not limited to, esters of alkylene, alkenylene and alkynylene groups. The ester cleavable linking group has the general formula —C(O) O— or —OC(O)—.

In some aspects, a linker combination can includes a phosphate-based cleavable linking group is cleaved by an agent that degrades or hydrolyzes phosphate groups. An example of an agent that cleaves intracellular phosphate groups is an enzyme such as intracellular phosphatase. Examples of phosphate-based linking groups are —O—P (O) (OR k)-O—, —O—P (S) (ORₖ)—O—, —O—P (S) (SRₖ)— O—, —S—P (O) (ORₖ)—O—, —O—P (O) (ORₖ)—S—, —S—P (O) (ORₖ)—S—, —O—P (S) (ORₖ)—S—, —SP (S) (ORₖ)—O—, —OP (O) (Rₖ)—O—, —OP (S) (Rₖ)—O—, —SP (O) (Rₖ)—O—, —SP (S) (Rₖ)—O—, —SP (O) (Rₖ)—S—, or —OP (S) (Rₖ)—S—.

In some aspects, Rₖ is any of the following: NH₂, BH₃, CH₃, C₁₋₆ alkyl, C₆₋₁₀ aryl, C₁₋₆ alkoxy and C₆₋₁₀ aryl-oxy. In some aspects, C₁₋₆ alkyl and C₆₋₁₀ aryl are unsubstituted. Further non-limiting examples include —O—P (O) (OH)—O—, —O—P (S) (OH)—O—, —O—P (S) (SH)—O—, —S—P (O) (OH)—O—, —O—P (O) (OH)—S—, —S—P (O) (OH)—S—, —O—P (S) (OH)—S—, —S—P (S) (OH)—O—, —O—P (O) (H)—O—, —O—P (S) (H)—O—, —S—P (O) (H)—O—, —SP (S) (H)—O—, —SP (O) (H)—S—, —OP (S) (H)—S—, or —O—P (O) (OH)—O—.

In some aspects, the combination linker comprises a photoactivated cleavable linker, e.g., a nitrobenzyl linker or a linker comprising a nitrobenzyl reactive group.

In some aspects, the linker comprises a non-cleavable linker.

### III. Producer Cell for Production of Engineered Exosomes

EVs, e.g., exosomes, of the present disclosure can be produced from a cell grown in vitro or a body fluid of a subject. When exosomes are produced from in vitro cell culture, various producer cells, e.g., HEK293 cells, CHO cells, and MSCs, can be used. In certain embodiments, a producer cell is not a dendritic cell, macrophage, B cell, mast cell, neutrophil, Kupffer-Browicz cell, cell derived from any of these cells, or any combination thereof.

The producer cell can be genetically modified to comprise one or more exogenous sequences (e.g., encoding an AAV, a scaffold protein, or a therapeutic protein) to produce exosomes described herein. The genetically-modified producer cell can contain the exogenous sequence by transient or stable transformation. The exogenous sequence can be transformed as a plasmid. The exogenous sequences can be stably integrated into a genomic sequence of the producer cell, at a targeted site or in a random site. In some embodiments, a stable cell line is generated for production of lumen-engineered exosomes.

The exogenous sequences can be inserted into a genomic sequence of the producer cell, located within, upstream (5′-end) or downstream (3′-end) of an endogenous sequence encoding an exosome protein. Various methods known in the art can be used for the introduction of the exogenous sequences into the producer cell. For example, cells modified using various gene editing methods (e.g., methods using a homologous recombination, transposon-mediated system, loxP-Cre system, CRISPR/Cas9 or TALEN) are within the scope of the present disclosure.

The exogenous sequences can comprise a sequence encoding a scaffold protein disclosed herein or a fragment or variant thereof. An extra copy of the sequence encoding a scaffold protein can be introduced to produce an exosome described herein (e.g., having a higher density of a scaffold protein on the surface, e.g., on the luminal and/or external surface of the EV, e.g., exosome). An exogenous sequence encoding a modification or a fragment of a scaffold protein can be introduced to produce a lumen-engineered and/or surface-engineered exosome containing the modification or the fragment of the scaffold protein.

In some embodiments, a producer cell can be modified, e.g., transfected, with one or more vectors encoding a scaffold protein linked to a capsid protein of an AAV, an AAV receptor, a binding partner of a chemically induced dimer, an antigen-binding domain (e.g., a nanobody), an Fc receptor, or any combination thereof.

In some embodiments, a producer cell disclosed herein is further modified to comprise an additional exogenous sequence. For example, an additional exogenous sequence can be introduced to modulate endogenous gene expression, or produce an exosome including a payload (e.g., AAV). In some embodiments, the producer cell is modified to comprise two exogenous sequences, one encoding a scaffold protein, or a variant or a fragment thereof, and the other encoding a payload (e.g., AAV). In certain embodiments, the producer cell can be further modified to comprise an additional exogenous sequence conferring additional functionalities to exosomes. In some embodiments, the producer cell is modified to comprise two exogenous sequences, one encoding a scaffold protein disclosed herein, or a variant or a fragment thereof, and the other encoding an AAV. In some embodiments, the producer cell is further modified to comprise one, two, three, four, five, six, seven, eight, nine, or ten or more additional exogenous sequences.

Any of the scaffold moieties described herein can be expressed from a plasmid, an exogenous sequence inserted into the genome or other exogenous nucleic acid, such as a synthetic messenger RNA (mRNA).

In some embodiments, the EV and the AAV are produced by a single cell or a single population of cell types. In some embodiments, the EV is produced by a first cell (or a first population of cells), and the AAV is produced by a second cell (or a second population of cells). In some embodiments, the first cell and the second cell are the same type of cell. In some embodiments, the first cell and the second cell are not the same type of cell.

### IV. Pharmaceutical Compositions

Provided herein are pharmaceutical compositions comprising an EV, e.g., exosome, of the present disclosure having the desired degree of purity, and a pharmaceutically acceptable carrier or excipient, in a form suitable for administration to a subject. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of extracellular vesicles. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

In some embodiments, a pharmaceutical composition comprises one or more therapeutic agents and an exosome described herein. In certain embodiments, the EVs, e.g., exosomes, are co-administered with of one or more additional therapeutic agents, in a pharmaceutically acceptable carrier. In some embodiments, the pharmaceutical composition comprising the EV, e.g., exosome is administered prior to administration of the additional therapeutic agents. In other embodiments, the pharmaceutical composition comprising the EV, e.g., exosome is administered after the administration of the additional therapeutic agents. In further embodiments, the pharmaceutical composition comprising the EV, e.g., exosome is administered concurrently with the additional therapeutic agents.

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the extracellular vesicles described herein, use thereof in the compositions is contemplated. Supplementary therapeutic agents can also be incorporated into the compositions. Typically, a pharmaceutical composition is formulated to be compatible with its intended route of administration. The EVs, e.g., exosomes, can be administered by parenteral, topical, intravenous, oral, subcutaneous, intra-arterial, intradermal, transdermal, rectal, intracranial, intraperitoneal, intranasal, intratumoral, intramuscular route or as inhalants. In certain embodiments, the pharmaceutical composition comprising exosomes is administered intravenously, e.g. by injection. The EVs, e.g., exosomes, can optionally be administered in combination with other therapeutic agents that are at least partly effective in treating the disease, disorder or condition for which the EVs, e.g., exosomes, are intended.

Solutions or suspensions can include the following components: a sterile diluent such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfate; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if water soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as mannitol, sorbitol, and sodium chloride can be added to the composition. Prolonged absorption of the injectable compositions can be brought about by including in the composition a compound which delays absorption, e.g., aluminum monostearate and gelatin.

Sterile injectable solutions can be prepared by incorporating the EVs, e.g., exosomes, in an effective amount and in an appropriate solvent with one or a combination of ingredients enumerated herein, as desired. Generally, dispersions are prepared by incorporating the EVs, e.g., exosomes, into a sterile vehicle that contains a basic dispersion medium and any desired other ingredients. In the case of sterile powders for the preparation of sterile injectable solutions, methods of preparation are vacuum drying and freeze-drying that yields a powder of the active ingredient plus any additional desired ingredient from a previously sterile-filtered solution thereof. The EVs, e.g., exosomes, can be administered in the form of a depot injection or implant preparation which can be formulated in such a manner to permit a sustained or pulsatile release of the EV, e.g., exosomes.

Systemic administration of compositions comprising exosomes can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of, e.g., nasal sprays.

In certain embodiments the pharmaceutical composition comprising exosomes is administered intravenously into a subject that would benefit from the pharmaceutical composition. In certain other embodiments, the composition is administered to the lymphatic system, e.g., by intralymphatic injection or by intranodal injection (see e.g., Senti et al., PNAS 105(46): 17908 (2008)), or by intramuscular injection, by subcutaneous administration, by intratumoral injection, by direct injection into the thymus, or into the liver.

In certain embodiments, the pharmaceutical composition comprising exosomes is administered as a liquid suspension. In certain embodiments, the pharmaceutical composition is administered as a formulation that is capable of forming a depot following administration. In certain preferred embodiments, the depot slowly releases the EVs, e.g., exosomes, into circulation, or remains in depot form.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically-acceptable carrier can be lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

The pharmaceutical compositions described herein comprise the EVs, e.g., exosomes, described herein and optionally a pharmaceutically active or therapeutic agent. The therapeutic agent can be a biological agent, a small molecule agent, or a nucleic acid agent.

Dosage forms are provided that comprise a pharmaceutical composition comprising the EVs, e.g., exosomes, described herein. In some embodiments, the dosage form is formulated as a liquid suspension for intravenous injection. In some embodiments, the dosage form is formulated as a liquid suspension for intratumoral injection.

In certain embodiments, the preparation of exosomes is subjected to radiation, e.g., X rays, gamma rays, beta particles, alpha particles, neutrons, protons, elemental nuclei, UV rays in order to damage residual replication-competent nucleic acids.

In certain embodiments, the preparation of exosomes is subjected to gamma irradiation using an irradiation dose of more than about 1, about 5, about 10, about 15, about 20, about 25, about 30, about 35, about 40, about 50, about 60, about 70, about 80, about 90, about 100, or more than about 100 kGy.

In certain embodiments, the preparation of exosomes is subjected to X-ray irradiation using an irradiation dose of more than about 0.1, about 0.5, about 1, about 5, about 10, about 15, about 20, about 25, about 30, about 35, about 40, about 50, about 60, about 70, about 80, about 90, about 100, about 200, about 300, about 400, about 500, about 600, about 700, about 800, about 900, about 1000, about 2000, about 3000, about 4000, about 5000, about 6000, about 7000, about 8000, about 9000, about 10000, or greater than about 10000 mSv.

### V. Kits

Also provided herein are kits comprising one or more exosomes described herein. In some embodiments, provided herein is a pharmaceutical pack or kit comprising one or more containers filled with one or more of the ingredients of the pharmaceutical compositions described herein, such as one or more exosomes provided herein, optional an instruction for use. In some embodiments, the kits contain a pharmaceutical composition described herein and any prophylactic or therapeutic agent, such as those described herein.

### VI. Methods of Producing Exosomes

In some aspects, the present disclosure is also directed to methods of producing exosomes described herein. In some embodiments, the method comprises: obtaining the EV, e.g., exosome, from a producer cell, and optionally isolating the obtained EV, e.g., exosome. In some embodiments, the method comprises: modifying a producer cell by introducing two or more components of an exosome disclosed herein (e.g., a scaffold protein and an AAV); obtaining the EV, e.g., exosome from the modified producer cell; and optionally isolating the obtained EV, e.g., exosome. In further embodiments, the method comprises: obtaining an exosome from a producer cell; isolating the obtained exosome; and modifying the isolated exosome (e.g., by inserting an AAV). In certain embodiments, the method further comprises formulating the isolated exosome into a pharmaceutical composition.

VI.A. Methods of Modifying a Producer Cell

As described supra, in some embodiments, a method of producing an exosome comprises modifying a producer cell with one or more moieties (e.g., a scaffold protein and/or an AAV). In certain embodiments, the one or more moieties comprise an AAV. In some embodiments, the one or more moieties further comprise a scaffold protein disclosed herein.

In some embodiments, the producer cell can be a mammalian cell line, a plant cell line, an insect cell line, a fungi cell line, or a prokaryotic cell line. In certain embodiments, the producer cell is a mammalian cell line. Non-limiting examples of mammalian cell lines include: a human embryonic kidney (HEK) cell line, a Chinese hamster ovary (CHO) cell line, an HT-1080 cell line, a HeLa cell line, a PERC-6 cell line, a CEVEC cell line, a fibroblast cell line, an amniocyte cell line, an epithelial cell line, a mesenchymal stem cell (MSC) cell line, and combinations thereof. In certain embodiments, the mammalian cell line comprises HEK-293 cells, BJ human foreskin fibroblast cells, fHDF fibroblast cells, AGE.HN® neuronal precursor cells, CAP® amniocyte cells, adipose mesenchymal stem cells, RPTEC/TERT1 cells, or combinations thereof. In some embodiments, the producer cell is a primary cell. In certain embodiments, the primary cell can be a primary mammalian cell, a primary plant cell, a primary insect cell, a primary fungi cell, or a primary prokaryotic cell.

In some embodiments, the producer cell is not an immune cell, such an antigen presenting cell, a T cell, a B cell, a natural killer cell (NK cell), a macrophage, a T helper cell, or a regulatory T cell (Treg cell). In other embodiments, the producer cell is not an antigen presenting cell (e.g., dendritic cells, macrophages, B cells, mast cells, neutrophils, Kupffer-Browicz cell, or a cell derived from any such cells).

In some embodiments, the one or more moieties can be a transgene or mRNA, and introduced into the producer cell by transfection, viral transduction, electroporation, extrusion, sonication, cell fusion, or other methods that are known to the skilled in the art.

In some embodiments, the one or more moieties is introduced to the producer cell by transfection. In some embodiments, the one or more moieties can be introduced into suitable producer cells using synthetic macromolecules, such as cationic lipids and polymers (Papapetrou et al., Gene Therapy 12: S118-S130 (2005)). In some embodiments, the cationic lipids form complexes with the one or more moieties through charge interactions. In some of these embodiments, the positively charged complexes bind to the negatively charged cell surface and are taken up by the cell by endocytosis. In some other embodiments, a cationic polymer can be used to transfect producer cells. In some of these embodiments, the cationic polymer is polyethylenimine (PEI). In certain embodiments, chemicals such as calcium phosphate, cyclodextrin, or polybrene, can be used to introduce the one or more moieties to the producer cells. The one or more moieties can also be introduced into a producer cell using a physical method such as particle-mediated transfection, “gene gun”, biolistics, or particle bombardment technology (Papapetrou et al., Gene Therapy 12: S118-S130 (2005)). A reporter gene such as, for example, beta-galactosidase, chloramphenicol acetyltransferase, luciferase, or green fluorescent protein can be used to assess the transfection efficiency of the producer cell.

In certain embodiments, the one or more moieties are introduced to the producer cell by viral transduction. A number of viruses can be used as gene transfer vehicles, including moloney murine leukemia virus (MMLV), adenovirus, adeno-associated virus (AAV), herpes simplex virus (HSV), lentiviruses, and spumaviruses. The viral mediated gene transfer vehicles comprise vectors based on DNA viruses, such as adenovirus, adeno-associated virus and herpes virus, as well as retroviral based vectors.

In certain embodiments, the one or more moieties are introduced to the producer cell by electroporation. Electroporation creates transient pores in the cell membrane, allowing for the introduction of various molecules into the cell. In some embodiments, DNA and RNA as well as polypeptides and non-polypeptide therapeutic agents can be introduced into the producer cell by electroporation.

In certain embodiments, the one or more moieties introduced to the producer cell by microinjection. In some embodiments, a glass micropipette can be used to inject the one or more moieties into the producer cell at the microscopic level.

In certain embodiments, the one or more moieties are introduced to the producer cell by extrusion.

In certain embodiments, the one or more moieties are introduced to the producer cell by sonication. In some embodiments, the producer cell is exposed to high intensity sound waves, causing transient disruption of the cell membrane allowing loading of the one or more moieties.

In certain embodiments, the one or more moieties are introduced to the producer cell by cell fusion. In some embodiments, the one or more moieties are introduced by electrical cell fusion. In other embodiments, polyethylene glycol (PEG) is used to fuse the producer cells. In further embodiments, sendai virus is used to fuse the producer cells.

In some embodiments, the one or more moieties are introduced to the producer cell by hypotonic lysis. In such embodiments, the producer cell can be exposed to low ionic strength buffer causing them to burst allowing loading of the one or more moieties. In other embodiments, controlled dialysis against a hypotonic solution can be used to swell the producer cell and to create pores in the producer cell membrane. The producer cell is subsequently exposed to conditions that allow resealing of the membrane.

In some embodiments, the one or more moieties are introduced to the producer cell by detergent treatment. In certain embodiments, producer cell is treated with a mild detergent which transiently compromises the producer cell membrane by creating pores allowing loading of the one or more moieties. After producer cells are loaded, the detergent is washed away thereby resealing the membrane.

In some embodiments, the one or more moieties introduced to the producer cell by receptor mediated endocytosis. In certain embodiments, producer cells have a surface receptor which upon binding of the one or more moieties induces internalization of the receptor and the associated moieties.

In some embodiments, the one or more moieties are introduced to the producer cell by filtration. In certain embodiments, the producer cells and the one or more moieties can be forced through a filter of pore size smaller than the producer cell causing transient disruption of the producer cell membrane and allowing the one or more moieties to enter the producer cell.

In some embodiments, the producer cell is subjected to several freeze thaw cycles, resulting in cell membrane disruption allowing loading of the one or more moieties.

VI.B. Methods of Modifying an Exosome

In some embodiments, a method of producing an exosome comprises modifying the isolated exosome by directly introducing one or more moieties into the EVs. In certain embodiments, the one or more moieties comprise an AAV. In some embodiments, the one or more moieties comprise a scaffold protein disclosed herein.

In certain embodiments, the one or more moieties are introduced to the exosome by transfection. In some embodiments, the one or more moieties can be introduced into the EV using synthetic macromolecules such as cationic lipids and polymers (Papapetrou et al., Gene Therapy 12: S118-S130 (2005)). In certain embodiments, chemicals such as calcium phosphate, cyclodextrin, or polybrene, can be used to introduce the one or more moieties to the EV.

In certain embodiments, the one or more moieties are introduced to the EV by electroporation. In some embodiments, exosomes are exposed to an electrical field which causes transient holes in the EV membrane, allowing loading of the one or more moieties.

In certain embodiments, the one or more moieties are introduced to the EV by microinjection. In some embodiments, a glass micropipette can be used to inject the one or more moieties directly into the EV at the microscopic level.

In certain embodiments, the one or more moieties are introduced to the EV by extrusion.

In certain embodiments, the one or more moieties are introduced to the EV by sonication. In some embodiments, EVs are exposed to high intensity sound waves, causing transient disruption of the EV membrane allowing loading of the one or more moieties.

In some embodiments, one or more moieties can be conjugated to the surface of the EV. Conjugation can be achieved chemically or enzymatically, by methods known in the art.

In some embodiments, the EV comprises one or more moieties that are chemically conjugated. Chemical conjugation can be accomplished by covalent bonding of the one or more moieties to another molecule, with or without use of a linker. The formation of such conjugates is within the skill of artisans and various techniques are known for accomplishing the conjugation, with the choice of the particular technique being guided by the materials to be conjugated. In certain embodiments, polypeptides are conjugated to the EV. In some embodiments, non-polypeptides, such as lipids, carbohydrates, nucleic acids, and small molecules, are conjugated to the EV.

In some embodiments, the one or more moieties are introduced to the EV by hypotonic lysis. In such embodiments, the EVs can be exposed to low ionic strength buffer causing them to burst allowing loading of the one or more moieties. In other embodiments, controlled dialysis against a hypotonic solution can be used to swell the EV and to create pores in the EV membrane. The EV is subsequently exposed to conditions that allow resealing of the membrane.

In some embodiments, the one or more moieties are introduced to the EV by detergent treatment. In certain embodiments, extracellular vesicles are treated with a mild detergent which transiently compromises the EV membrane by creating pores allowing loading of the one or more moieties. After EVs are loaded, the detergent is washed away thereby resealing the membrane.

In some embodiments, the one or more moieties are introduced to the EV by receptor mediated endocytosis. In certain embodiments, EVs have a surface receptor which upon binding of the one or more moieties induces internalization of the receptor and the associated moieties.

In some embodiments, the one or more moieties are introduced to the EV by mechanical firing. In certain embodiments, extracellular vesicles can be bombarded with one or more moieties attached to a heavy or charged particle such as gold microcarriers. In some of these embodiments, the particle can be mechanically or electrically accelerated such that it traverses the EV membrane.

In some embodiments, extracellular vesicles are subjected to several freeze thaw cycles, resulting in EV membrane disruption allowing loading of the one or more moieties.

VI.C. Methods of Isolating an EV, e.g., Exosome

In some embodiments, methods of producing EVs disclosed herein comprises isolating the EV from the producer cells. In certain embodiments, the EVs released by the producer cell into the cell culture medium. It is contemplated that all known manners of isolation of EVs are deemed suitable for use herein. For example, physical properties of EVs can be employed to separate them from a medium or other source material, including separation on the basis of electrical charge (e.g., electrophoretic separation), size (e.g., filtration, molecular sieving, etc.), density (e.g., regular or gradient centrifugation), Svedberg constant (e.g., sedimentation with or without external force, etc.). Alternatively, or additionally, isolation can be based on one or more biological properties, and include methods that can employ surface markers (e.g., for precipitation, reversible binding to solid phase, FACS separation, specific ligand binding, non-specific ligand binding, affinity purification etc.).

Isolation and enrichment can be done in a general and non-selective manner, typically including serial centrifugation. Alternatively, isolation and enrichment can be done in a more specific and selective manner, such as using EV or producer cell-specific surface markers. For example, specific surface markers can be used in immunoprecipitation, FACS sorting, affinity purification, and magnetic separation with bead-bound ligands.

In some embodiments, size exclusion chromatography can be utilized to isolate the EVs. Size exclusion chromatography techniques are known in the art. Exemplary, non-limiting techniques are provided herein. In some embodiments, a void volume fraction is isolated and comprises the EVs of interest. Further, in some embodiments, the EVs can be further isolated after chromatographic separation by centrifugation techniques (of one or more chromatography fractions), as is generally known in the art. In some embodiments, for example, density gradient centrifugation can be utilized to further isolate the extracellular vesicles. In certain embodiments, it can be desirable to further separate the producer cell-derived EVs from EVs of other origin. For example, the producer cell-derived EVs can be separated from non-producer cell-derived EVs by immunosorbent capture using an antigen antibody specific for the producer cell.

In some embodiments, the isolation of EVs can involve combinations of methods that include, but are not limited to, differential centrifugation, size-based membrane filtration, immunoprecipitation, FACS sorting, and magnetic separation.

### VII. Methods of Treatment

The present disclosure also provides methods of preventing and/or treating a disease or disorder in a subject in need thereof, comprising administering an EV disclosed herein to the subject. In some embodiments, a disease or disorder that can be treated with the present methods comprises a cancer, a hemophilia, diabetes, a growth factor deficiency, an eye disease, a Pompe disease, Gaucher, a lysosomal storage disorder, mucovicidosis, cystic fibrosis, Duchenne and Becker muscular dystrophy, transthyretin amyloidosis, hemophilia A, hemophilia B, adenosine-deaminase deficiency, Leber's congenital amaurosis, X-linked adrenoleukodystrophy, metachromatic leukodystrophy, OTC deficiency, glycogen storage disease 1A, Criggler-Najjar syndrome, primary hyperoxaluria type 1, acute intermittent porphyria, phenylketonuria, familial hypercholesterolemia, mucopolysaccharidosis type VI, al antitrypsin deficiency, Retts Syndrome, Dravet Syndrome, Angelman Syndrome, DM1 disease, Fragile X disease, Huntingtons Disease, Friedreichs ataxia, CMT disease (also known as Charcot-Marie-Tooth disease, hereditary motor and sensory neuropathy (HMSN), or peroneal muscular atrophy), CMT1X disease, catecholaminergic polymorphic ventricular tachycardia, spinocerebellar ataxia type 3 (SCA3) disease, limb-girdle muscular dystrophy, or a hypercholesterolemia. In some embodiments, the treatment is prophylactic.

In some embodiments, the disease or disorder comprises a cancer. In some embodiments, the cancer is advanced, locally advanced, or metastatic. In some embodiments, the cancer is recurrent. In some embodiments, the cancer is refractory to a prior therapy, e.g., a prior standard of care therapy.

In some embodiments, the disease or disorder is associated with a clotting factor deficiency. In some embodiments, the disease or disorder is a bleeding disease. In some embodiments, the disease or disorder is a hemophilia. In some embodiments, the disease or disorder is hemophilia A. In some embodiments, the disease or disorder is hemophilia B. In some embodiments, the disease or disorder is von Willebrand disease.

In some aspects, the disease or disorder is a neurodegenerative disease. In some aspects, the neurodegenerative disease is selected from Alzheimer's disease, Parkinson's disease, prion disease, motor neuron disease, Huntington's disease, spinocerebellar ataxia, spinal muscular atrophy, and any combination thereof.

In certain aspects, the disease or disorder comprises a muscular dystrophy. In some aspects, the muscular dystrophy is selected from Duchenne type muscular dystrophy (DMD), myotonic muscular dystrophy, facioscapulohumeral muscular dystrophy (FSHD), congenital muscular dystrophy, limb-girdle muscular dystrophy (including, but not limited to, LGMD2B, LGMD2D, LGNMD2L, LGMD2C, LGMD2E and LGMD2A), and any combination thereof. In some aspects, the AAV compri

In some aspects, the disease or disorder is selected from AADC deficiency (CNS), ADA-SCID, Alpha-1 antitrypsin deficiency, β-thalassemia (severe sickle cell), Cancer (head and neck squamous cell), Niemman-Pick Type C Disease, Cerebral ALD, Choroideremia, Congestive heart failure, Cystic Fibrosis, Duchenne muscular dystrophy (DMD), Fabry disease, Glaucoma, Glioma (cancer), Hemophilia A, Hemophilia B, HoFH (hypercholesterolemia), Huntington's Disease, Lipoprotein lipase deficiency, Leber hereditary optic neuropathy (LHON), Metachromatic leukodystrophy, MPS I (Hurler syndrome), MPS II (Hunter's syndrome), MPS III (Sanfilippo Syndrome), Parkinson's disease, Pompe Disease, Recessive Dystrophic Epidermolysis Bullosa, RPE65 deficiency (vision loss), Spinal Muscular Atrophy (SMA I), Wet AMD (retinal disease), Wiskott Aldrich syndrome (WAS), Mucopolysaccharidosis type IIIA (MPS IIIA), X-linked myotubular myopathy, X-linked retinitis pigmentosa, and any combination thereof.

In some aspects, the disease or disorder is selected from nephropathy, diabetes insipidus, diabetes type I, diabetes II, renal disease glomerulonephritis, bacterial or viral glomerulonephritides, IgA nephropathy, Henoch-Schonlein Purpura, membranoproliferative glomerulonephritis, membranous nephropathy, Sjogren's syndrome, nephrotic syndrome minimal change disease, focal glomerulosclerosis and related disorders, acute renal failure, acute tubulointerstitial nephritis, pyelonephritis, GU tract inflammatory disease, Pre-clampsia, renal graft rejection, leprosy, reflux nephropathy, nephrolithiasis, genetic renal disease, medullary cystic, medullar sponge, polycystic kidney disease, autosomal dominant polycystic kidney disease, autosomal recessive polycystic kidney disease, tuberous sclerosis, von Hippel-Lindau disease, familial thin-glomerular basement membrane disease, collagen III glomerulopathy, fibronectin glomerulopathy, Alport's syndrome, Fabry's disease, Nail-Patella Syndrome, congenital urologic anomalies, monoclonal gammopathies, multiple myeloma, amyloidosis and related disorders, febrile illness, familial Mediterranean fever, HIV infection-AIDS, inflammatory disease, systemic vasculitides, polyarteritis nodosa, Wegener's granulomatosis, polyarteritis, necrotizing and crecentic glomerulonephritis, polymyositis-dermatomyositis, pancreatitis, rheumatoid arthritis, systemic lupus erythematosus, gout, blood disorders, sickle cell disease, thrombotic thrombocytopenia purpura, Fanconi's syndrome, transplantation, acute kidney injury, irritable bowel syndrome, hemolytic-uremic syndrome, acute corticol necrosis, renal thromboembolism, trauma and surgery, extensive injury, burns, abdominal and vascular surgery, induction of anesthesia, side effect of use of drugs or drug abuse, circulatory disease myocardial infarction, cardiac failure, peripheral vascular disease, hypertension, coronary heart disease, non-atherosclerotic cardiovascular disease, atherosclerotic cardiovascular disease, skin disease, psoriasis, systemic sclerosis, respiratory disease, COPD, obstructive sleep apnoea, hypoia at high altitude or erdocrine disease, acromegaly, diabetes mellitus, and diabetes insipidus, or any combination thereof.

In some aspects, the disease or condition comprises a cancer, e.g., a cancer selected from cancers of the lung, ovarian, cervical, endometrial, breast, brain, colon, prostate, gastrointestinal cancer, head and neck cancer, non-small cell lung cancer, cancer of the nervous system, kidney cancer, retina cancer, skin cancer, liver cancer, pancreatic cancer, genital-urinary cancer and bladder cancer, melanoma, leukemia, brain cancer (e.g., glioma, astrocytomas, ependymomas, oligodendrogliomas, and tumors with mixtures of two or more cell types, called mixed gliomas, Acoustic Neuroma (Neurilemmoma, Schwannoma. Neurinoma), Adenoma, Astracytoma, Low-Grade Astrocytoma, giant cell astrocytomas, Mid- and High-Grade Astrocytoma, Recurrent tumors, Brain Stem Glioma, Chordoma, Choroid Plexus Papilloma, CNS Lymphoma (Primary Malignant Lymphoma), Cysts, Dermoid cysts, Epidermoid cysts, Craniopharyngioma, Ependymoma Anaplastic ependymoma, Gangliocytoma (Ganglioneuroma), Ganglioglioma, Glioblastoma Multiforme (GBM), Malignant Astracytoma, Glioma, Hemangioblastoma, Inoperable Brain Tumors, Lymphoma, Medulloblastoma (MDL), Meningioma, Metastatic Brain Tumors, Mixed Glioma, Neurofibromatosis, Oligodendroglioma. Optic Nerve Glioma, Pineal Region Tumors, Pituitary Adenoma, PNET (Primitive Neuroectodermal Tumor), Spinal Tumors, Subependymoma, and Tuberous Sclerosis (Bourneville's Disease), and any combination thereof.

In some embodiments, the disease or disorder is associated with a growth factor deficiency. In some embodiments, the growth factor is selected from the group consisting of adrenomedullin (AM), angiopoietin (Ang), autocrine motility factor, a bone morphogenetic protein (BMP) (e.g. BMP2, BMP4, BMP5, BMP7), a ciliary neurotrophic factor family member (e.g., ciliary neurotrophic factor (CNTF), leukemia inhibitory factor (LIF), interleukin-6 (IL-6)), a colony-stimulating factor (e.g., macrophage colony-stimulating factor (m-CSF), granulocyte colony-stimulating factor (G-CSF), granulocyte macrophage colony-stimulating factor (GM-CSF)), an epidermal growth factor (EGF), an ephrin (e.g., ephrin A1, ephrin A2, ephrin A3, ephrin A4, ephrin A5, ephrin B1, ephrin B2, ephrin B3), erythropoietin (EPO), a fibroblast growth factor (FGF) (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23), foetal bovine somatotrophin (FBS), a GDNF family member (e.g., glial cell line-derived neurotrophic factor (GDNF), neurturin, persephin, artemin), growth differentiation factor-9 (GDF9), hepatocyte growth factor (HGF), hepatoma-derived growth factor (HDGF), insulin, an insulin-like growth factors (e.g., insulin-like growth factor-1 (IGF-1) or IGF-2, an interleukin (IL) (e.g., IL-1, IL-2, IL-3, IL-4, IL-5, IL-6, IL-7), keratinocyte growth factor (KGF), migration-stimulating factor (MSF), macrophage-stimulating protein (MSP or hepatocyte growth factor-like protein (HGFLP)), myostatin (GDF-8), a neuregulin (e.g., neuregulin 1 (NRG1), NRG2, NRG3, NRG4), a neurotrophin (e.g., brain-derived neurotrophic factor (BDNF), nerve growth factor (NGF), a neurotrophin-3 (NT-3), NT-4, placental growth factor (PGF), platelet-derived growth factor (PDGF), renalase (RNLS), T-cell growth factor (TCGF), thrombopoietin (TPO), a transforming growth factor (e.g., transforming growth factor alpha (TGF-α), TGF-β, tumor necrosis factor-alpha (TNF-α), and vascular endothelial growth factor (VEGF).

In some embodiments, the disease or disorder is diabetes. In some embodiments, the disease or disorder is an eye disease or disorder. In some embodiments, the disease or disorder is Choroideremia (CHM).

In some embodiments, the EVs are administered intravenously to the circulatory system of the subject. In some embodiments, the EVs are infused in a suitable liquid and administered into a vein of the subject.

In some embodiments, the EVs are administered intra-arterialy to the circulatory system of the subject. In some embodiments, the EVs are infused in a suitable liquid and administered into an artery of the subject.

In some embodiments, the EVs are administered to the subject by intrathecal administration. In some embodiments, the EVs are administered via an injection into the spinal canal, or into the subarachnoid space so that it reaches the cerebrospinal fluid (CSF).

In some embodiments, the EVs are administered intratumorally into one or more tumors of the subject.

In some embodiments, the EVs are administered to the subject by intranasal administration. In some embodiments, the EVs can be insufflated through the nose in a form of either topical administration or systemic administration. In certain embodiments, the EVs are administered as nasal spray.

In some embodiments, the EVs are administered to the subject by intraperitoneal administration. In some embodiments, the EVs are infused in suitable liquid and injected into the peritoneum of the subject. In some embodiments, the intraperitoneal administration results in distribution of the EVs to the lymphatics. In some embodiments, the intraperitoneal administration results in distribution of the EVs to the thymus, spleen, and/or bone marrow. In some embodiments, the intraperitoneal administration results in distribution of the EVs to one or more lymph nodes. In some embodiments, the intraperitoneal administration results in distribution of the EVs to one or more of the cervical lymph node, the inguinal lymph node, the mediastinal lymph node, or the sternal lymph node. In some embodiments, the intraperitoneal administration results in distribution of the EVs to the pancreas.

In some embodiments, the EVs, e.g., exosomes, are administered to the subject by periocular administration. In some embodiments, the s are injected into the periocular tissues. Periocular drug administration includes the routes of subconjunctival, anterior sub-Tenon's, posterior sub-Tenon's, and retrobulbar administration.

In some embodiments, the EVs, e.g., exosomes, are administered intraocularly. Accordingly, the present disclosure provides methods of treating an eye disease or disorder in a subject in need thereof comprising administering an effective amount of a composition comprising an extracellular vesicle (EV), e.g., exosome, of the present disclosure which comprises a payload (e.g., an AVV) to the subject, wherein the administration of the composition is intraocular.

In some embodiments, the intraocular administration is selected from the group consisting of intravitreal administration, intracameral administration, subconjunctival administration, subretinal administration, sub scleral administration, intrachoroidal administration, and any combination thereof. In some embodiments, the intraocular administration comprises the injection of the EVs, e.g., exosomes, of the present disclosure. In some embodiments, the intraocular administration is intravitreal injection.

In some embodiments, the intraocular administration comprises the implantation of a delivery device comprising the EVs, e.g., exosomes, of the present disclosure. In some embodiments, the delivery device is an intraocular delivery device. In some embodiments, the intraocular delivery device is biodegradable. In some embodiments, the intraocular delivery device is an intravitreal implant or a scleral plug. In some embodiments, the delivery device is a sustained release delivery device.

In some embodiments, the composition comprising an EV, e.g., exosome, of the present disclosure is pre-treated with intravenous immunoglobulin (IVIg) prior to intraocular administration.

In some embodiments, the eye disease or disorder is selected from the group consisting of macular degeneration, cataract, diabetic retinopathy, glaucoma, amblyopia, strabismus, retinopathy, or any combination thereof. In some embodiments, the eye disease or disorder is, e.g., age-related macular degeneration (AMD), choroidal neovascularization (CNV), retinal detachment, diabetic retinopathy, retinal pigment epithelium atrophy, retinal pigment epithelium hypertrophy, retinal vein occlusion (RVO) disease, infection, intraocular tumor, ocular trauma, dry eye, conjunctivitis, neovascular glaucoma, retinopathy of prematurity (ROP), choroidal retinal vein occlusion, macular edema, anterior neovascularization, corneal neovascularization, subretinal edema, cystoid macular edema, macular hole, vascular striae, pigmented retinitis, Stuttgart disease, inflammatory eye conditions, refractory eye abnormalities, keratoconus, laser induced AMD, optical neuropathy, or senile cataract.

In some embodiments, the eye disease or disorder is an eye cancer. In some embodiments, the eye cancer is a secondary eye cancer (e.g., due to breast cancer or lung cancer metastasis). In some embodiments, the eye cancer is retinoblastoma, intraocular melanoma (e.g., uveal melanoma of the iris, choroid, or ciliary body, or conjunctival melanoma), non-Hodgkin primary intraocular lymphoma, medulloepithelioma, choroidal hemangioma, choroidal metastasis, choroidal nevus, choroidal osteoma, conjunctival Kaposi's sarcoma, epibulbar dermoid, pingueculum, pterygium, squamous carcinoma, or intraepithelial neoplasia of the conjunctiva.

In some embodiments, AMD is any stage of retinal disease, including but not limited to Category 2 (early stage), Category 3 (intermediate stage) and Category 4 (advanced stage) AMD.

In one embodiment, AMD is generally categorized into two types: a dry form and a wet form. The term “dry form” refers to one type of AMD, where alteration of the retina is accompanied by the formation of a small yellow deposit (drusen) under the macula. In some embodiments, dry form AMD is often accompanied by choroidal capillary atrophy, fibrosis, Bruch's thickening, and macular atrophy due to atrophy of the retinal pigment epithelium.

The term “wet form” refers to AMD with abnormal blood vessels that develop under the retina around the macula. Abnormal blood vessels, when broken and bleeding, can damage the macula and dislodge the macula from its base. Symptoms of wet form AMD include Bruch's membrane destruction, glass membrane, choroidal neovascularization (CNV), vascular invasion into the subretinal choroid, followed by serous or hemorrhagic circles This includes, but is not limited to, macular retinal pigment subepithelial or subepithelial vascular invasion, which causes plate-like detachment and eventually becomes a disc-like scar. According to clinical findings, the atrophic type can also change to a wet type.

In some embodiments, wet AMD is also referred to as choroidal neovascularization (“CNV”). CNV (or wet form) can be further classified into “classic” CNV and “occult” CNV. Classic CNV is generally characterized by a bright, highly fluorescent, well-defined region spanning the angiographic transition phase with leakage in the middle and late phase frames. The occult CNV includes fibrovascular pigment epithelial detachment. Neovascularization resulting from CNV has a tendency to leak blood and body fluids, causing stigma and symptoms of metamorphosis. This new blood vessel is accompanied by the growth of fibrous tissue. This complex of neovascular and fibrous tissue can destroy photoreceptors. This lesion can continue to grow across the macula and cause progressive, severe and irreversible blindness. When one individual's eye develops CNV, similar CNV lesions occur in the other eye with a probability of approximately 50% within 5 years.

In some embodiments, a CNV lesion of the present disclosure comprises an occult CNV. In one embodiment, the CNV lesion comprises, consists essentially of, or further consists of classic CNV. In another embodiment, the CNV lesion includes both classic and occult CNV.

The term “macular edema” refers to the ocular diseases cystoid macular edema (CME) or diabetic macular edema (DME). CME is an ocular disease which affects the central retina or macula of the eye. When this condition is present, multiple cyst-like (cystoid) areas a fluid appear in the macula and cause retinal swelling or edema. CME can accompany a variety of diseases such as retinal vein occlusion, uveitis, and/or diabetes. CME commonly Occurs after cataract surgery. DME occurs when blood vessels in the retina of patients with diabetes begin to leak into the macula. These leaks cause the macula to thicken and swell, progressively distorting acute vision. While the swelling may not lead to blindness, the effect can cause a severe loss in central vision.

The term “glaucoma” refers to an ocular disease in which the optic nerve is damaged in a characteristic pattern. This can permanently damage vision in the affected eye and lead to blindness if left untreated. It is normally associated with increased fluid pressure in the eye (aqueous humor). The term ocular hypertension is used for patients with consistently raised intraocular pressure (IOP) without any associated optic nerve damage. Conversely, the term normal tension or low tension glaucoma is used for those with optic nerve damage and associated visual field loss but normal or low IOP. The nerve damage involves loss of retinal ganglion cells in a characteristic pattern. There are many different subtypes of glaucoma, but they can all be considered to be a type of optic neuropathy. Raised intraocular pressure (e.g., above 21 mmHg or 2.8 kPa) is the most important and only modifiable risk factor for glaucoma. However, some can have high eye pressure for years and never develop damage, while others can develop nerve damage at a relatively low pressure. Untreated glaucoma can lead to permanent damage of the optic nerve and resultant visual field loss, which over time can progress to blindness.

The term “diabetic retinopathy” includes retinopathy (i.e., a disease of the retina) caused by complications of diabetes, which can eventually lead to blindness. Diabetic retinopathy can cause no symptoms, mild vision problems, or even blindness. Diabetic retinopathy is the result of microvascular retinal changes. Hyperglycemia-induced intramural pericyte death and thickening of the basement membrane lead to incompetence of the vascular walls. These damages change the formation of the blood-retinal barrier and also make the retinal blood vessels become more permeable.

In some embodiments, the present disclosure provides a pharmaceutical composition comprising an EV, e.g., exosome, of the present disclosure formulated for intraocular administration. The present disclosure also provides a kit comprising a pharmaceutical composition comprising an EV, e.g., exosome, of the present disclosure formulated for intraocular administration, and optionally instructions for use according to the methods disclosed herein, e.g., instructions to administer the pharmaceutical composition to treat a specific eye disease or disorder.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986);); Crooke, Antisense drug Technology: Principles, Strategies and Applications, 2^{nd} Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following examples are offered by way of illustration and not by way of limitation.

### EXAMPLES

### Example 1

### Luminal Loading Using a Scaffold Protein

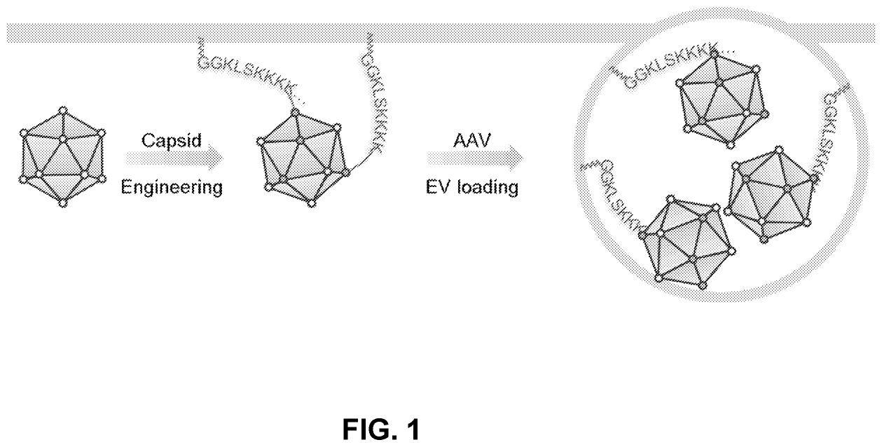

A modified AAV was produced, wherein an AAV capsid protein (e.g., VP1, VP2, or VP3) as fused to the intracellular domain of a scaffold protein (e.g., PTGFRN) or to the C-terminus of a scaffold protein comprising the minimal sequence GGKLSKK (SEQ ID NO: 17) (FIG. 1). The scaffold protein (e.g., PTGFRN) or to the scaffold protein comprising the minimal sequence GGKLSKK (SEQ ID NO: 17) was fused to either the N-terminus, C-terminus, or internal site of the capsid protein. The AAV is produced in cells co-producing exosomes, facilitating localization of the AAV to the exosome.

### Example 2

### Luminal Loading Using a Binding Partner of a Chemically Induced Dimer

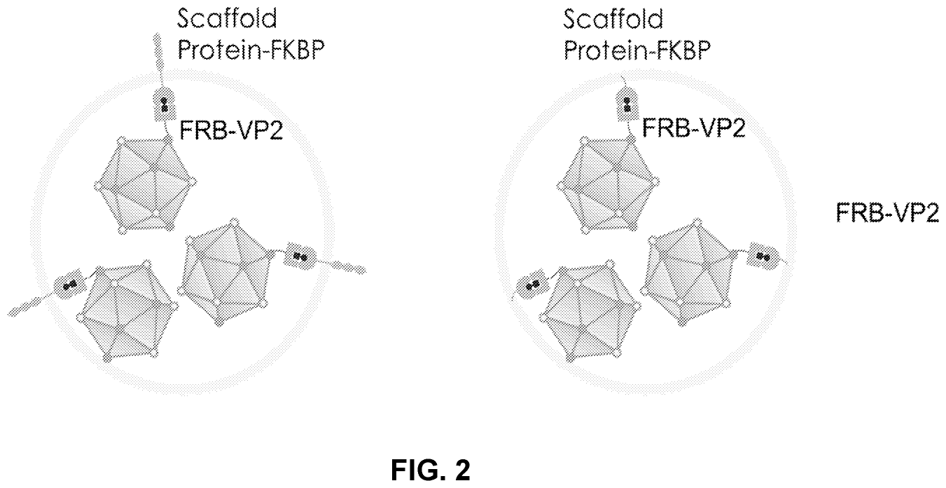

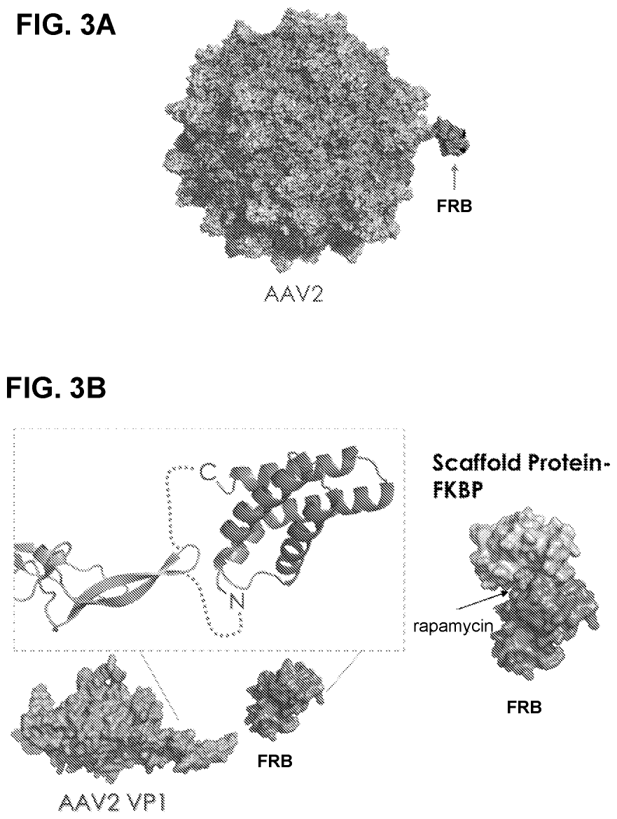

A modified AAV is produced, wherein an AAV capsid protein (e.g., VP1, VP2, or VP3) is fused to a binding partner of a chemically induced dimer (e.g., FRB) (FIG. 2). The binding partner is fused to either the N-terminus, C-terminus, or internal site of the capsid protein (FIG. 2) or inserted within an internal loop (e.g., VP1) at position 455 (FIGS. 3A-3B). The insertion is made such that the FRB replaces amino acid residue T455 (Relative to SEQ ID NO: 44) of VP1. The corresponding binding partner (e.g., FKBP) is then linked to the C-terminus of either PTGFRN or a scaffold protein comprising the minimal sequence GGKLSKK (SEQ ID NO: 17). The AAV is produced in cells co-producing exosomes in the presence of the necessary chemical to induce dimerization (e.g., in the presence of rapamycin to induce dimerization of the FRB and FKBP), facilitating localization of the AAV to the exosome.

A modified AAV is produced, wherein an AAV capsid protein (e.g., VP1, VP2, or VP3) is fused to a binding partner of a chemically induced dimer (e.g., FKBP, CalcineurinA, CyP-Fas, GyrB, GAI, GID1, Snap-tag, HaloTag, eDHFR, BCL-xL, or Fab (AZ1)). The binding partner is fused to either the N-terminus of the capsid protein or inserted within an internal loop (e.g., VP1 at position 455). The insertion is made such that the chemically induced binding partner (e.g., (e.g., FKBP, CalcineurinA, CyP-Fas, GyrB, GAI, GID1, Snap-tag, HaloTag, eDHFR, BCL-xL, or Fab (AZ1))) replaces amino acid residues T455 (Relative to SEQ ID NO: 45) of VP1. The corresponding binding partner (e.g., (e.g., FKBP, CalcineurinA, CyP-Fas, GyrB, GAI, GID1, Snap-tag, HaloTag, eDHFR, BCL-xL, or Fab (AZ1))) is then linked to the C-terminus of either PTGFRN (or a functional fragment thereof) or a scaffold protein comprising the minimal sequence GGKLSKK (SEQ ID NO: 17). The AAV is produced in cells co-producing exosomes in the presence of the necessary chemical to induce dimerization (e.g., in the presence of FK1012 to induce dimerization of the FKBP and FKBP; in the presence of FK506 to induce dimerization of the FKBP and CalcineurinA; in the presence of FKCsA to induce dimerization of the FKBP and CyP-Fas; in the presence of Coumermycin to induce dimerization of the GyrB and GyrB); in the presence of Gibberellin to induce dimerization of the GAI and GID1); in the presence of HaXS to induce dimerization of the Snap-tag and HaloTag); in the presence of TMP-HTag to induce dimerization of the eDHFR and HaloTag); in the presence of ABT-737 to induce dimerization of the BCL-xL and Fab (AZ1))), facilitating localization of the AAV to the exosome.

A modified AAV is produced, wherein an AAV capsid protein (e.g., VP1, VP2, or VP3) is fused to a binding partner of a chemically induced dimer (e.g., a first FKBP). The binding partner is fused to either the N-terminus of the capsid protein or inserted within an internal loop (e.g., VP1 at position 455). The insertion is made such that the chemically induced binding partner (e.g., the first FKBP) replaces amino acid residues T455 (Relative to SEQ ID NO: 45) of VP1. The corresponding binding partner (e.g., a second FKBP) is then linked to the C-terminus of either PTGFRN (or a functional fragment thereof) or a scaffold protein comprising the minimal sequence GGKLSKK (SEQ ID NO: 17). The AAV is produced in cells co-producing exosomes in the presence of the necessary chemical to induce dimerization (e.g., in the presence of FK1012 to induce dimerization of the first FKBP and the second FKBP), facilitating localization of the AAV to the exosome.

A modified AAV is produced, wherein an AAV capsid protein (e.g., VP1, VP2, or VP3) is fused to a binding partner of a chemically induced dimer (e.g., FKBP or CalcineurinA). The binding partner is fused to either the N-terminus of the capsid protein or inserted within an internal loop (e.g., VP1 at position 455). The insertion is made such that the chemically induced binding partner (e.g., FKBP or CalcineurinA) replaces amino acid residues T455 (Relative to SEQ ID NO: 45) of VP1. The corresponding binding partner (e.g., FKBP or CalcineurinA) is then linked to the C-terminus of either PTGFRN (or a functional fragment thereof) or a scaffold protein comprising the minimal sequence GGKLSKK (SEQ ID NO: 17). The AAV is produced in cells co-producing exosomes in the presence of the necessary chemical to induce dimerization (e.g., in the presence of FK506 to induce dimerization of the FKBP and CalcineurinA), facilitating localization of the AAV to the exosome.

A modified AAV is produced, wherein an AAV capsid protein (e.g., VP1, VP2, or VP3) is fused to a binding partner of a chemically induced dimer (e.g., FKBP or CyP-Fas). The binding partner is fused to either the N-terminus of the capsid protein or inserted within an internal loop (e.g., VP1 at position 455). The insertion is made such that the chemically induced binding partner (e.g., FKBP or CyP-Fas) replaces amino acid residues T455 (Relative to SEQ ID NO: 45) of VP1. The corresponding binding partner (e.g., FKBP or CyP-Fas) is then linked to the C-terminus of either PTGFRN (or a functional fragment thereof) or a scaffold protein comprising the minimal sequence GGKLSKK (SEQ ID NO: 17). The AAV is produced in cells co-producing exosomes in the presence of the necessary chemical to induce dimerization (e.g., in the presence of FKCsA to induce dimerization of the FKBP and CyP-Fas), facilitating localization of the AAV to the exosome.

A modified AAV is produced, wherein an AAV capsid protein (e.g., VP1, VP2, or VP3) is fused to a binding partner of a chemically induced dimer (e.g., GyrB). The binding partner is fused to either the N-terminus of the capsid protein or inserted within an internal loop (e.g., VP1 at position 455). The insertion is made such that the chemically induced binding partner (e.g., GyrB) replaces amino acid residues T₄₅₅ (Relative to SEQ ID NO: 45 of VP1). The corresponding binding partner (e.g., GyrB) is then linked to the C-terminus of either PTGFRN (or a functional fragment thereof) or a scaffold protein comprising the minimal sequence GGKLSKK (SEQ ID NO: 17). The AAV is produced in cells co-producing exosomes in the presence of the necessary chemical to induce dimerization (e.g., in the presence of Coumermycin to induce dimerization of the GyrB and GyrB), facilitating localization of the AAV to the exosome.

A modified AAV is produced, wherein an AAV capsid protein (e.g., VP1, VP2, or VP3) is fused to a binding partner of a chemically induced dimer (e.g., GAI or GID1). The binding partner is fused to either the N-terminus of the capsid protein or inserted within an internal loop (e.g., VP1 at position 455). The insertion is made such that the chemically induced binding partner (e.g., GAI or GID1) replaces amino acid residues T₄₅₅ (Relative to SEQ ID NO: 45 of VP1). The corresponding binding partner (e.g., GAI or GID1) is then linked to the C-terminus of either PTGFRN (or a functional fragment thereof) or a scaffold protein comprising the minimal sequence GGKLSKK (SEQ ID NO: 17). The AAV is produced in cells co-producing exosomes in the presence of the necessary chemical to induce dimerization (e.g., in the presence of Gibberellin to induce dimerization of the GAI and GID1), facilitating localization of the AAV to the exosome.

A modified AAV is produced, wherein an AAV capsid protein (e.g., VP1, VP2, or VP3) is fused to a binding partner of a chemically induced dimer (e.g., Snap-tag or HaloTag). The binding partner is fused to either the N-terminus of the capsid protein or inserted within an internal loop (e.g., VP1 at position 455). The insertion is made such that the chemically induced binding partner (e.g., Snap-tag or HaloTag) replaces amino acid residues T455 (Relative to SEQ ID NO: 45 of VP1). The corresponding binding partner (e.g., Snap-tag or HaloTag) is then linked to the C-terminus of either PTGFRN (or a functional fragment thereof) or a scaffold protein comprising the minimal sequence GGKLSKK (SEQ ID NO: 17). The AAV is produced in cells co-producing exosomes in the presence of the necessary chemical to induce dimerization (e.g., in the presence of HaXS to induce dimerization of the Snap-tag and HaloTag), facilitating localization of the AAV to the exosome.

A modified AAV is produced, wherein an AAV capsid protein (e.g., VP1, VP2, or VP3) is fused to a binding partner of a chemically induced dimer (e.g., eDHFR or HaloTag). The binding partner is fused to either the N-terminus of the capsid protein or inserted within an internal loop (e.g., VP1 at position 455). The insertion is made such that the chemically induced binding partner (e.g., eDHFR or HaloTag) replaces amino acid residues T₄₅₅ (Relative to SEQ ID NO: 45 of VP1). The corresponding binding partner (e.g., eDHFR or HaloTag) is then linked to the C-terminus of either PTGFRN (or a functional fragment thereof) or a scaffold protein comprising the minimal sequence GGKLSKK (SEQ ID NO: 17). The AAV is produced in cells co-producing exosomes in the presence of the necessary chemical to induce dimerization (e.g., in the presence of TMP-HTag to induce dimerization of the eDHFR and HaloTag), facilitating localization of the AAV to the exosome.

A modified AAV is produced, wherein an AAV capsid protein (e.g., VP1, VP2, or VP3) is fused to a binding partner of a chemically induced dimer (e.g., BCL-xL or Fab (AZ1)). The binding partner is fused to either the N-terminus of the capsid protein or inserted within an internal loop (e.g., VP1 at position 455). The insertion is made such that the chemically induced binding partner (e.g., BCL-xL or Fab (AZ1)) replaces amino acid residues T₄₅₅ (Relative to SEQ ID NO: 45) of VP1. The corresponding binding partner (e.g., BCL-xL or Fab (AZ1)) is then linked to the C-terminus of either PTGFRN (or a functional fragment thereof) or a scaffold protein comprising the minimal sequence GGKLSKK (SEQ ID NO: 17). The AAV is produced in cells co-producing exosomes in the presence of the necessary chemical to induce dimerization (e.g., in the presence of ABT-737 to induce dimerization of the BCL-xL and Fab (AZ1)), facilitating localization of the AAV to the exosome.

### Example 3

### Luminal Loading Using AAV Receptors

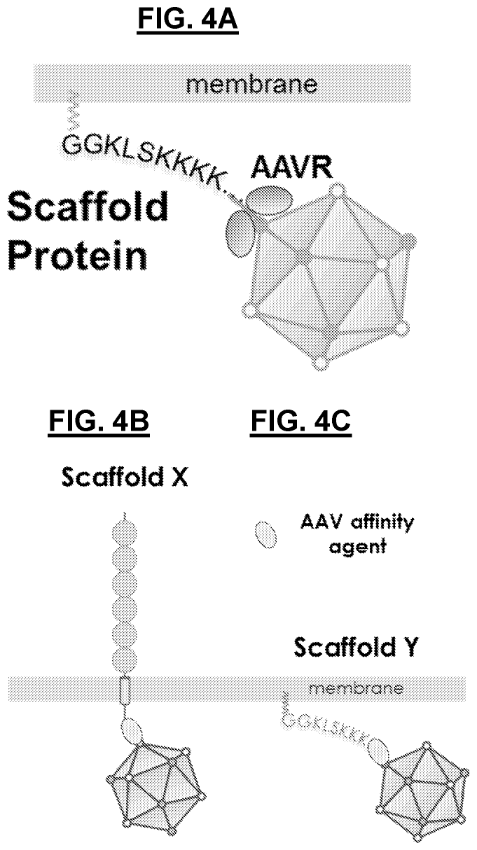

A modified exosome is generated, wherein the exosome comprises a scaffold protein (e.g., PTGFRN) or a scaffold protein comprising the minimal sequence GGKLSKK (SEQ ID NO: 17) linked to an AAV receptor (AAVR). AAVR can include PKD1-2 and single chain antibodies (FIG. 4). For luminal loading, the AAVR is linked to the intracellular domain of the scaffold protein (e.g., PTGFRN) or to the intracellular domain of the scaffold protein comprising the minimal sequence GGKLSKK (SEQ ID NO: 17). The exosome is produced in cells co-producing AAV, such that the AAV receptor facilitates localization of the AAV to the exosome.

### Example 4

### Exterior Surface Loading Using an Antigen Binding Polypeptide

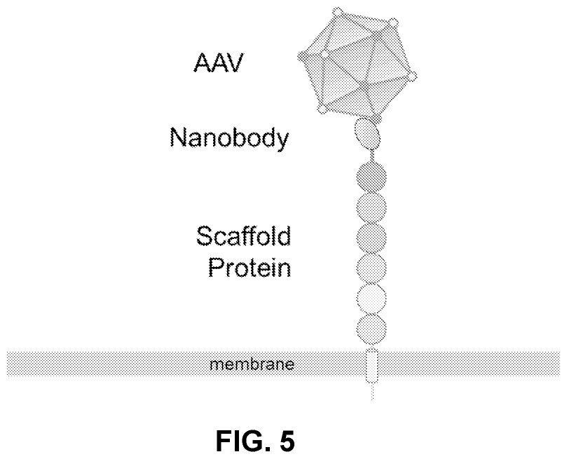

A modified exosome is produced, comprising a scaffold protein (e.g., PTGFRN) linked at an extracellular domain to an antigen binding domain (e.g., a nanobody; FIG. 5). The antigen-binding domain (e.g., nanobody) specifically binds an epitope on the AAV capsid. The exosomes and AAVs are produced and purified separately, from different producing cells. Purified exosomes expressing nanobody-scaffold protein are then incubated with purified AAVs to facilitate localization of the AAV to the exosome. Alternatively, the exosome is produced in cells co-producing AAV, such that the nanobody facilitates localization of the AAV to the exosome.

### Example 5

### Exterior Surface Loading Using Fc

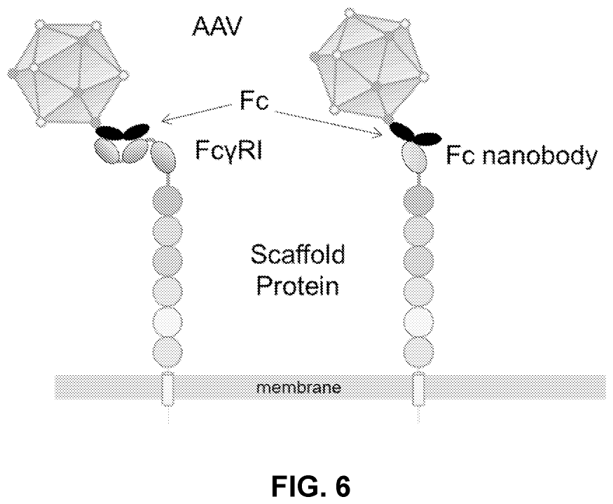

A modified AAV is produced, comprising a capsid protein (e.g., VP1, VP2, or VP3) linked to an Fc region of IgG. A modified exosome is generated comprising a scaffold protein (e.g., PTGFRN) linked to either an FcγR1 or a nanobody that specifically binds Fc (FIG. 6). The FcγR1 or the nanobody is linked to an extracellular domain of the scaffold protein (e.g., PTGFRN). The exosomes and AAVs are produced and purified separately, from different producing cells. Purified exosomes expressing scaffold fusion proteins are then incubated with purified AAVs to facilitate localization of the AAV to the exosome. Alternatively, the exosome is produced in cells co-producing AAV, such that the scaffold protein facilitates localization of the AAV to the exosome.

### Example 6

### Exterior Surface Loading Using AAV Receptor

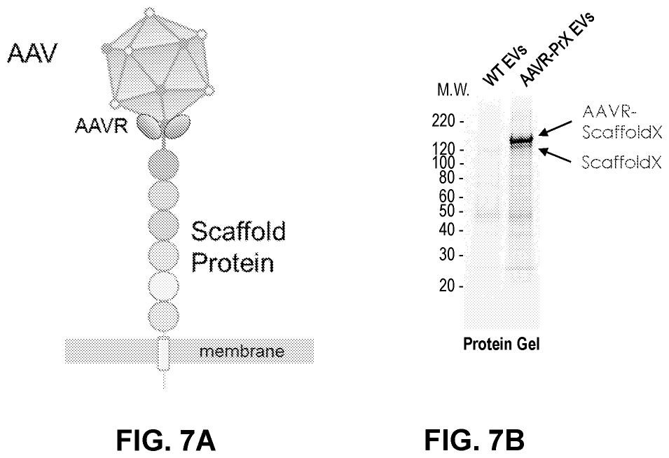

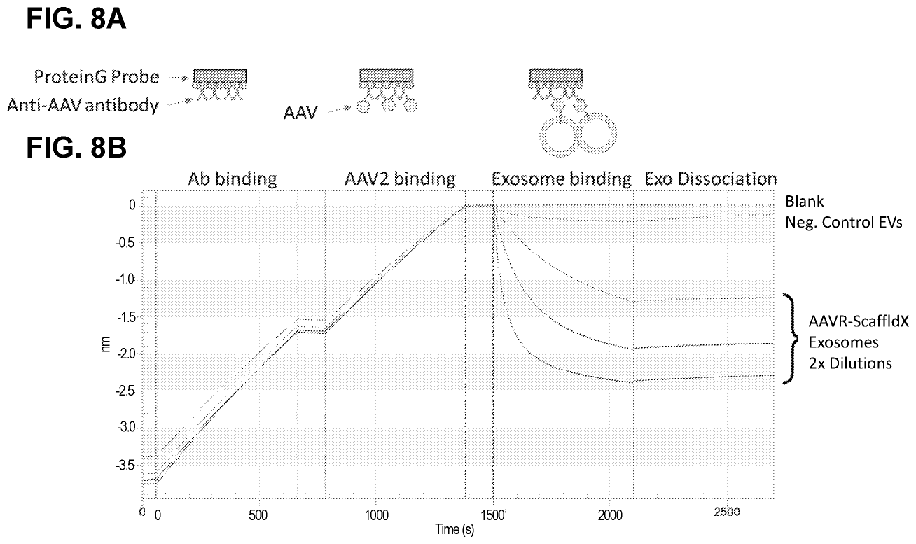

A modified exosome was generated, wherein the exosome comprises a scaffold protein (e.g., PTGFRN) linked to an AAV receptor (AAVR) (FIG. 7A). The AAVR is linked to the extracellular domain of the scaffold protein (e.g., PTGFRN). The exosomes and AAVs were produced and purified separately, from different producing cells. Purified exosomes expressing scaffold fusion proteins (FIG. 7B) were then incubated with purified AAVs to facilitate localization of the AAV to the exosome. Bio-layer interferometry (Octet) data shows that AAVR exosomes bind to immobilized AAV2, while control exosomes do not (FIGS. 8A-8B). Alternatively, the exosome is produced in cells co-producing AAV, such that the scaffold protein facilitates localization of the AAV to the exosome.

### Example 7

### AAV Fusion Constructs Retain Nuclear Localization

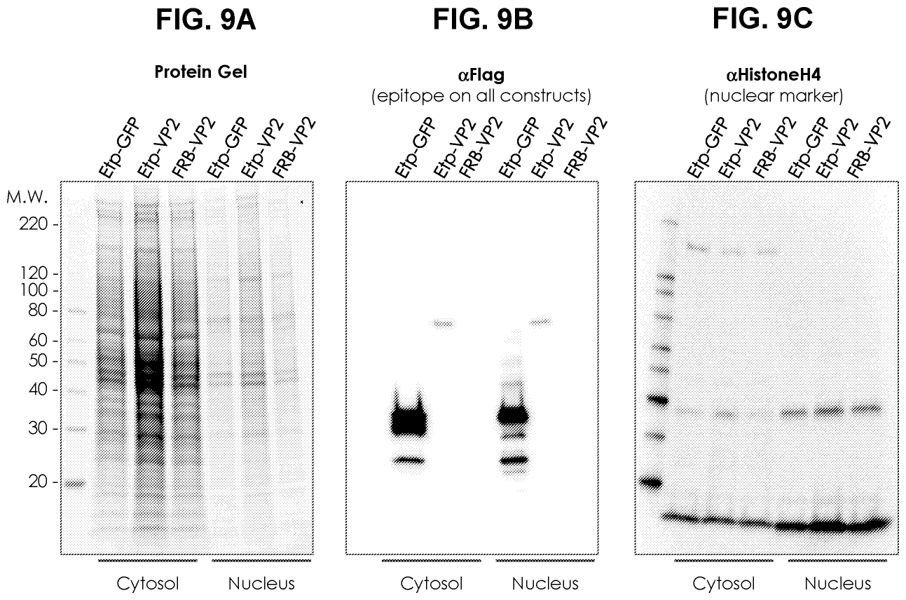

Various techniques described herein rely on fusion of a peptide sequence to a capsid protein of AAV. To assess AAV activity, modified AAV were generated, wherein the N-terminus of an AAV capsid protein VP2 was linked to a scaffold protein comprising the minimal sequence GGKLSKK (SEQ ID NO: 17) (“Etp”) or to the chemically inducible binding partner FRB or FKBP. Etp-GFP is a control with green fluorescent protein (GFP) substituted for VP2 capsid protein. To confirm that the modified AAV retain the ability to localize to the nucleus of producing cells, Western blotting was carried out on a purified cytosolic fraction and a purified nuclear fraction isolated from HEK293 cell lysates. As shown in FIG. 9A, equal amounts of cell lysate from the cytosol (left) and nucleus (right) were loaded on a denaturing polyacrylamide gel. Western blotting for Etp-GFP, Etp-VP2, and FRB-VP2 using antibodies specific for αFLAG tag (expressed on all constructs; FIG. 9B), and αHistone H4 (a nuclear marker; FIG. 9C) in both cytosol and nucleus lysates demonstrated that these exotope-VP2 constructs are expressed and localized to the nucleus of producing cells. Specifically, Etp-VP2 can be seen in the nucleus lysate using the αFLAG antibody probe (FIG. 9C). Because AAV capsid assembly and genome loading occurs in the nucleus, the demonstration that Etp-VP2 is enriched in the nucleus provides an opportunity for generating functional AAV capsids that carry a peptide tag that facilitates exosome loading. Furthermore, AAV carrying the etp-VP2 modification should be able to enter the nucleus of recipient cells to mediate gene transfer, as this modification does not inhibit nuclear entry.

### Example 8

### Expression of Exosome-AAV in Culture

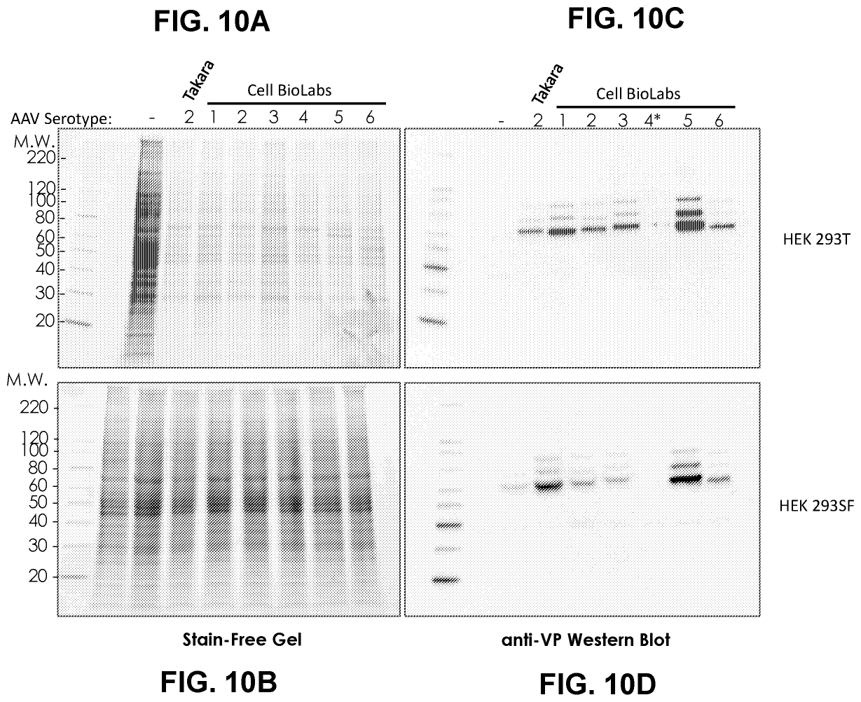

HEK293T cells were seeded and transfected via the triple transfection method to express AAV. This method typically involves a gene transfer vector (comprising a transgene flanked by ITR elements) to be packaged into AAV particles, an AAV helper function vector, and an accessory function vector, which contains sequences for capsid proteins and replication-associated proteins. FIG. 10A-10D shows the results of various AAV capsid serotypes transfected into HEK293T cells and HEK293 cells adapted for suspension culture (HEK293 SF). AAV expression constructs were obtained for AAV expression testing. FIGS. 10A-10D show that AAV1, AAV2, AAV3, AAV5, and AAV6 capsid are detected via Western Blot. The antibody probe used has been reported not or recognize AAV4, which can explain the lack of signal in the AAV4 lanes.

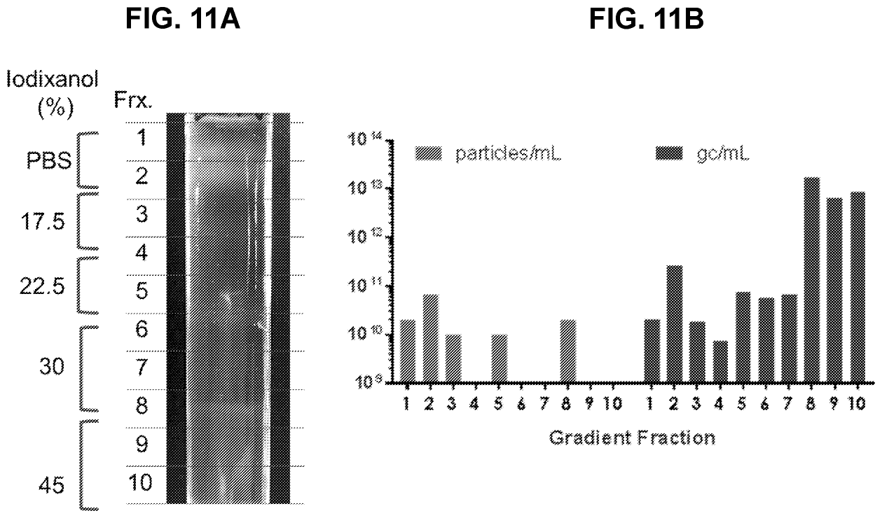

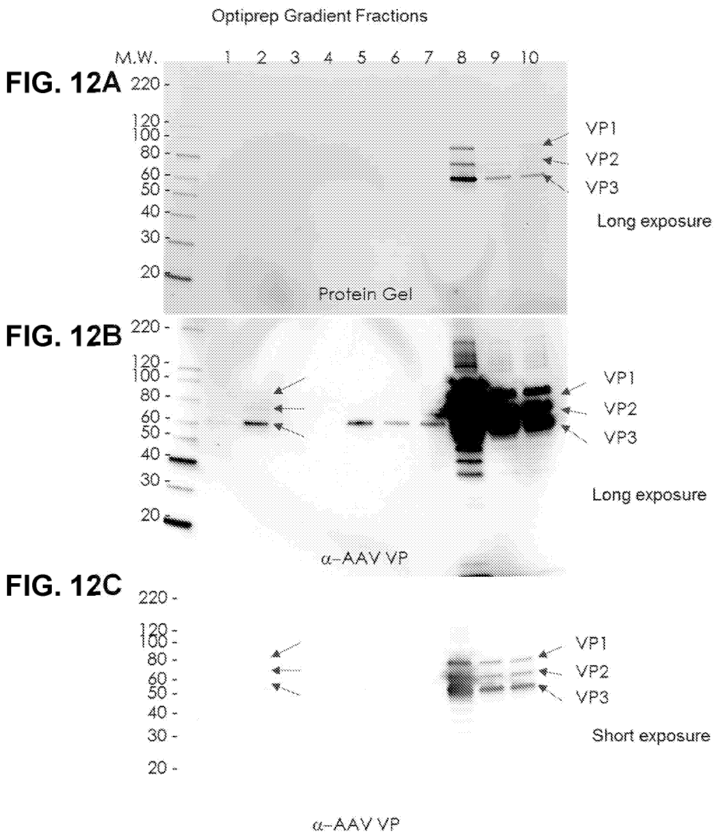

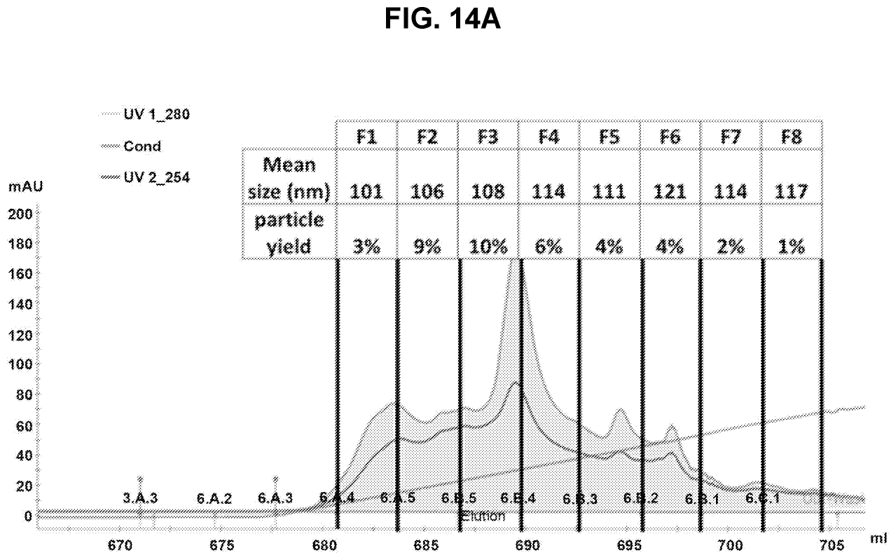

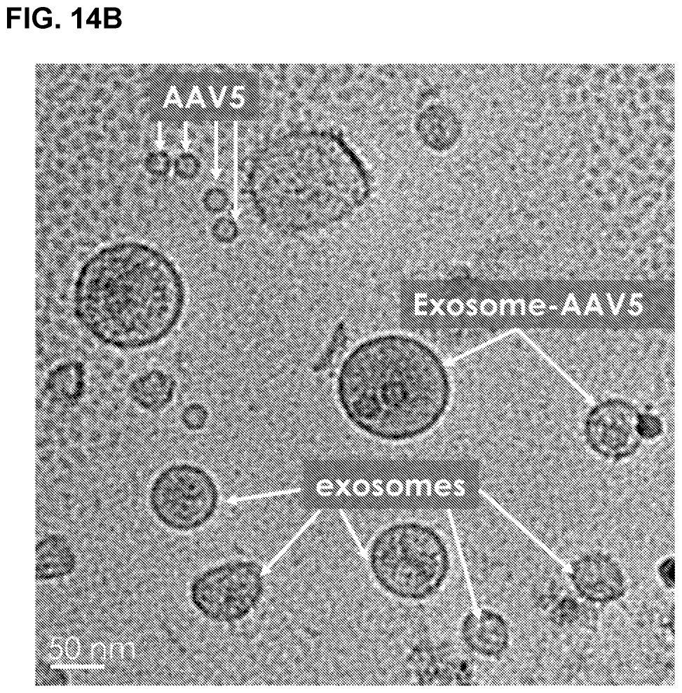

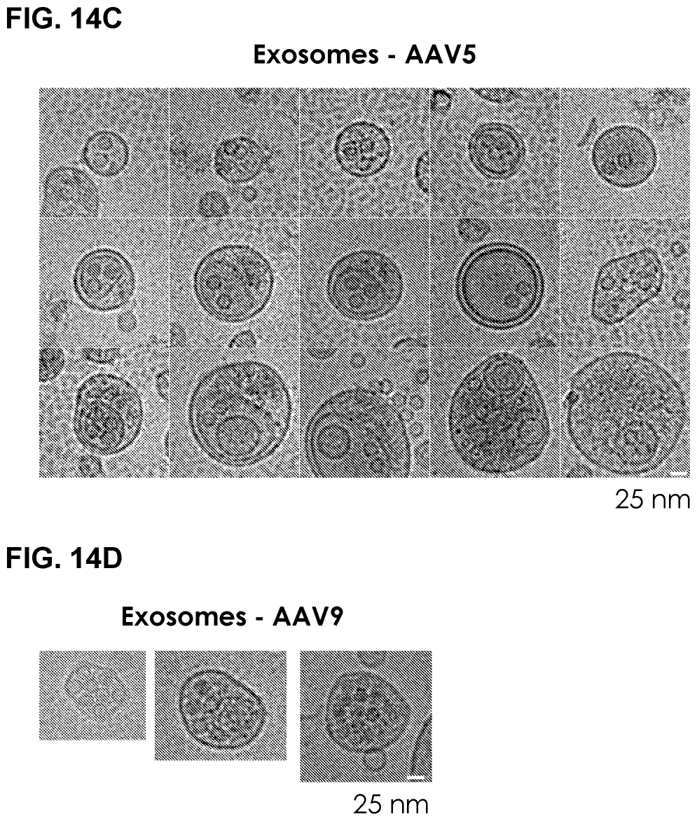

AAV9 was grown in adherent HEK293T cell using the triple transfection method. Harvest was filtered to remove cellular debris and then concentrated and diafiltered into a buffered 150 mM NaCl solution with tangential flow filtration. 50 mL of TFF concentrated (˜10×) cell supernatant was pelleted by ultracentrifugation (133,900×g) for three hours, and the resulting pellet resuspended in 1 mL PBS. Preparations were applied to an Optiprep density gradient (an iodixanol-based medium) employing 150,000×ultracentrifugation for 16 hours. Following separation, fractions 1-10 as seen in FIG. 14A were collected, diluted with PBS 10×, and pelleted via ultracentrifugation at 133,900 g for 3 hours followed by resuspension in a 100 uL volume of PBS. Fractions were analyzed via western blot to detect AAV capsid protein, particle counting (nanoparticle tracking analysis (NTA)) to detect exosomes, and qPCR to detect AAV DNA transgene genome copies. Some fractions did not contain detectable particles (by NTA) analysis. FIGS. 11A-11B details the results of the NTA (particle/mL) and qPCR (gene copies/mL (GC/mL)) results. Most AAV transgene DNA was found in free AAV not associated with exosomes in the denser fractions 8-10. Western blot analysis of the AAV capsid can be seen in FIGS. 12A-12C, showing that VP1, VP2, and VP3 polypeptides can be seen most prominently in fractions 8, 9, and 10, where they are not associated with exosomes. Fractions 1, 2, 3, and 5 also have detectable VP1, VP2, and VP3 and are found to be associated with higher exosome concentration in these fractions.

### Example 9

### Purification of Exosome-AAV

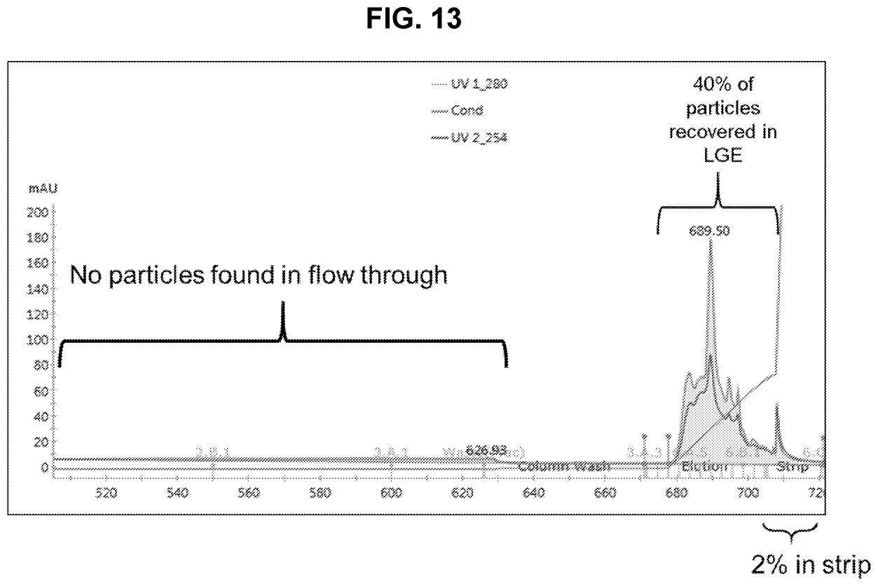

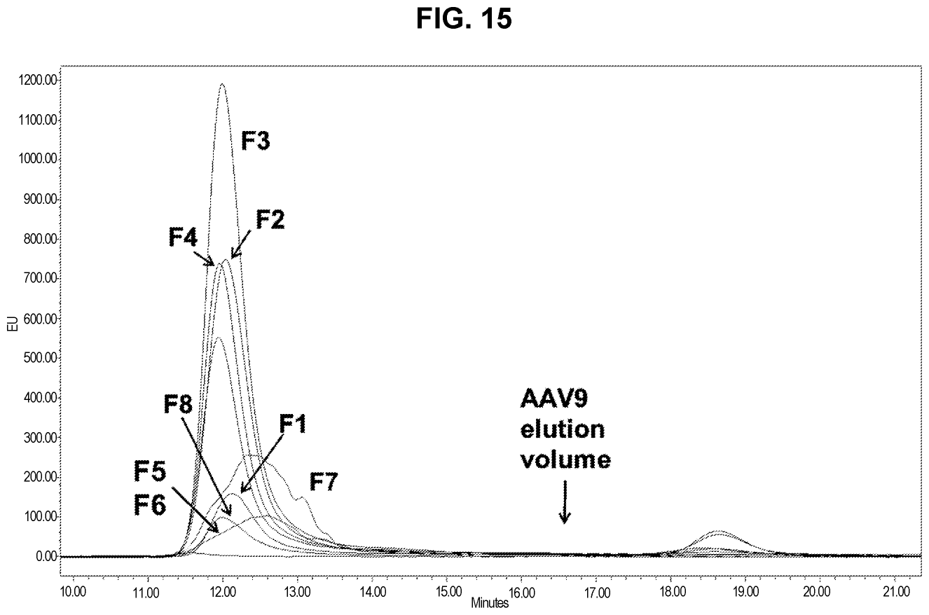

AAV9 was grown in adherent HEK293T cell using the triple transfection method. Harvest was filtered to remove cellular debris and then concentrated and diafiltered into a 150 mM NaCl, pH 7.4 solution (˜10 mS/cm) using tangential flow filtration. The preparation was then purified via bind-and-elute anion-exchange chromatography. A using a linear gradient elution (LGE) with increasing concentrations of NaCl from 150 mM NaCl to 1 M NaCl across 20 column volumes. The column was then stripped with 5 column volumes of 2 M NaCl, pH 7.4 before being cleaned and sanitized with 1 M NaOH. The purification chromatogram is provided in FIG. 13. Fractions were collected across the linear gradient and analyzed with NTA to determine exosome count and particle size, as seen in FIG. 14A. Fractions 2, 3, and 4 show the highest concentrations of particle elution as measured by NTA. FIGS. 14B-14D show example images of exosomes loaded with AAVS (FIGS. 14B-14C) and AAV9 (FIG. 14D). Each fraction was then purified via size-exclusion chromatography (SEC). Fractions 2, 3, and 4 eluted as a single peak near the void volume with little tailing, which is characteristic of particles such as exosomes and neither soluble proteins nor AAV9, which has been shown to elute at volume=16.5 mL. Fractions 2, 3, and 4 also contained the highest concentration of particles via NTA analysis (FIG. 15).

### Example 10

### Determining AAV9 Potency

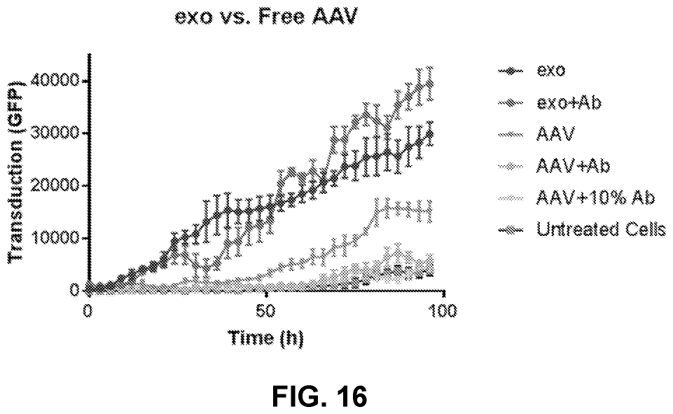

AAV9-GFP and Exosome-AAV9-GFP were isolated from HEK293 adherent cell culture transfected with a standard AAV triple plasmid system using density gradient ultracentrifugation or anion exchange chromatography as described in Example 6. Each sample was quantified in triplicate with qPCR to determine virus genomes per mL (GC/mL). HeLa cells were grown by standard cell culture procedures and seeded into 96-well IncuCyte Zoom plate wells at 5000 cells/well. Cells were then transfected with a fixed GC/well (fixed MOI: 6000) with either free or encapsulated AAV9-GFP in the presence of anti-AAV IgG. The concentration of IgG was systematically varied by dilution in PBS to generate a dose-titration study with half-log spacing on IgG concentrations. Following addition of sample to the wells, GFP expression as measured by fluorescence intensity was determined every three hours for a period of four days. Each condition was run in triplicate. A representative profile of F7, a sample isolated from anion exchange chromatography is described in FIG. 16.

More rapid transduction was observed with the exosome-AAV sample (“exo”) compared to the AAV only sample (“AAV”). The maximum potency achieved was also higher in the exosome-AAV (“exo”) sample, approaching 40,000 fluorescence units compared to the AAV only sample (“AAV”), which produced approximately 15,000 fluorescence units. These potency data demonstrate that exosome-AAV is able to match or exceed the potency and gene delivery capacity of free AAV9 even when delivered via exosome encapsulation.

### Example 11

### Resistance of Exosome-AAV to Neutralization by Antibodies

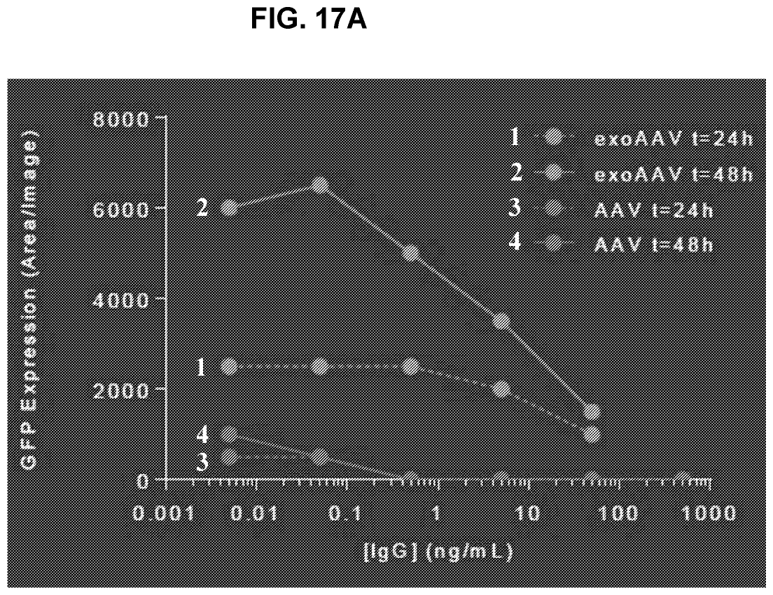

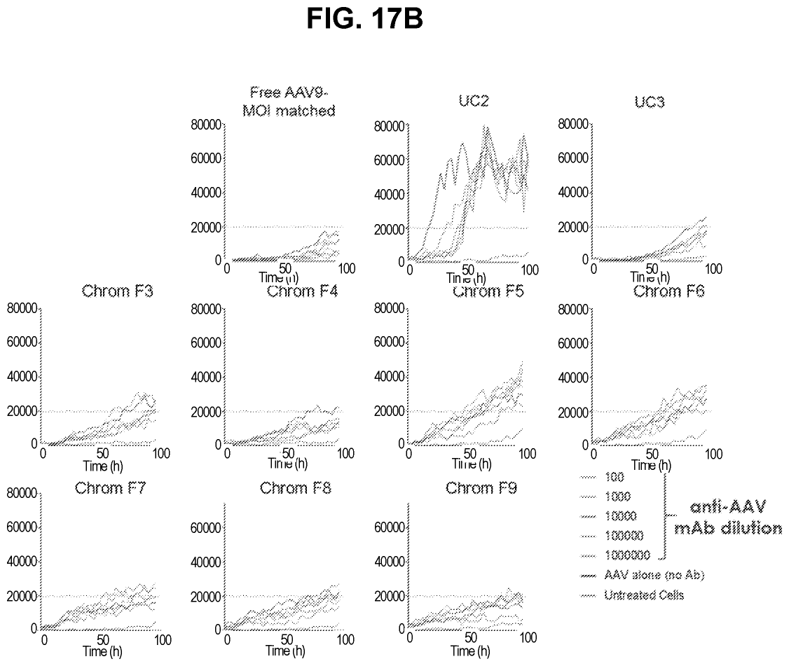

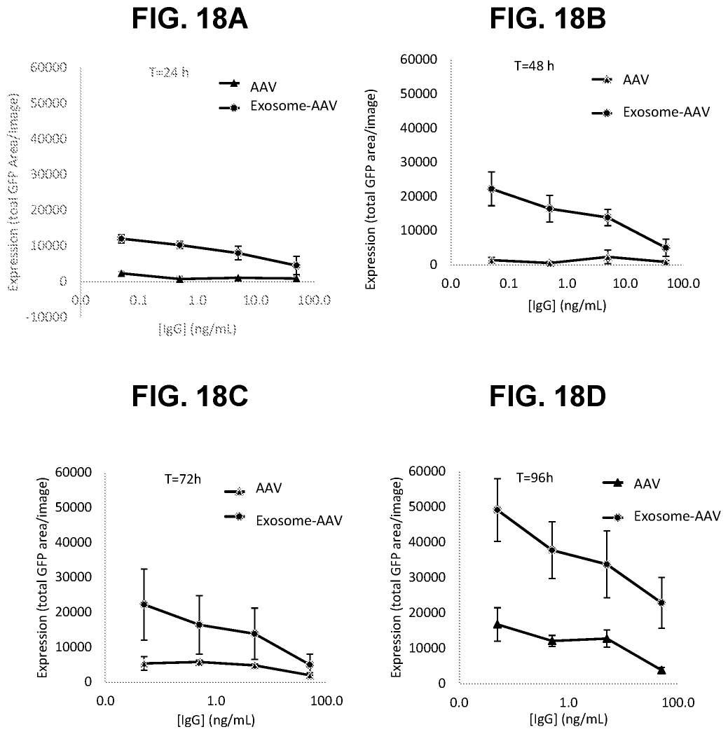

Anti-drug antibodies (ADA) are a common problem with the delivery of biologics. Interventions that require repeated administrations (or even a single administration) can be hindered by a host immune response against the biologic. To determine whether association with exosomes affected resistance to inhibitory antibodies, free AAV9 and exosome-associated AAV9 expressing GFP were incubated with HeLa cells across a range of anti-AAV9 nAb. Exosome-associated AAV9 was significantly superior to free AAV9 at two time points and all nAb concentrations (FIG. 17A). The resistance of exosome-AAV constructs (carrying a GFP transgene) to anti-AAV monoclonal antibodies as compared to free AAV9 was further investigated as described in Example 10 and can be seen in FIG. 17B. A control sample of AAV alone was tested against serial dilutions of an anti-AAV monoclonal antibody (mAb), and the GFP signal was measured. A GFP signal indicates successful AAV infection and delivery of the GFP transgene to the host cells. The results show that free AAV9 shows significant infectivity at a serial dilution of the anti-AAV mAb of 1000-fold or less. When matching for MOI as determined by gene copy qPCR analysis, the exosome-AAV samples were compared to the free AAV9-MOI matched samples. Samples such as Chrom. 3, Chrom. 5, and Chrom. 7 showed significant infectivity even in the presence of the anti-AAV mAb 100-fold dilution. After 100 hours, Chrom. 3, Chrom. 4, and Chrom. 5 all showed GFP signals well above 20,000, indicating a sustained capacity for infection. This signal is significantly larger than the 100-fold dilution signal seen in the free AAV9-MOI matched sample, which indicates that the free AAV9 was neutralized by the anti-AAV mAb but the exosome-AAV constructs were not. In FIGS. 18A-18D, the data for one of the samples derived from anion exchanged chromatography, F7, was extracted to enable head-to-head comparison with the AAV only sample as a function of neutralizing antibody titer. Exosome-AAV shows more rapid transduction kinetics, increased potency and enhanced immune evasion compared to AAV at t=24, 48, 72, and 96 hours following addition of the sample to the HeLa cell culture.

### Example 12

### Biodistribution of AAV9 and Exosomes Following Intravitreal Administration in Rats

Materials and Dose Formulation Specifics: AAV9 and exosomes are in a ready to use formulation. Two vials of 40 μL AAV9 and 40 μL exosome are provided. Intravenous immunoglobulin (IVIg) (25 mg vial; powder form) is also provided for addition to one AAV9 vial and one exosome vial using the following method: (i) reconstitute 25 mg IVIg powder in 0.5 mL PBS (target concentration of 50 mg/mL), and (ii) from the 50 mg/mL IVIg stock solution perform the following: add 0.8 μL IVIg to one AAV vial, mix by pipetting, and incubate at 4° C. for at least 1 hours prior to injection; and add 0.8 μL IVIg to one exosome vial, mix by pipetting, and incubate at 4° C. for at least 1 hours prior to injection.

Animal Model: CD rats (˜6-8 weeks old) will be used on study (n=15 purchased, n=10 on-study).

Imaging Study Design:

(1) Dose Administration:
a. Injection and route:
   i. Group 1, n=2: Intravitreal injection (˜5 μL) of PBS
   ii. Group 2, n=2: Intravitreal injection (˜5 μL) of AAV9
   iii. Group 3, n=2: Intravitreal injection (˜5 μL) of AAV9 pre-incubated with IVIg.
   iv. Group 4, n=2: Intravitreal injection (˜5 μL) of exosome-AAV9
   v. Group 5, n=2: Intravitreal injection (˜5 μL) of exosome-AAV9 pre-incubated with IVIg.
b. Both eyes will be injected with the control/test article (refer to dose group for specific compounds)

(2) Tissue Collection:
1. Terminal time point for tissue collection will be 2 weeks post-injection of each control/test article. The following tissues will be collected: Eyes (left and right)
2. Both eyes will be collected from all animals, flash frozen, and stored at −20° C. until shipped to the sponsor.

TABLE 7
Study Design Summary.

| | No & Type | Control/Test | Volume & Rt | Collection | Tissues |
|---|---|---|---|---|---|
| Group | of Animal | Article | of Injection | Time Point | Collected |
| 1 | 2 CD rats | PBS | 5 μL, IVT | 2 weeks post- | Eyes (right and |
| | | | | injection | left) |
| 2 | 2 CD rats | AAV9 | 5 μL, IVT | 2 weeks post- | Eyes (right and |
| | | | | injection | left) |
| 3 | 2 CD rats | AAV9 + IVIg | 5 μL, IVT | 2 weeks post- | Eyes (right and |
| | | | | injection | left) |
| 4 | 2 CD rats | Exosome-AAV9 | 5 μL, IVT | 2 weeks post- | Eyes (right and |
| | | | | injection | left) |
| 5 | 2 CD rats | Exosome AAV9 + | 5 μL, IVT | 2 weeks post- | Eyes (right and |
| | | IVIg | | injection | left) |

IVT—intravitreal

### Example 13

### Shielding of AAV9 from Neutralizing Antibodies by Exosome Encapsulation

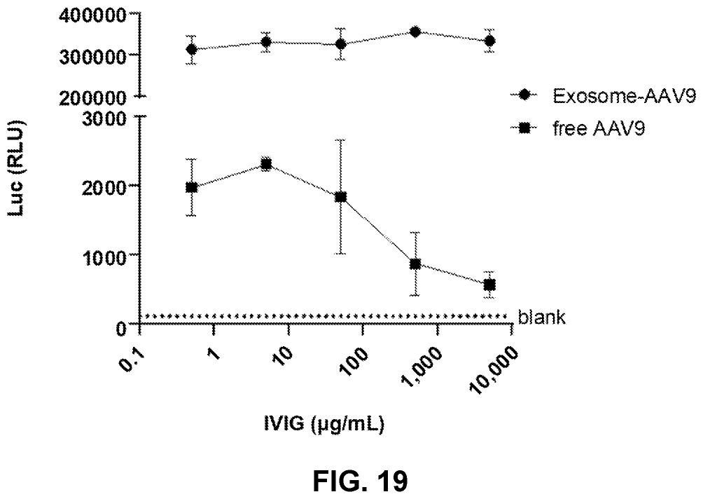

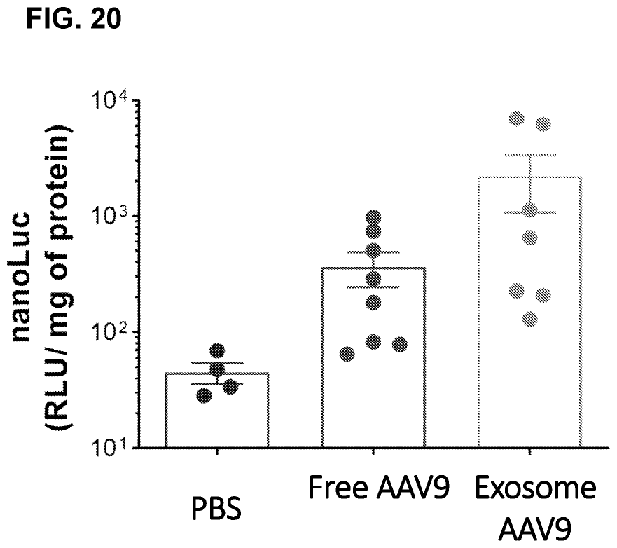

Neutralizing antibodies limit addressable patient populations because 20% to 50% have pre-existing neutralizing antibodies against AAV. Re-dosing with AAV is not currently possible because high titer cross-reactive anti-AAV antibodies are generated after AAV exposure. Accordingly, AAVs were shielded from neutralizing antibodies by exosome encapsulation (stochastic loading, e.g., random localization). As shown in FIG. 19, when AAV9 was shielded by exosomes, the luciferase signal was unaffected by increasing concentrations of neutralizing antibodies. In contrast, the luciferase signal considerably decayed in response to increasing concentrations of neutralizing antibodies. Even at the lowest antibody concentrations tested, the protective effect of the exosomes was substantial as indicated by the difference on luciferase signal, which was 150 times in the exosome encapsulated sample (approx. 300,000 RLU versus approx. 2,000).

### Example 14

### Administration of Exosome-AAV to Mice Yields Increased Expression of AAV-Encoded Reporter

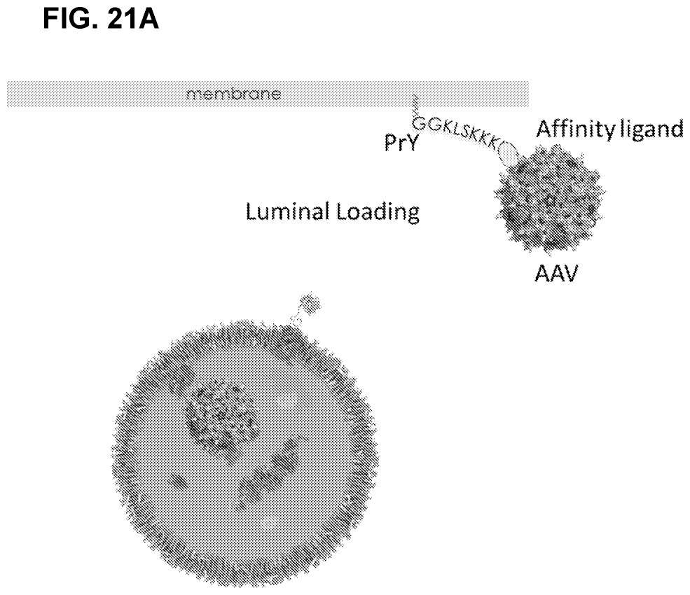

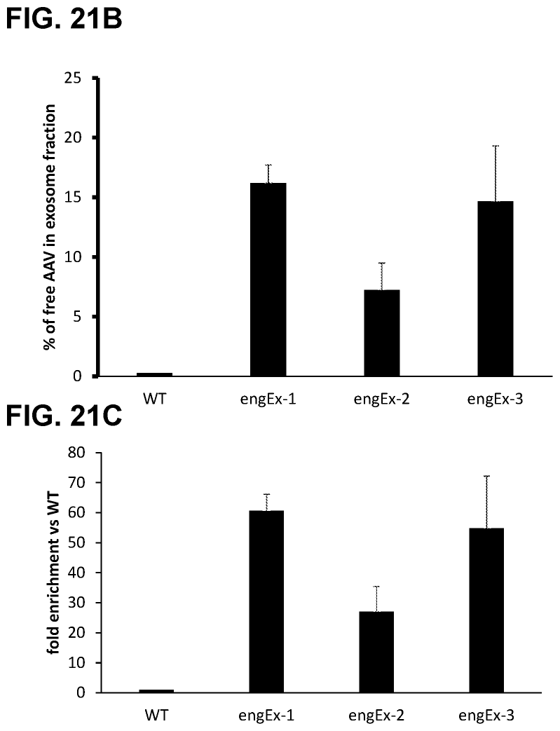

To test the expression of a reporter gene encoded by an AAV associated with an exosome, CD rats (6-8 weeks old) were injected intravitreally with 5 ul of free AAV9 or exosome-AAV9 (˜1e10 vg; illustrated in FIG. 21A), encoding for secreted nanoLuc (n=4 animals, 2 eyes per animal). Two weeks post-administration, eyes were collected, frozen, and subsequently homogenized. Luciferase and total protein levels were measured. A trend towards higher transgene expression is observed in the exosome-AAV group as compared with the free AAV9 group (FIGS. 21B-21C).

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, the above specification is not restrictive. It will be appreciated that various changes can be made without departing from the spirit and scope of the invention(s). Many variations will become apparent to those skilled in the art upon review of this specification.

What is claimed:

1. An extracellular vesicle (EV) comprising an adeno-associated virus (AAV) and a scaffold protein, wherein the AAV is in the lumen of the EV, and wherein the AAV in the EV has altered properties as compared to the AAV alone.

2. The EV of claim 1, wherein the altered property comprises a better therapeutic effect than AAV alone.

3. The EV of claim 1 or 2, wherein the better therapeutic effect comprises one or more of higher Infectivity, higerh activity, greater potency, faster transduction kinetics, and tolerance against immune invasion.

4. The EV of any one of claims 1 to 3, wherein the altered properties of the AAV allow the AAV to be administered to a subject through two or more doses, wherein the infectivity and/or activity of the AAV is retained in subsequent doses.

5. An EV comprising an AAV, wherein the EV comprises a scaffold protein and at least 5 AAV, wherein the at least 5 AAV are in the lumen of the EV.

6. The EV of claim 5, comprising at least 6 AAVs, at least 7 AAVs, at least 8 AAVs, at least 9 AAVs, at least 10 AAVs, at least 11 AAVs, at least 12 AAVs, at least 13 AAVs, at least 14 AAVs, at least 15 AAVs, at least 16 AAVs, at least 17 AAVs, at least 18 AAVs, at least 19 AAVs, at least 20 AAVs, at least 201 AAVs, at least 22 AAVs, at least 23 AAVs, at least 24 AAVs, at least 25 AAVs, at least 26 AAVs, at least 27 AAVs, at least 28 AAVs, at least 29 AAVs, at least 30 AAVs, at least 35 AAVs, at least 40 AAVs, at least 45 AAVs, at least 50 AAVs, at least 60 AAVs, at least 70 AAVs, at least 80 AAVs, at least 90 AAVs, or at least 100 AAVs in the lumen of the EV.

7. The EV of claim 5, comprising at least about 5 AAVs to at least about 100 AAVs, at least about 5 AAVs to at least about 75 AAVs, at least about 5 AAVs to at least about 50 AAVs, at least about 5 AAVs to at least about 45 AAVs, at least about 5 AAVs to at least about 40 AAVs, at least about 5 AAVs to at least about 35 AAVs, at least about 5 AAVs to at least about 30 AAVs, at least about 5 AAVs to at least about 25 AAVs, at least about 5 AAVs to at least about 20 AAVs, at least about 5 AAVs to at least about 15 AAVs, at least about 5 AAVs to at least about 10 AAVs, at least about 10 AAVs to at least about 100 AAVs, at least about 10 AAVs to at least about 75 AAVs, at least about 10 AAVs to at least about 50 AAVs, at least about 5 AAVs to at least about 45 AAVs, at least about 10 AAVs to at least about 40 AAVs, at least about 10 AAVs to at least about 35 AAVs, at least about 10 AAVs to at least about 30 AAVs, at least about 10 AAVs to at least about 25 AAVs, at least about 10 AAVs to at least about 20 AAVs, or at least about 10 AAVs to at least about 15 AAVs in the lumen of the EV.

8. The EV of any one of claims 5 to 7 comprising at least about 5 AAVs to at least about 20 AAVs.

9. The EV of any one of claims 1 to 8, wherein the EV comprises a bi-lipid membrane comprising a luminal surface and an external surface, wherein at least one of the AAVs is not linked to the luminal surface of the EV.

10. The EV of any one of claims 1 to 8, wherein the EV comprises a bi-lipid membrane comprising a luminal surface and an external surface, wherein at least one of the AAVs is linked to the luminal surface of the EV.

11. The EV of claim 10, wherein the at least one AAV is linked to the luminal surface of the EV by a covalent bond or a non-covalent bond.

12. The EV of claim 10 or 11, wherein the at least one AAV is linked to the luminal surface of the EV by both a covalent bond and a non-covalent bond.

13. The EV of any one of claims 1 to 12, wherein the AAV is linked to the scaffold protein.

14. The EV of any one of claims 1 to 13, wherein the scaffold protein comprises an N terminus domain (ND) and an effector domain (ED), wherein the ND and/or the ED are associated with the luminal surface of the EV.

15. The EV of claim 14, wherein the ND is associated with the luminal surface of the EV via myristoylation.

16. The EV of claim 14 or 15, wherein the ED is associated with the luminal surface of the EV by an ionic interaction.

17. The EV of any one of claims 14 to 16, wherein the ED comprises (i) a basic amino acid or (ii) two or more basic amino acids in sequence, wherein the basic amino acid is selected from the group consisting of Lys, Arg, His, and any combination thereof.

18. The EV of claim 17, wherein the basic amino acid is (Lys)n, wherein n is an integer between 1 and 10.

19. The EV of any one of claims 14 to 18, wherein the ED comprises Lys (K), KK, KKK, KKKK (SEQ ID NO: 11), KKKKK (SEQ ID NO: 12), Arg (R), RR, RRR, RRRR (SEQ ID NO: 13); RRRRR (SEQ ID NO: 14), KR, RK, KKR, KRK, RKK, KRR, RRK, (K/R)(K/R)(K/R)(K/R) (SEQ ID NO: 15), (K/R)(K/R)(K/R)(K/R)(K/R) (SEQ ID NO: 16), or any combination thereof.

20. The EV of any one of claims 14 to 19, wherein the ND comprises the amino acid sequence as set forth in G:X2:X3:X4:X5:X6, wherein G represents Gly; wherein “:” represents a peptide bond, wherein each of the X2 to the X6 is independently an amino acid, and wherein the X6 comprises a basic amino acid.

21. The EV of claim 20, wherein:
i. the X2 is selected from the group consisting of Pro, Gly, Ala, and Ser;
ii. the X4 is selected from the group consisting of Pro, Gly, Ala, Ser, Val, Ile, Leu, Phe, Trp, Tyr, Gln and Met;
iii. the X5 is selected from the group consisting of Pro, Gly, Ala, and Ser;
iv. the X6 is selected from the group consisting of Lys, Arg, and His; or
v. any combination of (i)-(iv).

22. The EV of any one of claims 14 to 21, wherein the ND comprises the amino acid sequence of G:X2:X3:X4:X5:X6, wherein
i. G represents Gly;
ii. “:” represents a peptide bond;
iii. the X2 is an amino acid selected from the group consisting of Pro, Gly, Ala, and Ser;
iv. the X3 is an amino acid;
v. the X4 is an amino acid selected from the group consisting of Pro, Gly, Ala, Ser, Val, Ile, Leu, Phe, Trp, Tyr, Gln and Met;
vi. the X5 is an amino acid selected from the group consisting of Pro, Gly, Ala, and Ser; and
vii. the X6 is an amino acid selected from the group consisting of Lys, Arg, and His.

23. The EV of any one of claims 18 to 22, wherein the X3 is selected from the group consisting of Asn, Gln, Ser, Thr, Asp, Glu, Lys, His, and Arg.

24. The EV of any one of claims 14 to 23, wherein the ND and the ED are joined by a linker.

25. The EV of claim 24, wherein the linker comprises a peptide bond or one or more amino acids.

26. The EV of any one of claims 14 to 25, wherein the ND comprises an amino acid sequence selected from the group consisting of (i) GGKLSKK (SEQ ID NO: 17), (ii) GAKLSKK (SEQ ID NO: 18), (iii) GGKQSKK (SEQ ID NO: 19), (iv) GGKLAKK (SEQ ID NO: 20), (v) GGKLSKK (SEQ ID NO: 21), or (vi) any combination thereof.

27. The EV of claim 26, wherein the ND comprises an amino acid sequence selected from the group consisting of (i) GGKLSKKK (SEQ ID NO: 22), (ii) GGKLSKKS (SEQ ID NO: 23), (iii) GAKLSKKK (SEQ ID NO: 24), (iv) GAKLSKKS (SEQ ID NO: 25), (v) GGKQSKKK (SEQ ID NO: 26), (vi) GGKQSKKS (SEQ ID NO: 27), (vii) GGKLAKKK (SEQ ID NO: 28), (viii) GGKLAKKS (SEQ ID NO: 29), (ix) GGKLSKKK (SEQ ID NO: 30), (x) GGKLSKKS (SEQ ID NO: 31), and (xi) any combination thereof.

28. The EV of any one of claims 14 to 27, wherein the ND comprises the amino acid sequence GGKLSKK (SEQ ID NO: 17).

29. The EV of any one of claims 1 to 28, wherein the scaffold protein is at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 105, at least about 110, at least about 120, at least about 130, at least about 140, at least about 150, at least about 160, at least about 170, at least about 180, at least about 190, or at least about 200 amino acids in length.

30. The EV of any one of claims 1 to 29, wherein the scaffold protein comprises (i) GGKLSKKKKGYNVN (SEQ ID NO: 32), (ii) GAKLSKKKKGYNVN (SEQ ID NO: 33), (iii) GGKQSKKKKGYNVN (SEQ ID NO: 34), (iv) GGKLAKKKKGYNVN (SEQ ID NO: 35), (v) GGKLSKKKKGYSGG (SEQ ID NO: 36), (vi) GGKLSKKKKGSGGS (SEQ ID NO: 37), (vii) GGKLSKKKKSGGSG (SEQ ID NO: 38), (viii) GGKLSKKKSGGSGG (SEQ ID NO: 39), (ix) GGKLSKKSGGSGGS (SEQ ID NO: 40), (x) GGKLSKSGGSGGSV (SEQ ID NO: 41), or (xi) GAKKSKKRFSFKKS (SEQ ID NO: 42).

31. The EV of any one of claims 14 to 30, wherein the scaffold protein does not comprise Met at the N terminus.

32. The EV of any one of claims 14 to 31, wherein the scaffold protein comprises a myristoylated amino acid residue at the N terminus of the scaffold protein.

33. The EV of claim 32, wherein the amino acid residue at the N terminus of the scaffold protein is Gly.

34. The EV of claim 32 or 33, wherein the amino acid residue at the N terminus of the scaffold protein is synthetic.

35. The EV of claim 33 or 34, wherein the amino acid residue at the N terminus of the scaffold protein is a glycine analog.

36. The EV of any one of claims 1 to 35, wherein the scaffold protein comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 8, SEQ ID NO: 9, or SEQ ID NO: 10.

37. The EV of any one of claims 14 to 36, wherein the EV further comprises a second scaffold protein, which comprises prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), CD13, aminopeptidase N (ANPEP), neprilysin (membrane metalloendopeptidase; MME), ectonucleotide pyrophosphatase/phosphodiesterase family member 1 (ENPP1), neuropilin-1 (NRP1), CD9, CD63, CD81, PDGFR, GPI anchor proteins, lactadherin, LAMP2, LAMP2B, a fragment thereof, and any combination thereof.

38. The EV of any one of claims 1 to 37, wherein the AAV comprises at least one capsid protein fused to the scaffold protein and/or the second scaffold protein.

39. The EV of claim 38, wherein the at least one capsid protein is selected from the group consisting of VP1, VP2, and VP3.

40. The EV of claim 38 or 39, wherein the AAV capsid protein comprises VP2.

41. The EV of any one of claims 38 to 40, wherein the AAV comprises at least one VP2 that is not fused to the scaffold protein and/or the second scaffold protein.

42. The EV of any one of claims 38 to 41, wherein the scaffold protein is fused to the N-terminus of the VP2.

43. The EV of claim 41 or 42, wherein the number of the VP2 fused to the scaffold protein is less than the number of the at least one VP2 not fused to the scaffold protein.

44. The EV of claim 41 or 42, wherein the number of the VP2 fused to the scaffold protein is about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 30 fold, about 35 fold, about 40 fold, about 46 fold, about 50 fold, or about 100 fold less than the number of the at least one VP2 not fused to the scaffold protein.

45. The EV of any one of claims 1 to 13, wherein the scaffold protein is a type I transmembrane protein or a type II transmembrane protein.

46. The EV of claim 45, wherein the a type I transmembrane protein comprises prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), CD13, aminopeptidase N (ANPEP), neprilysin (membrane metalloendopeptidase; MME), ectonucleotide pyrophosphatase/phosphodiesterase family member 1 (ENPP1), neuropilin-1 (NRP1), CD9, CD63, CD81, PDGFR, GPI anchor proteins, lactadherin, LAMP2, LAMP2B, a fragment thereof, and any combination thereof.

47. The EV of claim 45 or 46, wherein the C terminus of the type I transmembrane protein or the N terminus of the type II transmembrane protein is linked to a binding partner of a chemically induced dimer.

48. The EV of any one of claims 14 to 37, wherein the scaffold protein is linked to a binding partner of a chemically induced dimer.

49. The EV of claim 47 or 48, wherein the binding partner of the chemically induced dimer comprises one of binding partners selected from the group; consisting of (i) FKBP and FKBP (FK1012); (ii) FKBP and CalcineurinA (CNA) (FK506); (iii) FKBP and CyP-Fas (FKCsA); (iv) FKBP and FRB (Rapamycin); (v) GyrB and GyrB (Coumermycin); (vi) GAI and GID1 (Gibberellin); (vii) Snap-tag and HaloTag (HaXS); (viii) eDHFR and HaloTag (TMP-HTag); and (ix) BCL-xL and Fab (AZ1) (ABT-737).

50. The EV of any one of claims 47 to 49, wherein the chemically induced dimer comprises an FRB-FKBP fusion complex.

51. The EV of claim 50, wherein the FRB is the FRB of mTOR.

52. The EV of any one of claims 47 to 51, wherein the AAV comprises at least one capsid protein fused to one of the binding partners of the chemically induced dimer, thereby forming a dimer complex when the binding partners come in contact with the chemical compound.

53. The EV of claim 52, wherein the at least one capsid protein is selected from the group consisting of VP1, VP2, and VP3.

54. The EV of claim 52 or 53, wherein the AAV capsid protein comprises VP2.

55. The EV of any one of claims 52 to 54, wherein the AAV comprises at least one VP2 that is not fused to a binding partner of the chemically induced dimer.

56. The EV of claim 55, wherein the number of the VP2 fused to a binding partner of the chemically induced dimer is less than the at least one VPs that is not fused to a binding partner of the chemically induced dimer.

57. The EV of claim 56, wherein the number of the VP2 linked to the binding partner of the chemically induced dimer is about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 30 fold, about 35 fold, about 40 fold, about 46 fold, about 50 fold, or about 100 fold less than the number of the at least one VP2 not fused to the binding partner.

58. The EV of claim 52 or 53, wherein the binding partner of the chemically induced dimer is inserted within an internal loop of the AAV capsid protein.

59. The EV of claim 58, wherein the internal loop comprises the sequence GTTTQSR (SEQ ID NO: 43).

60. The EV of claim 58, wherein the internal loop comprises amino acid residues 453 to 459 of SEQ ID NO: 44.

61. The EV of claim 59 or 60, wherein at least one amino acid of the internal loop is replaced by a binding partner of the chemically induced dimer.

62. The EV of any one of claims 30 to 46, wherein the scaffold protein is linked to the binding partner of the chemically induced dimer by a linker.

63. The EV of any one of claims 38 to 44 or 52 to 62, wherein the AAV capsid protein is linked to the binding partner of the chemically induced dimer by a linker.

64. The EV of claim 62 or 63, wherein the linker comprises a covalent bond or one or more amino acids.

65. The EV of any one of claims 62 to 64, wherein the linker is a cleavable linker.

66. The EV of any one of claims 1 to 37 and 45 to 47, wherein the scaffold protein is linked to an affinity agent that specifically binds to the AAV.

67. The EV of claim 66, wherein the affinity agent is an AAV receptor, a nanobody, a camelid antibody, an IgNAR, a single-domain antibody, an antibody or an antigen-binding portion thereof, any functional fragment thereof, or any combination thereof.

68. The EV of claim 67, wherein the antigen-binding portion thereof comprises a single chain Fab.

69. The EV of any one of claims 66 to 68, wherein the affinity agent binds to one or more AAV capsid proteins.

70. The EV of claim 69, wherein the one or more AAV capsid proteins are AAV assembly activating proteins.

71. The EV of claim 70, wherein the affinity agent does not bind to an AAV capsid protein monomer.

72. The EV of any one of claims 1 to 71, wherein the AAV further comprises a genetic cassette comprising a heterologous sequence encoding a gene of interest.

73. The EV of claim 72, wherein the genetic cassette encodes a protein selected from the group consisting of a secreted protein, a receptor, a structural protein, a signaling protein, a sensory protein, a regulatory protein, a transport protein, a storage protein, a defense protein, a motor protein, a clotting factor, a growth factor, an antioxidant, a cytokine, a chemokine, an enzyme, a tumor suppressor gene, a DNA repair protein, a structural protein, a low-density lipoprotein receptor, an alpha glucosidase, a cystic fibrosis transmembrane conductance regulator, or any combination thereof.

74. The EV of claim 72 or 73, wherein the genetic cassette encodes a factor VIII protein or a factor IX protein.

75. The EV of claim 74, wherein the factor VIII protein is a wild-type factor VIII, a B-domain deleted factor VIII, a factor VIII fusion protein, or any combination thereof.

76. The EV of claim 72 or 73, wherein the gene of interest encodes a Rab proteins geranylgeranyltransferase component A 1 (REP1).

77. The EV of claim 76, wherein the REP1 comprises an amino acid sequence at least about 70%, at least about 75%, at least about at least about 80%, at least about 85%, at least about 90%, at least about 95%, at least about 96%, at least about 97%, at least about 98%, at least about 99%, or about 100% identical to SEQ ID NO: 45.

78. The EV of any one of claims 1 to 77, wherein the AAV is selected from the group consisting of AAV type 1, AAV type 2, AAV type 3A, AAV type 3B, AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, a synthetic AAV, an any combination thereof.

79. An AAV in the EV of any one of claims 1 to 78.

80. An AAV comprising VP2 linked to a scaffold protein comprising the amino acid sequence as set forth in G:X2:X3:X4:X5:X6, wherein G represents Gly; wherein “:” represents a peptide bond, wherein each of the X2 to the X6 is independently an amino acid, and wherein the X6 comprises a basic amino acid.

81. The AAV of claim 80, wherein the scaffold protein is the scaffold protein or the second scaffold protein set forth in any one of claims 14 to 37.

82. An AAV comprising VP2 linked to a binding partner of a chemically induced dimer.

83. The AAV of claim 82, wherein the binding partner of the chemically induced dimer comprises any one of the binding partners set forth in any one of claims 49 to 51.

84. An AAV comprising one or more capsid proteins specifically bound to an affinity agent.

85. The AAV of claim 84, wherein the affinity agent is set forth in any one of claims 67 to 71.

86. An extracellular vesicle (EV) comprising (i) an adeno-associated virus (AAV) and (ii) a scaffold protein, wherein the AAV is associated with the scaffold protein on the external surface of the EV.

87. The EV of claim 86, wherein the scaffold protein comprises an extracellular domain, and wherein the AAV is associated with the extracellular domain of the scaffold protein.

88. The EV of claim 86 or 87, wherein the scaffold protein further comprises a transmembrane region, wherein the transmembrane region is anchored to the membrane of the EV.

89. The EV of any one of claims 86 to 88, wherein the scaffold protein further comprises an intracellular domain.

90. The EV of any one of claims 86 to 89, wherein the scaffold protein comprises a heterologous polypeptide, wherein the heterologous polypeptide is fused to an extracellular domain of the scaffold protein, and wherein the heterologous polypeptide associates with the AAV.

91. The EV of any one of claims 86 to 90, wherein the scaffold protein is a type I transmembrane protein or a type II transmembrane protein.

92. The EV of claim 90 or 91, wherein the heterologous polypeptide is fused to the N-terminus or the C terminus of the extracellular domain of the scaffold protein.

93. The EV of any one of claims 90 to 92, wherein the heterologous polypeptide comprises a receptor, a ligand, an antigen-binding moiety, a substrate, a fragment thereof, or a combination thereof; and wherein the heterologous polypeptide specifically interacts with one or more proteins on the surface of the AAV.

94. The EV of claim 93, wherein the heterologous polypeptide comprises an antigen-binding moiety selected from the group consisting of an antigen-binding fragment of an antibody, a camelid antibody or an antigen-binding fragment thereof, a single-chain FAB, a nanobody, a shark IgNAR, and a combination thereof.

95. The EV of claim 93 or 94, wherein the antigen-binding moiety comprises a nanobody.

96. The EV of any one of claims 93 to 95, wherein the antigen binding moiety specifically binds the one or more proteins on the surface of the AAV.

97. The EV of any one of claims 93 to 96, wherein the one or more proteins on the surface of the AAV comprise a capsid protein selected from the group consisting of VP1, VP2, VP3, and any combination thereof.

98. The EV of any one of claims 93 to 97, wherein the one or more proteins on the surface of the AAV is a non-AAV sequence fused to a capsid protein of the AAV.

99. The EV of claim 98, wherein the capsid protein is selected from VP1, VP2, VP3, and any combination thereof.

100. The EV of claim 99, wherein the non-AAV sequence is fused to VP2.

101. The EV of claim 99 or 100, wherein the non-AAV sequence is fused to the N-terminus of VP2.

102. The EV of claim 99 or 101, wherein the non-AAV sequence is fused to an internal surface-exposed loop of VP2.

103. The EV of claim 99, wherein the non-AAV sequence is fused to VP3.

104. The EV of claim 99 or 103, wherein the non-AAV sequence is fused to the N-terminus of VP3.

105. The EV of claim 99 or 103, wherein the non-AAV sequence is fused to an internal surface-exposed loop of VP3.

106. The EV of claim 99, wherein the non-AAV sequence is fused to VP1.

107. The EV of claim 106, wherein the non-AAV sequence is fused to an internal surface-exposed loop of VP1.

108. The EV of any one of claims 86 to 93, wherein:
(i) the scaffold protein is fused to a heterologous polypeptide comprising an Fc receptor; and
(ii) the AAV comprises at least one capsid protein fused to an Fc region of an immunoglobulin constant region (Fc).

109. The EV of claim 108, wherein the Fc receptor is an Fc gamma receptor selected from Fc gamma receptor I (FcγR1), FcγRIIA, FcγIIB, FcγIIIA, and FcγIIIB; and wherein the Fc is an Fc of an IgG.

110. The EV of claim 108 or 109, wherein the Fc receptor is an FcγR1 and the Fc is an Fc of an IgG.

111. The EV of claim 108, wherein the Fc receptor is an Fc alpha receptor I (FcαR1), and wherein the Fc is an Fc of an IgA.

112. The EV of claim 108, wherein the Fc receptor is an Fc epsilon receptor selected from Fc epsilon receptor I (FcεRI) and FcεRII, and wherein the Fc is an Fc of an IgE.

113. The EV of any one of claims 86 to 93, wherein:
(i) the scaffold protein is fused to a heterologous polypeptide comprising a nanobody; and
(ii) the AAV comprises at least one capsid protein fused to an Fc region of an immunoglobulin constant region (Fc).

114. The EV of claim 113, wherein the nanobody specifically binds to the Fc fused to the capsid protein.

115. The EV of any one of claims 108 to 114, wherein the at least one capsid protein is selected from the group consisting of VP1, VP2, and VP3.

116. The EV of any one of claims 108 to 115, wherein the AAV comprises at least one VP2 fused to an Fc.

117. The EV of claim 116, wherein the AAV comprises at least one VP2 that is not fused to an Fc.

118. The EV of any one of claims 108 to 117, wherein the Fc is fused to the N-terminus of the at least one VP2.

119. The EV of any one of claims 108 to 117, wherein the Fc is fused to an internal surface-exposed loop of the at least one VP2.

120. The EV of any one of claims 108 to 119, wherein the AAV comprises at least one VP3 fused to an Fc.

121. The EV of claim 120, wherein the AAV comprises at least one VP3 that is not fused to the Fc.

122. The EV of any one of claims 108 to 121, wherein the Fc is fused to the N-terminus of the at least one VP3.

123. The EV of any one of claims 108 to 121, wherein the Fc is fused to an internal surface-exposed loop of the at least one VP3.

124. The EV of any one of claims 108 to 123, wherein the AAV comprises at least one VP1 fused to an Fc.

125. The EV of any one of claims 108 to 123, wherein the AAV comprises at least one VP1 that is not fused to an Fc.

126. The EV of claim 124, wherein the Fc is fused to a surface-exposed loop of VP1.

127. The EV of any one of claims 102, 105, 107, 119, 123, and 126, wherein the surface-exposed loop comprises the sequence GTTTQSR (SEQ ID NO: 43).

128. The EV of any one of claims 102, 105, 107, 119, 123, and 126, wherein the surface-exposed loop comprises amino acid residues 453 to 459 of VP1.

129. The EV of any one of claims 102, 105, 107, 119, 123, and 126 to 128, wherein the at least one amino acid of the surface-exposed loop is replaced by the Fc.

130. The EV of any one of claims 102, 105, 107, 119, 123, and 126 to 129, wherein the surface-exposed loop is replaced by the Fc.

131. The EV of any one of claims 86 to 96, wherein the scaffold protein is fused to an antigen-binding moiety, wherein the antigen-binding moiety specifically binds an antigen on the surface of an AAV.

132. The EV of any one of claims 86 to 93, wherein the scaffold protein is fused to a heterologous polypeptide comprising an AAV receptor.

133. The EV of any one of claims 86 to 132, wherein the AAV further comprises a nucleotide sequence comprising a gene of interest.

134. The EV of claim 133, wherein the gene of interest encodes a protein selected from the group consisting of a secreted protein, a receptor, a structural protein, a signaling protein, a sensory protein, a regulatory protein, a transport protein, a storage protein, a defense protein, a motor protein, a clotting factor, a growth factor, an antioxidant, a cytokine, a chemokine, an enzyme, a tumor suppressor gene, a DNA repair protein, a structural protein, a low-density lipoprotein receptor, an alpha glucosidase, a cystic fibrosis transmembrane conductance regulator, or any combination thereof.

135. The EV of claim 133 or 134, wherein the gene of interest encodes a factor VIII protein or a Factor IX protein.

136. The EV of claim 135, wherein the factor VIII protein is a wild-type factor VIII, a B-domain deleted factor VIII, a factor VIII fusion protein, or any combination thereof.

137. 52. The EV of claim 133 or 134, wherein the gene of interest encodes a Rab proteins geranylgeranyltransferase component A 1 (REP1)

138. 53. The EV of claim 137, wherein the REP1 comprises an amino acid sequence at least about 70%, at least about 75%, at least about at least about 80%, at least about 85%, at least about 90%, at least about 95%, at least about 96%, at least about 97%, at least about 98%, at least about 99%, or about 100% identical to SEQ ID NO: 45.

139. The EV of any one of claims 86 to 138, wherein the AAV is selected from the group consisting of AAV type 1, AAV type 2, AAV type 3A, AAV type 3B, AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, a synthetic AAV, an any combination thereof.

140. The EV of any one of claims 86 to 139, wherein the scaffold protein is selected from the group consisting of prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), CD13, aminopeptidase N (ANPEP), neprilysin (membrane metalloendopeptidase; MME), ectonucleotide pyrophosphatase/phosphodiesterase family member 1 (ENPP1), neuropilin-1 (NRP1), CD9, CD63, CD81, PDGFR, GPI anchor proteins, lactadherin, LAMP2, LAMP2B, a fragment thereof, and any combination thereof.

141. The EV of any one of claims 86 to 140, wherein the scaffold protein is PTGFRN.

142. The EV of claim 87, wherein the scaffold protein comprises an N terminus domain, an effector domain, and a transmembrane domain, wherein the ND is myristoylated, and wherein the N-terminus domain (ND) and/or the effector domain (ED) are associated with the luminal surface of the EV.

143. The EV of claim 142, wherein the ED is associated with the luminal surface of the EV by an ionic interaction.

144. The EV of claim 142 or 143, wherein the ED comprises (i) a basic amino acid or (ii) two or more basic amino acids next to each other in a sequence, wherein the basic amino acid is selected from the group consisting of Lys, Arg, His, and any combination thereof.

145. The EV of claim 144, wherein the basic amino acid is (Lys)n, wherein n is an integer between 1 and 10.

146. The EV of any one of claims 142 to 145, wherein the ED comprises Lys (K), KK, KKK, KKKK (SEQ ID NO: 11), KKKKK (SEQ ID NO: 12), RR, RRR, RRRR (SEQ ID NO: 13); RRRRR (SEQ ID NO: 14), KR, RK, KKR, KRK, RKK, KRR, RRK, (K/R)(K/R)(K/R)(K/R) (SEQ ID NO: 15), (K/R)(K/R)(K/R)(K/R)(K/R) (SEQ ID NO: 16), or any combination thereof.

147. The EV of any one of claims 142 to 146, wherein the ND comprises the amino acid sequence as set forth in G:X2:X3:X4:X5:X6, wherein G represents Gly; wherein “:” represents a peptide bond, wherein each of the X2 to the X6 is an amino acid, and wherein the X6 comprises a basic amino acid.

148. The EV of claim 147, wherein:
(i) the X6 is selected from the group consisting of Lys, Arg, and His;
(ii) the X5 is selected from the group consisting of Pro, Gly, Ala, and Ser;
(iii) the X2 is selected from the group consisting of Pro, Gly, Ala, and Ser;
(iv) the X4 is selected from the group consisting of Pro, Gly, Ala, Ser, Val, Ile, Leu, Phe, Trp, Tyr, Gln and Met; or
(v) any combination of (i)-(iv).

149. The EV of any one of claims 142 to 148, wherein the ND of the Scaffold protein comprises the amino acid sequence of G:X2:X3:X4:X5:X6, wherein
(i) G represents Gly;
(ii) “:” represents a peptide bond;
(iii) the X2 is an amino acid selected from the group consisting of Pro, Gly, Ala, and Ser;
(iv) the X3 is an amino acid;
(v) the X4 is an amino acid selected from the group consisting of Pro, Gly, Ala, Ser, Val, Ile, Leu, Phe, Trp, Tyr, Gln and Met;
(vi) the X5 is an amino acid selected from the group consisting of Pro, Gly, Ala, and Ser; and
(vii) the X6 is an amino acid selected from the group consisting of Lys, Arg, and His.

150. The EV of any one of claims 147 to 149, wherein the X3 is selected from the group consisting of Asn, Gln, Ser, Thr, Asp, Glu, Lys, His, and Arg.

151. The EV of any one of claims 142 to 150, wherein the ND and the ED are joined by a linker.

152. The EV of claim 151, wherein the linker comprises a peptide bond or one or more amino acids.

153. The EV of any one of claims 142 to 152, wherein the scaffold protein comprises an amino acid sequence selected from the group consisting of (i) GGKLSKK (SEQ ID NO: 17), (ii) GAKLSKK (SEQ ID NO: 18), (iii) GGKQSKK (SEQ ID NO: 19), (iv) GGKLAKK (SEQ ID NO: 20), (v) GGKLSKK (SEQ ID NO: 21), or (vi) any combination thereof.

154. The EV of claim 153, wherein the scaffold protein comprises an amino acid sequence selected from the group consisting of (i) GGKLSKKK (SEQ ID NO: 22), (ii) GGKLSKKS (SEQ ID NO: 23), (iii) GAKLSKKK (SEQ ID NO: 24), (iv) GAKLSKKS (SEQ ID NO: 25), (v) GGKQSKKK (SEQ ID NO: 26), (vi) GGKQSKKS (SEQ ID NO: 27), (vii) GGKLAKKK (SEQ ID NO: 28), (viii) GGKLAKKS (SEQ ID NO: 29), (ix) GGKLSKKK (SEQ ID NO: 30), (x) GGKLSKKS (SEQ ID NO: 31), and (xi) any combination thereof.

155. The EV of any one of claims 142 to 154, wherein the C-terminus of the scaffold protein and/or the second scaffold protein is linked to a capsid protein of the AAV.

156. The EV of any one of claims 1 to 78 and 86 to 155, which is an exosome.

157. An adeno-associated virus (AAV) comprising a capsid, wherein the capsid comprises at least one capsid protein selected from the group consisting of VP1, VP2, and VP3; wherein the at least one capsid protein is linked to a scaffold protein.

158. The AAV of claim 157, wherein the scaffold protein is selected from the group consisting of prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), CD13, aminopeptidase N (ANPEP), neprilysin (membrane metalloendopeptidase; MME), ectonucleotide pyrophosphatase/phosphodiesterase family member 1 (ENPP1), neuropilin-1 (NRP1), CD9, CD63, CD81, PDGFR, GPI anchor proteins, lactadherin, LAMP2, LAMP2B, a fragment thereof, and any combination thereof.

159. The AAV of claim 157, wherein the scaffold protein comprises an N terminus domain, an effector domain, and a transmembrane domain, wherein the ND is myristoylated, and wherein the N-terminus domain (ND) and/or the effector domain (ED) are associated with the luminal surface of the EV.

160. The AAV of claim 159, wherein the ED is associated with the luminal surface of the EV by an ionic interaction.

161. The AAV of claim 159 or 160, wherein the ED comprises (i) a basic amino acid or (ii) two or more basic amino acids next to each other in a sequence, wherein the basic amino acid is selected from the group consisting of Lys, Asp, His, and any combination thereof.

162. The AAV of claim 161, wherein the basic amino acid is (Lys)n, wherein n is an integer between 1 and 10.

163. The AAV of any one of claims 159 to 162, wherein the ED comprises Lys (K), KK, KKK, KKKK (SEQ ID NO: 11), KKKKK (SEQ ID NO: 12), Arg (R), RR, RRR, RRRR (SEQ ID NO: 13); RRRRR (SEQ ID NO: 14), KR, RK, KKR, KRK, RKK, KRR, RRK, (K/R)(K/R)(K/R)(K/R) (SEQ ID NO: 15), (K/R)(K/R)(K/R)(K/R)(K/R) (SEQ ID NO: 16), or any combination thereof.

164. The AAV of any one of claims 159 to 163, wherein the ND comprises the amino acid sequence as set forth in G:X2:X3:X4:X5:X6, wherein G represents Gly; wherein “:” represents a peptide bond, wherein each of the X2 to the X6 is an amino acid, and wherein the X6 comprises a basic amino acid.

165. The AAV of claim 164, wherein:
(i) the X6 is selected from the group consisting of Lys, Asp, and His;
(ii) the X5 is selected from the group consisting of Pro, Gly, Ala, and Ser;
(iii) the X2 is selected from the group consisting of Pro, Gly, Ala, and Ser;
(iv) the X4 is selected from the group consisting of Pro, Gly, Ala, Ser, Val, Ile, Leu, Phe, Trp, Tyr, Gln and Met; or
(v) any combination of (i)-(iv).

166. The AAV of any one of claims 160 to 165, wherein the ND of the Scaffold protein comprises the amino acid sequence of G:X2:X3:X4:X5:X6, wherein
(i) G represents Gly;
(ii) “:” represents a peptide bond;
(iii) the X2 is an amino acid selected from the group consisting of Pro, Gly, Ala, and Ser;
(iv) the X3 is an amino acid;
(v) the X4 is an amino acid selected from the group consisting of Pro, Gly, Ala, Ser, Val, Ile, Leu, Phe, Trp, Tyr, Gln, and Met;
(vi) the X5 is an amino acid selected from the group consisting of Pro, Gly, Ala, and Ser; and
(vii) the X6 is an amino acid selected from the group consisting of Lys, Arg, and His.

167. The AAV of any one of claims 164 to 166, wherein the X3 is selected from the group consisting of Asn, Gln, Ser, Thr, Asp, Glu, Lys, His, and Arg.

168. The AAV of any one of claims 159 to 167, wherein the ND and the ED are joined by a linker.

169. The AAV of claim 168, wherein the linker comprises a peptide bond or one or more amino acids.

170. The AAV of any one of claims 157 to 169, wherein the scaffold protein comprises an amino acid sequence selected from the group consisting of (i) GGKLSKK (SEQ ID NO: 17), (ii) GAKLSKK (SEQ ID NO: 18), (iii) GGKQSKK (SEQ ID NO: 19), (iv) GGKLAKK (SEQ ID NO: 20), (v) GGKLSKK (SEQ ID NO: 21), or (vi) any combination thereof.

171. The AAV of claim 170, wherein the scaffold protein comprises an amino acid sequence selected from the group consisting of (i) GGKLSKKK (SEQ ID NO: 22), (ii) GGKLSKKS (SEQ ID NO: 23), (iii) GAKLSKKK (SEQ ID NO: 24), (iv) GAKLSKKS (SEQ ID NO: 25), (v) GGKQSKKK (SEQ ID NO: 26), (vi) GGKQSKKS (SEQ ID NO: 27), (vii) GGKLAKKK (SEQ ID NO: 28), (viii) GGKLAKKS (SEQ ID NO: 29), (ix) GGKLSKKK (SEQ ID NO: 30), (x) GGKLSKKS (SEQ ID NO: 31), and (xi) any combination thereof.

172. The AAV of any one of claims 157 to 171, which is associated with an external surface of an extracellular vesicle (EV).

173. The AAV of any one of claims 157 to 172, which is associated with an external surface of an EV, wherein the scaffold protein or the second scaffold protein is linked to the EV.

174. The EV of claim 66, wherein the scaffold protein is linked to an affinity agent that specifically binds to the AAV by a cleavable linker.

175. A pharmaceutical composition comprising the EV of any one of claims 1 to 78, 86 to 156 and 174 or the AAV of any one of claims 79 to 85 and 157 to 173 and a pharmaceutically acceptable carrier.

176. A cell that produces the isolated EV of any one of claims 1 to 78, 86 to 156 and 174 or the AAV of any one of claims 79 to 85 and 157 to 173.

177. A cell comprising a first nucleotide sequence encoding an AAV protein linked to the scaffold protein set forth in any one of claims 1 to 78 86 to 156, and 174.

178. The cell of claim 88, further comprising a second nucleotide sequence comprising the gene of interest set forth in any one of claims 72 to 77 and 133 to 156.

179. A cell comprising a first nucleotide encoding a AAV protein linked to a binding partner of the chemically induced dimer set forth in any one of claims 47 to 67.

180. The cell of claim 179, further comprising a second nucleotide sequence encoding the corresponding binding partner of the chemically induced dimer of claim 90, which is linked to the scaffold protein set forth in any one of claims 1 to 47.

181. The cell of claim 180, further comprising a third nucleotide sequence comprising the gene of interest set forth in any one of claims 72 to 77.

182. A cell comprising a first nucleotide encoding an affinity agent set forth in any one of claims 66 to 71 linked to the scaffold protein set forth in any one of claims 1 to 47.

183. The cell of claim 182, further comprising a second nucleotide sequence comprising the gene of interest set forth in any one of claims 72 to 77.

184. A kit comprising (i) the isolated EV of any one of claims 1 to 79, 86 to 156, and 174 or the AAV of any one of claims 79 to 85 and 157 to 173 and (ii) instructions for use.

185. A method of making EVs comprising culturing the cell of any one of claims 179 to 183 under a suitable condition and obtaining the EVs.

186. A method of preventing or treating a disease in a subject in need thereof, comprising administering to the subject the EV of any one of claims 1 to 78, 86 to 156, and 174 or the AAV of any one of claims 79 to 85 or 157 to 173 or the pharmaceutical composition of claim 176.

187. The method of claim 186, wherein the disease is selected from a cancer, a hemophilia, diabetes, a growth factor deficiency, an eye disease, a Pompe disease, a lysosomal storage disorder, mucovicidosis, cystic fibrosis, Duchenne and Becker muscular dystrophy, transthyretin amyloidosis, hemophilia A, hemophilia B, adenosine-deaminase deficiency, Leber's congenital amaurosis, X-linked adrenoleukodystrophy, metachromatic leukodystrophy, OTC deficiency, glycogen storage disease 1A, Criggler-Najjar syndrome, primary hyperoxaluria type 1, acute intermittent porphyria, phenylketonuria, familial hypercholesterolemia, mucopolysaccharidosis type VI, al antitrypsin deficiency, and a hypercholesterolemia.

188. A method of delivering an AAV to a subject, comprising administering to the subject the EV of any one of claims 1 to 78, 86 to 156, and 174.

189. The method of any one of claims 186 to 188, wherein the EV is administered parenterally, orally, intravenously, intramuscularly, intra-tumorally, intranasally, subcutaneously, or intraperitoneally.

190. The method of any one of claims 186 to 188, wherein the EV administration is intraocular administration.

191. The method of claim 190, wherein the intraocular administration is intravitreal administration, intracameral administration, subconjunctival administration, subretinal administration, sub scleral administration, intrachoroidal administration, and any combination thereof.

192. The method of claim 190 or 191, wherein the intraocular administration comprises the injection of the EV.

193. The method of any one of claims 190 to 192, wherein the intraocular administration is intravitreal injection.

194. The method of any one of claims 190 to 193, wherein the intraocular administration comprises the implantation of a delivery device comprising the composition.

195. The method of claim 194, wherein the delivery device is an intraocular delivery device.

196. The method of claim 195, wherein the intraocular delivery device is an intravitreal implant or a scleral plug.

197. The method of any one of claims 194 to 196, wherein the delivery device is a sustained release delivery device.

198. The method of any one of claims 194 to 197, wherein the delivery device is biodegradable.

199. The method of any one of claims 190 to 198, wherein the intraocular administration of the EV is to treat a disease selected from the group consisting of selected from the group consisting of macular degeneration, cataract, diabetic retinopathy, glaucoma, amblyopia, strabismus, retinopathy, or any combination thereof.

200. The method of any one of claims 186 to 199, comprising administering an additional therapeutic agent.

201. An extracellular vesicle (EV) comprising an adeno-associated virus (AAV) and a scaffold protein, wherein the AAV is associated with the exosome, and wherein the AAV has altered properties as compared to the AAV alone.

202. The EV of claim 201, wherein the AAV is associated with the luminal surface of the EV.

203. The EV of claim 201, wherein the AAV is associated with the exterior surface of the EV.

204. The EV of any one of claims 201 to 203, wherein the EV is an EV of anyone of claims 1 to 78, 86 to 156, and 174.


SEQUENCE LISTING (216 sequences)

SEQ ID NO: 1 - protein, 879 aa, Artificial Sequence
  Synthetic Peptide
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 2 - protein, 192 aa, Artificial Sequence
  Synthetic Peptide
GPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEM

SEQ ID NO: 3 - protein, 385 aa, Artificial Sequence
  Synthetic Peptide
MAAALFVLLGFALLGTHGASGAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 4 - protein, 613 aa, Artificial Sequence
  Synthetic Peptide
MGALRPTLLPPSLPLLLLLMLGMGCWAREVLVPEGPLYRVAGTAVSISCNVTGYEGPAQQNFEWFLYRPEAPDTALGIVSTKDTQFSYAVFKSRVVAGEVQVQRLQGDAVVLKIARLQAQDAGIYECHTPSTDTRYLGSYSGKVELRVLPDVLQVSAAPPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 5 - protein, 748 aa, Artificial Sequence
  Synthetic Peptide
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 6 - protein, 1032 aa, Artificial Sequence [sequence omitted]

SEQ ID NO: 7 - protein, 630 aa, Artificial Sequence
  Synthetic Peptide
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 8 - protein, 332 aa, Artificial Sequence
  Synthetic Peptide
MGAQFSKTAAKGEAAAERPGEAAVASSPSKANGQENGHVKVNGDASPAAAESGAKEELQANGSAPAADKEEPAAAGSGAASPSAAEKGEPAAAAAPEAGASPVEKEAPAEGEAAEPGSPTAAEGEAASAASSTSSPKAEDGATPSPSNETPKKKKKRFSFKKSFKLSGFSFKKNKKEAGEGGEAEAPAAEGGKDEAAGGAAAAAAEAGAASGEQAAAPGEEAAAGEEGAAGGDPQEAKPQEAAVAPEKPPASDETKAAEEPSKVEEKKAEEAGASAAACEAPSAAGPGAPPEQEAAPAEEPAAAAASSACAAPSQEAQPECSPEAPPAEAAE

SEQ ID NO: 9 - protein, 195 aa, Artificial Sequence
  Synthetic Peptide
MGSQSSKAPRGDVTAEEAAGASPAKANGQENGHVKSNGDLSPKGEGESPPVNGTDEAAGATGDAIEPAPPSQGAEAKGEVPPKETPKKKKKFSFKKPFKLSGLSFKRNRKEGGGDSSASSPTEEEQEQGEIGACSDEGTAQEGKAAATPESQEPQAKGAEASAASEEEAGPQATEPSTPSGPESGPTPASAEQNE

SEQ ID NO: 10 - protein, 227 aa, Artificial Sequence
  Synthetic Peptide
MGGKLSKKKKGYNVNDEKAKEKDKKAEGAATEEEGTPKESEPQAAAEPAEAKEGKEKPDQDAEGKAEEKEGEKDAAAAKEEAPKAEPEKTEGAAEAKAEPPKAPEQEQAAPGPAAGGEAPKAAEAAAAPAESAAPAAGEEPSKEEGEPKKTEAPAAPAAQETKSDGAPASDSKPGSSEAAPSSKETPAATEAPSSTPKAQGPAASAEEPKPVEAPAANSDQTVTVKE

SEQ ID NO: 11 - protein, 4 aa, Artificial Sequence
  Synthetic Peptide
KKKK

SEQ ID NO: 12 - protein, 5 aa, Artificial Sequence
  Synthetic Peptide
KKKKK

SEQ ID NO: 13 - protein, 4 aa, Artificial Sequence
  Synthetic Peptide
RRRR

SEQ ID NO: 14 - protein, 5 aa, Artificial Sequence
  Synthetic Peptide
RRRRR

SEQ ID NO: 15 - protein, 4 aa, Artificial Sequence
  Synthetic Peptide
  misc_feature (1)..(1): Lys  may be  Arg
  misc_feature (2)..(2): Lys  may be  Arg
  misc_feature (3)..(3): Lys  may be  Arg
  misc_feature (4)..(4): Lys  may be  Arg
KKKK

SEQ ID NO: 16 - protein, 5 aa, Artificial Sequence
  Synthetic Peptide
  misc_feature (1)..(1): Lys  may be  Arg
  misc_feature (2)..(2): Lys  may be  Arg
  misc_feature (3)..(3): Lys  may be  Arg
  misc_feature (4)..(4): Lys  may be  Arg
  misc_feature (5)..(5): Lys  may be  Arg
KKKKK

SEQ ID NO: 17 - protein, 7 aa, Artificial Sequence
  Synthetic Peptide
GGKLSKK

SEQ ID NO: 18 - protein, 7 aa, Artificial Sequence
  Synthetic Peptide
GAKLSKK

SEQ ID NO: 19 - protein, 7 aa, Artificial Sequence
  Synthetic Peptide
GGKQSKK

SEQ ID NO: 20 - protein, 7 aa, Artificial Sequence
  Synthetic Peptide
GGKLAKK

SEQ ID NO: 21 - protein, 7 aa, Artificial Sequence
  Synthetic Peptide
GGKLSKK

SEQ ID NO: 22 - protein, 8 aa, Artificial Sequence
  Synthetic Peptide
GGKLSKKK

SEQ ID NO: 23 - protein, 8 aa, Artificial Sequence
  Synthetic Peptide
GGKLSKKS

SEQ ID NO: 24 - protein, 8 aa, Artificial Sequence
  Synthetic Peptide
GAKLSKKK

SEQ ID NO: 25 - protein, 8 aa, Artificial Sequence
  Synthetic Peptide
GAKLSKKS

SEQ ID NO: 26 - protein, 8 aa, Artificial Sequence
  Synthetic Peptide
GGKQSKKK

SEQ ID NO: 27 - protein, 8 aa, Artificial Sequence
  Synthetic Peptide
GGKQSKKS

SEQ ID NO: 28 - protein, 8 aa, Artificial Sequence
  Synthetic Peptide
GGKLAKKK

SEQ ID NO: 29 - protein, 8 aa, Artificial Sequence
  Synthetic Peptide
GGKLAKKS

SEQ ID NO: 30 - protein, 8 aa, Artificial Sequence
  Synthetic Peptide
GGKLSKKK

SEQ ID NO: 31 - protein, 8 aa, Artificial Sequence
  Synthetic Peptide
GGKLSKKS

SEQ ID NO: 32 - protein, 14 aa, Artificial Sequence
  Synthetic Peptide
GGKLSKKKKGYNVN

SEQ ID NO: 33 - protein, 14 aa, Artificial Sequence
  Synthetic Peptide
GAKLSKKKKGYNVN

SEQ ID NO: 34 - protein, 14 aa, Artificial Sequence
  Synthetic Peptide
GGKQSKKKKGYNVN

SEQ ID NO: 35 - protein, 14 aa, Artificial Sequence
  Synthetic Peptide
GGKLAKKKKGYNVN

SEQ ID NO: 36 - protein, 14 aa, Artificial Sequence
  Synthetic Peptide
GGKLSKKKKGYSGG

SEQ ID NO: 37 - protein, 14 aa, Artificial Sequence
  Synthetic Peptide
GGKLSKKKKGSGGS

SEQ ID NO: 38 - protein, 14 aa, Artificial Sequence
  Synthetic Peptide
GGKLSKKKKSGGSG

SEQ ID NO: 39 - protein, 14 aa, Artificial Sequence
  Synthetic Peptide
GGKLSKKKSGGSGG

SEQ ID NO: 40 - protein, 14 aa, Artificial Sequence
  Synthetic Peptide
GGKLSKKSGGSGGS

SEQ ID NO: 41 - protein, 14 aa, Artificial Sequence
  Synthetic Peptide
GGKLSKSGGSGGSV

SEQ ID NO: 42 - protein, 14 aa, Artificial Sequence
  Synthetic Peptide
GAKKSKKRFSFKKS

SEQ ID NO: 43 - protein, 7 aa, Artificial Sequence
  Synthetic Peptide
GTTTQSR

SEQ ID NO: 44 - protein, 735 aa, Artificial Sequence [sequence omitted]

SEQ ID NO: 45 - protein, 653 aa, Artificial Sequence
  Synthetic Peptide
MADTLPSEFDVIVIGTGLPESIIAAACSRSGRRVLHVDSRSYYGGNWASFSFSGLLSWLKEYQENSDIVSDSPVWQDQILENEEAIALSRKDKTIQHVEVFCYASQDLHEDVEEAGALQKNHALVTSANSTEAADSAFLPTEDESLSTMSCEMLTEQTPSSDPENALEVNGAEVTGEKENHCDDKTCVPSTSAEDMSENVPIAEDTTEQPKKNRITYSQIIKEGRRFNIDLVSKLLYSRGLLIDLLIKSNVSRYAEFKNITRILAFREGRVEQVPCSRADVFNSKQLTMVEKRMLMKFLTFCMEYEKYPDEYKGYEEITFYEYLKTQKLTPNLQYIVMHSIAMTSETASSTIDGLKATKNFLHCLGRYGNTPFLFPLYGQGELPQCFCRMCAVFGGIYCLRHSVQCLVVDKESRKCKAIIDQFGQRIISEHFLVEDSYFPENMCSRVQYRQISRAVLITDRSVLKTDSDQQISILTVPAEEPGTFAVRVIELCSSTMTCMKGTYLVHLTCTSSKTAREDLESVVQKLFVPYTEMEIENEQVEKPRILWALYFNMRDSSDISRSCYNDLPSNVYVCSGPDCGLGNDNAVKQAETLFQEICPNEDFCPPPPNPEDIILDGDSLQPEASESSAIPEANSETFKESTNLGNLEESSE

SEQ ID NO: 46 - protein, 2 aa, Artificial Sequence
  Synthetic Peptide
  misc_feature (1)..(1): Wherein  Gly may be  repeated  from an integer 1 to 100
  misc_feature (1)..(2): Wherein  the sequence may be repeated by any integer from 1 to 100
GS

SEQ ID NO: 47 - protein, 2 aa, Artificial Sequence
  Synthetic Peptide
  misc_feature (1)..(1): Wherein Gly is  repeated  from any  integer from 1 to 4
  misc_feature (2)..(2): Wherein Ser  may be repeated  0  or 1
  misc_feature (3)..(3): Wherein the sequence may  be repeated by an integer between 1 to 50 times
GS

SEQ ID NO: 48 - protein, 1 aa, Artificial Sequence
  Synthetic Peptide
  misc_feature (1)..(1): Wherein Gly may be repeated by an integer from 1 to 100
G

SEQ ID NO: 49 - protein, 2 aa, Artificial Sequence
  Synthetic Peptide
  misc_feature (1)..(2): Wherein the sequence may be repeated by 1 to 100
GA

SEQ ID NO: 50 - protein, 3 aa, Artificial Sequence
  Synthetic Peptide
  misc_feature (1)..(3): wherein the sequence may be repeated by an integer between 1 and 100
GGS

SEQ ID NO: 51 - protein, 6 aa, Artificial Sequence
  Synthetic Peptide
GGKLSK

SEQ ID NO: 52 - protein, 6 aa, Artificial Sequence
  Synthetic Peptide
GAKLSK

SEQ ID NO: 53 - protein, 6 aa, Artificial Sequence
  Synthetic Peptide
GGKQSK

SEQ ID NO: 54 - protein, 6 aa, Artificial Sequence
  Synthetic Peptide
GGKLAK

SEQ ID NO: 55 - protein, 4 aa, Artificial Sequence
  Synthetic Peptide
KKKG

SEQ ID NO: 56 - protein, 5 aa, Artificial Sequence
  Synthetic Peptide
KKKGY

SEQ ID NO: 57 - protein, 6 aa, Artificial Sequence
  Synthetic Peptide
KKKGYN

SEQ ID NO: 58 - protein, 7 aa, Artificial Sequence
  Synthetic Peptide
KKKGYNV

SEQ ID NO: 59 - protein, 8 aa, Artificial Sequence
  Synthetic Peptide
KKKGYNVN

SEQ ID NO: 60 - protein, 6 aa, Artificial Sequence
  Synthetic Peptide
KKKGYS

SEQ ID NO: 61 - protein, 6 aa, Artificial Sequence
  Synthetic Peptide
KKKGYG

SEQ ID NO: 62 - protein, 7 aa, Artificial Sequence
  Synthetic Peptide
KKKGYGG

SEQ ID NO: 63 - protein, 5 aa, Artificial Sequence
  Synthetic Peptide
KKKGS

SEQ ID NO: 64 - protein, 6 aa, Artificial Sequence
  Synthetic Peptide
KKKGSG

SEQ ID NO: 65 - protein, 6 aa, Artificial Sequence
  Synthetic Peptide
KKKGSG

SEQ ID NO: 66 - protein, 7 aa, Artificial Sequence
  Synthetic Peptide
KKKGSGS

SEQ ID NO: 67 - protein, 4 aa, Artificial Sequence
  Synthetic Peptide
KKKS

SEQ ID NO: 68 - protein, 5 aa, Artificial Sequence
  Synthetic Peptide
KKKSG

SEQ ID NO: 69 - protein, 6 aa, Artificial Sequence
  Synthetic Peptide
KKKSGG

SEQ ID NO: 70 - protein, 7 aa, Artificial Sequence
  Synthetic Peptide
KKKSGGS

SEQ ID NO: 71 - protein, 8 aa, Artificial Sequence
  Synthetic Peptide
KKKSGGSG

SEQ ID NO: 72 - protein, 8 aa, Artificial Sequence
  Synthetic Peptide
KKSGGSGG

SEQ ID NO: 73 - protein, 10 aa, Artificial Sequence
  Synthetic Peptide
KKKSGGSGGS

SEQ ID NO: 74 - protein, 8 aa, Artificial Sequence
  Synthetic Peptide
KRFSFKKS

SEQ ID NO: 75 - protein, 29 aa, Artificial Sequence
  Synthetic Peptide
GGKLSKKKKGYNVNDEKAKEKDKKAEGAA

SEQ ID NO: 76 - protein, 28 aa, Artificial Sequence
  Synthetic Peptide
GGKLSKKKKGYNVNDEKAKEKDKKAEGA

SEQ ID NO: 77 - protein, 27 aa, Artificial Sequence
  Synthetic Peptide
GGKLSKKKKGYNVNDEKAKEKDKKAEG

SEQ ID NO: 78 - protein, 26 aa, Artificial Sequence
  Synthetic Peptide
GGKLSKKKKGYNVNDEKAKEKDKKAE

SEQ ID NO: 79 - protein, 25 aa, Artificial Sequence
  Synthetic Peptide
GGKLSKKKKGYNVNDEKAKEKDKKA

SEQ ID NO: 80 - protein, 24 aa, Artificial Sequence
  Synthetic Peptide
GGKLSKKKKGYNVNDEKAKEKDKK

SEQ ID NO: 81 - protein, 23 aa, Artificial Sequence
  Synthetic Peptide
GGKLSKKKKGYNVNDEKAKEKDK

SEQ ID NO: 82 - protein, 22 aa, Artificial Sequence
  Synthetic Peptide
GGKLSKKKKGYNVNDEKAKEKD

SEQ ID NO: 83 - protein, 21 aa, Artificial Sequence
  Synthetic Peptide
GGKLSKKKKGYNVNDEKAKEK

SEQ ID NO: 84 - protein, 20 aa, Artificial Sequence
  Synthetic Peptide
GGKLSKKKKGYNVNDEKAKE

SEQ ID NO: 85 - protein, 19 aa, Artificial Sequence
  Synthetic Peptide
GGKLSKKKKGYNVNDEKAK

SEQ ID NO: 86 - protein, 18 aa, Artificial Sequence
  Synthetic Peptide
GGKLSKKKKGYNVNDEKA

SEQ ID NO: 87 - protein, 17 aa, Artificial Sequence
  Synthetic Peptide
GGKLSKKKKGYNVNDEK

SEQ ID NO: 88 - protein, 16 aa, Artificial Sequence
  Synthetic Peptide
GGKLSKKKKGYNVNDE

SEQ ID NO: 89 - protein, 15 aa, Artificial Sequence
  Synthetic Peptide
GGKLSKKKKGYNVND

SEQ ID NO: 90 - protein, 13 aa, Artificial Sequence
  Synthetic Peptide
GGKLSKKKKGYNV

SEQ ID NO: 91 - protein, 12 aa, Artificial Sequence
  Synthetic Peptide
GGKLSKKKKGYN

SEQ ID NO: 92 - protein, 11 aa, Artificial Sequence
  Synthetic Peptide
GGKLSKKKKGY

SEQ ID NO: 93 - protein, 10 aa, Artificial Sequence
  Synthetic Peptide
GGKLSKKKKG

SEQ ID NO: 94 - protein, 9 aa, Artificial Sequence
  Synthetic Peptide
GGKLSKKKK

SEQ ID NO: 95 - protein, 29 aa, Artificial Sequence
  Synthetic Peptide
GAKKSKKRFSFKKSFKLSGFSFKKNKKEA

SEQ ID NO: 96 - protein, 28 aa, Artificial Sequence
  Synthetic Peptide
GAKKSKKRFSFKKSFKLSGFSFKKNKKE

SEQ ID NO: 97 - protein, 27 aa, Artificial Sequence
  Synthetic Peptide
GAKKSKKRFSFKKSFKLSGFSFKKNKK

SEQ ID NO: 98 - protein, 26 aa, Artificial Sequence
  Synthetic Peptide
GAKKSKKRFSFKKSFKLSGFSFKKNK

SEQ ID NO: 99 - protein, 25 aa, Artificial Sequence
  Synthetic Peptide
GAKKSKKRFSFKKSFKLSGFSFKKN

SEQ ID NO: 100 - protein, 24 aa, Artificial Sequence
  Synthetic Peptide
GAKKSKKRFSFKKSFKLSGFSFKK

SEQ ID NO: 101 - protein, 23 aa, Artificial Sequence
  Synthetic Peptide
GAKKSKKRFSFKKSFKLSGFSFK

SEQ ID NO: 102 - protein, 22 aa, Artificial Sequence
  Synthetic Peptide
GAKKSKKRFSFKKSFKLSGFSF

SEQ ID NO: 103 - protein, 21 aa, Artificial Sequence
  Synthetic Peptide
GAKKSKKRFSFKKSFKLSGFS

SEQ ID NO: 104 - protein, 20 aa, Artificial Sequence
  Synthetic Peptide
GAKKSKKRFSFKKSFKLSGF

SEQ ID NO: 105 - protein, 19 aa, Artificial Sequence
  Synthetic Peptide
GAKKSKKRFSFKKSFKLSG

SEQ ID NO: 106 - protein, 18 aa, Artificial Sequence
  Synthetic Peptide
GAKKSKKRFSFKKSFKLS

SEQ ID NO: 107 - protein, 17 aa, Artificial Sequence
  Synthetic Peptide
GAKKSKKRFSFKKSFKL

SEQ ID NO: 108 - protein, 16 aa, Artificial Sequence
  Synthetic Peptide
GAKKSKKRFSFKKSFK

SEQ ID NO: 109 - protein, 15 aa, Artificial Sequence
  Synthetic Peptide
GAKKSKKRFSFKKSF

SEQ ID NO: 110 - protein, 13 aa, Artificial Sequence
  Synthetic Peptide
GAKKSKKRFSFKK

SEQ ID NO: 111 - protein, 12 aa, Artificial Sequence
  Synthetic Peptide
GAKKSKKRFSFK

SEQ ID NO: 112 - protein, 11 aa, Artificial Sequence
  Synthetic Peptide
GAKKSKKRFSF

SEQ ID NO: 113 - protein, 10 aa, Artificial Sequence
  Synthetic Peptide
GAKKSKKRFS

SEQ ID NO: 114 - protein, 9 aa, Artificial Sequence
  Synthetic Peptide
GAKKSKKRF

SEQ ID NO: 115 - protein, 8 aa, Artificial Sequence
  Synthetic Peptide
GAKKSKKR

SEQ ID NO: 116 - protein, 7 aa, Artificial Sequence
  Synthetic Peptide
GAKKSKK

SEQ ID NO: 117 - protein, 29 aa, Artificial Sequence
  Synthetic Peptide
GAKKAKKRFSFKKSFKLSGFSFKKNKKEA

SEQ ID NO: 118 - protein, 28 aa, Artificial Sequence
  Synthetic Peptide
GAKKAKKRFSFKKSFKLSGFSFKKNKKE

SEQ ID NO: 119 - protein, 27 aa, Artificial Sequence
  Synthetic Peptide
GAKKAKKRFSFKKSFKLSGFSFKKNKK

SEQ ID NO: 120 - protein, 26 aa, Artificial Sequence
  Synthetic Peptide
GAKKAKKRFSFKKSFKLSGFSFKKNK

SEQ ID NO: 121 - protein, 25 aa, Artificial Sequence
  Synthetic Peptide
GAKKAKKRFSFKKSFKLSGFSFKKN

SEQ ID NO: 122 - protein, 24 aa, Artificial Sequence
  Synthetic Peptide
GAKKAKKRFSFKKSFKLSGFSFKK

SEQ ID NO: 123 - protein, 23 aa, Artificial Sequence
  Synthetic Peptide
GAKKAKKRFSFKKSFKLSGFSFK

SEQ ID NO: 124 - protein, 22 aa, Artificial Sequence
  Synthetic Peptide
GAKKAKKRFSFKKSFKLSGFSF

SEQ ID NO: 125 - protein, 21 aa, Artificial Sequence
  Synthetic Peptide
GAKKAKKRFSFKKSFKLSGFS

SEQ ID NO: 126 - protein, 20 aa, Artificial Sequence
  Synthetic Peptide
GAKKAKKRFSFKKSFKLSGF

SEQ ID NO: 127 - protein, 19 aa, Artificial Sequence
  Synthetic Peptide
GAKKAKKRFSFKKSFKLSG

SEQ ID NO: 128 - protein, 18 aa, Artificial Sequence
  Synthetic Peptide
GAKKAKKRFSFKKSFKLS

SEQ ID NO: 129 - protein, 17 aa, Artificial Sequence
  Synthetic Peptide
GAKKAKKRFSFKKSFKL

SEQ ID NO: 130 - protein, 16 aa, Artificial Sequence
  Synthetic Peptide
GAKKAKKRFSFKKSFK

SEQ ID NO: 131 - protein, 15 aa, Artificial Sequence
  Synthetic Peptide
GAKKAKKRFSFKKSF

SEQ ID NO: 132 - protein, 14 aa, Artificial Sequence
  Synthetic Peptide
GAKKAKKRFSFKKS

SEQ ID NO: 133 - protein, 13 aa, Artificial Sequence
  Synthetic Peptide
GAKKAKKRFSFKK

SEQ ID NO: 134 - protein, 12 aa, Artificial Sequence
  Synthetic Peptide
GAKKAKKRFSFK

SEQ ID NO: 135 - protein, 11 aa, Artificial Sequence
  Synthetic Peptide
GAKKAKKRFSF

SEQ ID NO: 136 - protein, 10 aa, Artificial Sequence
  Synthetic Peptide
GAKKAKKRFS

SEQ ID NO: 137 - protein, 9 aa, Artificial Sequence
  Synthetic Peptide
GAKKAKKRF

SEQ ID NO: 138 - protein, 8 aa, Artificial Sequence
  Synthetic Peptide
GAKKAKKR

SEQ ID NO: 139 - protein, 7 aa, Artificial Sequence
  Synthetic Peptide
GAKKAKK

SEQ ID NO: 140 - protein, 28 aa, Artificial Sequence
  Synthetic Peptide
GAQESKKKKKKRFSFKKSFKLSGFSFKK

SEQ ID NO: 141 - protein, 27 aa, Artificial Sequence
  Synthetic Peptide
GAQESKKKKKKRFSFKKSFKLSGFSFK

SEQ ID NO: 142 - protein, 26 aa, Artificial Sequence
  Synthetic Peptide
GAQESKKKKKKRFSFKKSFKLSGFSF

SEQ ID NO: 143 - protein, 25 aa, Artificial Sequence
  Synthetic Peptide
GAQESKKKKKKRFSFKKSFKLSGFS

SEQ ID NO: 144 - protein, 24 aa, Artificial Sequence
  Synthetic Peptide
GAQESKKKKKKRFSFKKSFKLSGF

SEQ ID NO: 145 - protein, 23 aa, Artificial Sequence
  Synthetic Peptide
GAQESKKKKKKRFSFKKSFKLSG

SEQ ID NO: 146 - protein, 22 aa, Artificial Sequence
  Synthetic Peptide
GAQESKKKKKKRFSFKKSFKLS

SEQ ID NO: 147 - protein, 21 aa, Artificial Sequence
  Synthetic Peptide
GAQESKKKKKKRFSFKKSFKL

SEQ ID NO: 148 - protein, 20 aa, Artificial Sequence
  Synthetic Peptide
GAQESKKKKKKRFSFKKSFK

SEQ ID NO: 149 - protein, 19 aa, Artificial Sequence
  Synthetic Peptide
GAQESKKKKKKRFSFKKSF

SEQ ID NO: 150 - protein, 18 aa, Artificial Sequence
  Synthetic Peptide
GAQESKKKKKKRFSFKKS

SEQ ID NO: 151 - protein, 17 aa, Artificial Sequence
  Synthetic Peptide
GAQESKKKKKKRFSFKK

SEQ ID NO: 152 - protein, 16 aa, Artificial Sequence
  Synthetic Peptide
GAQESKKKKKKRFSFK

SEQ ID NO: 153 - protein, 15 aa, Artificial Sequence
  Synthetic Peptide
GAQESKKKKKKRFSF

SEQ ID NO: 154 - protein, 14 aa, Artificial Sequence
  Synthetic Peptide
GAQESKKKKKKRFS

SEQ ID NO: 155 - protein, 13 aa, Artificial Sequence
  Synthetic Peptide
GAQESKKKKKKRF

SEQ ID NO: 156 - protein, 12 aa, Artificial Sequence
  Synthetic Peptide
GAQESKKKKKKR

SEQ ID NO: 157 - protein, 11 aa, Artificial Sequence
  Synthetic Peptide
GAQESKKKKKK

SEQ ID NO: 158 - protein, 10 aa, Artificial Sequence
  Synthetic Peptide
GAQESKKKKK

SEQ ID NO: 159 - protein, 9 aa, Artificial Sequence
  Synthetic Peptide
GAQESKKKK

SEQ ID NO: 160 - protein, 8 aa, Artificial Sequence
  Synthetic Peptide
GAQESKKK

SEQ ID NO: 161 - protein, 7 aa, Artificial Sequence
  Synthetic Peptide
GAQESKK

SEQ ID NO: 162 - protein, 30 aa, Artificial Sequence
  Synthetic Peptide
GSQSSKKKKKKFSFKKPFKLSGLSFKRNRK

SEQ ID NO: 163 - protein, 29 aa, Artificial Sequence
  Synthetic Peptide
GSQSSKKKKKKFSFKKPFKLSGLSFKRNR

SEQ ID NO: 164 - protein, 28 aa, Artificial Sequence
  Synthetic Peptide
GSQSSKKKKKKFSFKKPFKLSGLSFKRN

SEQ ID NO: 165 - protein, 27 aa, Artificial Sequence
  Synthetic Peptide
GSQSSKKKKKKFSFKKPFKLSGLSFKR

SEQ ID NO: 166 - protein, 26 aa, Artificial Sequence
  Synthetic Peptide
GSQSSKKKKKKFSFKKPFKLSGLSFK

SEQ ID NO: 167 - protein, 25 aa, Artificial Sequence
  Synthetic Peptide
GSQSSKKKKKKFSFKKPFKLSGLSF

SEQ ID NO: 168 - protein, 24 aa, Artificial Sequence
  Synthetic Peptide
GSQSSKKKKKKFSFKKPFKLSGLS

SEQ ID NO: 169 - protein, 23 aa, Artificial Sequence
  Synthetic Peptide
GSQSSKKKKKKFSFKKPFKLSGL

SEQ ID NO: 170 - protein, 22 aa, Artificial Sequence
  Synthetic Peptide
GSQSSKKKKKKFSFKKPFKLSG

SEQ ID NO: 171 - protein, 21 aa, Artificial Sequence
  Synthetic Peptide
GSQSSKKKKKKFSFKKPFKLS

SEQ ID NO: 172 - protein, 20 aa, Artificial Sequence
  Synthetic Peptide
GSQSSKKKKKKFSFKKPFKL

SEQ ID NO: 173 - protein, 19 aa, Artificial Sequence
  Synthetic Peptide
GSQSSKKKKKKFSFKKPFK

SEQ ID NO: 174 - protein, 18 aa, Artificial Sequence
  Synthetic Peptide
GSQSSKKKKKKFSFKKPF

SEQ ID NO: 175 - protein, 17 aa, Artificial Sequence
  Synthetic Peptide
GSQSSKKKKKKFSFKKP

SEQ ID NO: 176 - protein, 16 aa, Artificial Sequence
  Synthetic Peptide
GSQSSKKKKKKFSFKK

SEQ ID NO: 177 - protein, 15 aa, Artificial Sequence
  Synthetic Peptide
GSQSSKKKKKKFSFK

SEQ ID NO: 178 - protein, 14 aa, Artificial Sequence
  Synthetic Peptide
GSQSSKKKKKKFSF

SEQ ID NO: 179 - protein, 13 aa, Artificial Sequence
  Synthetic Peptide
GSQSSKKKKKKFS

SEQ ID NO: 180 - protein, 12 aa, Artificial Sequence
  Synthetic Peptide
GSQSSKKKKKKF

SEQ ID NO: 181 - protein, 11 aa, Artificial Sequence
  Synthetic Peptide
GSQSSKKKKKK

SEQ ID NO: 182 - protein, 10 aa, Artificial Sequence
  Synthetic Peptide
GSQSSKKKKK

SEQ ID NO: 183 - protein, 9 aa, Artificial Sequence
  Synthetic Peptide
GSQSSKKKK

SEQ ID NO: 184 - protein, 8 aa, Artificial Sequence
  Synthetic Peptide
GSQSSKKK

SEQ ID NO: 185 - protein, 7 aa, Artificial Sequence
  Synthetic Peptide
GSQSSKK

SEQ ID NO: 186 - protein, 731 aa, Artificial Sequence
  Synthetic Peptide
PSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 187 - protein, 611 aa, Artificial Sequence
  Synthetic Peptide
VATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 188 - protein, 485 aa, Artificial Sequence
  Synthetic Peptide
SPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 189 - protein, 343 aa, Artificial Sequence
  Synthetic Peptide
KPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 190 - protein, 217 aa, Artificial Sequence
  Synthetic Peptide
VRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 191 - protein, 66 aa, Artificial Sequence
  Synthetic Peptide
SKPVFITVKMDVLNAFKYPLLIGVGLSTVIGLLSCLIGYCSSHWCCKKEVQETRRERRRLMSMEMD

SEQ ID NO: 192 - protein, 21 aa, Artificial Sequence
  Synthetic Peptide
MGRLASRPLLLALLSLALCRG

SEQ ID NO: 193 - protein, 247 aa, Artificial Sequence
  Synthetic Peptide
PGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 194 - protein, 168 aa, Artificial Sequence
  Synthetic Peptide
HGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 195 - protein, 66 aa, Artificial Sequence
  Synthetic Peptide
SHLAALWPFLGIVAEVLVLVTIIFIYEKRRKPEDVLDDDDAGSAPLKSSGQHQNDKGKNVRQRNSS

SEQ ID NO: 196 - protein, 18 aa, Artificial Sequence
  Synthetic Peptide
MAAALFVLLGFALLGTHG

SEQ ID NO: 197 - protein, 456 aa, Artificial Sequence
  Synthetic Peptide
APPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 198 - protein, 320 aa, Artificial Sequence
  Synthetic Peptide
AHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 199 - protein, 179 aa, Artificial Sequence
  Synthetic Peptide
REEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 200 - protein, 24 aa, Artificial Sequence
  Synthetic Peptide
VALVTGATVLGTITCCFMKRLRKR

SEQ ID NO: 201 - protein, 27 aa, Artificial Sequence
  Synthetic Peptide
MGALRPTLLPPSLPLLLLLMLGMGCWA

SEQ ID NO: 202 - protein, 1021 aa, Artificial Sequence
  Synthetic Peptide
MAGISYVASFFLLLTKLSIGQREVTVQKGPLFRAEGYPVSIGCNVTGHQGPSEQHFQWSVYLPTNPTQEVQIISTKDAAFSYAVYTQRVRSGDVYVERVQGNSVLLHISKLQMKDAGEYECHTPNTDEKYYGSYSAKTNLIVIPDTLSATMSSQTLGKEEGEPLALTCEASKATAQHTHLSVTWYLTQDGGGSQATEIISLSKDFILVPGPLYTERFAASDVQLNKLGPTTFRLSIERLQSSDQGQLFCEATEWIQDPDETWMFITKKQTDQTTLRIQPAVKDFQVNITADSLFAEGKPLELVCLVVSSGRDPQLQGIWFFNGTEIAHIDAGGVLGLKNDYKERASQGELQVSKLGPKAFSLKIFSLGPEDEGAYRCVVAEVMKTRTGSWQVLQRKQSPDSHVHLRKPAARSVVMSTKNKQQVVWEGETLAFLCKAGGAESPLSVSWWHIPRDQTQPEFVAGMGQDGIVQLGASYGVPSYHGNTRLEKMDWATFQLEITFTAITDSGTYECRVSEKSRNQARDLSWTQKISVTVKSLESSLQVSLMSRQPQVMLTNTFDLSCVVRAGYSDLKVPLTVTWQFQPASSHIFHQLIRITHNGTIEWGNFLSRFQKKTKVSQSLFRSQLLVHDATEEETGVYQCEVEVYDRNSLYNNRPPRASAISHPLRIAVTLPESKLKVNSRSQVQELSINSNTDIECSILSRSNGNLQLAIIWYFSPVSTNASWLKILEMDQTNVIKTGDEFHTPQRKQKFHTEKVSQDLFQLHILNVEDSDRGKYHCAVEEWLLSTNGTWHKLGEKKSGLTELKLKPTGSKVRVSKVYWTENVTEHREVAIRCSLESVGSSATLYSVMWYWNRENSGSKLLVHLQHDGLLEYGEEGLRRHLHCYRSSSTDFVLKLHQVEMEDAGMYWCRVAEWQLHGHPSKWINQASDESQRMVLTVLPSEPTLPSRICSSAPLLYFLFICPFVLLLLLLISLLCLYWKARKLSTLRSNTRKEKALWVDLKEAGGVTTNRREDEEEDEGN

SEQ ID NO: 203 - protein, 1195 aa, Artificial Sequence
  Synthetic Peptide
MKCFFPVLSCLAVLGVVSAQRQVTVQEGPLYRTEGSHITIWCNVSGYQGPSEQNFQWSIYLPSSPEREVQIVSTMDSSFPYAIYTQRVRGGKIFIERVQGNSTLLHITDLQARDAGEYECHTPSTDKQYFGSYSAKMNLVVIPDSLQTTAMPQTLHRVEQDPLELTCEVASETIQHSHLSVAWLRQKVGEKPVEVISLSRDFMLHSSSEYAQRQSLGEVRLDKLGRTTFRLTIFHLQPSDQGEFYCEAAEWIQDPDGSWYAMTRKRSEGAVVNVQPTDKEFTVRLETEKRLHTVGEPVEFRCILEAQNVPDRYFAVSWAFNSSLIATMGPNAVPVLNSEFAHREARGQLKVAKESDSVFVLKIYHLRQEDSGKYNCRVTEREKTVTGEFIDKESKRPKNIPIIVLPLKSSISVEVASNASVILEGEDLRFSCSVRTAGRPQGRFSVIWQLVDRQNRRSNIMWLDRDGTVQPGSSYWERSSFGGVQMEQVQPNSFSLGIFNSRKEDEGQYECHVTEWVRAVDGEWQIVGERRASTPISITALEMGFAVTAISRTPGVTYSDSFDLQCIIKPHYPAWVPVSVTWRFQPVGTVEFHDLVTFTRDGGVQWGDRSSSFRTRTAIEKAESSNNVRLSISRASDTEAGKYQCVAELWRKNYNNTWTRLAERTSNLLEIRVLQPVTKLQVSKSKRTLTLVENKPIQLNCSVKSQTSQNSHFAVLWYVHKPSDADGKLILKTTHNSAFEYGTYAEEEGLRARLQFERHVSGGLFSLTVQRAEVSDSGSYYCHVEEWLLSPNYAWYKLAEEVSGRTEVTVKQPDSRLRLSQAQGNLSVLETRQVQLECVVLNRTSITSQLMVEWFVWKPNHPERETVARLSRDATFHYGEQAAKNNLKGRLHLESPSPGVYRLFIQNVAVQDSGTYSCHVEEWLPSPSGMWYKRAEDTAGQTALTVMRPDASLQVDTVVPNATVSEKAAFQLDCSIVSRSSQDSRFAVAWYSLRTKAGGKRSSPGLEEQEEEREEEEEEEEDDDDDDPTERTALLSVGPDAVFGPEGSPWEGRLRFQRLSPVLYRLTVLQASPQDTGNYSCHVEEWLPSPQKEWYRLTEEESAPIGIRVLDTSPTLQSIICSNDALFYFVFFYPFPIFGILIITILLVRFKSRNSSKNSDGKNGVPLLWIKEPHLNYSPTCLEPPVLSIHPGAID

SEQ ID NO: 204 - protein, 1023 aa, Artificial Sequence
  Synthetic Peptide
MGKGVGRDKYEPAAVSEQGDKKGKKGKKDRDMDELKKEVSMDDHKLSLDELHRKYGTDLSRGLTSARAAEILARDGPNALTPPPTTPEWIKFCRQLFGGFSMLLWIGAILCFLAYSIQAATEEEPQNDNLYLGVVLSAVVIITGCFSYYQEAKSSKIMESFKNMVPQQALVIRNGEKMSINAEEVVVGDLVEVKGGDRIPADLRIISANGCKVDNSSLTGESEPQTRSPDFTNENPLETRNIAFFSTNCVEGTARGIVVYTGDRTVMGRIATLASGLEGGQTPIAAEIEHFIHIITGVAVFLGVSFFILSLILEYTWLEAVIFLIGIIVANVPEGLLATVTVCLTLTAKRMARKNCLVKNLEAVETLGSTSTICSDKTGTLTQNRMTVAHMWFDNQIHEADTTENQSGVSFDKTSATWLALSRIAGLCNRAVFQANQENLPILKRAVAGDASESALLKCIELCCGSVKEMRERYAKIVEIPFNSTNKYQLSIHKNPNTSEPQHLLVMKGAPERILDRCSSILLHGKEQPLDEELKDAFQNAYLELGGLGERVLGFCHLFLPDEQFPEGFQFDTDDVNFPIDNLCFVGLISMIDPPRAAVPDAVGKCRSAGIKVIMVTGDHPITAKAIAKGVGIISEGNETVEDIAARLNIPVSQVNPRDAKACVVHGSDLKDMTSEQLDDILKYHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLIFIIANIPLPLGTVTILCIDLGTDMVPAISLAYEQAESDIMKRQPRNPKTDKLVNERLISMAYGQIGMIQALGGFFTYFVILAENGFLPIHLLGLRVDWDDRWINDVEDSYGQQWTYEQRKIVEFTCHTAFFVSIVVVQWADLVICKTRRNSVFQQGMKNKILIFGLFEETALAAFLSYCPGMGVALRMYPLKPTWWFCAFPYSLLIFVYDEVRKLIIRRRPGGWVEKETYY

SEQ ID NO: 205 - protein, 240 aa, Artificial Sequence
  Synthetic Peptide
MGRGAGREYSPAATTAENGGGKKKQKEKELDELKKEVAMDDHKLSLDELGRKYQVDLSKGLTNQRAQDVLARDGPNALTPPPTTPEWVKFCRQLFGGFSILLWIGAILCFLAYGIQAAMEDEPSNDNLYLGVVLAAVVIVTGCFSYYQEAKSSKIMDSFKNMVPQQALVIREGEKMQINAEEVVVGDLVEVKGGDRVPADLRIISSHGCKVDNSSLTGESEPQTRSPEFTHENPLETRNI

SEQ ID NO: 206 - protein, 780 aa, Artificial Sequence
  Synthetic Peptide
CFFSTNCVEGTARGIVIATGDRTVMGRIATLASGLEVGRTPIAMEIEHFIQLITGVAVFLGVSFFVLSLILGYSWLEAVIFLIGIIVANVPEGLLATVTVCLTLTAKRMARKNCLVKNLEAVETLGSTSTICSDKTGTLTQNRMTVAHMWFDNQIHEADTTEDQSGATFDKRSPTWTALSRIAGLCNRAVFKAGQENISVSKRDTAGDASESALLKCIELSCGSVRKMRDRNPKVAEIPFNSTNKYQLSIHEREDSPQSHVLVMKGAPERILDRCSTILVQGKEIPLDKEMQDAFQNAYMELGGLGERVLGFCQLNLPSGKFPRGFKFDTDELNFPTEKLCFVGLMSMIDPPRAAVPDAVGKCRSAGIKVIMVTGDHPITAKAIAKGVGIISEGNETVEDIAARLNIPMSQVNPREAKACVVHGSDLKDMTSEQLDEILKNHTEIVFARTSPQQKLIIVEGCQRQGAIVAVTGDGVNDSPALKKADIGIAMGISGSDVSKQAADMILLDDNFASIVTGVEEGRLIFDNLKKSIAYTLTSNIPEITPFLLFIIANIPLPLGTVTILCIDLGTDMVPAISLAYEAAESDIMKRQPRNSQTDKLVNERLISMAYGQIGMIQALGGFFTYFVILAENGFLPSRLLGIRLDWDDRTMNDLEDSYGQEWTYEQRKVVEFTCHTAFFASIVVVQWADLIICKTRRNSVFQQGMKNKILIFGLLEETALAAFLSYCPGMGVALRMYPLKVTWWFCAFPYSLLIFIYDEVRKLILRRYPGGWVEKETYY

SEQ ID NO: 207 - protein, 1026 aa, Artificial Sequence
  Synthetic Peptide
MGSGGSDSYRIATSQDKKDDKDSPKKNKGKERRDLDDLKKEVAMTEHKMSVEEVCRKYNTDCVQGLTHSKAQEILARDGPNALTPPPTTPEWVKFCRQLFGGFSILLWIGAILCFLAYGIQAGTEDDPSGDNLYLGIVLAAVVIITGCFSYYQEAKSSKIMESFKNMVPQQALVIREGEKMQVNAEEVVVGDLVEIKGGDRVPADLRIISAHGCKVDNSSLTGESEPQTRSPDCTHDNPLETRNITFFSTNCVEGTARGVVVATGDRTVMGRIATLASGLEVGKTPIAIEIEHFIQLITGVAVFLGVSFFILSLILGYTWLEAVIFLIGIIVANVPEGLLATVTVCLTLTAKRMARKNCLVKNLEAVETLGSTSTICSDKTGTLTQNRMTVAHMWFDNQIHEADTTEDQSGTSFDKSSHTWVALSHIAGLCNRAVFKGGQDNIPVLKRDVAGDASESALLKCIELSSGSVKLMRERNKKVAEIPFNSTNKYQLSIHETEDPNDNRYLLVMKGAPERILDRCSTILLQGKEQPLDEEMKEAFQNAYLELGGLGERVLGFCHYYLPEEQFPKGFAFDCDDVNFTTDNLCFVGLMSMIDPPRAAVPDAVGKCRSAGIKVIMVTGDHPITAKAIAKGVGIISEGNETVEDIAARLNIPVSQVNPRDAKACVIHGTDLKDFTSEQIDEILQNHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLLFIMANIPLPLGTITILCIDLGTDMVPAISLAYEAAESDIMKRQPRNPRTDKLVNERLISMAYGQIGMIQALGGFFSYFVILAENGFLPGNLVGIRLNWDDRTVNDLEDSYGQQWTYEQRKVVEFTCHTAFFVSIVVVQWADLIICKTRRNSVFQQGMKNKILIFGLFEETALAAFLSYCPGMDVALRMYPLKPSWWFCAFPYSFLIFVYDEIRKLILRRNPGGWVEKETYY

SEQ ID NO: 208 - protein, 1029 aa, Artificial Sequence
  Synthetic Peptide
MGLWGKKGTVAPHDQSPRRRPKKGLIKKKMVKREKQKRNMEELKKEVVMDDHKLTLEELSTKYSVDLTKGHSHQRAKEILTRGGPNTVTPPPTTPEWVKFCKQLFGGFSLLLWTGAILCFVAYSIQIYFNEEPTKDNLYLSIVLSVVVIVTGCFSYYQEAKSSKIMESFKNMVPQQALVIRGGEKMQINVQEVVLGDLVEIKGGDRVPADLRLISAQGCKVDNSSLTGESEPQSRSPDFTHENPLETRNICFFSTNCVEGTARGIVIATGDSTVMGRIASLTSGLAVGQTPIAAEIEHFIHLITVVAVFLGVTFFALSLLLGYGWLEAIIFLIGIIVANVPEGLLATVTVCLTLTAKRMARKNCLVKNLEAVETLGSTSTICSDKTGTLTQNRMTVAHMWFDMTVYEADTTEEQTGKTFTKSSDTWFMLARIAGLCNRADFKANQEILPIAKRATTGDASESALLKFIEQSYSSVAEMREKNPKVAEIPFNSTNKYQMSIHLREDSSQTHVLMMKGAPERILEFCSTFLLNGQEYSMNDEMKEAFQNAYLELGGLGERVLGFCFLNLPSSFSKGFPFNTDEINFPMDNLCFVGLISMIDPPRAAVPDAVSKCRSAGIKVIMVTGDHPITAKAIAKGVGIISEGTETAEEVAARLKIPISKVDASAAKAIVVHGAELKDIQSKQLDQILQNHPEIVFARTSPQQKLIIVEGCQRLGAVVAVTGDGVNDSPALKKADIGIAMGISGSDVSKQAADMILLDDNFASIVTGVEEGRLIFDNLKKSIMYTLTSNIPEITPFLMFIILGIPLPLGTITILCIDLGTDMVPAISLAYESAESDIMKRLPRNPKTDNLVNHRLIGMAYGQIGMIQALAGFFTYFVILAENGFRPVDLLGIRLHWEDKYLNDLEDSYGQQWTYEQRKVVEFTCQTAFFVTIVVVQWADLIISKTRRNSLFQQGMRNKVLIFGILEETLLAAFLSYTPGMDVALRMYPLKITWWLCAIPYSILIFVYDEIRKLLIRQHPDGWVERETYY

SEQ ID NO: 209 - protein, 279 aa, Artificial Sequence
  Synthetic Peptide
MTKNEKKSLNQSLAEWKLFIYNPTTGEFLGRTAKSWGLILLFYLVFYGFLAALFSFTMWVMLQTLNDEVPKYRDQIPSPGLMVFPKPVTALEYTFSRSDPTSYAGYIEDLKKFLKPYTLEEQKNLTVCPDGALFEQKGPVYVACQFPISLLQACSGMNDPDFGYSQGNPCILVKMNRIIGLKPEGVPRIDCVSKNEDIPNVAVYPHNGMIDLKYFPYYGKKLHVGYLQPLVAVQVSFAPNNTGKEVTVECKIDGSANLKSQDDRDKFLGRVMFKITARA

SEQ ID NO: 210 - protein, 1258 aa, Artificial Sequence
  Synthetic Peptide
MGDMANNSVAYSGVKNSLKEANHDGDFGITLAELRALMELRSTDALRKIQESYGDVYGICTKLKTSPNEGLSGNPADLERREAVFGKNFIPPKKPKTFLQLVWEALQDVTLIILEIAAIVSLGLSFYQPPEGDNALCGEVSVGEEEGEGETGWIEGAAILLSVVCVVLVTAFNDWSKEKQFRGLQSRIEQEQKFTVIRGGQVIQIPVADITVGDIAQVKYGDLLPADGILIQGNDLKIDESSLTGESDHVKKSLDKDPLLLSGTHVMEGSGRMVVTAVGVNSQTGIIFTLLGAGGEEEEKKDEKKKEKKNKKQDGAIENRNKAKAQDGAAMEMQPLKSEEGGDGDEKDKKKANLPKKEKSVLQGKLTKLAVQIGKAGLLMSAITVIILVLYFVIDTFWVQKRPWLAECTPIYIQYFVKFFIIGVTVLVVAVPEGLPLAVTISLAYSVKKMMKDNNLVRHLDACETMGNATAICSDKTGTLTMNRMTVVQAYINEKHYKKVPEPEAIPPNILSYLVTGISVNCAYTSKILPPEKEGGLPRHVGNKTECALLGLLLDLKRDYQDVRNEIPEEALYKVYTFNSVRKSMSTVLKNSDGSYRIFSKGASEIILKKCFKILSANGEAKVFRPRDRDDIVKTVIEPMASEGLRTICLAFRDFPAGEPEPEWDNENDIVTGLTCIAVVGIEDPVRPEVPDAIKKCQRAGITVRMVTGDNINTARAIATKCGILHPGEDFLCLEGKDFNRRIRNEKGEIEQERIDKIWPKLRVLARSSPTDKHTLVKGIIDSTVSDQRQVVAVTGDGTNDGPALKKADVGFAMGIAGTDVAKEASDIILTDDNFTSIVKAVMWGRNVYDSISKFLQFQLTVNVVAVIVAFTGACITQDSPLKAVQMLWVNLIMDTLASLALATEPPTESLLLRKPYGRNKPLISRTMMKNILGHAFYQLVVVFTLLFAGEKFFDIDSGRNAPLHAPPSEHYTIVFNTFVLMQLFNEINARKIHGERNVFEGIFNNAIFCTIVLGTFVVQIIIVQFGGKPFSCSELSIEQWLWSIFLGMGTLLWGQLISTIPTSRLKFLKEAGHGTQKEEIPEEELAEDVEEIDHAERELRRGQILWFRGLNRIQTQMDVVNAFQSGSSIQGALRRQPSIASQHHDVTNISTPTHIRVVNAFRSSLYEGLEKPESRSSIHNFMTHPEFRIEDSEPHIPLIDDTDAEDDAPTKRNSSPPPSPNKNNNAVDSGIHLTIEMNKSATSSSPGSPLHSLETSL

SEQ ID NO: 211 - protein, 1272 aa, Artificial Sequence
  Synthetic Peptide
MGDMTNSDFYSKNQRNESSHGGEFGCTMEELRSLMELRGTEAVVKIKETYGDTEAICRRLKTSPVEGLPGTAPDLEKRKQIFGQNFIPPKKPKTFLQLVWEALQDVTLIILEIAAIISLGLSFYHPPGEGNEGCATAQGGAEDEGEAEAGWIEGAAILLSVICVVLVTAFNDWSKEKQFRGLQSRIEQEQKFTVVRAGQVVQIPVAEIVVGDIAQVKYGDLLPADGLFIQGNDLKIDESSLTGESDQVRKSVDKDPMLLSGTHVMEGSGRMLVTAVGVNSQTGIIFTLLGAGGEEEEKKDKKGVKKGDGLQLPAADGAAASNAADSANASLVNGKMQDGNVDASQSKAKQQDGAAAMEMQPLKSAEGGDADDRKKASMHKKEKSVLQGKLTKLAVQIGKAGLVMSAITVIILVLYFTVDTFVVNKKPWLPECTPVYVQYFVKFFIIGVTVLVVAVPEGLPLAVTISLAYSVKKMMKDNNLVRHLDACETMGNATAICSDKTGTLTTNRMTVVQAYVGDVHYKEIPDPSSINTKTMELLINAIAINSAYTTKILPPEKEGALPRQVGNKTECGLLGFVLDLKQDYEPVRSQMPEEKLYKVYTFNSVRKSMSTVIKLPDESFRMYSKGASEIVLKKCCKILNGAGEPRVFRPRDRDEMVKKVIEPMACDGLRTICVAYRDFPSSPEPDWDNENDILNELTCICVVGIEDPVRPEVPEAIRKCQRAGITVRMVTGDNINTARAIAIKCGIIHPGEDFLCLEGKEFNRRIRNEKGEIEQERIDKIWPKLRVLARSSPTDKHTLVKGIIDSTHTEQRQVVAVTGDGTNDGPALKKADVGFAMGIAGTDVAKEASDIILTDDNFSSIVKAVMWGRNVYDSISKFLQFQLTVNVVAVIVAFTGACITQDSPLKAVQMLWVNLIMDTFASLALATEPPTETLLLRKPYGRNKPLISRTMMKNILGHAVYQLALIFTLLFVGEKMFQIDSGRNAPLHSPPSEHYTIIFNTFVMMQLFNEINARKIHGERNVFDGIFRNPIFCTIVLGTFAIQIVIVQFGGKPFSCSPLQLDQWMWCIFIGLGELVWGQVIATIPTSRLKFLKEAGRLTQKEEIPEEELNEDVEEIDHAERELRRGQILWFRGLNRIQTQIEVVNTFKSGASFQGALRRQSSVTSQSQDIRVVKAFRSSLYEGLEKPESRTSIHNFMAHPEFRIEDSQPHIPLIDDTDLEEDAALKQNSSPPSSLNKNNSAIDSGINLTTDTSKSATSSSPGSPIHSLETSL

SEQ ID NO: 212 - protein, 874 aa, Artificial Sequence
  Synthetic Peptide
MGDMANSSIEFHPKPQQQRDVPQAGGFGCTLAELRTLMELRGAEALQKIEEAYGDVSGLCRRLKTSPTEGLADNTNDLEKRRQIYGQNFIPPKQPKTFLQLVWEALQDVTLIILEVAAIVSLGLSFYAPPGEESEACGNVSGGAEDEGEAEAGWIEGAAILLSVICVVLVTAFNDWSKEKQFRGLQSRIEQEQKFTVIRNGQLLQVPVAALVVGDIAQVKYGDLLPADGVLIQANDLKIDESSLTGESDHVRKSADKDPMLLSGTHVMEGSGRMVVTAVGVNSQTGIIFTLLGAGGEEEEKKDKKGKQQDGAMESSQTKAKKQDGAVAMEMQPLKSAEGGEMEEREKKKANAPKKEKSVLQGKLTKLAVQIGKAGLVMSAITVIILVLYFVIETFVVEGRTWLAECTPVYVQYFVKFFIIGVTVLVVAVPEGLPLAVTISLAYSVKKMMKDNNLVRHLDACETMGNATAICSDKTGTLTTNRMTVVQSYLGDTHYKEIPAPSALTPKILDLLVHAISINSAYTTKILPPEKEGALPRQVGNKTECALLGFVLDLKRDFQPVREQIPEDKLYKVYTFNSVRKSMSTVIRMPDGGFRLFSKGASEILLKKCTNILNSNGELRGFRPRDRDDMVRKIIEPMACDGLRTICIAYRDFSAGQEPDWDNENEVVGDLTCIAVVGIEDPVRPEVPEAIRKCQRAGITVRMVTGDNINTARAIAAKCGIIQPGEDFLCLEGKEFNRRIRNEKGEIEQERLDKVWPKLRVLARSSPTDKHTLVKGIIDSTTGEQRQVVAVTGDGTNDGPALKKADVGFAMGIAGTDVAKEASDIILTDDNFTSIVKAVMWGRNVYDSISKFLQFQLTVNVVAVIVAFTGACIT

SEQ ID NO: 213 - protein, 28 aa, Artificial Sequence
  Synthetic Peptide
MQRVNMIMAESPGLITICLLGYLLSAEC

SEQ ID NO: 214 - protein, 18 aa, Artificial Sequence
  Synthetic Peptide
TVFLDHENANKILNRPKR

SEQ ID NO: 215 - protein, 415 aa, Artificial Sequence
  Synthetic Peptide
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 216 - protein, 415 aa, Artificial Sequence
  Synthetic Peptide
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLT